(19) United States
(12) Patent Application Publication
(10) Pub. No.: US 2023/0123589 A1
(43) Pub. Date: Apr. 20, 2023

(54) MOLECULAR MOBILITY ENHANCER OR MOLECULAR DRYING ENHANCER

(71) Applicant: TEKDRY INTERNATIONAL, INC., Lafayette, CO (US)

(72) Inventors: Daniel STOREY, Longmont, CO (US); Christina K. THOMAS, Iron River, WI (US); Adam R. COOKSON, Broomfield, CO (US)

(21) Appl. No.: 17/794,726

(22) PCT Filed: Jan. 28, 2021
(86) PCT No.: PCT/US2021/015440

§ 371 Date: Jul. 22, 2022

Related U.S. Application Data
(60) Provisional application No. 62/966,742, filed on Jan. 28, 2020.
(60) Provisional application No. 62/966,799, filed on Jan. 28, 2020.
(60) Provisional application No. 62/966,839, filed on Jan. 28, 2020.

(51) Int. Cl. A61L 2/20; A01N 25/02; A01N 59/00; A01P 1/00; A01N 25/18; F26B 5/04; F26B 3/20

(52) U.S. Cl. CPC A61L 2/208; A01N 25/02; A01N 59/00; A01P 1/00; A01N 25/18; F26B 5/04; F26B 3/20; A61L 2202/11; A61L 2202/122; A61L 2202/24

(57) ABSTRACT

Method and apparatus for generating, providing diffusion limited transport and evacuating gaseous phase material in a sub-atmospheric environment employing a molecular mobility enhancer (MME) component under reduced pressure to rapidly vaporize a liquid and/or solid, to transport generated vapor including a transport moiety into a diffusion processing zone and through diffusion restricted objects and to effectively evacuate non-consumed vapor from apparatus. Compositions, including solids, for delivery of MME-transport moiety vapor in a sub-atmospheric environment. Including those with improved storage and stability. Additionally, methods and apparatus for drying surfaces of articles in a sub-atmospheric environment. Compositions for generating a vapor in a sub-atmospheric environment for use in drying such articles. Methods and compositions herein useful for cleaning, sanitizing and sterilizing substrates, implements and devices including medical and electronic devices.

### CROSS-REFERNCE TO RELATE APPLICATIONS

This application claims the benefit of U.S. provisional applications 62/966,742, filed Jan. 28, 2020, 62/966,799, filed Jan. 28, 2020; and 62/966,839, filed Jan. 28, 2020, each of which is incorporated by reference herein in its entirety.

### BACKGROUND

The invention provides a method and apparatus for generating, providing diffusion limited transport and evacuating gaseous phase material in a sub-atmospheric environment. The method uses a molecular mobility enhancer (MME) component under reduced pressure to rapidly vaporize a liquid and/or solid, to transport generated vapor into a diffusion processing zone and through diffusion restricted objects and to effectively evacuate non-consumed vapor from apparatus. Also provided are molecular mobility enhancer-transport moiety (MME-transport moiety) compositions for delivery of MME-transport moiety vapor in a sub-atmospheric environment. MME compositions, particularly solid MME compositions, are provided. MME compositions include a carrier, and optionally a surfactant as well as any number of other optional additives, such as, but not limited to, a binder, solubilizer, bufferent, thickener and/or a barrier material. The carrier and various additives can be in different combinations. The MME and MME-transport moiety compositions of the present invention can also be used for improved storage and stability of the MME and MME-transport moiety.

In embodiments, an article, such as a substrate(s), device(s) and/or instrument(s) is in fluid connection with the vapor generated in the diffusion system under reduced pressure. In an embodiment, contact of the generated vapor with the article results in treatment of the article. In embodiments, treatment includes, cleaning, sanitizing, sterilization and/or coating.

The invention provides materials (e.g., compositions), an apparatus and process for providing enhanced vapor flow through articles, particularly those containing diffusion restricted areas, such as medical devices (e.g. lumens, etc.) and electronic components (e.g. 3D protoboards, etc.) and to methods for expediting the vaporization of and evacuation of gaseous materials in a sub-atmospheric environment.

In another aspect, the invention provides materials, methods and apparatus for drying surfaces of articles in a sub-atmospheric environment. More specifically the invention relates to compositions for generating a vapor in a sub-stmospheric environment for use in drying such articles. The compositions of the invention comprise a molecular drying enhancer (MDE). MDEs are used in combination with a sub-atmospheric environment to generate MDE vapor(s) to dry the surfaces of articles, including substrates, devices (including medical devices and electronic devices), implements and related equipment, among others, using said vapor. The use of MDE vapor to dry surfaces enhances drying time and effectiveness.

With resepct to this aspect, the invention also relates to compositions containing molecular drying enhancers (MDEs). In embodiments, the MDEs are liquids at normal temperature and pressure (NPT, 20° C. and 1 atm (760 torr). In embodiments, the MDEs have vapor pressure of greater of equal to 10 torr and more preferably greater or equal to 100 torr or more at operating temperatures. In embodiments, operating temperatures range from ambient room temperature to 150° C. More specifically, operating temperatures range from ambient troom temperature to 100° C. More specifically, operating temperatures range from ambient troom temperature to 60° C. More specifically, operating temperatures range from ambient troom temperature to 50° C. More specifically, operating temperatures range from ambient room temperature to 40° C. In particular, the present disclosure provides volatile compositions of MDEs that outgass under reduced pressure to provide a method for effective and rapid drying of a variety of different articles in a vacuum system.

Expeditiously vaporizing materials, such as liquids, in vacuum in the presence of articles such as medical devices and/or electronic components that have strict temperature and process (e.g. time, concentration, etc.) requirements has always been a challenge. A variety of methods are reported to accelerate the formation of gaseous material from liquid in vacuum, such as raising temperature to increase vapor pressure, decreasing concentration of the liquid being evaporated and/or increasing heat flow to the liquid. To the potential detriment of the articles being treated, one or more treatment variables (temperature, concentration, etc.) must be adversely adjusted to achieve rapid vaporization. Numerous device and component materials are not compatible with high temperatures and, as such, can lead to failure of or shorten the useful lifetime of the device, material and/or electronics. A number of vacuum processes involve vaporization and transport of a liquid component. Vapor hydrogen peroxide sterilization/sanitization, for example, requires a threshold vapor sterilant concentration to effectively sterilize the surface of substrates. By lowering the concentration of the sterilant liquid, one effectively lowers the concentration of the sterilant vapor effectively rendering the sterilization/sanitization process ineffective. This can lead to contamination and possible patient infection and/or death.

Development of materials, apparatus and methods for generating, transporting, particularly through diffusion restricted spaces, and evacuating gaseous material in a sub-atmospheric environment are reported for a number of different applications. However, processes for successfully generating, transporting and evacuating gaseous material under vacuum and within one system are nonexistent. Attempts to improve vapor flow through tubes, items with narrow openings and/or 3D objects have been directed towards complicated processes and machinery as well as potential material incompatibility. In order to improve transport of hydrogen peroxide vapor, for example, through lumens, efforts have been reported to force hydrogen peroxide liquid into the ends of a lumen by a method of condensation followed by re-vaporizing the liquid to generate and transport vapor through to interstices of the lumen. Though this laborious method may help hydrogen peroxide vapor move through the outer edges of the tube, this method fails to provide a successful and reproducible method by which gaseous material can reach the entire inner length of the object. Failure of sterilant vapor to reach all surfaces of a substrate or device to be sterilized can result in failed sterilization. Such method provides no calculated way by which the condensed vapor can be delivered to the edges of the lumen. As such, the volume and location of the liquid as it condenses into or onto the lumen is random and not reproducible. Equally detrimental, exposure of liquid chemical(s), such as hydrogen peroxide, to the surface of a lumen may be detrimental to the integrity of the device. Products treated by such method can have numerous device failures.

Improving evacuation of surface adsorbed materials on vapor sterilized articles and in the fabrication of micro electro-mechanical systems (MEMS) and other electronic components, for example, under vacuum, have been reported. Efforts to improve desorption of gases/vapors from surfaces of materials in vacuum is necessary to maintain cleanliness and material integrity. Removal of unwanted volatile components and/or water from a processing chamber is vital for reducing surface adsorption. Attempts at increasing removal of residual gas molecules as well as increasing desorption rate have been reported by increasing temperature and/or involving extended high vacuum level times. Though these methods may be capable of assisting desorption and evacuation, material restrictions limit their full working capacity. A large number of materials are not compatible with very high temperatures and long vacuum exposure times are not suitable for high-throughput manufacturing processes. Adverse exposure to high temperatures and residual surface chemicals can also cause device failure, reduce their lifetime and/or inadvertent user injury.

With respect to the use of MDE in methods herein, there are several steps in the preparation of object(s), instrument(s), device(s) in industries such as the semiconductor and medical device industry that require drying of rinsing fluids. These liquids must be completely removed to reduce contamination of the device. Vacuum drying has been used as a technique for remove undesired fluid, such as water or aqueous solutions, from surfaces following a rinsing step. Traditionally, evaporation of water under vacuum poses difficulties such as freezing which leads to long drying times and incomplete drying processes. Incomplete drying processes also may generate undesirable spotting or streaking on device surfaces such as semiconductors or bacterial or viral contamination of the inside of endoscopes, for example.

MME and MME-transport moieties are provided in liquid and solid form. Solid carriers for MME-transport moiety compositions offer potential advantages over liquid formulations. MME-transport moiety components, such as hydrogen peroxide (H₂O₂)-isopropanol (IPA), present several challenges due to their processing, chemical reactivity and stability and/or storage limitations. Liquid MME-transport moiety compositions can present handing and packaging challenges due to chemical incompatibilities and component evaporation. Moreover, liquid form MME-transport moiety compositions may be prone to chemical instability.

MME and MME-transport moiety compositions that avoid bulk packaging, such as plastic bottles, envelopes, etc., and address issues of chemical reactivity and instability are particularly desirable. It is also desirable that the MME-transport moiety composition be contained in an easy to handle form that is designed to emit an MME-transport moiety vapor on demand in a sub-atmospheric environment. This requires, therefore, that the MME-transport moiety be contained within any facile, low-bulk solid vessel that 1) acts to irreversibly trap the MME-transport moiety molecules in a solid matrix until activated and released under reduced pressure; and 2) preferably acts to stabilize the chemical components of the MME-transport moiety composition to prevent problems that can occur as a result of chemical degradation and/or incompatibility, such as unexpected low dosing, package leaking and/or potential hazardous exposure.

In embodiments, the methods described herein employing MME-transport compositions for cleaning, sterilizing and sanitizing can be combined with methods describedherein employing MDE for drying.

Thus, there remains a need for straightforward and effective methods for affecting gaseous material by accelerating vaporization, improving transport in diffusion restricted areas and enhancing evacuation from sub-atmospheric apparatus under low temperature conditions. There is also a need in the art for compositions to effectively facilitate such methods. There is a particular need for a solid form of the MME or MME-transport moiety to provide enhanced efficacy and ease of handling.

The aspect of the invention related to the use of MDEs, addresses the shortcomings of current sub-atmospheric drying processes by finding a solution to problems that result from conventional drying methods under vacuum. The invention provides a drying agent composition whose vapor, under reduced pressure, provides a new drying method.

### SUMMARY

The invention provides materials, methods and apparatus for generating and transporting gaseous material in a reduced pressure environment. Enhanced transport can be achieved by using molecular mobility enhancer (MME) chemistry. An MME component can be blended with a transport moiety to be vaporized. In embodiments, the MME can be a liquid at NPT (normal temperature and pressure, 20° C. and 1 atm (760 torr)). In embodiments, the transport moiety can be a liquid at NPT (normal temperature and pressure, 20° C. and 1 atm (760 torr)). An MME-transport moiety blend (mixture) can be used in a vacuum system to carry out the method of this invention. There are a variety of apparatus and/or method permutations in which the method of this invention can be carried out. In general, the MME-transport moiety blend is inserted into the diffusion system, by way of the process chamber and/or chamber extension. In embodiments, substrate(s), device(s) and/or instrument(s) to be treated are placed into the process chamber of the diffusion system. Thereafter, the pressure of the diffusion system is reduced below atmosphere and MME-transport moiety vapor is generated. Even though heat may be used it is not required. The transport moiety vapor is transported through the diffusion system and into diffusion restricted spaces, such as lumens or 3D objects, by way of MME vapor. Even though a non-reactive carrier gas may be used it is not required. After sufficient processing time has allowed the desired vapors to come into contact with the substrate(s), device(s) and/or instrument(s) the gaseous material remaining in the diffusion system is evacuated. Any part of this process may be repeated or alternated, etc. until processing is achieved. In embodiments, the vapor generated is useful in cleaning, sanitizing, sterilizing and coating applications, among others.

The apparatus is found to be well-suited for executing the method of the invention. The apparatus is a diffusion system that includes a processing chamber that may be fluidly connected to a chamber extension where substrate(s), device(s) and/or instrument(s) are processed and/or where MME-transport moiety vapor is generated. The diffusion system is configured with multiple ports. These ports may be connected to a pump for reducing pressure or for exhausting evacuated gaseous material of the diffusion system. Additional ports of the diffusion chamber may include those that connect the processing chamber with the chamber extension and/or other items such as carrier gas couplers, sensors, gauges, etc. The apparatus or diffusion system may also be outfitted with heaters that may be controlled by region. Substrate holders and manifolds may be configured as part of the diffusion system if required. The apparatus may be manually or automatically controlled.

In embodiments, the invention provides a method for providing a transport moiety to an enclosure, the method comprising exposing a mixture comprising a molecular mobility enhancer (MME) and the transport moiety to the enclosure at a sub-atmospheric pressure condition, thereby providing the transport moiety as a vapor in the enclosure. In an embodiment, the exposing step provides the transport moiety and the MME as the vapor in the enclosure. In an embodiment, the exposing step results in transport of the transport moiety within the enclosure. In an embodiment, the exposing step results in contact of the transport moiety with surfaces of the enclosure and/or with surfaces of an article within the enclosure.

In an embodiment, the method for providing a transport moiety to an enclosure comprises treating the enclosure and/or an article provided within the enclosure. In an embodiment, the treatment comprises sterilizing the enclosure and/or an article provided within the enclosure. In an embodiment, the treatment comprises sanitizing the enclosure and/or an article provided within the enclosure.

In embodiments, the molecular mobility enhancer is one or more of an alcohol, alkane, carboxylic acid, ester, ether, ketone and any combination thereof. In embodiments, the molecular mobility enhancer is one or more of: a C1-C20 alcohol, C5-C20 alkane, C1-C20 carboxylic acid, C3-C20 ester, C4-C20 ether, C3-C20 ketone and any combination thereof. In embodiments, the molecular mobility enhancer is a C1-C3 alcohol or a combination thereof. In embodiment, the molecular mobility enhancer is methanol.

In embodiments, the transport moiety is one or more of a peroxide, peroxyacid, alcohol, chlorine-containing compound, a phenolic compound and any combination thereof. In embodiments, the transport moiety is one or more of hydrogen peroxide, ethanol, isopropanol, hypochlorite, hypochlorous acid, chloride dioxide, ethylene oxide, propylene oxide, formaldehyde, glutaraldehyde, iodophor, ortho-phthaladehyde, ozone, peracetic acid, performic acid, phenol/phenate, beta-propiolactone and any combination of these. In embodiments, the transport moiety is one or more sanitizer and/or sterilizer.

The molecular mobility enhance is a compound that is different from the transport moiety.

In a specific embodiment, the MME is methanol and the transport moiety of the mixture is hydrogen peroxide.

In embodiments, the weight ratio of MME to transport moiety in compositions herein ranges from 0.01 to 1. In more specific embodiments, the weight ratio of MME to transport moiety in compositions herein ranges from 0.01 to 0.8, or 0.01 to 0.5 or 0.01 to 0.25, or 0.01 to 0.2 or 0.01 to 0.1, or 0.01 to 0.05, or 0.05 to 1 or 0.05 to 0.8 or 0.05 to 0.5 or 0.05 to 0.25 or 0.05 to 0.2 or 0.05 to 0.1, or 0.1 to 1, or 0.1 to 0.8 or 0.1 to 0.5, or 0.1 to 0.25 or 0.1 to 0.2.

In embodiments, the method further comprises heating the mixture during the exposing step. In embodiment, the method further comprised providing a carrier gas flow in fluid communication with the mixture and flowing the carrier gas flow in fluid communication with the mixture into the enclosure. In embodiments, the method further comprises maintaining the enclosure at a pressure selected over the range of 0.1 torr to 200 torr for a time period selected from the range of 1 minute to 24 hours. In an embodiment, the exposing step comprises providing the mixture in fluid communication with the sub-atmospheric enclosure, thereby providing for transport of the transport moiety into the enclosure. In an embodiment, the exposing step comprises providing the mixture in the enclosure followed by decreasing the pressure of the enclosure to below 760 torr. In an embodiment, the pressure of the enclosure is decreased to a pressure selected over the range of 0.1 torr to 200 torr.

In embodiments, the enclosure is a vacuum chamber or a processing chamber. In embodiments, the enclosure is for disinfecting, sanitizing or sterilizing an article provided within the enclosure. In an embodiment, the article provided within the enclosure for disinfecting, sanitizing or sterilizing is a medical device or component thereof. In embodiments, the medical device or component thereof comprises one or more lumens. In embodiments, the medical device is an endoscope or component thereof.

The invention provides liquid and solid compositions useful in carrying out the invention containing one or more MME and or one or more transport moieties. MME compositions include one or more MME and/or transport moieties in combination with a liquid or solid carrier. Compositions optionally comprise one or more surfactant, binder, solubilizer, bufferent, thickener and/or barrier material. The carrier and additives can be in different combinations.

In embodiments, the invention provide solid MME and MME-transport moiety compositions as well as methods of making and using such compositions. The invention further provides solid MME-transport moiety compositions that allow rapid outgassing of the MME-transport moiety from the solid under sub-atmospheric conditions. Outgassing provides a vapor comprising the transport moiety. In embodiments, outgassing provides a vapor comprising the transport moiety and the MME. The invention also provides a solid MME-transport moiety composition having increased MME-transport moiety chemical stability. More specifically, the invention provides solid MME-transport moiety compositions for rapid outgassing of a variety of MME-transport moieties from a wide variety of solid components, contained therein, in a sub-atmospheric environment. The invention also provides compositions for effectively processing and storing a chemically stability MME-transport moiety composition in solid form.

Solid MME-transport moiety compositions of the invention can be employed to generate vapors comprising the transport moiety or vapors comprising the transport moiety and the MME which are useful in various treatment applications. Treatment applications relate, among others, to applications in which one or more articles are to be treated with the transport moiety. In specific embodiments, treatment of articles is achieved by contacting the one or more articles with a vapor generated from the solid MME-transport moiety. In an embodiment, a vapor comprising the transport moiety and optionally the MME is generated in a chamber to which reduced pressure (sub-atmospheric) is applied.

In embodiments, solid MME-transport moiety compositions described in the present invention comprise one or more transport moiety, one or more MME and a carrier, and optionally comprise a surfactant and/or any number of other additives, such as a binder, solubilizer, bufferent, thickener and/or a barrier material. In an embodiment, the carrier is a solid which can include a gel. The carrier may include any one or combination of any synthetic, semi-synthetic, and/or natural polymer, ceramic, glass, metallic, hybrid and/or composite material. The composition may include an ionic, non-ionic, hydrophilic or lipophilic surfactant.

In embodiments, the invention provides a solid composition comprising a mixture of a MME and a transport and a carrier. In embodiments, the carrier is a solid including a gel. In embodiments, the carrier is a polymer, ceramic, glass, metallic, hybrid, or composite material or a mixture of such materials. In embodiments, the carrier is a polymer or mixture of polymers. In embodiment, the polymer is a homopolymer, copolymer, cross-linked polymer or a combination thereof. In embodiments, the carrier is a polyamide, polysiloxane or a polyamide/polysiloxane blend. In embodiments, the carrier is a polyacrylamide gel, polyacrylic acid gel, or a poly(acrylamide-co-methacrylic acid) gel. In embodiments, the carrier is polyethylene glycol.

In embodiments, the carrier is a poly(DL-lactide-co-glycolide), or a poly(DL-lactide-co-glycolide)-co-polyethylene glycol. In embodiments, the carrier is maltodextrin sorbitol or a mixture thereof.

In embodiments, the composition is in the form of a powder, a wax, a gel, or a particle or a plurality of particles

In embodiments, the transport moiety is a cleaning agent, a sanitizing agent, a sterilizing agent or a coating agent. In specific embodiments, the transport moiety is a sterilizing agent. In specific embodiments, the transport moiety is hydrogen peroxide, a peracid or a mixture thereof. In specific embodiments, the peracid is performic acid or peracetic acid.

In specific embodiments, the MME is an ether or an alcohol. In more specific embodiments, the ether is an ether having 2-12 carbon atoms or 2-8 carbon atoms. In specific embodiments, the ether is of formula R—O—R′ where R and R′ are the same or different and are selected from linear or branched alkyl, alkenyl, or alkynyl groups, cycloalkyl, or cycloalkenyl groups, aryl, alkaryl or arylalkyl groups. In specific embodiments, the MME is diethyl ether. In more specific embodiments, the MME is an alcohol of formula R″OH having 1-12 carbon atoms or 1-8 carbon atoms, including all isomeric forms thereof, where R″ is selected from linear or branched alkyl, alkenyl, or alkynyl groups, cycloalkyl, or cycloalkenyl groups, aryl, alkaryl or arylalkyl groups. In specific embodiments, the MME is methanol, ethanol, n-propanol, iso-propanol, n-butanol, iso-butanol, or sec-butanol. More specifically, the MME is iso-propanol.

In embodiments, the invention provides a method for delivery of a MME-transport moiety blend into an apparatus capable of achieving reduced pressure. In embodiments, the MME-transport moiety blend is delivered in the form of a vapor comprising the transport moiety or a mixture of the MME and the transport moiety. The vapor generated optionally comprises water. In embodiments, the method comprises introducing the MME-transport moiety composition as described herein into the apparatus and reducing the pressure in at least a portion of the apparatus to generate the vapor comprising transport moiety in at least a portion of the apparatus. In embodiments, a selected pressure of the generated vapor is maintained in at least a portion of the apparatus for a selected time. The pressure of the vapor maintained and the time the vapor is maintained are adapted to a given application of the vapor. In embodiments, the selected pressure maintained in the apparatus ranges from 0.1 torr to 200 torr. More specifically, the selected pressure maintained in the apparatus ranges from 0.5 torr to 10 torr. More specifically, the selected pressure maintained in the apparatus ranges from 0.5 torr to 3 torr. More specifically, the selected pressure maintained in the apparatus in about 1 torr+/−10%. Generally, the pressure is maintained at +/−10% of the selected pressure. In embodiments, the selected pressure is maintained for 1 minute to 24 hours. In embodiments, the selected pressure is maintained for 5 minutes to 1 hour. In embodiments, the selected time is 5 minutes to 30 minutes. In a specific embodiment, the selected time is 15 minutes+/−10%.

In embodiments, an article to be treated is present in the apparatus and the vapor generated is in contact with the article to achieve desired treatment. In embodiments, the article to be treated is to be sterilized. In an embodiment, the article is to be coated. In an embodiment, the article to be treated comprises diffusion resistant surfaces where it is intended that the transport moiety contact all surfaces of the article including any such diffusion resistant surfaces.

Other ingredients of the composition, in addition to form and processing the described compositions will become more fully apparent from the following description and claims or may be learned by using the materials as described herein and/or practicing the methods as disclosed herein.

The solid MME-transport moieties disclosed herein are of particular use in sanitizing and sterilization methods that employ sub-atmospheric pressure. Exemplary methods and apparatus in which the compositions of the invention can be employed include those described in published PCT application WO2018/175455, published Sep. 27, 2018, U.S. published application US2018/0289846, each of which is incorporated by reference herein in its entirety. U.S. provisional application 62/797,789, filed Jan. 28, 2019 and corresponding PCT application Attorney Docket No: 338240: 161-19 WO filed Jan. 28, 2020 as well as U.S. provisional application Attorney Docket No. 338255: 3-20P US provide examples of MME compositions and MME-transport moiety compositions, particularly for use in sanitation and sterilization application. Each of these applications is also incorporated by reference herein in its entirety.

Exceptional drying of a wide range of object(s), instrument(s) and/or device(s) can be achieved using an MDE vapor in a vacuum based drying system. The present disclosure provides MDE composition that can be used to generate vapors useful for drying objects, such as those with diffusion restricted internal channels (e.g. endoscopes) and/or 3D structures (e.g. electronic components) in a sub-atmospheric drying system. The disclosure provides MDE compositions for rapid outgassing of a variety of MDE moieties from a wide variety of liquid components, contained herein, in a sub-atmospheric drying system. The present disclosure also provides compositions for effectively processing the MDE composition in liquid form.

The vacuum-based drying system converts an MDE solution to a vapor using an outgassing process that deliberately and in a controlled manner facilitates the removal of unwanted liquid, particularly water, from the surface and/or inter areas of object(s), instrument(s) and/or device(s). An MDE vapor is generated in the the drying system under reduced pressure by an outgassing process that may or may not include heat. MDE vapor flow is directed through the vacuum process chamber under reduced pressure, with or without the use of heat, where it acts to provide a method of drying.

Among other things, this disclosure addresses the shortcomings of conventional vacuum drying techniques, such as incomplete and laborious drying times. It is known that the process of displacing residual moisture is difficult to accomplish under under reduced pressure due to complicated object, instrument and/or device design and the tendency of liquids (e.g. water) to freeze under reduced pressure. Methods for overcoming these limitations are described in the present disclosure.

Compositions for generating MDE vapors used for carrying out a drying process in a vacuum process chamber comprise one or more volatile hydrocarbon, alcohol, ketone, nitrile, carboxylic acid, ester, ether, glycol ether, polysiloxane and/or aldehyde. The above list of MDE groups is a subset, not an exhaustive list, of groups of volatile compounds that can be used in the present disclosure. The above-mentioned moieties exhibit exceptional volatilization and penetrating capability, which is desirable for drying object(s) described herein. Additionally, they are well suited for altering the freezing point and vapor pressure of the liquid to be dried or removed from the the object(s) as described herein.

In embodiments, the disclsoure provides a method for removing undesired liquid from an article which comprises contacting the article with a vapor comprising a molecular drying enhancer (MDE). In an embodiment, the vapor is generated in a sub-atmospheric environment. In an embodiment, the vapor is generated in a processing chamber capable of achieving a reduced pressure environment. In an embodiment, the article from which liquid is to be removed is positioned in the process chamber. In an embodiment, the vapor comprising the MDE is generated by reducing the pressure in at least a portion of the process chamber to a level that is sub-atmosheric. In embodiments, the pressure is reduced to 200 torr or less or 10 torr or less or 1 torr or less or 0.1 torr or less.

In embodiments, the MDE is selected from the group consisting of one or more optionally substituted hydrocarbons, ketones, nitriles, esters, ethers, glycolethers, aldehydes, carboxylic acids or siloxanes or mixtures thereof. In embodiments, the MDE is selected from the group consisting of one or more optionally substituted hydrocarbons, ketones, nitriles, esters, ethers, glycolethers, aldehydes, carboxylic acids or siloxanes or miscible mixtures thereof. In an embodiment, the MDE is a compound that has 1-10 carbon atoms. In an embodiment, the MDE is a compound that has a vapor pressure of 10 torr or more at operating temperatures of the method. In embodiments, operating tempertures of the method range from ambient room temperature to 150° C. More preferably operating temperature of the method range from ambient room temperature to 100° C. Preferable operating temperature of the method range from ambient room temperature to 40° C., or 50° C. or 60° C., or 70° C. or 80° C.

In embodiments, the liquid to be removed in water or an aqueous solution. In embodiments, the liquid to be removed is a miscible mixture of watrer and an organic solvent.

In additional embodiments, the MDE is selected from the group consisting of optionally substituted alkanes, alkenes, alkynes, ketones, nitriles, esters, ethers, glycoethers, aldehydes, or carboxylic acids or mixtures thereof. In embodiments, MDE mixtures are miscible mixtures at opeating temperatures of the method. In embodiments, each MDE is a liquid at NPT.

In additional embodiments, the MDE is selected from a C1-C3 alcohol, a C3-05 ketone, a C4-C6 ester, a C2-C6 nitrile, a C2-C6 carboxylic acid, an ester of ethylene glycol, or mixtures thereof, wherein the ester of ethylene glycol is preferably a C1-C3 alkyl ester. In embodiments, the MDE is selected from methanol, isopropanol, acetone, ethylacetate, acetonitrile, acetic acid or ethylene glycol monomethyl ester.

In embodiments, the time to achieve drying ranges from 1 minute to 24 hours. In embodiments, the time to achieve drying ranges from 10 minutes to 24 hours. In embodiments, the time to achieve drying ranges from 1 minutes to 10 hours. In embodiments, the time to achieve drying ranges from 10 minutes to 10 hours. In embodiments, the time to achieve drying ranges from 10 minutes to 1 hour. In embodiments, the time to achieve drying ranges from 10 minutes to 2 hours. In embodiments, the time to achieve drying ranges from 30 minutes to 2 hours.

In embodiments, the method for drying is conducted at a temperature above ambient temperature to 100° C. In embodiments, the method is conducted at a temperature above ambient temperature to 50° C.

In embodiments, the methods for sanitization and sterilization employing MME can be combined with methods of drying employing MDE. For example, a selected item or device can be washed conventionally using an appropriate cleaning agent after which the item or device can be rinsed with a selected solvent, such as water. The item or device can then be dried employing the methods herein employing MDE. Thereafter the dried item or device can be further sanitized or sterilized employing the methods herein employing MME.

Other embodiments and implementation of the compositions, methods, devices and applications of this invention will be apparent to one of ordinary skill in the art on review of the description and examples herein. The examples provided are not intended to be limiting.

### BRIEF DESCRIPTION OF THE DRAWINGS

FIG. 1 shows a block diagram of the sterilization/sanitization process 100 in the Sterilizing System 105. This system includes a number of subsystems that create the entire system. It is understood this is a representation of one embodiment and it is not necessary to have all the subsystems in place for the entire system to function in other methods. A Heating Subsystem 140, Monitoring Subsystem 160, Pressurizing Subsystem 130, User Interaction Subsystem 150, Sterilizing Subassembly 170 and an MME Subassembly 180 are all connected physically and/or electronically to the Sterilizing Chamber 110 through normal means. The Device 120 is placed into the Sterilizing Chamber by the User 103 and the sterilizing/sanitizing process is started by the User 103 through the Communication Subsystem 190. The process is controlled through a set of pre-programmed routines via the Controller 180 which is typically a programmable logic controller (PLC) that can be purchased off the shelf.

FIG. 2 shows a diagram of a typical, but not limiting, sterilizer and how the subsystems connect to the process chamber 110. In this diagram, the part to be sterilized 120 is placed in the vacuum Sterilization Chamber 110 on the radiative/conductive heater 115. The lid 315 to the Sterilization Chamber 110 is closed and sanitization or sterilization routine is chosen by choosing the appropriate cycle on the touch screen 330. The chosen routine, once chosen, is then implemented automatically through the User Interaction Subsystem 150 to the Communications Subsystem 190 and then to the controller 180, which is typically a PLC. Once the chosen cycle is started, the Pressurizing Subsystem 130 in engaged and the pressure and humidity in the system is monitored through the Monitoring Subsystem 130 which is connected to a Vacuum Sensor 365 and a Humidity Sensor 363. Additionally, the Heating Subsystem 140 is a closed loop controlled through a proportional-integral derivative controller (PID) that will set the temperature at the appropriate set point as dictated by the Controller 180 through the heating block 115. Once the pressure and humidity is at the appropriate levels as dictated by the Controller 180, the Sterilizing Subassembly and MME Subassembly 190 are engaged per the cycle parameters, with the vaporized sterilant entering the Process Chamber 110 through the sterilant injector head 375.

FIG. 3 shows a block diagram flow chart 300 of the generic process for sterilization of a device using the MME. Note: this is the same generic process for sanitization, with an adjustment of the specific process parameters for the sanitization versus sterilization.

### DETAILED DESCRIPTION

It is to be understood that the apparatus and methods of the invention may be applicable to a number of different vapor transport processes. For the purpose of illustration and not to be limiting, a vapor sterilization/sanitization process will be referred to specifically. It is also to be understood that, while in practice there is a difference between sanitization and sterilization, for purposes of this application the two terms may be used interchangeably for purposes of simplicity. For example, the Sterilizing System 105 referenced in the drawings can be understood to also be a sanitization system. Materials and methods herein can be used to generate a vapor of transport moiety in a chamber under reduced pressure. Such a vapor is useful for a variety of applications. In embodiments, such a vapor is useful for cleaning, sanitizing, sterilization and coating applications. In embodiments, the vapor is used to treat an article. In an embodiment, the vapor is used to treat an article placed within the chamber.

Transporting vapors into or through a sub-atmospheric pressure environment and/or diffusion restricted space(s) pose a challenge in various types of apparatus that take advantage of the movement of gaseous phase material.

The phrase “diffusion restricted”, in reference to the apparatus and method described in the invention, is understood to mean, but is not limited to, an object or area on or within an object that contains a material and/or configuration such that the physical and/or chemical properties of the material and/or configuration retards or slows the rate at which the movement of anything (e.g. vapor molecules, etc.) can move through the material and/or configuration of the object and/or area on or within the object.

Movement of vapor sterilant into diffusion restricted spaces, such as lumens, or into 3D objects, such as complicated electronics, in a reduced pressure and low temperature environment provides difficulties due to poor transport of the sterilant vapor throughout the apparatus as well as into the inner areas of the objects to be sterilized. The invention addresses the shortcomings of prior art techniques by utilizing an MME in combination with a transport moiety (e.g. sterilant) to not only transport gaseous material into and through diffusion restricted spaces but also to rapidly generate and/or evacuate unused transport moiety vapor in a sub-atmospheric pressure environment. The MME may comprise a liquid or solid, such as an alcohol or an ether. The transport moiety may comprise a liquid or solid agent, such as a peroxide or a peroxy acid.

As indicated above, the method and apparatus of the invention makes use of MMEs to overcome the challenges of prior vapor transport techniques by facilitating the vaporization of molecules from liquids, improving the mobility and transport of the gaseous phase materials in a sub-atmospheric pressure environment and facilitating the evacuation of unused or unwanted vapor molecules from the sub-atmospheric pressure system.

The term “molecular mobility enhancer (MME)” refers to a chemical component of a mixture whose volatilization and mechanism of transport properties are sufficient to expedite the release of molecules, particularly transport moieties as described herein, from solids and to improve the movement of vapor molecules in a sub-atmospheric pressure environment by preventing molecular aggregation and/or stagnation. Some non-limiting examples of MMEs include any volatile alcohol (e.g. methanol, ethanol, isopropanol, etc.), alkane (e.g. pentane, hexane, heptane, etc.), carboxylic acid (e.g. formic acid, acetic acid, propionic acid, etc.), ester (e.g. ethyl acetate, isopentyl acetate, etc.), ether (e.g. diethyl ether, methyl phenyl ether, tetrahydrofuran, etc.), ketone (e.g. acetone, diacetyl, cyclobutanone, etc.), etc. Additional examples of MMEs are provided in U.S. provisional application 62/797,789, filed Jan. 28, 2019 and PCT application Attorney Docket No. 338240: 161-19 WO filed on Jan. 28, 2020. In embodiments, the MMEs in general have a vapor pressure of greater than or equal to 10 torr and more preferably greater than or equal to 100 torr at the operating temperature of the apparatus. In embodiments, the MME is a liquid or a solid at normal temperature and pressure (20° C. and 1 atm (760 torr). In embodiments, the transport moiety is a liquid at normal temperature and pressure (20° C. and 1 atm (760 torr). In embodiments, the vapor pressure of the MME is greater than or equal to the vapor pressure of the transport moiety at the apparatus operating temperatures. For the purpose of this invention, the MME must have a vapor pressure that increases the vapor pressure of the transport moiety upon blending at a concentration such that the vapor-liquid ratio of the blend at room temperature and at 10 torr is between 1 and 90 wt %, inclusive, more preferably at less than or equal to 50 wt %. The MME-transport moiety blend with a vapor-liquid ratio under the conditions described above can favorably change the vapor pressure of the desired transport moiety such that the transport moiety vaporizes faster and at lower temperatures. Generally, an MME with a higher vapor pressure should be added to the transport moiety to increase the vapor pressure of the transport moiety but, in some implementations, an MME with a lower vapor pressure may be added to the transport moiety so as to also advantageously affect vaporization kinetics and vapor pressure of the transport moiety. Generally speaking, a lower vapor pressure MME, when added to a transport moiety may readily break the attractive forces of the transport moiety molecules effectively causing the transport moiety to readily evaporate ultimately raising the vapor pressure of the transport moiety. One skilled in the art understands that the temperature, pressure and/or vapor-liquid ratio of the MME-transport moiety blend can be adjusted to complement desired MME-transport moiety blend properties and/or process conditions.

In embodiments, the weight ratio of MME to transport moiety in compositions herein ranges from 0.01 to 1. In more specific embodiments, the weight ratio of MME to transport moiety in compositions herein ranges from 0.01 to 0.8, or 0.01 to 0.5 or 0.01 to 0.25, or 0.01 to 0.2 or 0.01 to 0.1, or 0.01 to 0.05, or 0.05 to 1 or 0.05 to 0.8 or 0.05 to 0.5 or 0.05 to 0.25 or 0.05 to 0.2 or 0.05 to 0.1, or 0.1 to 1, or 0.1 to 0.8 or 0.1 to 0.5, or 0.1 to 0.25 or 0.1 to 0.2.

Most generally the mixture or blend of MME and transport moiety can be provided as a liquid or solid at NPT.

The MME is a component whose volatilization and mechanism of transport properties are sufficient to carry out the methods of this invention. The volatilization properties of an MME not only expedite the release of molecules from liquid or solids but also improve the movement of vapor molecules in a sub-atmospheric pressure environment by preventing molecular aggregation and/or stagnation. For example, take hydrogen peroxide vapor in a reduced pressure sterilization/sanitization system. Hydrogen peroxide vapor is considered to be a “lazy” gas and has a tendency to absorb to all types of surfaces, such as the inner walls of the apparatus and/or other objects being processed. The velocity of hydrogen peroxide gaseous material may be considered to be near zero at the surface of a substrate, for example a lumen(s), due to the fact that hydrogen peroxide vapor inherently moves slowly and may be consumed within close proximity of the lumen(s) due to absorption before reaching the object to be sterilized. As such, little to no hydrogen peroxide vapor will pass through into the inner area of the lumen(s). MME vapor helps by negating the effects of transport moiety aggregation and/or stagnation by providing a transport conduit by which the transport moiety vapor experiences increased velocity, improved laminar flow and deliverability to target surfaces. Improvement in mobility of transport moiety vapor to a target surface makes it possible to direct the flow of the desired gaseous material into diffusion restricted areas. MME gaseous material serves to prevent irregularities in the pattern of vapor flow at different parts of an object or a device by enabling the user to control the speed at which a transport moiety vapor passes over a material. It may be preferable to decrease or increase the rate of vapor exposure depending on the nature of the transport moiety vapor. For example, if a user was employing a more reactive and mobile sterilant, such as performic acid, it may be advantageous to increase the rate of vapor exposure. It should be noted that the rate of vapor exposure can be controlled by adjusting the MME-transport moiety blend as well as process condition.

The MME may be selected from, but is not limited to, one or more of any alcohol, any alkane, any carboxylic acid, any ester, any ether and/or any ketone or any combination thereof. Alcohols may include, but are not limited to, any linear, branched, cyclic, primary, secondary, tertiary alcohol, polyol and/or isomeric form of a C1-C20 alcohol or in more specific embodiments a C1-C12, a C1-C6 or a C1-C4 alcohol. Some examples may include methanol, ethanol, isopropanol, etc. All combinations and subcombinations of alcohols are included (e.g. alcohols that comprise mixtures such as ethanol and isopropanol).

Alkanes may include, but are not limited to, any linear, branched, cyclic, saturated, unsaturated, polymeric and/or isomeric form of a C5-C20 alkane, or in more specific embodiments a C5-C10 alkane. Some examples may include pentane, hexane, heptane, etc. All combinations and subcombinations of alkanes are included (e.g. alkanes that comprise mixtures such as pentane and hexane, etc.).

Carboxylic acid may include, but are not limited to, any linear, branched, cyclic, saturated, unsaturated, polycarboxylic acid, hydroxy and keto acid and/or any amino acid having 1-20 carbon atoms, or in more specific embodiments, those having 1-12 carbon atoms, those having 1-6 carbon atoms or those having 1-3 carbon atoms. Some examples may include formic acid, acetic acid, propionic acid. All combinations and subcombinations of carboxylic acids are included (e.g. carboxylic acids that comprise mixtures such as acetic acid and citric acid).

Esters may include, but are not limited to, any linear, branched, cyclic, saturated, unsaturated, poly-ester, and/or isomeric form of a C3-C20 ester, or in more specific embodiments, C3-C12 esters, C3-C8 esters or C3 to C6 esters. Some examples may include ethyl acetate, methyl butyrate, methyl anthranilate. All combinations and subcombinations of esters are included (e.g. esters that comprise mixtures such as ethyl acetate and isopentyl acetate).

Ethers may include, but are not limited to, any linear, branched and/or cyclic, saturated, unsaturated, molecules containing multiple ether groups, and/or isomeric forms of a C4-C20 ether or in more specific embodiments C4-C12 ethers or C4-C8 ethers. Some examples may include diethyl ether, methyl phenyl ether, tetrahydrofuran, etc. All combinations and subcombinations of ethers are included (e.g. ethers that comprise mixtures such as cyclopropyl methyl ether and 1,4-dioxane).

Ketones may include, but are not limited to, linear, branched, cyclic, saturated, unsaturated, polyketones (e.g. acetyl, dimedone, etc.), and/or isomeric forms of a C3-C20 ketone or in specific embodiments C3-C12, C3-C8 or C3 to C6 ketones. Some examples may include acetone, diacetyl, cyclobutanone, etc. All combinations and subcombinations of ketones are included (e.g. ketones that comprise mixtures such as cyclopropenone and cyclobutanone, etc.)

The list of MMEs described is not limiting because one skilled in the art, based on the knowledge of compounds having similar properties and/or activities in the apparatus described herein, may select other molecules to carry out the described methods of the invention. Additional sources of MME may include any aldehyde, alkene, alkyne, amide, amine, aniline, aromatic compound, halogen containing compound, nitriles, other nitrogen containing compound, phenol, thiol, sulfide, etc. The MME may also include any structural analog or structural derivative of a described compound such that any component or combination of components are sufficiently volatile under the conditions described in the invention to carry out the functions of the invention. A “structural analog” or “structural derivative” described herein may be defined as a compound with a structure that is similar to that of an alternative compound that differs from it with respect to a certain component. It may vary with respect to one or more atoms, functional groups or substructures which are replaced with alternative atoms, functional groups or substructures. For examples, a structural analog of methanol may include silanol.

Alternatively, an MME may be generated in situ prior to blending with the transport moiety. Any suitable alternative source that when in contact with a solution of solvent, such as water, may be used to generate the MME in situ may be used for the present method. For example, an alternative source may be a salt of an alkoxide, e.g. sodium ethoxide, which in the presence of water generates the alcohol ethanol. In another example, an alternative source may be a salt of a carboxylic acid, e.g. sodium formate, which in the presence of water generates the carboxylic acid formic acid. It should be appreciated that while a limited number of methods for generating MMEs in situ from alternative sources have been described herein other methods and/or alternative sources may be suitably used to generate MMEs in situ.

Additionally, additives may be added to the MME, the transport moiety and/or the MME-transport moiety blend. These additives may include functional ingredients such as acidulants, buffers, carriers, catalysts, sterilants, sanitizers or disinfectants, stabilizing agents (e.g. chelators or sequestrants) and/or wetting agents. A person skilled in the art will understand that though it is not necessary to add additives to carry out the invention the method can be implemented using an MME, transport moiety and/or MME-transport moiety blend that contains any amount of additive.

The term “transport moiety” as used herein refers to any agent, such as a solid, liquid and/or vapor, which exhibits improved volatilization and molecular transport in a sub-atmospheric environment as a result of being in composition with an MME. In embodiments, the transport moiety is an agent used to subject an article to any treatment, such as cleaning, sanitizing or sterilization, among others, or is an agent that is coated upon an article. In an embodiment, the agent is intended to contact substantially all surfaces of the article including any internal surfaces of the article, such as those associated with a crevice, notch, hole, indentation, channel, lumen or the like. In an embodiment, the article to be treated comprises one or more surface to be treated which is diffusion restricted. Some non-limiting examples include cleaning agents, sanitizing agents, sterilizing agents, coating agents and the like. More specifically, agents include hydrogen peroxide (H2O2), peroxyacids (peracetic, performic, etc.), alcohols (e.g. isopropanol, methanol, etc.). Examples of sterilization agents are provided in U.S. provisional application 62/797,789, filed Jan. 28, 2019 and PCT application Attorney Docket No. 338240: 161-19 WO filed on Jan. 28, 2020. In embodiments, the transport moiety has a vapor pressure of greater than or equal to 10 torr and more preferably greater than or equal to 100 torr at the operating temperature of the apparatus. In embodiments, the transport moiety is a liquid or a solid at normal temperature and pressure (NPT, 20° C. and 1 atm (760 torr).

In embodiments, the transport moiety is a liquid at NPT and can include any class of sanitizer or sterilizer than can be used in a sub-atmospheric environment to treat a substrate, article, device or other object. The agents can include, but are not limited to one or a mixture of the following, alcohols, such as ethanol and/or isopropanol, chlorine and chlorine compounds, such as hypochlorite, hypochlorous acid and/or chloride dioxide, ethylene oxide, propylene oxide, formaldehyde, glutaraldehyde, hydrogen peroxide, iodophors, ortho-phthaladehyde, ozone, peroxy acids, such as peracetic acid and/or performic acid, phenolics, such as phenol/phenate, and/or beta-propiolactone.

The invention generally provides MME and MME-transport moiety compositions for improved delivery of the MME and/or MME-transport moiety blend in an apparatus capable of achieving reduced pressures. Liquid and solid compositions of the invention comprise an MME and a carrier. In embodiments, liquid and solid compositions of the invention comprise a transport moiety, an MME and a carrier. Compositions optionally comprise a surfactant and one or more additional additives that may provide improved attributes to the composition (e.g. binders, solublizers, bufferants, thickeners and/or barrier material (e.g. packaging, coating, etc.)). Carriers for liquid compositions include appropriate solvents.

In embodiments, the invention provides solid MME-transport moiety compositions for improved delivery of the MME-transport moiety blend in an apparatus capable of achieving reduced pressures. The solid compositions provided in this disclosure also provide improved storage and stability of MME-transport moieties.

In embodiments, compositions of the invention comprise a transport moiety, an MME and a carrier. Compositions optionally comprise a surfactant and one or more additional additives that may provide improved attributes to the composition (e.g. binders, solublizers, bufferants, thickeners and/or barrier material (e.g. packaging, coating, etc.)).

In embodiments, solid provides release of transport moiety vapor or a mixture of MME and transport moiety vapor in a reduced pressure system and can be formulated to control the release rate of the MME-transport moiety vapor for the desired amount of time within a reduced pressure system. The solid may also be formulated to hold as little or as much of the mixture of MME and transport moiety so as to provide the user with multiple uses in the reduced pressure system. The vapor generated from the solid may serve a number of purposes. For example, the vapor may be used for cleaning, sanitation, sterilization, or coating applications. Exemplary methods and apparatus in which the compositions herein can be employed include those described in published PCT application WO2018/175455, published Sep. 27, 2018, U.S. published application US2018/0289846 and U.S. provisional applications 62/473,543, filed Mar. 20, 2017 and 62/598,004, filed Dec. 13, 2017, each of which is incorporated by reference herein in its entirety. U.S. provisional application 62/797,789, filed Jan. 28, 2019 and corresponding PCT application Attorney Docket No: 338240: 161-19 WO filed Jan. 28, 2020. Each of the listed applications is incorporated by reference herein in its entirety. The solid also provides enhanced storage of the MME-transport moiety blend liquid so as to provide additional stability and/or enhanced storage capabilities.

In embodiments, the compositions of the disclosure are used to generate a vapor in an apparatus, e.g., a diffusion chamber in an apparatus. The vapor comprises the transport moiety or a combination of the transport moiety and the MME. The relative amounts of the transport moiety and the MME in the vapor generated may be the same as the relative amounts of these components in the composition, but most often will be different. The amounts and relative amounts of components in the vapors generated depend generally upon the relative vapor pressures of the components of the composition.

In embodiments, the vapor is generated in the apparatus at ambient room temperature such that the apparatus is not heated. In embodiments, the vapor is generated in the apparatus at a selected temperature above ambient room temperature.

In specific embodiments, the vapor pressure of the transport moiety at the operating temperature of the apparatus is greater than or equal to 10 torr. In embodiments, the transport moiety is a liquid at NPT (see definition below). In specific embodiments, the vapor pressure of the MME at the operating temperature of the apparatus is greater than or equal to 10 torr. In embodiments, the MME is a liquid at NPT. In specific embodiments, the vapor pressure of the transport moiety is less than the vapor pressure of the MME at the operating temperatures of the apparatus.

In more specific embodiments, the vapor pressure of the MME is 20 torr or higher, 30 torr or higher or 40 torr or higher at the operating temperatures of the apparatus.

The attributes described herein refer to compositions and methods for preparing and utilizing a solid for delivering an MME-transport moiety vapor in a reduced pressure environment and/or for improving shelf storage and/or transport of the MME-transport moiety blend and/or component(s). Such attributes are meant to be exemplary and a number of variations and modifications are apparent to one skilled in the art. All related variations and modifications are considered to be within the scope of the description herein.

### Composition Components

### Carriers

The liquid and solid compositions comprise one or more carriers. Carriers for liquid compositions include any appropriate solvents for the MME and/or transport moiety. Useful solvents are volatile at operational temperature and pressure. Solvents are selected for a given application to minimize detriment to objects that are treated.

In solid compositions, the carrier can be a powder, a wax, a gel, or a particle (or plurality of particles), such as a granule, a pellet, a bead, a spherule, a beadlet, a microcapsule, a millisphere, a minitablet, a table or a capsule. Carriers can be formed from a variety of different materials known in the art, such as any synthetic, semi-synthetic, and/or natural polymer, ceramic, glass, metallic, hybrid and/or composite material. Polymers used in the compositions herein may be linear, branched, cyclic, cross-linked or in a network and/or of any isomeric form. They may also be in the form of copolymers, such as but not limited to, diblock, triblock, random, and/or any multi-arm or dendrimeric configuration. In some implementations, the polymer may be crosslinked. Polymers useful in this disclosure may be hydrophilic, hydrophobic or amphipathic such that it is selected to maximize MME-transport moiety liquid compatibility with the carrier. In some implementations, the polymer may be partially or completely insoluble in the MME-transport-moiety liquid such that it only swells when in the presence of the liquid so as to form a hydrogel.

A number of different polymers may be used as a carrier in this disclosure. Examples of groups of synthetic and semi-synthetic polymers include, but are not limited to, any polyacrylamide, polyacrylate, polyamide, polyanhydride, polycarbonate, polycyanoacrylate, polydiene, polyepoxide, polyester, polyether, polyfumarate, polyimide, polyitaconate, polyketone, polynitrile, polyolefin, polyphenylene, polyphenylether, polyphosphazene, polyphosphoester, polysiloxane, polystyrene, polysulfide, polysulfone, polyurethane, polyvinyl alcohol, polyvinyl ester, polyvinylether, polyvinylketone, polyvinylsulfide, protein (e.g. gelatin, etc.), poly(aminoacid), carbohydrate (e.g. saccharides, polysaccharides, oligosaccharides, starches/derivatived starches, cellulose/derivatized cellulose, chitosan, chitins, dextrans, agar, etc), other natural polymers (lignin, etc.), etc. A number of different non-polymeric materials may also be used as a carrier for composition of this disclosure. Examples of non-polymeric materials include, but are not limited to, any ceramic (e g aluminum oxide, zirconium oxide, silicon dioxide, magnesium oxide, titanium oxide, aluminum nitride, silicon nitride, boron nitride, silicon carbide, etc.). A carrier may be porous. Composite materials include but are not limited to, combinations of two or more of the carrier materials described herein.

### Polyacrylamides

### Poly(Methacrylamides)

The compositions of the present disclosure may include a carrier selected from the group consisting of polyacrylamide polymers which may include any type of polyacrylamide represented by the general structure comprising of recurring units represented by formula 22:

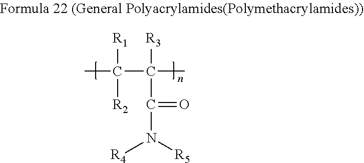

In formula 22, n is an integer representing the number of repeating units and n is most generally an integer between 1 and 20,000. R₁, R₂, R₃, R₄ and/or R₅ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁, R₂, R₃, R₄ and/or R₅, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Groups defined by R₁, R₂, R₃, R₄ and/or R₅ may include heteroatoms.

R₁, R₂, R₃, R₄ and/or R₅ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like) sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl). R₁, R₂, R₃, R₄ and/or R₅ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁, R₂, R₃, R₄ and/or R₅ may also be selected from any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboxyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In embodiments, n of the above formula ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

In some implementations, the polymeric backbone of the polyacrylamide may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyacrylamide backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl). The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

Polyacrylamide polymers can be prepared by any process known in the art. It is intended that the definition of polyacrylamide polymer(s) includes any copolymer. The polyacrylamide may be prepared in the presence of other chemicals or polymers (e.g. surface-active agents, initiators, chain termination agents, polymers, solvents, catalysts, etc.). The polyacrylamide may be treated post polymerization (e.g. grafting, crosslinking, other modifications or functionalizations, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyacrylamides may be utilized for compositions of this disclosure. Some types of polyacrylamides may include, but are not limited to, poly(acrylamide), poly(N-isopropyl acrylamide), poly(N-octyl acrylamide), poly(N-tert-butyl acrylamide), poly(N-phenyl acrylamide), poly(N-sec-butyl acrylamide), poly(N-acetyl methacrylamide), poly(N-benzyl methacrylamide), poly(N-tert-butyl methacrylamide), etc.

### Polyacrylates

### (Polymethacrylate)

The present disclosure may include a carrier selected from the group consisting of polyacrylate polymers which may include any type of polyacrylate represented by the general structure comprising of recurring units represented by formula 23:

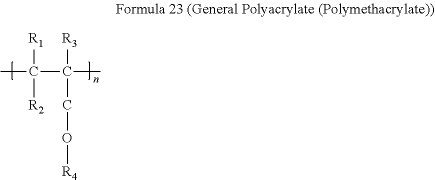

In formula 22, n is an integer representing the number of repeating units and n is most generally an integer between 1 and 20,000 and R₁, R₂, R₃ and/or R₄ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁, R₂, R₃ and/or R₄, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Groups defined by R₁, R₂, R₃ and/or R₄ may include heteroatoms. One or more of each R₁, R₂, or R₃ may be a hydrogen. In an embodiment, not all of R1, R2, or R3 are hydrogens.

R₁, R₂, R₃ and/or R₄ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁, R₂, R₃ and/or R₄ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁, R₂, R₃ and/or R₄ may also be selected independently from any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In embodiments, n of the above formula ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

In some implementations, the polymeric backbone of the polyacrylamide may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyacrylamide backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl). The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

Polyacrylate polymers can be prepared by any process known in the art. It is intended that the definition of polyacrylate polymer(s) includes any copolymer. The polyacrylate may be prepared in the presence of other chemicals or polymers (e.g. surface-active agents, initiators, chain termination agents, polymers, solvents, catalysts, etc.). The polyacrylate may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyacrylates may be utilized for the compositions of this disclosure. Some types of polyacrylates may include, but are limited to, poly(acrylic acid), poly(benzyl acrylate), poly(butyl acrylate), poly(4-chlorophenyl acrylate), poly(2-cyanoacrylate), poly(cyano methyl acrylate), poly(cyclohexyl acrylate), poly(ethyl acrylate), poly(2-ethylhexyl acrylate), poly(hexyl acrylate), poly(isobutyl acrylate), poly(isopropyl acrylate), poly(methyl acrylate), poly(octyl acrylate), poly(propyl acrylate), poly(sec-butyl acrylate), polystearyl acrylate, poly(tert-butyl acrylate, poly(2,2,3,3-tetrafluoropropyl acrylate), poly(methacrylic acid), poly(benzyl methacrylate), poly(butyl methacrylate), poly(cyclohexyl methacrylate), poly(decyl methacrylate), poly(dodecyl methacrylate), poly(2-ethoxy ethyl methacrylate), poly(ethyl methacrylate), poly(hexyl methacrylate), poly(2-hydroxyethyl methacrylate), poly(2-hydroxypropyl methacrylate), poly(isobutyl methacrylate), poly(isopropyl methacrylate), poly(methyl methacrylate), poly(octadecyl methacrylate), poly(octyl methacrylate), poly(2-phenyl ethyl methacrylate), poly(phenyl methacrylate), poly(propyl methacrylate), poly(2-chloroethyl methacrylate), poly(sec-butyl methacrylate), poly(4-tert-butyl cyclohexyl methacrylate), poly(tert-butyl methacrylate), poly(2,2,3,3-tetrafluoro propyl methacrylate), etc.

### Polyamide

The compositions of the present invention may include a carrier selected from the group consisting of polyamide polymers which may include any type of polyamide represented by the general structures comprising of recurring units represented by Formula 24:

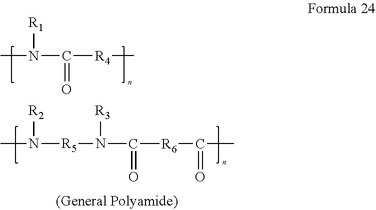

In formula 24, n represents the number of repeating units and generally is an integer ranging from 1-20,000 inclusive, R₁-R₃ are defined below and R₄, R₅, and/or R₆ may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 arylenediyl, C3-200 alkarylenediyl, C3-200 aralkylenediyl. To further define R₄, R₅, and/or R₆, any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any arylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable. Groups defined by R₁, R₂, R₃, R₄, R₅, and/or R₆ may include heteroatoms.

R₄, R₅, and/or R₆ may also be independently selected from a haloalkanediyl; haloalkenediyl; haloalkynediyl; halocycloalkanediyl; halocycloalkenediyl; halocycloalkynediyl; haloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxyarylenediyl; alkoxyalkarylenediyl; alkoxyaralkylenediyl; aminoalkanediyl; aminoalkenediyl; aminoalkynediyl; aminocycloalkanediyl; aminocycloalkenediyl; aminocycloalkynediyl; aminoarylenediyl; aminoalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfoarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphoarylenediyl; phosphoalkarylenediyl; phosphoaralkylenediyl; mono-, di- and trialkylamino-(alkanediyl, alkenediyl, alkynediyl, cycloalkanediyl, cycloalkenediyl, cycloalkynediyl, arylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, arylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, arylenediyl, alkarylenediyl or aralkylenediyl); arylamino-(alkanediyl, alkenediyl, alkynediyl, cycloalkanediyl, cycloalkenediyl, cycloalkynediyl, arylenediyl, alkarylenediyl or aralkylenediyl); arylsulfo-(alkanediyl, alkenediyl, alkynediyl, cycloalkanediyl, cycloalkenediyl, cycloalkynediyl, arylenediyl, alkarylenediyl or aralkylenediyl); arylphospho-(alkanediyl, alkenediyl, alkynediyl, cycloalkanediyl, cycloalkenediyl, cycloalkynediyl, arylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, arylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, arylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, arylenediyl, alkarylenediyl or aralkylenediyl).

R₄, R₅, and/or R₆ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; arylenediyl; alkarylenediyl or aralkylenediyl; acylamino; acylsulfo; acylphospho; alkylamino, alkylsulfo, alkylphospho, and the like.

R₄, R₅, and/or R₆ may also be selected from any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In embodiments, n of the above formula ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

In certain implementations, R₄, R₅, and/or R₆ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M.

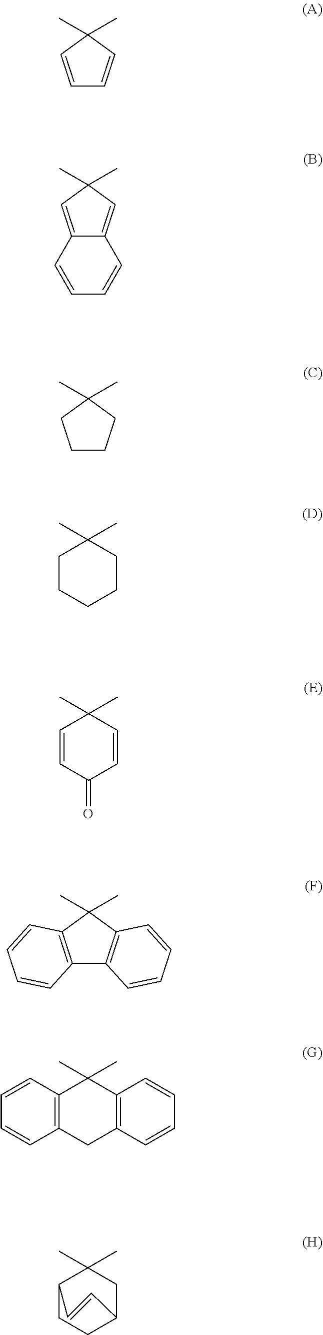

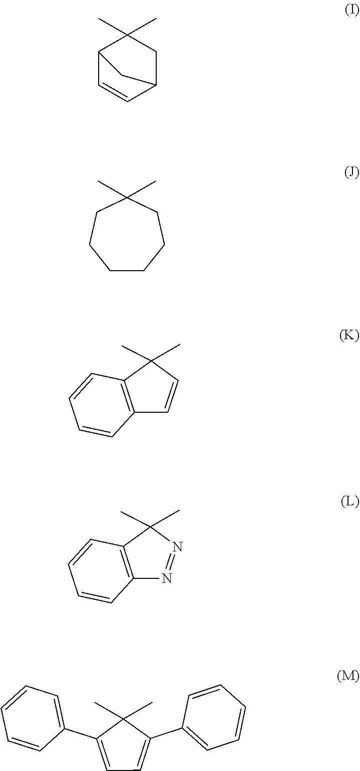

Other non-limiting bridging group examples may include those in formulas N through X as well as other such as, but not limited to, —(CH2)x-, —C(═O)—, —O—, —S(═O)₂—, —C(CF₃)₂—, —Si(CH₃)₂—, —C(CH₃)₂, —O(CH₂)XO—, —(CH₂O)x-, etc., where x is an integer between 1 and 20,000, inclusive. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

Further, in formula 24, R₁, R₂ and/or R₃ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁, R₂ and/or R₃, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable.

R₁, R₂ and/or R₃ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁, R₂ and/or R₃ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁, R₂ and/or R₃ may also be selected from hydrogen, any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁-R₃ are hydrogen. In embodiments, not all of R₁-R₃ are hydrogens.

In some implementations, the polymeric backbone of the polyamide may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyamide backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like) amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like) sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, n of the above formula ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyamide polymers can be prepared by any process known in the art. It is intended that the definition of polyamide polymer(s) includes any copolymer. The polyamide may be prepared in the presence of other chemicals or polymers (e.g. surface-active agents, initiators, chain termination agents, polymers, solvents, catalysts, etc.). The polyamide may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyamides may be utilized for this invention. Some types of polyamides may include, but are limited to, poly(propiolactam), poly(caprolactam), poly(capryllactam), poly(decano-10-lactam), poly(undecano-11-lactam), poly(dodecano-12-lactam), poly(hexamethylene adipamide), poly(hexamethylene azelamide), poly(hexamethylene sebacamide), poly(hexamethylene dodecanediamide), poly(decamethylene sebacamide), poly(hexamethylene isophthalamide), poly(hexamethylene teraphthalamide), polyaramide, poly(m-phenylene terephthalamide), poly(nonanmethylene teraphthalamide), etc.

### Polyanhydride

The composition of the present disclosure may include a carrier selected from the group consisting of polyanhydride polymers which may include any type of polyanhydride represented by the general structure comprising of recurring units represented by formula 25:

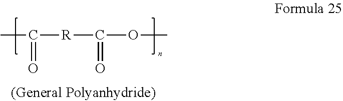

In formula 25, n is an integer representing the number of repeating units and generally can range from 1 to 20,000 and R may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 arylenediyl, C3-200 alkarylenediyl, C3-200 aralkylenediyl. To further define R, any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any arylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable. Groups defined by R may include heteroatoms. R may also be independently selected from a haloalkanediyl; haloalkenediyl; haloalkynediyl; halocycloalkanediyl; halocycloalkenediyl; halocycloalkynediyl; haloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxyarylenediyl; alkoxyalkarylenediyl; alkoxyaralkylenediyl; aminoalkanediyl; aminoalkenediyl; aminoalkynediyl; aminocycloalkanediyl; aminocycloalkenediyl; aminocycloalkynediyl; aminoarylenediyl; aminoalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfoarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphoarylenediyl; phosphoalkarylenediyl; phosphoaralkylenediyl; mono-, di- and trialkylamino-(alkanediyl, alkenediyl, alkynediyl, cycloalkanediyl, cycloalkenediyl, cycloalkynediyl, arylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, arylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, arylenediyl, alkarylenediyl or aralkylenediyl); arylamino-(alkanediyl, alkenediyl, alkynediyl, cycloalkanediyl, cycloalkenediyl, cycloalkynediyl, arylenediyl, alkarylenediyl or aralkylenediyl); arylsulfo-(alkanediyl, alkenediyl, alkynediyl, cycloalkanediyl, cycloalkenediyl, cycloalkynediyl, arylenediyl, alkarylenediyl or aralkylenediyl); arylphospho-(alkanediyl, alkenediyl, alkynediyl, cycloalkanediyl, cycloalkenediyl, cycloalkynediyl, arylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, arylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, arylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, arylenediyl, alkarylenediyl or aralkylenediyl). R may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; arylenediyl; alkarylenediyl or aralkylenediyl; acylamino; acylsulfo; acylphospho; alkylamino, alkylsulfo, alkylphospho, and the like. R may also be selected from any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In certain implementations, R may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through X as well as other such as, but not limited to, —(CH₂)x-, —C(═O)—, —O—, —S(═O)₂—, —C(CF₃)₂—, —Si(CH₃)₂—, —C(CH₃)₂, —O(CH₂)XO—, —(CH₂O)x-, etc., where x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

In some implementations, the polymeric backbone of the polyanhydride may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyanhydride backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl). The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, n of the above formula ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyanhydride polymers can be prepared by any process known in the art. It is intended that the definition of polyanhydride polymer(s) includes any copolymer. The polyanhydride may be prepared in the presence of other chemicals or polymers (e.g. surface-active agents, initiators, chain termination agents, polymers, solvents, catalysts, etc.). The polyanhydride may be treated post polymerization (e.g. grafting, crosslinking, other modifications or functionalizations, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyanhydrides may be utilized for this invention. Some types of polyanhydrides may include, but are limited to, poly(p-pentamethylenedibenzoic anhydride), poly(p-tetramethylenedibenzoic anhydride), poly(sebacic anhydride), poly(azelaic anhydride), etc.

Polycarbonate

The compositions of the present invention may include a carrier selected from the group consisting of polycarbonate polymers which may include any type of polycarbonate represented by the general structure comprising of recurring units represented by Formula 26:

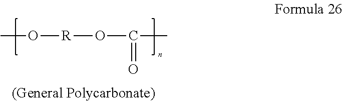

In formula 26, n is an integer representing the number of repeating units and is most generally 1-20,000, inclusively, and R may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 arylenediyl, C3-200 alkarylenediyl, C3-200 aralkylenediyl. To further define R, any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any arylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable. Groups defined by R may include heteroatoms. R may also be independently selected from a haloalkanediyl; haloalkenediyl; haloalkynediyl; halocycloalkanediyl; halocycloalkenediyl; halocycloalkynediyl; haloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkanediyl; alkoxyarylenediyl; alkoxyalkarylenediyl; alkoxyaralkylenediyl; aminoalkanediyl; aminoalkenediyl; aminoalkynediyl; aminocycloalkanediyl; aminocycloalkenediyl; aminocycloalkynediyl; aminoarylenediyl; aminoalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfoarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphoarylenediyl; phosphoalkarylenediyl; phosphoaralkylenediyl; mono-, di- and trialkylamino-(alkanediyl, alkenediyl, alkynediyl, cycloalkanediyl, cycloalkenediyl, cycloalkynediyl, arylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, arylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, arylenediyl, alkarylenediyl or aralkylenediyl); arylamino-(alkanediyl, alkenediyl, alkynediyl, cycloalkanediyl, cycloalkenediyl, cycloalkynediyl, arylenediyl, alkarylenediyl or aralkylenediyl); arylsulfo-(alkanediyl, alkenediyl, alkynediyl, cycloalkanediyl, cycloalkenediyl, cycloalkynediyl, arylenediyl, alkarylenediyl or aralkylenediyl); arylphospho-(alkanediyl, alkenediyl, alkynediyl, cycloalkanediyl, cycloalkenediyl, cycloalkynediyl, arylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, arylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, arylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, arylenediyl, alkarylenediyl or aralkylenediyl).

R may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; arylenediyl; alkarylenediyl or aralkylenediyl; acylamino; acylsulfo; acylphospho; alkylamino, alkylsulfo, alkylphospho, and the like.

R may also be selected from any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In certain implementations, R may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through X as well as other such as, but not limited to, —(CH₂)x-, —C(═O)—, —S(═O)₂—, —C(CF₃)₂—, —Si(CH₃)₂—, —C(CH₃)₂, —O(CH₂)XO—, —(CH₂O)x-, etc., where x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

In some implementations, the polymeric backbone of the polycarbonate may be substituted within the backbone with groups that may include, but are not limited to, any form (e.g. divalent, etc.) of those listed above. It may also be understood that the backbone(s) of branches off of the main polymer chain may also be substituted with similar groups. Any part of the polycarbonate backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc.

Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, n of the above formula ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polycarbonate polymers can be prepared by any process known in the art. It is intended that the definition of polycarbonate polymer(s) includes any copolymer. The polycarbonate may be prepared in the presence of other chemicals or polymers (e.g. surface-active agents, initiators, chain termination agents, polymers, solvents, catalysts, etc.). The polycarbonate may be treated post polymerization (e.g. grafting, crosslinking, other modifications or functionalizations, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polycarbonates may be utilized for this invention. Some types of polycarbonates may include, but are limited to, poly(bisphenol A carbonate), poly(4,4′-thiodiphenylene carbonate), poly(bisphenol B carbonate), poly(bisphenol F carbonate), poly(ethylene carbonate), polypropylene carbonate), poly(2,6,3′,5′-tetrachlorobisphenol A carbonate), poly(tetramethyl bisphenol A carbonate), etc.

Polycyanoacrylate

The compositions of the present disclosure may include a carrier selected from the group consisting of polycyanoacrylate polymers which may include any type of polycyanoacrylate represented by the general structure comprising of recurring units represented by Formula 27:

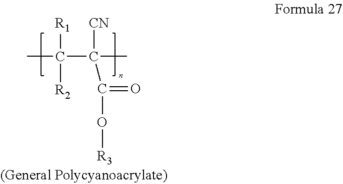

In formula 27, n is an integer representing the number of repeating units and is most generally 1-20,000, and R₁, R₂ and/or R₃ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁, R₂ and/or R₃, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Groups defined by R₁, R₂ and/or R₃ may include heteroatoms.

R₁, R₂ and/or R₃ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁, R₂ and/or R₃ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁, R₂ and/or R₃ may also be selected from hydrogen, or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁-R₃ is hydrogen. In embodiments, not all R₁-R₃ are hydrogens.

In some implementations, the polymeric backbone of the polycyanoacrylate may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyacrylamide backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc.

Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, n of the above formula ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polycyanoacrylate polymers can be prepared by any process known in the art. It is intended that the definition of polycyanoacrylate polymer(s) includes any copolymer. The polycyanoacrylate may be prepared in the presence of other chemicals or polymers (e.g. surface-active agents, initiators, chain termination agents, polymers, solvents, catalysts, etc.). The polycyanoacrylate may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polycyanoacrylate may be utilized for this invention. Some types of polycyanoacrylates may include, but are limited to, poly(methyl cyanoacrylate), poly(ethyl cyanoacrylate), poly(butyl cyanoacrylate), poly(hexyl cyanoacrylate), poly(octyl cyanoacrylate), etc.

Polydienes

The present invention may include a carrier selected from the group consisting of polydiene polymers which may include any type of polydiene represented by the general structure comprising of recurring units represented by Formula 28:

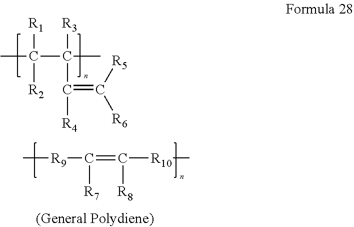

In formula 28, each n is independently an integer representing the number of repeating unit and each n independently most generally ranges from 1-20,0000 and R₉ and/or R₁₀ may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 arylenediyl, C3-200 alkarylenediyl, C3-200 aralkylenediyl. To further define R₉ and/or R₁₀, any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any arylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable. Groups defined by R₁, R₂, R₃, R₄, R₅, R₆, R₇, R₈, R₉ and/or R₁₀ may include heteroatoms.

R₉ and/or R₁₀ may also be independently selected from a haloalkanediyl; haloalkenediyl; haloalkynediyl; halocycloalkanediyl; halocycloalkenediyl; halocycloalkynediyl; haloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxyarylenediyl; alkoxyalkarylenediyl; alkoxyaralkylenediyl; aminoalkanediyl; aminoalkenediyl; aminoalkynediyl; aminocycloalkanediyl; aminocycloalkenediyl; aminocycloalkynediyl; aminoarylenediyl; aminoalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfoarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphoarylenediyl; phosphoalkarylenediyl; phosphoaralkylenediyl; mono-, di- and trialkylamino-(alkanediyl, alkenediyl, alkynediyl, cycloalkanediyl, cycloalkenediyl, cycloalkynediyl, arylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, arylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, arylenediyl, alkarylenediyl or aralkylenediyl); arylamino-(alkanediyl, alkenediyl, alkynediyl, cycloalkanediyl, cycloalkenediyl, cycloalkynediyl, arylenediyl, alkarylenediyl or aralkylenediyl); arylsulfo-(alkanediyl, alkenediyl, alkynediyl, cycloalkanediyl, cycloalkenediyl, cycloalkynediyl, arylenediyl, alkarylenediyl or aralkylenediyl); arylphospho-(alkanediyl, alkenediyl, alkynediyl, cycloalkanediyl, cycloalkenediyl, cycloalkynediyl, arylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, arylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, arylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, arylenediyl, alkarylenediyl or aralkylenediyl). R9 and/or R10 may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; arylenediyl; alkarylenediyl or aralkylenediyl; acylamino; acylsulfo; acylphospho; alkylamino, alkylsulfo, alkylphospho, and the like.

R₉ and/or R₁₀ may also be selected from any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In certain implementations, R₉ and/or R₁₀ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through W as well as other such as, but not limited to, —(CH₂)λ-, —C(═O)—, —O—, —S(═O)₂—, —C(CF₃)₂—, —Si(CH₃)₂—, —C(CH₃)₂, —O(CH₂)XO—, —(CH₂O)x-, etc., where x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

R₁, R₂, R₃, R₄, R₅, R₆, R₇ and/or R₈ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁, R₂, R₃, R₄, R₅, R₆, R₇ and/or R₈, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable.

Groups R₁, R₂, R₃, R₄, R₅, R₆, R₇ and/or R₈ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁, R₂, R₃, R₄, R₅, R₆, R₇ and/or R₈ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁, R₂, R₃, R₄, R₅, R₆, R₇ and/or R₈ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁, R₂, R₃, R₄, R₅, R₆, R₇ and/or R₈ is hydrogen. In embodiments, not all of R₁, R₂, R₃, R₄, R₅, R₆, R₇ and/or R₈ is hydrogen. In embodiments, one or more of R₁, R₂, R₃, R₄, R₅, R₆, R₇ and/or R₈ is hydrogen.

In some implementations, the polymeric backbone of the polydiene may be substituted within the backbone with groups that may include, but are not limited to, any form (e.g. divalent, etc.) of those listed above. It may also be understood that the backbone(s) of branches off of the main polymer chain may also be substituted with similar groups. Any part of the polydiene backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pendant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polydiene polymers can be prepared by any process known in the art. It is intended that the definition of polydiene polymer(s) includes any copolymer. The polydiene may be prepared in the presence of other chemicals or polymers (e.g. surface-active agents, initiators, chain termination agents, polymers, solvents, catalysts, etc.). The polydiene may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polydiene may be utilized for this invention. Some types of polydienes may include, but are limited to, poly(1,2-butadiene), poly(1,4-butadiene), polycyclopentene, poly(l-ethyl-1,4-butadiene), 1,4-polyisoprene, poly(1,4-pentadiene), poly(l-pentenylene), etc.

Polyepoxides (Polyhydroxyethers)

The compositions of the present disclosure may include a carrier selected from the group consisting of polyepoxide polymers which may include any type of polyepoxide represented by the general structure comprising of recurring units represented by Formula 29:

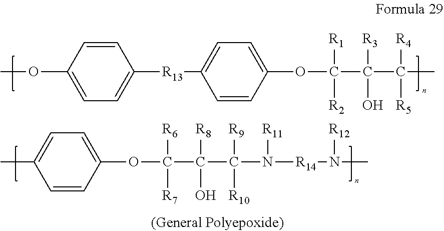

In formula 29, each n is an integer that independently represents the number of repeating units and most generally ranges from 1-20,000, inclusive, and R₁₃ and/or R₁₄ may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 arylenediyl, C3-200 alkarylenediyl, C3-200 aralkylenediyl. To further define R₁₃ and/or R₁₄, any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any arylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable.

Groups defined by R₁, R₂, R₃, R₄, R₅, R₆, R₇, R₈, R₉, R₁₀, R₁₁, R₁₂, R₁₃ and/or R₁₄ may include heteroatoms. R₁₃ and/or R₁₄ may also be independently selected from a haloalkanediyl; haloalkenediyl; haloalkynediyl; halocycloalkanediyl; halocycloalkenediyl; halocycloalkynediyl; haloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxyarylenediyl; alkoxyalkarylenediyl; alkoxyaralkylenediyl; aminoalkanediyl; aminoalkenediyl; aminoalkynediyl; aminocycloalkanediyl; aminocycloalkenediyl; aminocycloalkynediyl; aminoarylenediyl; aminoalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfoarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphoarylenediyl; phosphoalkarylenediyl; phosphoaralkylenediyl; mono-, di- and trialkylamino-(alkanediyl, alkenediyl, alkynediyl, cycloalkanediyl, cycloalkenediyl, cycloalkynediyl, arylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, arylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, arylenediyl, alkarylenediyl or aralkylenediyl); arylamino-(alkanediyl, alkenediyl, alkynediyl, cycloalkanediyl, cycloalkenediyl, cycloalkynediyl, arylenediyl, alkarylenediyl or aralkylenediyl); arylsulfo-(alkanediyl, alkenediyl, alkynediyl, cycloalkanediyl, cycloalkenediyl, cycloalkynediyl, arylenediyl, alkarylenediyl or aralkylenediyl); arylphospho-(alkanediyl, alkenediyl, alkynediyl, cycloalkanediyl, cycloalkenediyl, cycloalkynediyl, arylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, arylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, arylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, arylenediyl, alkarylenediyl or aralkylenediyl).

R₉ and/or R₁₀ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; arylenediyl; alkarylenediyl or aralkylenediyl; acylamino; acylsulfo; acylphospho; alkylamino, alkylsulfo, alkylphospho, and the like.

R₁₃ and/or R₁₄ may also be selected from any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In certain implementations, R₁₃ and/or R₁₄ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through X as well as other such as, but not limited to, —(CH₂)ₓ—, —C(═O)—, —O—, —S(═O)₂—, —C(CF₃)₂—, —Si(CH₃)₂—, —C(CH₃)₂, —O(CH₂)_{X}O—, —(CH₂O)ₓ—, etc. x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

R₁, R₂, R₃, R₄, R₅, R₆, R₇, R₈, R₉, R₁₀, R₁₁, and/or R₁₂ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁, R₂, R₃, R₄, R₅, R₆, R₇, R₈, R₉, R₁₀, R₁₁, and/or R₁₂, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable.

Groups R₁, R₂, R₃, R₄, R₅, R₆, R₇, R₈, R₉, R₁₀, R₁₁, and/or R₁₂ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁, R₂, R₃, R₄, R₅, R₆, R₇, R₈, R₉, R₁₀, R₁₁, and/or R₁₂ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁, R₂, R₃, R₄, R₅, R₆, R₇, R₈, R₉, R₁₀, R₁₁, and/or R₁₂ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁, R₂, R₃, R₄, R₅, R₆, R₇, R₈, R₉, R₁₀, R₁₁, and/or R₁₂ are hydrogen. In embodiments, not all of R₁, R₂, R₃, R₄, R₅, R₆, R₇, R₈, R₉, R₁₀, R₁₁, and/or R₁₂ are hydrogen. In embodiments, one or more of R₁, R₂, R₃, R₄, R₅, R₆, R₇, R₈, R₉, R₁₀, R₁₁, and/or R₁₂ is a hydrogen.

In some implementations, the polymeric backbone of the polyepoxide may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyepoxide backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl). The pendant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc.

Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyepoxide polymers can be prepared by any process known in the art. It is intended that the definition of polyepoxide polymer(s) includes any copolymer. The polyepoxide may be prepared in the presence of other chemicals or polymers (e.g. surface-active agents, initiators, chain termination agents, polymers, solvents, catalysts, etc.). The polyepoxide may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyepoxides may be utilized for this invention. Some types of polyepoxides may include, but are limited to, bisphenol-A diglycidyl ether epoxy resin, bisphenol-F diglycidyl ether epoxy resin, poly(bis-A diglycidyl ether-alt-ethylene diamine), poly(bis-A diglycidyl ether-alt-hexamethylene diamine), poly(bis-A diglycidyl ether-alt-octamethylene diamine), etc.

Polyesters

The present invention may include a carrier selected from the group consisting of polyester polymers which may include any type of polyester represented by the general structure comprising of recurring units represented by formula 30:

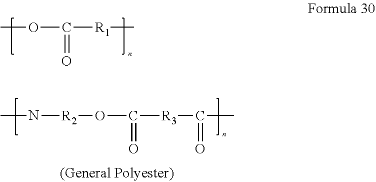

In formula 30, each n independently represents the number of repeating units and most generally ranges from 1-20,000, and R₁ R₂ and/or R₃ may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 arylenediyl, C3-200 alkarylenediyl, C3-200 aralkylenediyl. To further define R₁ R₂ and/or R₃, any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any arylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable. Groups defined by R₁, R₂ and/or R₃ may include heteroatoms.

R₁ R₂ and/or R₃ may also be independently selected from a haloalkanediyl; haloalkenediyl; haloalkynediyl; halocycloalkanediyl; halocycloalkenediyl; halocycloalkynediyl; haloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxyarylenediyl; alkoxyalkarylenediyl; alkoxyaralkylenediyl; aminoalkanediyl; aminoalkenediyl; aminoalkynediyl; aminocycloalkanediyl; aminocycloalkenediyl; aminocycloalkynediyl; aminoarylenediyl; aminoalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfoarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphoarylenediyl; phosphoalkarylenediyl; phosphoaralkylenediyl; mono-, di- and trialkylamino-(alkanediyl, alkenediyl, alkynediyl, cycloalkanediyl, cycloalkenediyl, cycloalkynediyl, arylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, arylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, arylenediyl, alkarylenediyl or aralkylenediyl); arylamino-(alkanediyl, alkenediyl, alkynediyl, cycloalkanediyl, cycloalkenediyl, cycloalkynediyl, arylenediyl, alkarylenediyl or aralkylenediyl); arylsulfo-(alkanediyl, alkenediyl, alkynediyl, cycloalkanediyl, cycloalkenediyl, cycloalkynediyl, arylenediyl, alkarylenediyl or aralkylenediyl); arylphospho-(alkanediyl, alkenediyl, alkynediyl, cycloalkanediyl, cycloalkenediyl, cycloalkynediyl, arylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, arylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, arylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, arylenediyl, alkarylenediyl or aralkylenediyl). R₁ R₂ and/or R₃ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; arylenediyl; alkarylenediyl or aralkylenediyl; acylamino; acylsulfo; acylphospho; alkylamino, alkylsulfo, alkylphospho, and the like. R₁ R₂ and/or R₃ may also be selected from any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In certain implementations, R₁ R₂ and/or R₃ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through X as well as other such as, but not limited to, —(CH₂)ₓ—, —C(═O)—, —O—, —S(═O)₂—, —C(CF₃)₂—, —Si(CH₃)₂—, —C(CH₃)₂, —O(CH₂)_{X}O—, —(CH₂O)ₓ—, etc. x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

In some implementations, the polymeric backbone of the polyester may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyester backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl). The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) can include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyester polymers can be prepared by any process known in the art. It is intended that the definition of polyester polymer(s) includes any copolymer. The polyester may be prepared in the presence of other chemicals or polymers (e.g. surface-active agents, initiators, chain termination agents, polymers, solvents, catalysts, etc.). The polyester may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyesters may be utilized for this invention. Some types of polyesters may include, but are limited to, poly(bisphenol A isophthalate), poly(bisphenol A terephthalate), poly(butylene adipate), poly(butylene isophthalate), poly(butylene sebacate), poly(butylene succinate), poly(butylene terephthalate), poly(ethylene sebacate), poly(ethylene succinate), poly(caprolactone), poly(cyclohexylene dimethylene terephthalate), poly(ethylene adipate), poly(ethylene isophthalate), poly(ethylene naphthalate), poly(ethylene phthalate), poly(ethylene terephthalate), poly(glycolide), poly(hexylene sebacate), poly(hexylene succinate), poly(3-hydroxybutyrate), poly(4-hydroxybutyrate), polylactic acid, polypropylene adipate), poly(trimethylene succinate), poly(trimethylene terephthalate), etc.

Polyethers

The compositions of the present disclosure may include a carrier selected from the group consisting of polyether polymers which may include any type of polyether represented by the general structure comprising of recurring units represented by formula 31.

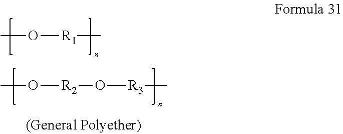

In formula 31, each n independently is an integer representing the number of repeating units and is most generally 1-20,000 and R₁ R₂ and/or R₃ may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 arylenediyl, C3-200 alkarylenediyl, C3-200 aralkylenediyl. To further define R₁ R₂ and/or R₃, any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any arylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable.

Groups defined by R₁, R₂ and/or R₃ may include heteroatoms. R₁ R₂ and/or R₃ may also be independently selected from a haloalkanediyl; haloalkenediyl; haloalkynediyl; halocycloalkanediyl; halocycloalkenediyl; halocycloalkynediyl; haloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxyarylenediyl; alkoxyalkarylenediyl; alkoxyaralkylenediyl; aminoalkanediyl; aminoalkenediyl; aminoalkynediyl; aminocycloalkanediyl; aminocycloalkenediyl; aminocycloalkynediyl; aminoarylenediyl; aminoalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfoarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphoarylenediyl; phosphoalkarylenediyl; phosphoaralkylenediyl; mono-, di- and trialkylamino-(alkanediyl, alkenediyl, alkynediyl, cycloalkanediyl, cycloalkenediyl, cycloalkynediyl, arylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, arylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, arylenediyl, alkarylenediyl or aralkylenediyl); arylamino-(alkanediyl, alkenediyl, alkynediyl, cycloalkanediyl, cycloalkenediyl, cycloalkynediyl, arylenediyl, alkarylenediyl or aralkylenediyl); arylsulfo-(alkanediyl, alkenediyl, alkynediyl, cycloalkanediyl, cycloalkenediyl, cycloalkynediyl, arylenediyl, alkarylenediyl or aralkylenediyl); arylphospho-(alkanediyl, alkenediyl, alkynediyl, cycloalkanediyl, cycloalkenediyl, cycloalkynediyl, arylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, arylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, arylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, arylenediyl, alkarylenediyl or aralkylenediyl). R₁ R₂ and/or R₃ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; arylenediyl; alkarylenediyl or aralkylenediyl; acylamino; acylsulfo; acylphospho; alkylamino, alkylsulfo, alkylphospho, and the like.

R₁, R₂ and/or R₃ may also be selected from any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In certain implementations, R₁ R₂ and/or R₃ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through X as well as other such as, but not limited to, —(CH₂)ₓ—, —C(═O)—, —O—, —S(═O)₂—, —C(CF₃)₂—, —Si(CH₃)₂—, —C(CH₃)₂, —O(CH₂)_{X}O—, —(CH₂O)ₓ—, etc., where x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

In some implementations, the polymeric backbone of the polyether may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyether backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyether polymers can be prepared by any process known in the art. It is intended that the definition of polyether polymer(s) includes any copolymer. The polyether may be prepared in the presence of other chemicals or polymers (e.g. surface-active agents, initiators, chain termination agents, polymers, solvents, catalysts, etc.). The polyether may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyethers may be utilized for this invention. Some types of polyethers may include, but are not limited to, polyacetal, poly(3-butoxypropylene oxide), poly(epichlorohydrin), poly(ethylene glycol), poly(hexamethylene oxide), poly(3-methoxypropylene oxide), poly[oxy(hexyloxymethyl)ethylene], poly(oxymethylene-oxyethylene), poly(oxymethylene-oxytetramethylene), poly(propylene glycol), poly(tetrahydrofuran), poly(trimethylene glycol), poly[1,1-bis(chloromethyl)trimethylene oxide], etc.

Polyfumarates (Polybutenedioates)

The compositions of the present disclosure may include a carrier selected from the group consisting of polyfumarate polymers which may include any type of polyfumarate represented by the general structure comprising of recurring units represented by Formula 32:

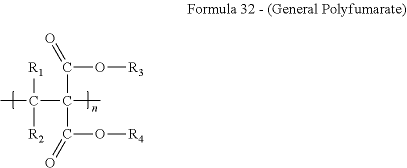

In formula 32, n is an integer representing the number of repeating units and is most generally 1-20,000 inclusive, and R₁, R₂, R₃ and/or R₄ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁, R₂, R₃ and/or R₄, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Groups defined by R₁, R₂, R₃ and/or R₄ may include heteroatoms.

R₁, R₂, R₃ and/or R₄ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁, R₂, R₃ and/or R₄ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁, R₂, R₃ and/or R₄ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁-R₄ is hydrogen. In embodiments, not all of R₁-R₄ is hydrogen.

In some implementations, the polymeric backbone of the polyfumarate may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyfumarate backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyfumarate polymers can be prepared by any process known in the art. It is intended that the definition of polyfumarate polymer(s) includes any copolymer. The polyfumarate may be prepared in the presence of other chemicals or polymers (e.g. surface-active agents, initiators, chain termination agents, polymers, solvents, catalysts, etc.). The polyfumarate may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyfumarates may be utilized for this invention. Some types of polyfumarates may include, but are limited to, poly(dimethyl fumarate), poly(dibutyl fumarate), poly(diethyl fumarate), poly(dipropyl fumarate), etc.

Polyimide

The compositions of the present disclosure may include a carrier selected from the group consisting of polyimide polymers which may include any type of polyimide represented by the general structures comprising of recurring units represented by Formula 33:

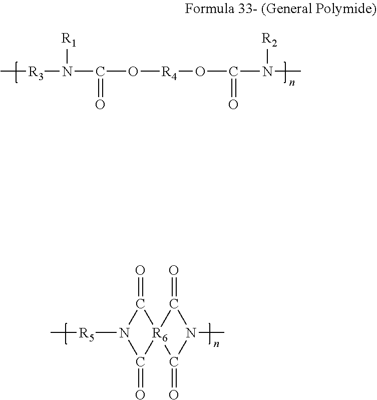

In formula 33, each n independently represents the number of repeating units and is most generally 1-20,000 and R₃, R₄, R₅ and/or R₆ may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 arylenediyl, C3-200 alkarylenediyl, C3-200 aralkylenediyl. To further define R₃, R₄, R₅ and/or R₆, any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any arylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable. Groups defined by R₁, R₂, R₃, R₄, R₅, and/or R₆ may include heteroatoms.

R₃, R₄, R₅ and/or R₆ may also be independently selected from a haloalkanediyl; haloalkenediyl; haloalkynediyl; halocycloalkanediyl; halocycloalkenediyl; halocycloalkynediyl; haloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxyarylenediyl; alkoxyalkarylenediyl; alkoxyaralkylenediyl; aminoalkanediyl; aminoalkenediyl; aminoalkynediyl; aminocycloalkanediyl; aminocycloalkenediyl; aminocycloalkynediyl; aminoarylenediyl; aminoalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfoarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphoarylenediyl; phosphoalkarylenediyl; phosphoaralkylenediyl; mono-, di- and trialkylamino-(alkanediyl, alkenediyl, alkynediyl, cycloalkanediyl, cycloalkenediyl, cycloalkynediyl, arylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, arylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, arylenediyl, alkarylenediyl or aralkylenediyl); arylamino-(alkanediyl, alkenediyl, alkynediyl, cycloalkanediyl, cycloalkenediyl, cycloalkynediyl, arylenediyl, alkarylenediyl or aralkylenediyl); arylsulfo-(alkanediyl, alkenediyl, alkynediyl, cycloalkanediyl, cycloalkenediyl, cycloalkynediyl, arylenediyl, alkarylenediyl or aralkylenediyl); arylphospho-(alkanediyl, alkenediyl, alkynediyl, cycloalkanediyl, cycloalkenediyl, cycloalkynediyl, arylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, arylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, arylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, arylenediyl, alkarylenediyl or aralkylenediyl).

R₃, R₄, R₅ and/or R₆ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; arylenediyl; alkarylenediyl or aralkylenediyl; acylamino; acylsulfo; acylphospho; alkylamino, alkylsulfo, alkylphospho, and the like.

R₃, R₄, R₅ and/or R₆ may also be selected from any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In certain implementations, R₃, R₄, R₅ and/or R₆ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through X as well as other such as, but not limited to, —(CH₂)ₓ—, —C(═O)—, —O—, —S(═O)₂—, —C(CF₃)₂—, —Si(CH₃)₂—, —C(CH₃)₂, —O(CH₂)_{X}O—, —(CH₂O)ₓ—, etc. x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

R₁ and/or R₂ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁ and/or R₂, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable.

R₁ and/or R₂ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁ and/or R₂ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁ and/or R₂ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In an embodiment, not all of each R₁ and R₂ is hydrogen. In embodiments, not all of R₁ and R₂ are hydrogen.

In some implementations, the polymeric backbone of the polyimide may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyimide backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pendant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc.

Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

R₆ may be selected from any tetra-valent radical from any group consisting of a greater than two carbon aliphatic radical, monoaromatic radical, condensed polyaromatic radical, noncondensed polyaromatic radical, etc. Some nonlimiting examples of R₆ may be represented by the following formulas:

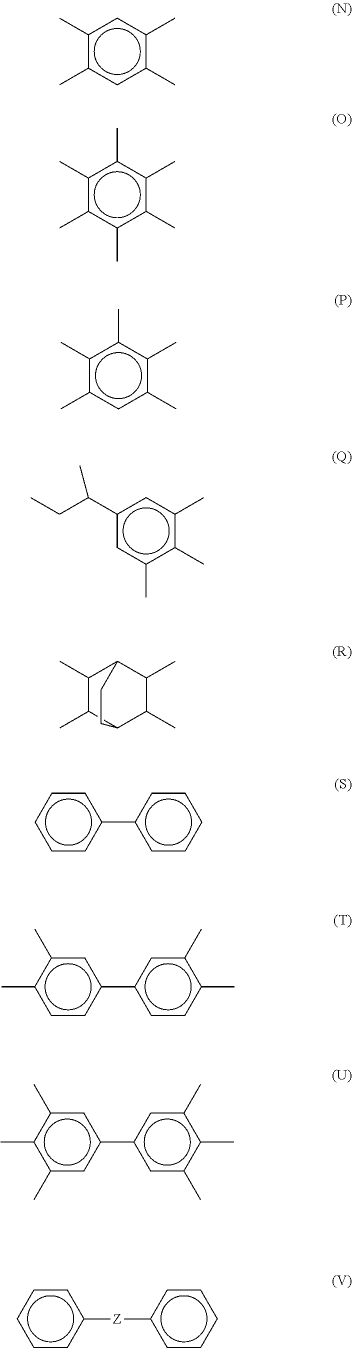

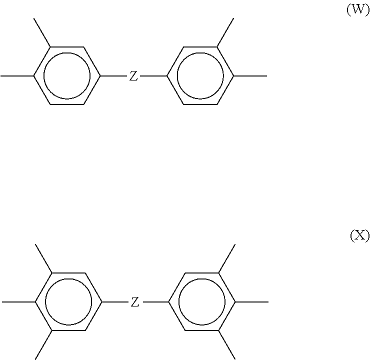

Some non-limiting examples of Z may be —(CH₂)ₓ—, —C(═O)—, —O—, —S(═O)₂—, —C(CF₃)₂—, —Si(CH₃)₂—, —C(CH₃)₂, etc. x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

Polyimide polymers can be prepared by any process known in the art. It is intended that the definition of polyimide polymer(s) includes any copolymer. The polyimide may be prepared in the presence of other chemicals or polymers (e.g. surface-active agents, initiators, chain termination agents, polymers, solvents, catalysts, etc.). The polyimide may be treated post polymerization (e.g. grafting, crosslinking, other modifications or functionalizations, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

Polyitaconates (Polymethylene Succinates)

The compositions of the present disclosure may include a carrier selected from the group consisting of polyitaconate polymers which may include any type of polyitaconate represented by the general structure comprising of recurring units represented by formula 33:

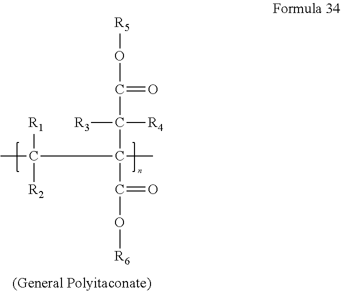

In formula 34, n is an integer representing the number of repeating units and is most generally 1-20,000, inclusive, and R₁, R₂, R₃, R₄, R₅ and/or R₆ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl.

To further define R₁, R₂, R₃, R₄, R₅ and/or R₆, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Groups defined by R₁, R₂, R₃, R₄, R₅ and/or R₆ may include heteroatoms.

R₁, R₂, R₃, R₄, R₅ and/or R₆ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁, R₂, R₃, R₄, R₅ and/or R₆ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁, R₂, R₃, R₄, R₅ and/or R₆ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In an embodiment, not all of each R₁-R₆ is hydrogen. In an embodiment, not all of R₁-R₆ are hydrogens.

In some implementations, the polymeric backbone of the polyitaconate may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyitaconate backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyitaconate polymers can be prepared by any process known in the art. It is intended that the definition of polyitaconate polymer(s) includes any copolymer. The polyitaconate may be prepared in the presence of other chemicals or polymers (e.g. surface-active agents, initiators, chain termination agents, polymers, solvents, catalysts, etc.). The polyitaconate may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyitaconate may be utilized for this invention. Some types of polyitaconates may include, but are limited to, poly(dimethyl itaconate), poly(di(n-propyl) itaconate)], poly[di(n-butyl) itaconate, poly[di(n-hexyl) itaconate], etc.

Polyketones (Polyetherketones)

The present invention may include a carrier selected from the group consisting of polyketone polymers which may include any type of polyketone represented by the general structure comprising of recurring units represented by Formula 35:

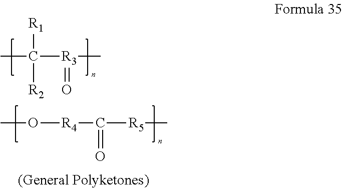

In formula 35, each n independently represents the number of repeating units and is most generally 1-20,000, inclusively, and R₃, R₄ and/or R₅ may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 arylenediyl, C3-200 alkarylenediyl, C3-200 aralkylenediyl. To further define R₃, R₄ and/or R₅, any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any arylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable. Groups defined by R₁, R₂, R₃, R₄ and/or R₅ may include heteroatoms

R₃, R₄ and/or R₅ may also be independently selected from a haloalkanediyl; haloalkenediyl; haloalkynediyl; halocycloalkanediyl; halocycloalkenediyl; halocycloalkynediyl; haloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxyarylenediyl; alkoxyalkarylenediyl; alkoxyaralkylenediyl; aminoalkanediyl; aminoalkenediyl; aminoalkynediyl; aminocycloalkanediyl; aminocycloalkenediyl; aminocycloalkynediyl; aminoarylenediyl; aminoalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfoarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphoarylenediyl; phosphoalkarylenediyl; phosphoaralkylenediyl; mono-, di- and trialkylamino-(alkanediyl, alkenediyl, alkynediyl, cycloalkanediyl, cycloalkenediyl, cycloalkynediyl, arylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, arylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, arylenediyl, alkarylenediyl or aralkylenediyl); arylamino-(alkanediyl, alkenediyl, alkynediyl, cycloalkanediyl, cycloalkenediyl, cycloalkynediyl, arylenediyl, alkarylenediyl or aralkylenediyl); arylsulfo-(alkanediyl, alkenediyl, alkynediyl, cycloalkanediyl, cycloalkenediyl, cycloalkynediyl, arylenediyl, alkarylenediyl or aralkylenediyl); arylphospho-(alkanediyl, alkenediyl, alkynediyl, cycloalkanediyl, cycloalkenediyl, cycloalkynediyl, arylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, arylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, arylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, arylenediyl, alkarylenediyl or aralkylenediyl).

R₃, R₄ and/or R₅ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; arylenediyl; alkarylenediyl or aralkylenediyl; acylamino; acylsulfo; acylphospho; alkylamino, alkylsulfo, alkylphospho, and the like.

R₃, R₄ and/or R₅ may also be selected from any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In certain implementations, R₃, R₄ and/or R₅ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through X as well as other such as, but not limited to, —(CH₂)ₓ—, —C(═O)—, —O—, —S(═O)₂—, —C(CF₃)₂—, —Si(CH₃)₂—, —C(CH₃)₂, —O(CH₂)_{X}O—, —(CH₂O)ₓ—, etc. x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

R₁ and/or R₂ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁ and/or R₂, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable.

Groups R₁ and/or R₂ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁ and/or R₂ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁ and/or R₂ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In an embodiment, not all of each R₁ or R₂ is hydrogen. In an embodiment, not all of R₁ and R₂ are hydrogens.

In some implementations, the polymeric backbone of the polyketone may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyketone backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyketone polymers can be prepared by any process known in the art. It is intended that the definition of polyketone includes any copolymer. The polyketones may be prepared in the presence of other chemicals or polymers (e.g. surface-active agents, initiators, chain termination agents, polymers, solvents, catalysts, etc.). The polyketones may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyketones may be utilized for this invention. Some types of polyketones include, but are not limited to, poly(ether ketone), poly(ether ketone ketone), poly(ether ether ketone), poly(ethyleneketone), poly(propyleneketone), etc.

Polynitrile

The compositions of the present disclosure may include a carrier selected from the group consisting of polynitrile polymers which may include any type of polynitrile represented by the general structure comprising of recurring units represented by Formula 36:

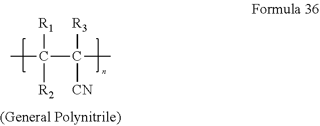

In formula 36, n is an integer that represents the number of repeating units and is most generally 1-20,000 inclusive, and R₁, R₂ and/or R₃ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁, R₂ and/or R₃, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Groups defined by R₁, R₂ and/or R₃ may include heteroatoms.

R₁, R₂ and/or R₃ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁, R₂ and/or R₃ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁, R₂ and/or R₃ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In an embodiment, not all of each R₁-R₃ is hydrogen. In an embodiment, not all of R₁-R₃ are hydrogen.

In some implementations, the polymeric backbone of the polynitrile may be substituted within the backbone with groups that may include, but are not limited to, any form (e.g. divalent, etc.) of those listed above. It may also be understood that the backbone(s) of branches off of the main polymer chain may also be substituted with similar groups. Any part of the polynitrile backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc.

Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polynitrile polymers can be prepared by any process known in the art. It is intended that the definition of polynitrile polymer(s) includes any copolymer. The polynitriles may be prepared in the presence of other chemicals or polymers (e.g. surface-active agents, initiators, chain termination agents, polymers, solvents, catalysts, etc.). The polynitriles may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polynitriles may be utilized for this invention. Some types of polynitriles include, but are not limited to, poly(acrylonitrile), poly(methacrylonitrile), etc.

Polyolefins

The present invention may include a carrier selected from the group consisting of polyolefin polymers which may include any type of polyolefin represented by the general structure comprising of recurring units represented by formula 37:

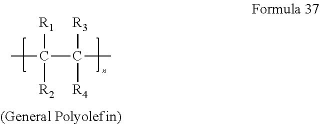

In formula 37, n is an integer representing the number of repeating units and is most generally 1-20,000 inclusive, and R₁, R₂, R₃ and/or R₄ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁, R₂, R₃ and/or R₄, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Groups defined by R₁, R₂, R₃ and/or R₄ may include heteroatoms.

R₁, R₂, R₃ and/or R₄ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁, R₂, R₃ and/or R₄ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁, R₂, R₃ and/or R₄ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁-R₄ is hydrogen. In embodiments, not all of R₁-R₄ are hydrogen.

In some implementations, the polymeric backbone of the polyolefin may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyolefin backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc.

Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyolefin polymers can be prepared by any process known in the art. It is intended that the definition of polyolefin polymer(s) includes any copolymer. The polyolefin may be prepared in the presence of other chemicals or polymers (e.g. surface-active agents, initiators, chain termination agents, polymers, solvents, catalysts, etc.). The polyolefin may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyolefins may be utilized for this invention. Some types of polyolefin include, but are not limited to, poly(butylene), poly(butyl ethylene), poly(cyclo hexyl ethylene), poly(ethylene), poly(heptyl ethylene), poly(hexyl ethylene), poly(isobutene), poly(isobutyl ethylene), poly(isopropyl ethylene), poly(2-methylbutene), poly(octylethylene), poly(pentylethylene), poly(propylene), poly(propylethylene), poly(tert-butyl ethylene), etc.

### Polyphenylenes

The present invention may include a carrier selected from the group consisting of polyphenylene polymers which may include any type of polyphenylene represented by the general structure comprising of recurring units represented by Formula 38:

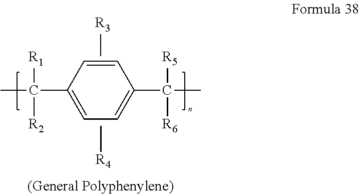

In formula 38, n is an integer representing the number of repeating units and is most generally 1-20,000, inclusively, and R₁, R₂, R₃, R₄, R₅ and/or R₆ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁, R₂, R₃, R₄, R₅ and/or R₆, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Groups defined by R₁, R₂, R₃, R₄, R₅ and/or R₆ may include heteroatoms.

R₁, R₂, R₃, R₄, R₅ and/or R₆ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁, R₂, R₃, R₄, R₅ and/or R₆ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁, R₂, R₃, R₄, R₅ and/or R₆ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁-R₆ is hydrogen. In embodiments, not all of R₁-R₆ are hydrogen.

In some implementations, the polymeric backbone of the polyphenylene may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyphenylene backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pendant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc.

Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyphenylene polymers can be prepared by any process known in the art. It is intended that the definition of polyphenylene polymer(s) includes any copolymer. The polyphenylene may be prepared in the presence of other chemicals or polymers (e.g. surface-active agents, initiators, chain termination agents, polymers, solvents, catalysts, etc.). The polyphenylene may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyphenylenes be utilized for this invention. Some types of polyphenylene include, but are not limited to, poly(p-phenylene), polyp-xylene), poly(2-chloro-p-xylene), etc.

Polyphenylethers (Polyphenleneoxide)

The compositions of the present disclosure may include a carrier selected from the group consisting of polyphenylether polymers which may include any type of polyphenylether represented by the general structure comprising of recurring units represented by formula 38:

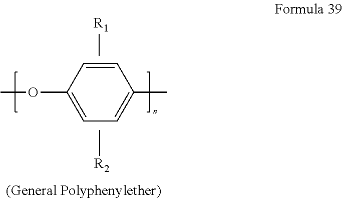

In formula 39, R₁ and/or R₂ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁ and/or R₂, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Groups defined by R₁ and/or R₂ may include heteroatoms.

R₁ and/or R₂ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁ and/or R₂ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁ and/or R₂ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁ and/or R₂ is hydrogen. In embodiments, not all of R₁ and R₂ are hydrogen. In a specific embodiment, one or both of R₁ and R₂ are hydrogens.

In some implementations, the polymeric backbone of the polyphenylether may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyphenylether backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyphenylether polymers can be prepared by any process known in the art. It is intended that the definition of polyphenylether polymer(s) includes any copolymer. The polyphenylether may be prepared in the presence of other chemicals or polymers (e.g. surface-active agents, initiators, chain termination agents, polymers, solvents, catalysts, etc.). The polyphenylether may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyphenylethers may be utilized for this invention. Some types of polyphenylether polymers include, but are not limited to, poly(2,6-dimethyl-p-phenylene oxide), poly(2,6-diphenyl-p-phenylene oxide), poly)p-phenylene oxide), etc.

Polyphosphazenes

(Polyorganophosphazenes)

The compositions of the present disclosure include a carrier selected from the group consisting of polyphosphazene polymers which may include any type of polyphosphazene represented by the general structure comprising of recurring units represented by formula 40:

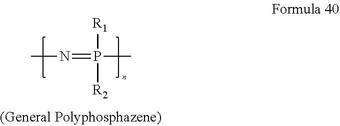

In formula 40, n is an integer representing the number of repeating units and is most generally 1-20,000, inclusive, and R₁ and/or R₂ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁ and/or R₂, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Groups defined by R₁ and/or R₂ may include heteroatoms.

R₁ and/or R₂ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁ and/or R₂ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁ and/or R₂ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁ and R₂ is hydrogen. In embodiments, not all of R₁ and R₂ are hydrogen.

In some implementations, the polymeric backbone of the polyphosphazene may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyphosphazene backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyphosphazene polymers can be prepared by any process known in the art. It is intended that the definition of polyphosphazene polymer(s) includes any copolymer. The polyphosphazene may be prepared in the presence of other chemicals or polymers (e.g. surface-active agents, initiators, chain termination agents, polymers, solvents, catalysts, etc.). The polyphosphazene may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyphosphazenes may be utilized for this disclosure. Some types of polyphosphazene polymers include, but are not limited to, poly(dichlorophosphazene), poly[bis(2-hydroxyethylmethacrylate)phosphazene, poly(bis(phenoxy)phosphazene, poly(2,2′-dioxybiphenyl)phosphazene, poly[di(carboxylatophenoxy)phosphazene, poly(bis(ethylglycinate)phosphazene, poly[(imidazoyl)(methylphenoxy)phosphazene], poly[(p-methylphenoxy)(ethylglycinato)]phosphazene, poly[bis(carboxyphenoxy)]phosphazene, poly[bis(methylamino)]phosphazene, poly[bis(trifluoroethoxy)]phosphazene, poly[(2-dimethylaminoethyamino)]phosphazene, poly[(imidazole)(DMAEA)]phosphazene, etc.

Polyphosphoester

(Polyphosphate/Polyphosphonate/Polyphosphite)

The polyphosphoester or polyphosphate/polyphosphonate/polyphosphite polymer composition comprises of the recurring monomeric structural units in formula 41.

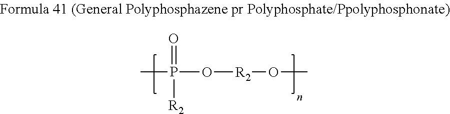

In formula 41, n is an integer representing the number of repeating units and is most generally 1-20,000 inclusive, and R₂ may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 arylenediyl, C3-200 alkarylenediyl, C3-200 aralkylenediyl. To further define R₂, any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any arylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable. Groups defined by R₁ and/or R₂ may include heteroatoms.

R₂ may also be independently selected from a haloalkanediyl; haloalkenediyl; haloalkynediyl; halocycloalkanediyl; halocycloalkenediyl; halocycloalkynediyl; haloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxyarylenediyl; alkoxyalkarylenediyl; alkoxyaralkylenediyl; aminoalkanediyl; aminoalkenediyl; aminoalkynediyl; aminocycloalkanediyl; aminocycloalkenediyl; aminocycloalkynediyl; aminoarylenediyl; aminoalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfoarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphoarylenediyl; phosphoalkarylenediyl; phosphoaralkylenediyl; mono-, di- and trialkylamino-(alkanediyl, alkenediyl, alkynediyl, cycloalkanediyl, cycloalkenediyl, cycloalkynediyl, arylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, arylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, arylenediyl, alkarylenediyl or aralkylenediyl); arylamino-(alkanediyl, alkenediyl, alkynediyl, cycloalkanediyl, cycloalkenediyl, cycloalkynediyl, arylenediyl, alkarylenediyl or aralkylenediyl); arylsulfo-(alkanediyl, alkenediyl, alkynediyl, cycloalkanediyl, cycloalkenediyl, cycloalkynediyl, arylenediyl, alkarylenediyl or aralkylenediyl); arylphospho-(alkanediyl, alkenediyl, alkynediyl, cycloalkanediyl, cycloalkenediyl, cycloalkynediyl, arylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, arylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, arylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, arylenediyl, alkarylenediyl or aralkylenediyl).

R₂ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; arylenediyl; alkarylenediyl or aralkylenediyl; acylamino; acylsulfo; acylphospho; alkylamino, alkylsulfo, alkylphospho, and the like.

R₂ may also be selected from any amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In certain implementations, R₂ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through X as well as other such as, but not limited to, —(CH₂)ₓ—, —C(═O)—, —O—, —S(═O)₂—, —C(CF₃)₂—, —Si(CH₃)₂—, —C(CH₃)₂, —O(CH₂)_{X}O—, —(CH₂O)ₓ—, etc. x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

R₁ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Group R₁ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. R₁ and R₂.

In some implementations, the polymeric backbone of the polyphosphoester may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyphosphoester backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

In some implementations, R₂ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. A single ring structure may include between 5 and 7 members. Some non-limiting examples include those listed in formulas A through M. Other non-limiting bridging group examples may include those in formulas N through X and Y—CC as well as other such as, but not limited to, —(CH₂)ₓ—, —C(═O)—, —O—, —S(═O)₂—, —C(CF₃)₂—, —Si(CH₃)₂—, —C(CH₃)₂, —O(CH₂)_{X}O—, —(CH₂O)ₓ—, etc. x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

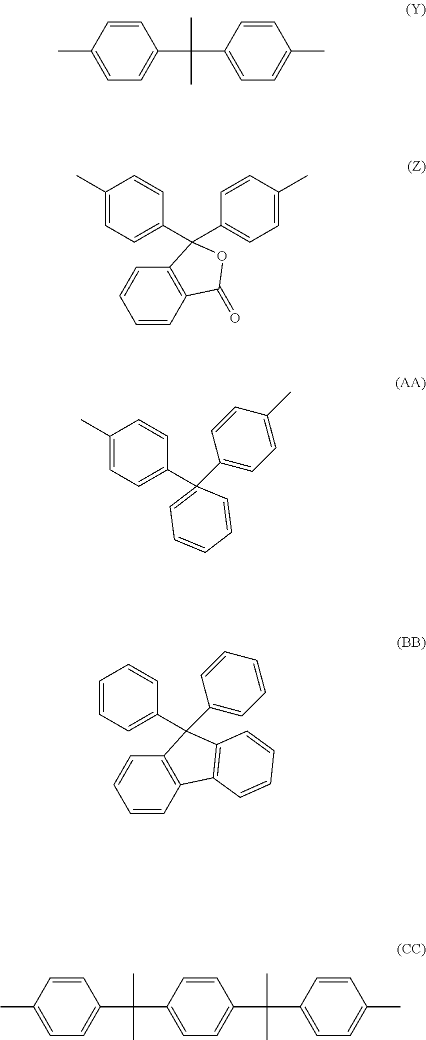

Polyphosphoester polymers can be prepared by any process known in the art. It is intended that the definition of polyphosphoester includes any copolymer. The polyphosphoester may be prepared in the presence of other chemicals or polymers (e.g. surface-active agents, initiators, chain termination agents, polymers, solvents, catalysts, etc.). The polyphosphoester may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

Polysiloxane

The compositions of the present disclosure may include a carrier selected from the group consisting of polysiloxane polymers which may include any type of polysiloxane represented by the general structure comprising of recurring units represented by Formula 42:

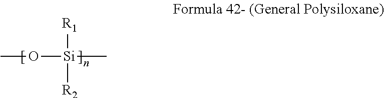

In formula 42, n is an integer representing the number of repeating units and most generally is 1-20,000 inclusive, and R₁ and/or R₂ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁ and/or R₂, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Groups defined by R₁ and/or R₂ may include heteroatoms.

R₁ and/or R₂ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁ and/or R₂ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁ and/or R₂ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. One or more of each R₁, or R₂ may be a hydrogen. In an embodiment, not each of R₁, or R₂ are hydrogens. In an embodiment, not all of R₁ or R₂ are hydrogens.

In some implementations, the polymeric backbone of the polysiloxane may be substituted within the backbone with groups that may include, but are not limited to, any form (e.g. divalent, etc.) of those listed above. It may also be understood that the backbone(s) of branches off of the main polymer chain may also be substituted with similar groups. Any part of the polysiloxane backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polysiloxane polymers can be prepared by any process known in the art. It is intended that the definition of polysiloxane polymer(s) includes any copolymer. The polysiloxane may be prepared in the presence of other chemicals or polymers (e.g. surface-active agents, initiators, chain termination agents, polymers, solvents, catalysts, etc.). The polysiloxane may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polysiloxanes may be utilized for this invention. Some types of polysiloxane polymers include, but are not limited to, poly(diethylsiloxane), poly(dimethylsiloxane), poly(methyphenylsiloxane), etc.

Polystyrenes (Polyphenylethylenes)

The present invention may include a carrier selected from the group consisting of polystyrene polymers which may include any type of polystyrene represented by the general structure comprising of recurring units represented by Formula 43:

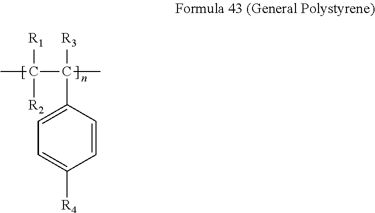

In formula 43, n is an integer representing the number of repeating units and is most generally 1-20,000 and R₁, R₂ and/or R₃ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 aryl, C3-200 aralkyl or C3-200 alkaryl. To further define R₁, R₂ and/or R₃, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any aryl moiety with a carbon backbone between 3 and 30 is preferable; any aralkyl moiety with a carbon backbone between 5 and 30 is preferable; any alkaryl moiety with a carbon backbone between 5 and 30 is preferable. Groups defined by R₁, R₂ and/or R₃ may include heteroatoms.

R₁, R₂ and/or R₃ may also be independently selected from a haloalkyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl; alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl or alkaryl); aminoacyl; sulfoacyl; phosphoacyl; N-(aryl, aryloxy, arylthiol, arylsulfinyl, arylsufonyl, arylphosphino or arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, aryl, aralkyl or alkaryl).

R₁, R₂ and/or R₃ may also be independently selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; acylamino; acylsulfo; acylphospho; alkylamino, alkylsulfo, alkylphospho, and the like.

R₁, R₂ and/or R₃ may also be selected from hydrogen or any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In an embodiment, one or more of R₁-R₃ are hydrogens. In an embodiment, not all of each R₁, R₂ and R₃ are hydrogens. In an embodiment, not all of R₁, R₂ and R₃ are hydrogens.

In some implementations, the polymeric backbone of the polystyrene may be substituted within the backbone with groups that may include, but are not limited to, any form (e.g. divalent, etc.) of those listed above. It may also be understood that the backbone(s) of branches off of the main polymer chain may also be substituted with similar groups. Any part of the polystyrene backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 aryl; C3-200 aralkyl; C3-200 alkaryl; haloalkanyl; haloalkenyl; haloalkynyl; halocycloalkyl; halocycloalkenyl; halocycloalkynyl; haloaryl; haloaralkyl; haloalkaryl; alkoxyalkyl; alkoxyalkenyl; alkoxyalkynyl; alkoxycycloalkyl: alkoxycycloalkenyl; alkoxycycloalkynyl; alkoxyaryl; alkoxyaralkyl; alkoxyalkaryl; aminoalkyl; aminoalkenyl; aminoalkynyl; aminocycloalkyl; aminocycloalkenyl; aminocycloalkynyl; aminoaryl; aminoaralkyl; aminoalkaryl; sulfoalkyl; sulfoalkenyl; sulfoalkynyl; sulfocycloalkyl; sulfocycloalkenyl; sulfocycloalkynyl; sulfoaryl; sulfoaralkyl; sulfoalkaryl; phosphoalkyl; phosphoalkenyl; phosphoalkynyl; phosphocycloalkyl; phosphocycloalkenyl; phosphocycloalkynyl; phosphoaryl; phosphoaralkyl; phosphoalkaryl; mono-, di- and trialkylamino-(alkyl, alkenyl, alkynyl, cycloalkyl, cycloalkenyl, cycloalkynyl, aryl, aralkyl or alkaryl); mono-, di- and trialkylsulfo-(alkyl, alkenyl, alkynyl, cycloalkyl, cycloalkenyl, cycloalkynyl, aryl, aralkyl or alkaryl); mono-, di- and trialkylphospho-(alkyl, alkenyl, alkynyl, cycloalkyl, cycloalkenyl, cycloalkynyl, aryl, aralkyl or alkaryl); arylamino-(alkyl, alkenyl, alkynyl, cycloalkyl, cycloalkenyl, cycloalkynyl, aryl, aralkyl or alkaryl); arylsulfo-(alkyl, alkenyl, alkynyl, cycloalkyl, cycloalkenyl, cycloalkynyl, aryl, aralkyl or alkaryl); arylphospho-(alkyl, alkenyl, alkynyl, cycloalkyl, cycloalkenyl, cycloalkynyl, aryl, aralkyl, or alkaryl); aminoacyl; sulfoacyl; phosphoacyl; N-(aryl, aryloxy, arylthiol, arylsulfinyl, arylsufonyl, arylphosphino, arylphosphono, and the like)amino-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)sulfo-(alkyl, alkenyl, alkynyl, cycloalkyl, cycloalkenyl, cycloalkynyl, aryl, aralkyl or alkaryl); N-(aryl, aryloxy, arylthiol, arylsulfinyl, arylsufonyl, arylphosphino, arylphosphono, and the like)phosphoro-(alkyl, alkenyl, alkynyl, cycloalkyl, cycloalkenyl, cycloalkynyl, aryl, aralkyl or alkaryl).

The pedant group may also be selected from any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, aryl, aralkyl or alkaryl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polystyrene polymers can be prepared by any process known in the art. It is intended that the definition of polystyrene polymer(s) includes any copolymer. The polystyrene may be prepared in the presence of other chemicals or polymers (e.g. surface-active agents, initiators, chain termination agents, polymers, solvents, catalysts, etc.). The polystyrene may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polystyrenes may be utilized for this invention. Some types of polystyrene polymers include, but are not limited to, poly(2-methoxystyrene), poly(α-methylstyrene), poly(methylstyrene), poly(3-methylstyrene), poly(4-methylstyrene), polystyrene, poly)4-tert-butylstyrene), etc.

Polysulfides (Polythioethers)

The compositions of the present disclosure may include a carrier selected from the group consisting of polysulfide polymers which may include any type of polysulfide represented by the general structure comprising of recurring units represented by Formula 44:

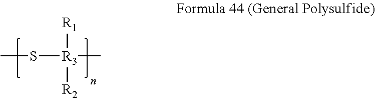

In formula 44, n is an integer representing the number of repeating units and is most generally 1-20,000 inclusively and R₃ may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 cycloarylenediyl, C3-200 alkarylenediyl, C3-200 aralkylenediyl. To further define R₃ any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any cycloarylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable. R₃ groups may include heteroatoms.

R₃ may also be independently selected from a haloalkanediyl; haloalkenediyl; haloalkynediyl; halocycloalkanediyl; halocycloalkenediyl; halocycloalkynediyl; halocycloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxycycloarylenediyl; alkoxyalkarylenediyl; alkoxyaralyenediyl; aminoalkanediyl; aminoalkenediyl; aminoalkynediyl; aminocycloalkanediyl; aminocycloalkenediyl; aminocycloalkynediyl; aminocycloarylenediyl; aminocycloalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfocycloarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphocycloarylenediyl; phosphoalkyarylenediyl; phosphoaralkylenediyl; mono-, di- and trialkylamino-(alkanediyl, alkenediyl, alkynediyl, cycloalkanediyl, cycloalkenediyl, cycloalkynediyl, cycloarylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, cycloarylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, cycloarylenediyl, alkarylenediyl or aralkylenediyl); arylamino-(alkanediyl, alkenediyl, alkynediyl, cycloalkanediyl, cycloalkenediyl, cycloalkynediyl, cycloarylenediyl, alkarylenediyl or aralkylenediyl); arylsulfo-(alkanediyl, alkenediyl, alkynediyl, cycloalkanediyl, cycloalkenediyl, cycloalkynediyl, cycloarylenediyl, alkarylenediyl or aralkylenediyl); arylphospho-(alkanediyl, alkenediyl, alkynediyl, cycloalkanediyl, cycloalkenediyl, cycloalkynediyl, cycloarylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, cycloarylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, cycloarylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, cycloarylenediyl, alkarylenediyl or aralkylenediyl).

R₃ may also be independently selected from any divalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; cycloarylenediyl; alkarylenediyl or aralkylenediyl; acylamino; acylsulfo; acylphospho; alkylamino, alkylsulfo, alkylphospho, and the like.

R₃ may also be selected from any divalent radical form of any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In some implementations, R₃ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through X or Y through CC as well as other such as, but not limited to, —(CH₂)ₓ—, —C(═O)—, —O—, —S(═O)₂—, —C(CF₃)₂—, —Si(CH₃)₂—, —C(CH₃)₂, —O(CH₂)_{X}O—, —(CH₂O)ₓ—, etc. x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

In formula 44, R₁ and/or R₂ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 cycloarylenyl, C3-200 alkarylenyl, C3-200 aralkylenyl. To further define R₁ and/or R₂, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any cycloarylenyl moiety with a carbon backbone between 3 and 30 is preferable; any alkarylenyl moiety with a carbon backbone between 5 and 30 is preferable; any aralkylenyl moiety with a carbon backbone between 3 and 30 is preferable. Groups defined by R₁ and/or R₂ may include heteroatoms.

R₁ and/or R₂ may also be independently selected from a haloalkyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkyl; alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloaryleneyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkanyl, cycloalkenyl, cycloalkynyl, cycloarylendiyl, alkarylenyl or aralkylenyl); mono-, di- and trialkylsulfo-(alkyl, alkenyl, alkynyl, cycloalkanyl, cycloalkenyl, cycloalkynyl, cycloarylenyl, alkarylenyl or aralkylenyl); mono-, di- and trialkylphospho-(alkyl, alkenyl, alkynyl, cycloalkanyl, cycloalkenyl, cycloalkynyl, cycloarylenyl, alkarylenyl or aralkylenyl); arylamino-(alkyl, alkenyl, alkynyl, cycloalkanyl, cycloalkenyl, cycloalkynyl, cycloarylenyl, alkarylenyl or aralkylenyl); arylsulfo-(alkyl, alkenyl, alkynyl, cycloalkanyl, cycloalkenyl, cycloalkynyl, cycloarylenyl, alkarylenyl or aralkylenyl); arylphospho-(alkyl, alkenyl, alkynyl, cycloalkanyl, cycloalkenyl, cycloalkynyl, cycloarylenyl, alkarylenyl or aralkylenyl); aminoacyl; sulfoacyl; phosphoacyl; N-(aryl, aryloxy, arylthiol, arylsulfinyl, arylsufonyl, arylphosphino, or arylphosphono, and the like)amino-(alkyl, alkenyl, alkynyl, cycloalkyl, cycloalkenyl, cycloalkynyl, cycloarylenyl, alkarylenyl or aralkylenyl); N-(aryl, aryloxy, arylthiol, arylsulfinyl, arylsufonyl, arylphosphino, or arylphosphono, and the like)sulfo-(alkyl, alkenyl, alkyndiyl, cycloalkyl, cycloalkenyl, cycloalkynyl, cycloarylenyl, alkarylenyl or aralkylenyl, and the like); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, cycloarylenyl, alkarylenyl or aralkylenyl).

R₁ and/or R₂ may also be independently selected from any monovalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; alkenyl; alkynyl; cycloalkyl; cycloalkenyl; cycloalkynyl; cycloarylenyl; alkarylenyl or aralkylenyl; acylamino; acylsulfo; acylphospho; alkylamino, alkylsulfo, alkylphospho, and the like.

R₁ and/or R₂ may also be selected from hydrogen or any monovalent radical form of any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁ and R₂ is hydrogen. In embodiments, not all of R₁ and R₂ are hydrogen. In embodiments, one or more of R₁ and R₂ is hydrogen.

In some implementations, the polymeric backbone of the polysulfide may be substituted within the backbone with groups that may include, but are not limited to, any form (e.g. divalent, etc.) of those listed above. It may also be understood that the backbone(s) of branches off of the main polymer chain may also be substituted with similar groups. Any part of the polysulfide backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 cycloarylenyl; C3-200 alkarylenyl; C3-200 aralkylenyl; haloalkanyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkanyl: alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloarylenyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkyl, cycloalkenyl, cycloalkynyl, cycloarylenyl, alkarylenyl or aralkylenyl); mono-, di- and trialkylsulfo-(alkyl, alkenyl, alkynyl, cycloalkyl, cycloalkenyl, cycloalkyne, cycloarylene, alkarylene or aralkylene); mono-, di- and trialkylphospho-(alkane, alkene, alkyne, cycloalkane, cycloalkene, cycloalkyne, cycloarylene, alkarylene or aralkylene); arylamino-(alkane, alkene, alkyne, cycloalkane, cycloalkene, cycloalkyne, cycloarylene, alkarylene or aralkylene); arylsulfo-(alkane, alkene, alkyne, cycloalkane, cycloalkene, cycloalkyne, cycloarylene, alkarylene or aralkylene); arylphospho-(alkane, alkene, alkyne, cycloalkane, cycloalkene, cycloalkyne, cycloarylene, alkarylene or aralkylene); aminoacyl; sulfoacyl; phosphoacyl; N-(aryl, aryloxy, arylthiol, arylsulfinyl, arylsufonyl, arylphosphino, arylphosphono, and the like)amino-(alkane, alkene, alkyne, cycloalkaned, cycloalkene, cycloalkyne, cycloarylene, alkarylene or aralkylene); N-(aryl, aryloxy, arylthiol, arylsulfinyl, arylsufonyl, arylphosphino, arylphosphono, and the like)sulfo-(alkane, alkene, alkyne, cycloalkane, cycloalkene, cycloalkyne, cycloarylene, alkarylene or aralkylene); N-(aryl, aryloxy, arylthiol, arylsulfinyl, arylsufonyl, arylphosphino, arylphosphono, and the like)phosphoro-alkane, alkene, alkyne, cycloalkane, cycloalkene, cycloalkyned, cycloarylene, alkarylene or aralkylene.

The pedant group may also be selected from any monovalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, cycloarylenyll, alkarylenyl or aralkylenyl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc.

Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc. n is an independent integer between 1 and 20,000.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polysulfide polymers can be prepared by any process known in the art. It is intended that the definition of polysulfide polymer(s) includes any copolymer. The polysulfides may be prepared in the presence of other chemicals or polymers (e.g. surface-active agents, initiators, chain termination agents, polymers, solvents, catalysts, etc.). The polysulfides may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polysulfides may be utilized for this invention. Some types of polysulfide polymers include, but are not limited to, poly(thio-1,4-phenylene), poly(ethylene sulfide), polypropylene sulfide), etc.

Polyethersulfones (Polysulfones)

The present invention may include a carrier selected from the group consisting of polyethersulfone polymers which may include any type of polyethersulfone represented by the general structure comprising of recurring units represented by Formula 45:

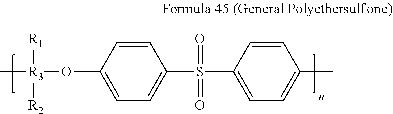

In formula 45, n is an integer representing the number of repeating units and most generally ranges from 1-20,000, inclusive, and R₃ may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 cycloarylenediyl, C3-200 alkarylenediyl, C3-200 aralkylenediyl. To further define R₃ any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any cycloarylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable. Groups defined by R₁, R₂ and/or R₃ may include heteroatoms.

R₃ may also be independently selected from a haloalkanediyl; haloalkenediyl; haloalkynediyl; halocycloalkanediyl; halocycloalkenediyl; halocycloalkynediyl; halocycloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxycycloarylenediyl; alkoxyalkarylenediyl; alkoxyaralyenediyl; aminoalkanediyl; aminoalkenediyl; aminoalkynediyl; aminocycloalkanediyl; aminocycloalkenediyl; aminocycloalkynediyl; aminocycloarylenediyl; aminocycloalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfocycloarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphocycloarylenediyl; phosphoalkyarylenediyl; phosphoaralkylenediyl; mono-, di- and trialkylamino-(alkanediyl, alkenediyl, alkynediyl, cycloalkanediyl, cycloalkenediyl, cycloalkynediyl, cycloarylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, cycloarylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, cycloarylenediyl, alkarylenediyl or aralkylenediyl); arylamino-(alkanediyl, alkenediyl, alkynediyl, cycloalkanediyl, cycloalkenediyl, cycloalkynediyl, cycloarylenediyl, alkarylenediyl or aralkylenediyl); arylsulfo-(alkanediyl, alkenediyl, alkynediyl, cycloalkanediyl, cycloalkenediyl, cycloalkynediyl, cycloarylenediyl, alkarylenediyl or aralkylenediyl); arylphospho-(alkanediyl, alkenediyl, alkynediyl, cycloalkanediyl, cycloalkenediyl, cycloalkynediyl, cycloarylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, cycloarylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, cycloarylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, cycloarylenediyl, alkarylenediyl or aralkylenediyl).

R₃ may also be independently selected from any divalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; cycloarylenediyl; alkarylenediyl or aralkylenediyl; acylamino; acylsulfo; acylphospho; alkylamino, alkylsulfo, alkylphospho, and the like.

R₃ may also be selected from any divalent radical form of any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In some implementations, R₃ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through X and Y through CC as well as other such as, but not limited to, —(CH₂)ₓ—, —C(═O)—, —O—, —S(═O)₂—, —C(CF₃)₂—, —Si(CH₃)₂—, —C(CH₃)₂, —O(CH₂)_{X}O—, —(CH₂O)ₓ—, etc. x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

In formula 45, R₁ and/or R₂ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 cycloarylenyl, C3-200 alkarylenyl, C3-200 aralkylenyl. To further define R₁ and/or R₂, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyll moiety with a carbon backbone between 7 and 30 is preferable; any cycloalkynyl moiety with a carbon backbone between 7 and 30 is preferable; any cycloarylenyl moiety with a carbon backbone between 3 and 30 is preferable; any alkarylenyl moiety with a carbon backbone between 5 and 30 is preferable; any aralkylenyl moiety with a carbon backbone between 3 and 30 is preferable.

R₁ and/or R₂ may also be independently selected from a haloalkyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkyl; alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloaryleneyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkanyl, cycloalkenyl, cycloalkynyl, cycloarylendiyl, alkarylenyl or aralkylenyl); mono-, di- and trialkylsulfo-(alkyl, alkenyl, alkynyl, cycloalkanyl, cycloalkenyl, cycloalkynyl, cycloarylenyl, alkarylenyl or aralkylenyl); mono-, di- and trialkylphospho-(alkyl, alkenyl, alkynyl, cycloalkanyl, cycloalkenyl, cycloalkynyl, cycloarylenyl, alkarylenyl or aralkylenyl); arylamino-(alkyl, alkenyl, alkynyl, cycloalkanyl, cycloalkenyl, cycloalkynyl, cycloarylenyl, alkarylenyl or aralkylenyl); arylsulfo-(alkyl, alkenyl, alkynyl, cycloalkanyl, cycloalkenyl, cycloalkynyl, cycloarylenyl, alkarylenyl or aralkylenyl); arylphospho-(alkyl, alkenyl, alkynyl, cycloalkanyl, cycloalkenyl, cycloalkynyl, cycloarylenyl, alkarylenyl or aralkylenyl); aminoacyl; sulfoacyl; phosphoacyl; N-(aryl, aryloxy, arylthiol, arylsulfinyl, arylsufonyl, arylphosphino, or arylphosphono, and the like)amino-(alkyl, alkenyl, alkynyl, cycloalkyl, cycloalkenyl, cycloalkynyl, cycloarylenyl, alkarylenyl or aralkylenyl); N-(aryl, aryloxy, arylthiol, arylsulfinyl, arylsufonyl, arylphosphino, or arylphosphono, and the like)sulfo-(alkyl, alkenyl, alkyndiyl, cycloalkyl, cycloalkenyl, cycloalkynyl, cycloarylenyl, alkarylenyl or aralkylenyl, and the like); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, cycloarylenyl, alkarylenyl or aralkylenyl).

R₁ and/or R₂ may also be independently selected from any monovalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; alkenyl; alkynyl; cycloalkyl; cycloalkenyl; cycloalkynyl; cycloarylenyl; alkarylenyl or aralkylenyl; acylamino; acylsulfo; acylphospho; alkylamino, alkylsulfo, alkylphospho, and the like.

R₁ and/or R₂ may also be selected from hydrogen any monovalent radical form of any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁ and R₂ is hydrogen. In embodiments, not all of R₁, and R₂ are hydrogen. In embodiments, one or more of R₁ and R₂ is hydrogen.

In some implementations, the polymeric backbone of the polyethersulfone may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyethersulfone backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 cycloarylenyl; C3-200 alkarylenyl; C3-200 aralkylenyl; haloalkanyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkanyl: alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloarylenyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkyl, cycloalkenyl, cycloalkynyl, cycloarylenyl, alkarylenyl or aralkylenyl); mono-, di- and trialkylsulfo-(alkyl, alkenyl, alkynyl, cycloalkyl, cycloalkenyl, cycloalkyne, cycloarylene, alkarylene or aralkylene); mono-, di- and trialkylphospho-(alkane, alkene, alkyne, cycloalkane, cycloalkene, cycloalkyne, cycloarylene, alkarylene or aralkylene); arylamino-(alkane, alkene, alkyne, cycloalkane, cycloalkene, cycloalkyne, cycloarylene, alkarylene or aralkylene); arylsulfo-(alkane, alkene, alkyne, cycloalkane, cycloalkene, cycloalkyne, cycloarylene, alkarylene or aralkylene); arylphospho-(alkane, alkene, alkyne, cycloalkane, cycloalkene, cycloalkyne, cycloarylene, alkarylene or aralkylene); aminoacyl; sulfoacyl; phosphoacyl; N-(aryl, aryloxy, arylthiol, arylsulfinyl, arylsufonyl, arylphosphino, arylphosphono, and the like)amino-(alkane, alkene, alkyne, cycloalkaned, cycloalkene, cycloalkyne, cycloarylene, alkarylene or aralkylene); N-(aryl, aryloxy, arylthiol, arylsulfinyl, arylsufonyl, arylphosphino, arylphosphono, and the like)sulfo-(alkane, alkene, alkyne, cycloalkane, cycloalkene, cycloalkyne, cycloarylene, alkarylene or aralkylene); N-(aryl, aryloxy, arylthiol, arylsulfinyl, arylsufonyl, arylphosphino, arylphosphono, and the like)phosphoro-alkane, alkene, alkyne, cycloalkane, cycloalkene, cycloalkyned, cycloarylene, alkarylene or aralkylene.

The pedant group may also be selected from any monovalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, cycloarylenyll, alkarylenyl or aralkylenyl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc.

Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyethersulfone polymers can be prepared by any process known in the art. It is intended that the definition of polyethersulfone polymer(s) includes any copolymer. The polyethersulfone may be prepared in the presence of other chemicals or polymers (e.g. surface-active agents, initiators, chain termination agents, polymers, solvents, catalysts, etc.). The polyethersulfone may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyethersulfones may be utilized for this invention. Some types of polyethersulfone polymers include, but are not limited to, poly(ether sulfone), poly(ethersulfone), poly(phenylsulfone), bisphenol A polysulfone, etc.

Polyurethanes

The present invention may include a carrier selected from the group consisting of polyurethane polymers which may include any type of polyurethane represented by the general structure comprising of recurring units represented by Formula 46:

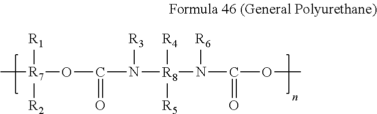

In formula 46, n is an integer representing the number of repeating units and is most generally 1-20,000 inclusive, and R₇ and/or R₈ may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 cycloarylenediyl, C3-200 alkarylenediyl, C3-200 aralkylenediyl. To further define R₇ and/or R₈ any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any cycloarylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable. Groups defined by R₁, R₂, R₃, R₄, R₅, R₆, R₇ and/or R₈ may include heteroatoms.

R₇ and/or R₈ may also be independently selected from a haloalkanediyl; haloalkenediyl; haloalkynediyl; halocycloalkanediyl; halocycloalkenediyl; halocycloalkynediyl; halocycloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxycycloarylenediyl; alkoxyalkarylenediyl; alkoxyaralyenediyl; aminoalkanediyl; aminoalkenediyl; aminoalkynediyl; aminocycloalkanediyl; aminocycloalkenediyl; aminocycloalkynediyl; aminocycloarylenediyl; aminocycloalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfocycloarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphocycloarylenediyl; phosphoalkyarylenediyl; phosphoaralkylenediyl; mono-, di- and trialkylamino-(alkanediyl, alkenediyl, alkynediyl, cycloalkanediyl, cycloalkenediyl, cycloalkynediyl, cycloarylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, cycloarylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, cycloarylenediyl, alkarylenediyl or aralkylenediyl); arylamino-(alkanediyl, alkenediyl, alkynediyl, cycloalkanediyl, cycloalkenediyl, cycloalkynediyl, cycloarylenediyl, alkarylenediyl or aralkylenediyl); arylsulfo-(alkanediyl, alkenediyl, alkynediyl, cycloalkanediyl, cycloalkenediyl, cycloalkynediyl, cycloarylenediyl, alkarylenediyl or aralkylenediyl); arylphospho-(alkanediyl, alkenediyl, alkynediyl, cycloalkanediyl, cycloalkenediyl, cycloalkynediyl, cycloarylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, cycloarylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, cycloarylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, cycloarylenediyl, alkarylenediyl or aralkylenediyl).

R₇ and/or R₈ may also be independently selected from any divalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; cycloarylenediyl; alkarylenediyl or aralkylenediyl; acylamino; acylsulfo; acylphospho; alkylamino, alkylsulfo, alkylphospho, and the like.

R₇ and/or R₈ may also be selected from any divalent form of any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In some implementations, R₇ and/or R₈ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through W as well as other such as, but not limited to, —(CH₂)ₓ—, —C(═O)—, —O—, —S(═O)₂—, —C(CF₃)₂—, —Si(CH₃)₂—, —C(CH₃)₂, —O(CH₂)_{X}O—, —(CH₂O)ₓ—, etc. x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

In formula 46, R₁, R₂, R₃, R₄, R₅ and/or R₆ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 cycloarylenyl, C3-200 alkarylenyl, C3-200 aralkylenyl. To further define R₁, R₂, R₃, R₄, R₅ and/or R₆, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any cycloarylenyl moiety with a carbon backbone between 3 and 30 is preferable; any alkarylenyl moiety with a carbon backbone between 5 and 30 is preferable; any aralkylenyl moiety with a carbon backbone between 3 and 30 is preferable. R₁, R₂, R₃, R₄, R₅ and/or R₆ may also be independently selected from a haloalkyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkyl; alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloaryleneyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkanyl, cycloalkenyl, cycloalkynyl, cycloarylendiyl, alkarylenyl or aralkylenyl); mono-, di- and trialkylsulfo-(alkyl, alkenyl, alkynyl, cycloalkanyl, cycloalkenyl, cycloalkynyl, cycloarylenyl, alkarylenyl or aralkylenyl); mono-, di- and trialkylphospho-(alkyl, alkenyl, alkynyl, cycloalkanyl, cycloalkenyl, cycloalkynyl, cycloarylenyl, alkarylenyl or aralkylenyl); arylamino-(alkyl, alkenyl, alkynyl, cycloalkanyl, cycloalkenyl, cycloalkynyl, cycloarylenyl, alkarylenyl or aralkylenyl); arylsulfo-(alkyl, alkenyl, alkynyl, cycloalkanyl, cycloalkenyl, cycloalkynyl, cycloarylenyl, alkarylenyl or aralkylenyl); arylphospho-(alkyl, alkenyl, alkynyl, cycloalkanyl, cycloalkenyl, cycloalkynyl, cycloarylenyl, alkarylenyl or aralkylenyl); aminoacyl; sulfoacyl; phosphoacyl; N-(aryl, aryloxy, arylthiol, arylsulfinyl, arylsufonyl, arylphosphino, or arylphosphono, and the like)amino-(alkyl, alkenyl, alkynyl, cycloalkyl, cycloalkenyl, cycloalkynyl, cycloarylenyl, alkarylenyl or aralkylenyl); N-(aryl, aryloxy, arylthiol, arylsulfinyl, arylsufonyl, arylphosphino, or arylphosphono, and the like)sulfo-(alkyl, alkenyl, alkyndiyl, cycloalkyl, cycloalkenyl, cycloalkynyl, cycloarylenyl, alkarylenyl or aralkylenyl, and the like); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, cycloarylenyl, alkarylenyl or aralkylenyl).

R₁, R₂, R₃, R₄, R₅ and/or R₆ may also be independently selected from any form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; alkenyl; alkynyl; cycloalkyl; cycloalkenyl; cycloalkynyl; cycloarylenyl; alkarylenyl or aralkylenyl; acylamino; acylsulfo; acylphospho; alkylamino, alkylsulfo, alkylphospho, and the like.

R₁, R₂, R₃, R₄, R₅ and/or R₆ may also be selected from hydrogen or any monovalent form of any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁, R₂, R₃, R₄, R₅ and/or R₆ is hydrogen. In embodiments, not all of R₁, R₂, R₃, R₄, R₅, and/or R₆ is hydrogen. In embodiments, one or more of R₁, R₂, R₃, R₄, R₅ and/or R₆ is hydrogen.

In some implementations, the polymeric backbone of the polyurethane may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyurethane backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 cycloarylenyl; C3-200 alkarylenyl; C3-200 aralkylenyl; haloalkanyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkanyl; alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloarylenyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkyl, cycloalkenyl, cycloalkynyl, cycloarylenyl, alkarylenyl or aralkylenyl); mono-, di- and trialkylsulfo-(alkyl, alkenyl, alkynyl, cycloalkyl, cycloalkenyl, cycloalkyne, cycloarylene, alkarylene or aralkylene); mono-, di- and trialkylphospho-(alkane, alkene, alkyne, cycloalkane, cycloalkene, cycloalkyne, cycloarylene, alkarylene or aralkylene); arylamino-(alkane, alkene, alkyne, cycloalkane, cycloalkene, cycloalkyne, cycloarylene, alkarylene or aralkylene); arylsulfo-(alkane, alkene, alkyne, cycloalkane, cycloalkene, cycloalkyne, cycloarylene, alkarylene or aralkylene); arylphospho-(alkane, alkene, alkyne, cycloalkane, cycloalkene, cycloalkyne, cycloarylene, alkarylene or aralkylene); aminoacyl; sulfoacyl; phosphoacyl; N-(aryl, aryloxy, arylthiol, arylsulfinyl, arylsufonyl, arylphosphino, arylphosphono, and the like)amino-(alkane, alkene, alkyne, cycloalkaned, cycloalkene, cycloalkyne, cycloarylene, alkarylene or aralkylene); N-(aryl, aryloxy, arylthiol, arylsulfinyl, arylsufonyl, arylphosphino, arylphosphono, and the like)sulfo-(alkane, alkene, alkyne, cycloalkane, cycloalkene, cycloalkyne, cycloarylene, alkarylene or aralkylene); N-(aryl, aryloxy, arylthiol, arylsulfinyl, arylsufonyl, arylphosphino, arylphosphono, and the like)phosphoro-alkane, alkene, alkyne, cycloalkane, cycloalkene, cycloalkyned, cycloarylene, alkarylene or aralkylene.

The pedant group may also be selected from any monovalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, cycloarylenyll, alkarylenyl or aralkylenyl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc.

Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyurethane polymers can be prepared by any process known in the art. It is intended that the definition of polyurethane polymer(s) includes any copolymer. The polyurethane may be prepared in the presence of other chemicals or polymers (e.g. surface-active agents, initiators, chain termination agents, polymers, solvents, catalysts, etc.). The polyurethane may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyurethanes may be utilized for this invention. Some types of polyurethane polymers include, but are not limited to, poly[(diethylene glycol)-alt-(1,6-hexamethylene diisocyanate)], poly[(tetraethyene glycol)-alt-(1,6-hexamethylene diisocyanate)], poly[(1,4-butanediol)-alt-(4,4′-diphenylmethane diisocyanate)], poly[(ethylene glycol)-alt-(4,4′-diphenyl methane diisocyanate)], poly[(polytetrahydrofuran 1000)-alt-(4,4′-diphenylmethane diisocyanate)], etc.

Polyvinyl Alcohol

The compositions of the present disclosure may include a carrier selected from the group consisting of polyvinyl alcohol polymers which may include any type of polyvinyl alcohol represented by the general structure comprising of recurring units represented by Formula 47:

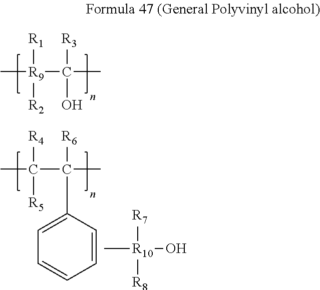

In formula 47, each n independently is an integer and represents the number of repeating units and is most generally 1-20,000, inclusive, and R₉ and/or R₁₀ may be independently selected from a group that includes, but is not limited to, any C1-200 alkanediyl, C2-200 alkenediyl, C2-200 alkynediyl, C3-200 cycloalkanediyl, C3-200 cycloalkenediyl, C3-C200 cycloalkynediyl, C3-200 cycloarylenediyl, C3-200 alkarylenediyl, C3-200 aralkylenediyl. To further define R₉ and/or R₁₀ any alkanediyl moiety with a carbon backbone between 1 and 20 is preferable; any alkenediyl moiety with a carbon backbone between 2 and 20 is preferable; any alkynediyl moiety with a carbon backbone between 2 and 20 is preferable; any cycloalkanediyl moiety with a carbon backbone between 4 and 30 is preferable; any cycloalkenediyl moiety with a carbon backbone between 7 and 30 is preferable; any cycloalkynediyl moiety with a carbon backbone between 7 and 30 is preferable; any cycloarylenediyl moiety with a carbon backbone between 3 and 30 is preferable; any alkarylenediyl moiety with a carbon backbone between 5 and 30 is preferable; any aralkylenediyl moiety with a carbon backbone between 3 and 30 is preferable. Groups defined by R₁, R₂, R₃, R₄, R₅, R₆, R₇, R₈, R₉ and/or R₁₀ may include heteroatoms.

R₉ and/or R₁₀ may also be independently selected from a haloalkanediyl; haloalkenediyl; haloalkynediyl; halocycloalkanediyl; halocycloalkenediyl; halocycloalkynediyl; halocycloarylenediyl; haloalkarylenediyl; haloaralkylenediyl; alkoxyalkanediyl; alkoxyalkenediyl; alkoxyalkynediyl; alkoxycycloalkanediyl; alkoxycycloalkenediyl; alkoxycycloalkynediyl; alkoxycycloarylenediyl; alkoxyalkarylenediyl; alkoxyaralyenediyl; aminoalkanediyl; aminoalkenediyl; aminoalkynediyl; aminocycloalkanediyl; aminocycloalkenediyl; aminocycloalkynediyl; aminocycloarylenediyl; aminocycloalkarylenediyl; aminoaralkylenediyl; sulfoalkanediyl; sulfoalkenediyl; sulfoalkynediyl; sulfocycloalkanediyl; sulfocycloalkenediyl; sulfocycloalkynediyl; sulfocycloarylenediyl; sulfoalkarylenediyl; sulfoaralkylenediyl; phosphoalkanediyl; phosphoalkenediyl; phosphoalkynediyl; phosphocycloalkanediyl; phosphocycloalkenediyl; phosphocycloalkynediyl; phosphocycloarylenediyl; phosphoalkyarylenediyl; phosphoaralkylenediyl; mono-, di- and trialkylamino-(alkanediyl, alkenediyl, alkynediyl, cycloalkanediyl, cycloalkenediyl, cycloalkynediyl, cycloarylenediyl, alkarylenediyl or aralkylenediyl); mono-, di- and trialkylsulfo-(alkanediyl, alkenediyl, alkynediyl, cycloalkanediyl, cycloalkenediyl, cycloalkynediyl, cycloarylenediyl, alkarylenediyl or aralkylenediyl); mono-, di- and trialkylphospho-(alkanediyl, alkenediyl, alkynediyl, cycloalkanediyl, cycloalkenediyl, cycloalkynediyl, cycloarylenediyl, alkarylenediyl or aralkylenediyl); arylamino-(alkanediyl, alkenediyl, alkynediyl, cycloalkanediyl, cycloalkenediyl, cycloalkynediyl, cycloarylenediyl, alkarylenediyl or aralkylenediyl); arylsulfo-(alkanediyl, alkenediyl, alkynediyl, cycloalkanediyl, cycloalkenediyl, cycloalkynediyl, cycloarylenediyl, alkarylenediyl or aralkylenediyl); arylphospho-(alkanediyl, alkenediyl, alkynediyl, cycloalkanediyl, cycloalkenediyl, cycloalkynediyl, cycloarylenediyl, alkarylenediyl or aralkylenediyl); aminoacyl; sulfoacyl; phosphoacyl; N-(aryl, aryloxy, arylthiol, arylsulfinyl, arylsufonyl, arylphosphino, or arylphosphono, and the like)amino-(alkanediyl, alkenediyl, alkynediyl, cycloalkanediyl, cycloalkenediyl, cycloalkynediyl, cycloarylenediyl, alkarylenediyl or aralkylenediyl); N-(aryl, aryloxy, arylthiol, arylsulfinyl, arylsufonyl, arylphosphino, or arylphosphono, and the like)sulfo-(alkanediyl, alkenediyl, alkynediyl, cycloalkanediyl, cycloalkenediyl, cycloalkynediyl, cycloarylenediyl, alkarylenediyl or aralkylenediyl, and the like); N-(aryl, aryloxy, arylthiol, arylsulfinyl, arylsufonyl, arylphosphino, or arylphosphono, and the like)phosphoro-(alkanediyl, alkenediyl, alkynediyl, cycloalkanediyl, cycloalkenediyl, cycloalkynediyl, cycloarylenediyl, alkarylenediyl or aralkylenediyl).

R₉ and/or R₁₀ may also be independently selected from any divalent form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkanediyl; alkenediyl; alkynediyl; cycloalkanediyl; cycloalkenediyl; cycloalkynediyl; cycloarylenediyl; alkarylenediyl or aralkylenediyl; acylamino; acylsulfo; acylphospho; alkylamino, alkylsulfo, alkylphospho, and the like.

R₉ and/or R₁₀ may also be selected from any divalent form of any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

In some implementations, R₉ and/or R₁₀ may be a group having at least one ring structure. In formulas with more than one ring structure, polycyclic aromatic radicals may be fused to one another or bonded to one another by way of a C—C bond or by other bridging groups. The bridging groups may be other ring structures. Ring structures may include any aromatic ring, aliphatic ring, heterocyclic ring, etc. and is not limited by the type of element used in the backbone of the ring. The single ring structures may include between 5 and 7 members. Some non-limiting examples include those listed in formulas A through M. Other non-limiting bridging group examples may include those in formulas N through W as well as other such as, but not limited to, —(CH₂)ₓ—, —C(═O)—, —O—, —S(═O)₂—, —C(CF₃)₂—, —Si(CH₃)₂—, —C(CH₃)₂, —O(CH₂)_{X}O—, —(CH₂O)ₓ—, etc., where x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

In formula 47, R₁, R₂, R₃, R₄, R₅, R₆, R₇ and/or R₈ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 cycloarylenyl, C3-200 alkarylenyl, C3-200 aralkylenyl. To further define R₁, R₂, R₃, R₄, R₅, R₆, R₇ and/or R₈, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any cycloarylenyl moiety with a carbon backbone between 3 and 30 is preferable; any alkarylenyl moiety with a carbon backbone between 5 and 30 is preferable; any aralkylenyl moiety with a carbon backbone between 3 and 30 is preferable.

R₁, R₂, R₃, R₄, R₅, R₆, R₇ and/or R₈ may also be independently selected from a haloalkyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkyl; alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloaryleneyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkanyl, cycloalkenyl, cycloalkynyl, cycloarylendiyl, alkarylenyl or aralkylenyl); mono-, di- and trialkylsulfo-(alkyl, alkenyl, alkynyl, cycloalkanyl, cycloalkenyl, cycloalkynyl, cycloarylenyl, alkarylenyl or aralkylenyl); mono-, di- and trialkylphospho-(alkyl, alkenyl, alkynyl, cycloalkanyl, cycloalkenyl, cycloalkynyl, cycloarylenyl, alkarylenyl or aralkylenyl); arylamino-(alkyl, alkenyl, alkynyl, cycloalkanyl, cycloalkenyl, cycloalkynyl, cycloarylenyl, alkarylenyl or aralkylenyl); arylsulfo-(alkyl, alkenyl, alkynyl, cycloalkanyl, cycloalkenyl, cycloalkynyl, cycloarylenyl, alkarylenyl or aralkylenyl); arylphospho-(alkyl, alkenyl, alkynyl, cycloalkanyl, cycloalkenyl, cycloalkynyl, cycloarylenyl, alkarylenyl or aralkylenyl); aminoacyl; sulfoacyl; phosphoacyl; N-(aryl, aryloxy, arylthiol, arylsulfinyl, arylsufonyl, arylphosphino, or arylphosphono, and the like)amino-(alkyl, alkenyl, alkynyl, cycloalkyl, cycloalkenyl, cycloalkynyl, cycloarylenyl, alkarylenyl or aralkylenyl); N-(aryl, aryloxy, arylthiol, arylsulfinyl, arylsufonyl, arylphosphino, or arylphosphono, and the like)sulfo-(alkyl, alkenyl, alkyndiyl, cycloalkyl, cycloalkenyl, cycloalkynyl, cycloarylenyl, alkarylenyl or aralkylenyl, and the like); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, cycloarylenyl, alkarylenyl or aralkylenyl).

R₁, R₂, R₃, R₄, R₅, R₆, R₇ and/or R₈ may also be independently selected from any monovalent form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; alkenyl; alkynyl; cycloalkyl; cycloalkenyl; cycloalkynyl; cycloarylenyl; alkarylenyl or aralkylenyl; acylamino; acylsulfo; acylphospho; alkylamino, alkylsulfo, alkylphospho, and the like.

R₁, R₂, R₃, R₄, R₅, R₆, R₇ and/or R₈ may also be selected from hydrogen or any monovalent form of any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁, R₂, R₃, R₄, R₅, R₆, R₇ and/or R₈ is hydrogen. In embodiments, not all of R₁, R₂, R₃, R₄, R₅, R₆, R₇ and/or R₈ is hydrogen. In embodiments, one or more of R₁, R₂, R₃, R₄, R₅, R₆, R₇ and/or R₈ is hydrogen.

In some implementations, the polymeric backbone of the polyvinylalcohol may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyvinylalcohol backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 cycloarylenyl; C3-200 alkarylenyl; C3-200 aralkylenyl; haloalkanyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkanyl; alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloarylenyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkyl, cycloalkenyl, cycloalkynyl, cycloarylenyl, alkarylenyl or aralkylenyl); mono-, di- and trialkylsulfo-(alkyl, alkenyl, alkynyl, cycloalkyl, cycloalkenyl, cycloalkyne, cycloarylene, alkarylene or aralkylene); mono-, di- and trialkylphospho-(alkane, alkene, alkyne, cycloalkane, cycloalkene, cycloalkyne, cycloarylene, alkarylene or aralkylene); arylamino-(alkane, alkene, alkyne, cycloalkane, cycloalkene, cycloalkyne, cycloarylene, alkarylene or aralkylene); arylsulfo-(alkane, alkene, alkyne, cycloalkane, cycloalkene, cycloalkyne, cycloarylene, alkarylene or aralkylene); arylphospho-(alkane, alkene, alkyne, cycloalkane, cycloalkene, cycloalkyne, cycloarylene, alkarylene or aralkylene); aminoacyl; sulfoacyl; phosphoacyl; N-(aryl, aryloxy, arylthiol, arylsulfinyl, arylsufonyl, arylphosphino, arylphosphono, and the like)amino-(alkane, alkene, alkyne, cycloalkaned, cycloalkene, cycloalkyne, cycloarylene, alkarylene or aralkylene); N-(aryl, aryloxy, arylthiol, arylsulfinyl, arylsufonyl, arylphosphino, arylphosphono, and the like)sulfo-(alkane, alkene, alkyne, cycloalkane, cycloalkene, cycloalkyne, cycloarylene, alkarylene or aralkylene); N-(aryl, aryloxy, arylthiol, arylsulfinyl, arylsufonyl, arylphosphino, arylphosphono, and the like)phosphoro-alkane, alkene, alkyne, cycloalkane, cycloalkene, cycloalkyned, cycloarylene, alkarylene or aralkylene.

The pendant group may also be selected from any monovalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, cycloarylenyll, alkarylenyl or aralkylenyl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyvinyl alcohol polymers can be prepared by any process known in the art. It is intended that the definition of polyvinyl alcohol polymer(s) includes any copolymer. The polyvinyl alcohol may be prepared in the presence of other chemicals or polymers (e.g. surface-active agents, initiators, chain termination agents, polymers, solvents, catalysts, etc.). The polyvinyl alcohol may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyvinyl alcohols may be utilized for this invention. Some types of polyvinyl alcohol polymers include, but are not limited to, polyvinyl alcohol polymers include, but are not limited to, poly(vinyl alcohol), poly(4-vinyl phenol), etc.

Polyvinylesters

The present invention may include a carrier selected from the group consisting of polyvinyl ester polymers which may include any type of polyvinyl ester represented by the general structure comprising of recurring units represented by Formula 48:

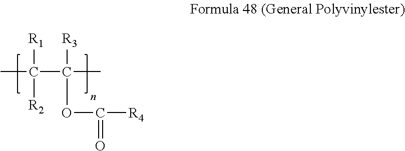

In formula 48, n is an integer representing the number of repeating units and is most generally 1-20,000, inclusive, and R₁, R₂, R₃ and/or R₄ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 cycloarylenyl, C3-200 alkarylenyl, C3-200 aralkylenyl. To further define R₁, R₂, R₃ and/or R₄, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any cycloarylenyl moiety with a carbon backbone between 3 and 30 is preferable; any alkarylenyl moiety with a carbon backbone between 5 and 30 is preferable; any aralkylenyl moiety with a carbon backbone between 3 and 30 is preferable.

R₁, R₂, R₃ and/or R₄ may also be independently selected from a haloalkyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkyl; alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloaryleneyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkanyl, cycloalkenyl, cycloalkynyl, cycloarylendiyl, alkarylenyl or aralkylenyl); mono-, di- and trialkylsulfo-(alkyl, alkenyl, alkynyl, cycloalkanyl, cycloalkenyl, cycloalkynyl, cycloarylenyl, alkarylenyl or aralkylenyl); mono-, di- and trialkylphospho-(alkyl, alkenyl, alkynyl, cycloalkanyl, cycloalkenyl, cycloalkynyl, cycloarylenyl, alkarylenyl or aralkylenyl); arylamino-(alkyl, alkenyl, alkynyl, cycloalkanyl, cycloalkenyl, cycloalkynyl, cycloarylenyl, alkarylenyl or aralkylenyl); arylsulfo-(alkyl, alkenyl, alkynyl, cycloalkanyl, cycloalkenyl, cycloalkynyl, cycloarylenyl, alkarylenyl or aralkylenyl); arylphospho-(alkyl, alkenyl, alkynyl, cycloalkanyl, cycloalkenyl, cycloalkynyl, cycloarylenyl, alkarylenyl or aralkylenyl); aminoacyl; sulfoacyl; phosphoacyl; N-(aryl, aryloxy, arylthiol, arylsulfinyl, arylsufonyl, arylphosphino, or arylphosphono, and the like)amino-(alkyl, alkenyl, alkynyl, cycloalkyl, cycloalkenyl, cycloalkynyl, cycloarylenyl, alkarylenyl or aralkylenyl); N-(aryl, aryloxy, arylthiol, arylsulfinyl, arylsufonyl, arylphosphino, or arylphosphono, and the like)sulfo-(alkyl, alkenyl, alkyndiyl, cycloalkyl, cycloalkenyl, cycloalkynyl, cycloarylenyl, alkarylenyl or aralkylenyl, and the like); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, cycloarylenyl, alkarylenyl or aralkylenyl).

R₁, R₂, R₃ and/or R₄ may also be independently selected from any monovalent form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; alkenyl; alkynyl; cycloalkyl; cycloalkenyl; cycloalkynyl; cycloarylenyl; alkarylenyl or aralkylenyl; acylamino; acylsulfo; acylphospho; alkylamino, alkylsulfo, alkylphospho, and the like.

R₁, R₂, R₃ and/or R₄ may also be selected from hydrogen or any monovalent form of any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁, R₂, R₃ and/or R₄ is hydrogen. In embodiments, not all of R₁, R₂, R₃ and/or R₄ is hydrogen. In embodiments, one or more of R₁, R₂, R₃ and/or R₄ is hydrogen.

In some implementations, the polymeric backbone of the polyvinylester may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyvinylester backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 cycloarylenyl; C3-200 alkarylenyl; C3-200 aralkylenyl; haloalkanyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkanyl; alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloarylenyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkyl, cycloalkenyl, cycloalkynyl, cycloarylenyl, alkarylenyl or aralkylenyl); mono-, di- and trialkylsulfo-(alkyl, alkenyl, alkynyl, cycloalkyl, cycloalkenyl, cycloalkyne, cycloarylene, alkarylene or aralkylene); mono-, di- and trialkylphospho-(alkane, alkene, alkyne, cycloalkane, cycloalkene, cycloalkyne, cycloarylene, alkarylene or aralkylene); arylamino-(alkane, alkene, alkyne, cycloalkane, cycloalkene, cycloalkyne, cycloarylene, alkarylene or aralkylene); arylsulfo-(alkane, alkene, alkyne, cycloalkane, cycloalkene, cycloalkyne, cycloarylene, alkarylene or aralkylene); arylphospho-(alkane, alkene, alkyne, cycloalkane, cycloalkene, cycloalkyne, cycloarylene, alkarylene or aralkylene); aminoacyl; sulfoacyl; phosphoacyl; N-(aryl, aryloxy, arylthiol, arylsulfinyl, arylsufonyl, arylphosphino, arylphosphono, and the like)amino-(alkane, alkene, alkyne, cycloalkaned, cycloalkene, cycloalkyne, cycloarylene, alkarylene or aralkylene); N-(aryl, aryloxy, arylthiol, arylsulfinyl, arylsufonyl, arylphosphino, arylphosphono, and the like)sulfo-(alkane, alkene, alkyne, cycloalkane, cycloalkene, cycloalkyne, cycloarylene, alkarylene or aralkylene); N-(aryl, aryloxy, arylthiol, arylsulfinyl, arylsufonyl, arylphosphino, arylphosphono, and the like)phosphoro-alkane, alkene, alkyne, cycloalkane, cycloalkene, cycloalkyned, cycloarylene, alkarylene or aralkylene.

The pendant group may also be selected from any monovalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, cycloarylenyll, alkarylenyl or aralkylenyl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyvinylester polymers can be prepared by any process known in the art. It is intended that the definition of polyvinylester polymer(s) includes any copolymer. The polyvinylester may be prepared in the presence of other chemicals or polymers (e.g. surface-active agents, initiators, chain termination agents, polymers, solvents, catalysts, etc.). The polyvinylester may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyvinyl esters may be utilized for this invention. Some types of polyvinyl ester polymers include, but are not limited to, poly(vinyl acetate), poly(vinyl benzoate), poly(vinyl butyrate), poly(vinyl caproate), poly(vinyl formate), poly(vinyl propionate), poly(vinyl stearate), poly(vinyl valerate), etc.

Polyvinylethers

The present invention may include a carrier selected from the group consisting of polyvinylether polymers which may include any type of polyvinylether represented by the general structure comprising of recurring units represented by Formula 49:

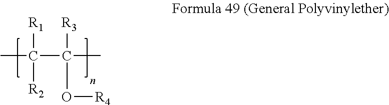

In formula 49, n is an integer representing the number of repeating units and is most generally 1-20,000, inclusive, and R₁, R₂, R₃ and/or R₄ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 cycloarylenyl, C3-200 alkarylenyl, C3-200 aralkylenyl. To further define R₁, R₂, R₃ and/or R₄, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any cycloarylenyl moiety with a carbon backbone between 3 and 30 is preferable; any alkarylenyl moiety with a carbon backbone between 5 and 30 is preferable; any aralkylenyl moiety with a carbon backbone between 3 and 30 is preferable. R₁, R₂, R₃ and/or R₄ may also be independently selected from a haloalkyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkyl; alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloaryleneyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkanyl, cycloalkenyl, cycloalkynyl, cycloarylendiyl, alkarylenyl or aralkylenyl); mono-, di- and trialkylsulfo-(alkyl, alkenyl, alkynyl, cycloalkanyl, cycloalkenyl, cycloalkynyl, cycloarylenyl, alkarylenyl or aralkylenyl); mono-, di- and trialkylphospho-(alkyl, alkenyl, alkynyl, cycloalkanyl, cycloalkenyl, cycloalkynyl, cycloarylenyl, alkarylenyl or aralkylenyl); arylamino-(alkyl, alkenyl, alkynyl, cycloalkanyl, cycloalkenyl, cycloalkynyl, cycloarylenyl, alkarylenyl or aralkylenyl); arylsulfo-(alkyl, alkenyl, alkynyl, cycloalkanyl, cycloalkenyl, cycloalkynyl, cycloarylenyl, alkarylenyl or aralkylenyl); arylphospho-(alkyl, alkenyl, alkynyl, cycloalkanyl, cycloalkenyl, cycloalkynyl, cycloarylenyl, alkarylenyl or aralkylenyl); aminoacyl; sulfoacyl; phosphoacyl; N-(aryl, aryloxy, arylthiol, arylsulfinyl, arylsufonyl, arylphosphino, or arylphosphono, and the like)amino-(alkyl, alkenyl, alkynyl, cycloalkyl, cycloalkenyl, cycloalkynyl, cycloarylenyl, alkarylenyl or aralkylenyl); N-(aryl, aryloxy, arylthiol, arylsulfinyl, arylsufonyl, arylphosphino, or arylphosphono, and the like)sulfo-(alkyl, alkenyl, alkyndiyl, cycloalkyl, cycloalkenyl, cycloalkynyl, cycloarylenyl, alkarylenyl or aralkylenyl, and the like); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, cycloarylenyl, alkarylenyl or aralkylenyl).

R₁, R₂, R₃ and/or R₄ may also be independently selected from any form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; alkenyl; alkynyl; cycloalkyl; cycloalkenyl; cycloalkynyl; cycloarylenyl; alkarylenyl or aralkylenyl; acylamino; acylsulfo; acylphospho; alkylamino, alkylsulfo, alkylphospho, and the like.

R₁, R₂, R₃ and/or R₄ may also be selected from hydrogen or any monovalent form of any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁, R₂, R₃, and/or R₄ is hydrogen. In embodiments, not all of R₁, R₂, R₃, and/or R₄ is hydrogen. In embodiments, one or more of R₁, R₂, R₃ and/or R₄ is hydrogen.

In some implementations, the polymeric backbone of the polyvinylether may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyvinylether backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 cycloarylenyl; C3-200 alkarylenyl; C3-200 aralkylenyl; haloalkanyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkanyl; alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloarylenyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkyl, cycloalkenyl, cycloalkynyl, cycloarylenyl, alkarylenyl or aralkylenyl); mono-, di- and trialkylsulfo-(alkyl, alkenyl, alkynyl, cycloalkyl, cycloalkenyl, cycloalkyne, cycloarylene, alkarylene or aralkylene); mono-, di- and trialkylphospho-(alkane, alkene, alkyne, cycloalkane, cycloalkene, cycloalkyne, cycloarylene, alkarylene or aralkylene); arylamino-(alkane, alkene, alkyne, cycloalkane, cycloalkene, cycloalkyne, cycloarylene, alkarylene or aralkylene); arylsulfo-(alkane, alkene, alkyne, cycloalkane, cycloalkene, cycloalkyne, cycloarylene, alkarylene or aralkylene); arylphospho-(alkane, alkene, alkyne, cycloalkane, cycloalkene, cycloalkyne, cycloarylene, alkarylene or aralkylene); aminoacyl; sulfoacyl; phosphoacyl; N-(aryl, aryloxy, arylthiol, arylsulfinyl, arylsufonyl, arylphosphino, arylphosphono, and the like)amino-(alkane, alkene, alkyne, cycloalkaned, cycloalkene, cycloalkyne, cycloarylene, alkarylene or aralkylene); N-(aryl, aryloxy, arylthiol, arylsulfinyl, arylsufonyl, arylphosphino, arylphosphono, and the like)sulfo-(alkane, alkene, alkyne, cycloalkane, cycloalkene, cycloalkyne, cycloarylene, alkarylene or aralkylene); N-(aryl, aryloxy, arylthiol, arylsulfinyl, arylsufonyl, arylphosphino, arylphosphono, and the like)phosphoro-alkane, alkene, alkyne, cycloalkane, cycloalkene, cycloalkyned, cycloarylene, alkarylene or aralkylene.

The pendant group may also be selected from any monovalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, cycloarylenyll, alkarylenyl or aralkylenyl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyvinylether polymers can be prepared by any process known in the art. It is intended that the definition of polyvinyl ther polymer(s) includes any copolymer. The polyvinylether may be prepared in the presence of other chemicals or polymers (e.g. surface-active agents, initiators, chain termination agents, polymers, solvents, catalysts, etc.). The polyvinylether may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyvinylethers may be utilized for this invention. Some types of polyvinylether polymers include, but are not limited to, poly(butyl vinyl ether), poly(ethyl vinyl ether), poly(hexyl vinyl ether), poly(isobutyl vinyl ether), poly(isopropyl vinyl ether), poly(octyl vinyl ether), poly(propyl vinyl ether), poly(vinyl butyral), poly(vinyl formal), etc.

Polyvinyl Ketones (and Poly(Vinyl Pyrrolidone))

The compositions of the present disclosure may include a carrier selected from the group consisting of polyvinyl ketone polymers which may include any type of polyvinyl ketone represented by the general structure comprising of recurring units represented by Formula 50:

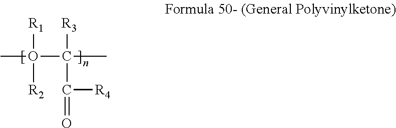

In formula 50, n is an integer representing the number of repeating units and is most generally 1-20,000, inclusive, and R₁, R₂, R₃ and/or R₄ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 cycloarylenyl, C3-200 alkarylenyl, C3-200 aralkylenyl. To further define R₁, R₂, R₃ and/or R₄, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any cycloarylenyl moiety with a carbon backbone between 3 and 30 is preferable; any alkarylenyl moiety with a carbon backbone between 5 and 30 is preferable; any aralkylenyl moiety with a carbon backbone between 3 and 30 is preferable.

R₁, R₂, R₃ and/or R₄ may also be independently selected from a haloalkyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkyl; alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloaryleneyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkanyl, cycloalkenyl, cycloalkynyl, cycloarylendiyl, alkarylenyl or aralkylenyl); mono-, di- and trialkylsulfo-(alkyl, alkenyl, alkynyl, cycloalkanyl, cycloalkenyl, cycloalkynyl, cycloarylenyl, alkarylenyl or aralkylenyl); mono-, di- and trialkylphospho-(alkyl, alkenyl, alkynyl, cycloalkanyl, cycloalkenyl, cycloalkynyl, cycloarylenyl, alkarylenyl or aralkylenyl); arylamino-(alkyl, alkenyl, alkynyl, cycloalkanyl, cycloalkenyl, cycloalkynyl, cycloarylenyl, alkarylenyl or aralkylenyl); arylsulfo-(alkyl, alkenyl, alkynyl, cycloalkanyl, cycloalkenyl, cycloalkynyl, cycloarylenyl, alkarylenyl or aralkylenyl); arylphospho-(alkyl, alkenyl, alkynyl, cycloalkanyl, cycloalkenyl, cycloalkynyl, cycloarylenyl, alkarylenyl or aralkylenyl); aminoacyl; sulfoacyl; phosphoacyl; N-(aryl, aryloxy, arylthiol, arylsulfinyl, arylsufonyl, arylphosphino, or arylphosphono, and the like)amino-(alkyl, alkenyl, alkynyl, cycloalkyl, cycloalkenyl, cycloalkynyl, cycloarylenyl, alkarylenyl or aralkylenyl); N-(aryl, aryloxy, arylthiol, arylsulfinyl, arylsufonyl, arylphosphino, or arylphosphono, and the like)sulfo-(alkyl, alkenyl, alkyndiyl, cycloalkyl, cycloalkenyl, cycloalkynyl, cycloarylenyl, alkarylenyl or aralkylenyl, and the like); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, cycloarylenyl, alkarylenyl or aralkylenyl).

R₁, R₂, R₃ and/or R₄ may also be independently selected from any form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; alkenyl; alkynyl; cycloalkyl; cycloalkenyl; cycloalkynyl; cycloarylenyl; alkarylenyl or aralkylenyl; acylamino; acylsulfo; acylphospho; alkylamino, alkylsulfo, alkylphospho, and the like.

R₁, R₂, R₃ and/or R₄ may also be selected from hydrogen or any monovalent form of any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁, R₂, R₃ and/or R₄ is hydrogen. In embodiments, not all of R₁, R₂, R₃ and/or R₄ is hydrogen. In embodiments, one or more of R₁, R₂, R₃ and/or R₄ is hydrogen.

In some implementations, the polymeric backbone of the polyvinylketone may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyvinylketone backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 cycloarylenyl; C3-200 alkarylenyl; C3-200 aralkylenyl; haloalkanyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkanyl: alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloarylenyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkyl, cycloalkenyl, cycloalkynyl, cycloarylenyl, alkarylenyl or aralkylenyl); mono-, di- and trialkylsulfo-(alkyl, alkenyl, alkynyl, cycloalkyl, cycloalkenyl, cycloalkyne, cycloarylene, alkarylene or aralkylene); mono-, di- and trialkylphospho-(alkane, alkene, alkyne, cycloalkane, cycloalkene, cycloalkyne, cycloarylene, alkarylene or aralkylene); arylamino-(alkane, alkene, alkyne, cycloalkane, cycloalkene, cycloalkyne, cycloarylene, alkarylene or aralkylene); arylsulfo-(alkane, alkene, alkyne, cycloalkane, cycloalkene, cycloalkyne, cycloarylene, alkarylene or aralkylene); arylphospho-(alkane, alkene, alkyne, cycloalkane, cycloalkene, cycloalkyne, cycloarylene, alkarylene or aralkylene); aminoacyl; sulfoacyl; phosphoacyl; N-(aryl, aryloxy, arylthiol, arylsulfinyl, arylsufonyl, arylphosphino, arylphosphono, and the like)amino-(alkane, alkene, alkyne, cycloalkaned, cycloalkene, cycloalkyne, cycloarylene, alkarylene or aralkylene); N-(aryl, aryloxy, arylthiol, arylsulfinyl, arylsufonyl, arylphosphino, arylphosphono, and the like)sulfo-(alkane, alkene, alkyne, cycloalkane, cycloalkene, cycloalkyne, cycloarylene, alkarylene or aralkylene); N-(aryl, aryloxy, arylthiol, arylsulfinyl, arylsufonyl, arylphosphino, arylphosphono, and the like)phosphoro-alkane, alkene, alkyne, cycloalkane, cycloalkene, cycloalkyned, cycloarylene, alkarylene or aralkylene.

The pendant group may also be selected from any monovalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, cycloarylenyll, alkarylenyl or aralkylenyl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyvinylketone polymers can be prepared by any process known in the art. It is intended that the definition of polyvinylketone polymer(s) includes any copolymer. The polyvinylketone may be prepared in the presence of other chemicals or polymers (e.g. surface-active agents, initiators, chain termination agents, polymers, solvents, catalysts, etc.). The polyvinylketone may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyvinylketone may be utilized for this invention. Some types of polyvinyl ketone polymers include, but are not limited to, poly(vinyl ethyl ketone), poly(vinyl methyl ketone), poly(methyl isopropenyl ketone), poly(vinyl phenyl ketone), poly(vinyl pyrrolidone), etc.

Polyvinyl Sulfides (Polyvinyl Thioethers)

The present invention may include a carrier selected from the group consisting of polyvinyl sulfide polymers which may include any type of polyvinylsulfide represented by the general structure comprising of recurring units represented by Formula 51:

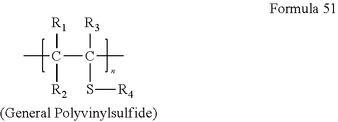

In formula 51, n is an integer representing the number of repeating units and is most generally 1-20,000 inclusive, and R₁, R₂, R₃ and/or R₄ may be independently selected from a group that includes, but is not limited to, any C1-200 alkyl, C2-200 alkenyl, C2-200 alkynyl, C3-200 cycloalkyl, C3-200 cycloalkenyl, C3-C200 cycloalkynyl, C3-200 cycloarylenyl, C3-200 alkarylenyl, C3-200 aralkylenyl. To further define R₁, R₂, R₃ and/or R₄, any alkyl moiety with a carbon backbone between 1 and 20 is preferable; any alkenyl moiety with a carbon backbone between 2 and 20 is preferable; any alkynyl moiety with a carbon backbone between 2 and 20 is preferable; any cycloalkyl moiety with a carbon backbone between 4 and 30 is preferable; any cycloalkenyl moiety with a carbon backbone between 7 and 30 is preferable; any cycloalkynyl moiety with a carbon backbone between 7 and 30 is preferable; any cycloarylenyl moiety with a carbon backbone between 3 and 30 is preferable; any alkarylenyl moiety with a carbon backbone between 5 and 30 is preferable; any aralkylenyl moiety with a carbon backbone between 3 and 30 is preferable.

R₁, R₂, R₃ and/or R₄ may also be independently selected from a haloalkyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkyl; alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloaryleneyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkanyl, cycloalkenyl, cycloalkynyl, cycloarylendiyl, alkarylenyl or aralkylenyl); mono-, di- and trialkylsulfo-(alkyl, alkenyl, alkynyl, cycloalkanyl, cycloalkenyl, cycloalkynyl, cycloarylenyl, alkarylenyl or aralkylenyl); mono-, di- and trialkylphospho-(alkyl, alkenyl, alkynyl, cycloalkanyl, cycloalkenyl, cycloalkynyl, cycloarylenyl, alkarylenyl or aralkylenyl); arylamino-(alkyl, alkenyl, alkynyl, cycloalkanyl, cycloalkenyl, cycloalkynyl, cycloarylenyl, alkarylenyl or aralkylenyl); arylsulfo-(alkyl, alkenyl, alkynyl, cycloalkanyl, cycloalkenyl, cycloalkynyl, cycloarylenyl, alkarylenyl or aralkylenyl); arylphospho-(alkyl, alkenyl, alkynyl, cycloalkanyl, cycloalkenyl, cycloalkynyl, cycloarylenyl, alkarylenyl or aralkylenyl); aminoacyl; sulfoacyl; phosphoacyl; N-(aryl, aryloxy, arylthiol, arylsulfinyl, arylsufonyl, arylphosphino, or arylphosphono, and the like)amino-(alkyl, alkenyl, alkynyl, cycloalkyl, cycloalkenyl, cycloalkynyl, cycloarylenyl, alkarylenyl or aralkylenyl); N-(aryl, aryloxy, arylthiol, arylsulfinyl, arylsufonyl, arylphosphino, or arylphosphono, and the like)sulfo-(alkyl, alkenyl, alkyndiyl, cycloalkyl, cycloalkenyl, cycloalkynyl, cycloarylenyl, alkarylenyl or aralkylenyl, and the like); N-(aryl, aryloxy, arylthiol, arylsulfinyl, arylsufonyl, arylphosphino, or arylphosphono, and the like)phosphoro-(alkyl, alkenyl, alkynyl, cycloalkyl, cycloalkenyl, cycloalkynyl, cycloarylenyl, alkarylenyl or aralkylenyl).

R₁, R₂, R₃ and/or R₄ may also be independently selected from any monovalent form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy; alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-alkyl; alkenyl; alkynyl; cycloalkyl; cycloalkenyl; cycloalkynyl; cycloarylenyl; alkarylenyl or aralkylenyl; acylamino; acylsulfo; acylphospho; alkylamino, alkylsulfo, alkylphospho, and the like.

R₁, R₂, R₃ and/or R₄ may also be selected from hydrogen or any monovalent form of any halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc. In embodiments, not all of each R₁, R₂, R₃ and/or R₄ is hydrogen. In embodiments, not all of R₁, R₂, R₃ and/or R₄ is hydrogen. In embodiments, one or more of R₁, R₂, R₃ and/or R₄ is hydrogen.

In some implementations, the polymeric backbone of the polyvinylsulfide may be substituted within the backbone with groups that may include, but are not limited to, any form (e.g. divalent, etc.) of those listed above. It may also be understood that the backbone(s) of branches off of the main polymer chain may also be substituted with similar groups. Any part of the polyvinylsulfide backbone may be configured with any pendant group(s) that may include, but is not limited to, any C1-200 alkyl; C2-200 alkenyl; C2-200 alkynl: C3-200 cycloalkyl; C3-200 cycloalkenyl; C3-C200 cycloalkynyl; C3-200 cycloarylenyl; C3-200 alkarylenyl; C3-200 aralkylenyl; haloalkanyl; haloalkenyl; haloalkynyl; halocycloalkyl; halocycloalkenyl; halocycloalkynyl; halocycloarylenyl; haloalkarylenyl; haloaralkylenyl; alkoxyalkyl; alkoxyalkenyl; alkoxyalkynyl; alkoxycycloalkanyl: alkoxycycloalkenyl; alkoxycycloalkynyl; alkoxycycloarylenyl; alkoxyalkarylenyl; alkoxyaralyenyl; aminoalkyl; aminoalkenyl; aminoalkynyl; aminocycloalkyl; aminocycloalkenyl; aminocycloalkynyl; aminocycloarylenyl; aminocycloalkarylenyl; aminoaralkylenyl; sulfoalkyl; sulfoalkenyl; sulfoalkynyl; sulfocycloalkyl; sulfocycloalkenyl; sulfocycloalkynyl; sulfocycloarylenyl; sulfoalkarylenyl; sulfoaralkylenyl; phosphoalkyl; phosphoalkenyl; phosphoalkynyl; phosphocycloalkyl; phosphocycloalkenyl; phosphocycloalkynyl; phosphocycloarylenyl; phosphoalkyarylenyl; phosphoaralkylenyl; mono-, di- and trialkylamino-(alkyl, alkenyl, alkynyl, cycloalkyl, cycloalkenyl, cycloalkynyl, cycloarylenyl, alkarylenyl or aralkylenyl); mono-, di- and trialkylsulfo-(alkyl, alkenyl, alkynyl, cycloalkyl, cycloalkenyl, cycloalkyne, cycloarylene, alkarylene or aralkylene); mono-, di- and trialkylphospho-(alkane, alkene, alkyne, cycloalkane, cycloalkene, cycloalkyne, cycloarylene, alkarylene or aralkylene); arylamino-(alkane, alkene, alkyne, cycloalkane, cycloalkene, cycloalkyne, cycloarylene, alkarylene or aralkylene); arylsulfo-(alkane, alkene, alkyne, cycloalkane, cycloalkene, cycloalkyne, cycloarylene, alkarylene or aralkylene); arylphospho-(alkane, alkene, alkyne, cycloalkane, cycloalkene, cycloalkyne, cycloarylene, alkarylene or aralkylene); aminoacyl; sulfoacyl; phosphoacyl; N-(aryl, aryloxy, arylthiol, arylsulfinyl, arylsufonyl, arylphosphino, arylphosphono, and the like)amino-(alkane, alkene, alkyne, cycloalkaned, cycloalkene, cycloalkyne, cycloarylene, alkarylene or aralkylene); N-(aryl, aryloxy, arylthiol, arylsulfinyl, arylsufonyl, arylphosphino, arylphosphono, and the like)sulfo-(alkane, alkene, alkyne, cycloalkane, cycloalkene, cycloalkyne, cycloarylene, alkarylene or aralkylene); N-(aryl, aryloxy, arylthiol, arylsulfinyl, arylsufonyl, arylphosphino, arylphosphono, and the like)phosphoro-alkane, alkene, alkyne, cycloalkane, cycloalkene, cycloalkyned, cycloarylene, alkarylene or aralkylene.

The pendant group may also be selected from any monovalent radical form of any aminoaryl; sulfoaryl; phosphoaryl; alkoxy; alkenyloxy; alkynyloxy; cycloalkoxy; cycloalkenyloxy; cycloalkynyloxy; haloalkoxy; aralkoxy; alkoxyaryloxy, alkoxyalkoxy; aminoalkoxy; sulfoalkoxy; phosphoalkoxy; mono-, di- and trialkylaminoalkoxy; mono-, di- and trialkylsulfoalkoxy; mono-, di- and trialkylphosphoroalkoxy; arylaminoalkoxy; arylsulfoalkoxy; arylphosphoalkoxy; N-(aryl, aryloxy, arythiol, arylsulfinyl, arylsulfonyl, arylphosphosphino or arylphosphono, and the like)-N-alkylamino-alkoxy; N-(aryl, aryloxy, arythiol, arylsulfinyl, arylsulfonyl, arylphosphosphino or arylphosphono, and the like)-N-alkylsulfo-alkoxy; N-(aryl, aryloxy, arythiol, arylsulfinyl, arylsulfonyl, arylphosphosphino or arylphosphono, and the like)-N-alkylphospho-alkoxy; acyloxy; acyloxyalkyl; acylaminoalkyl; acylsulfoalkyl; acylphosphoalkyl; N-acyl-imino-(alkyl, alkenyl, alkynyl, cycloalkyl, cycloalkenyl, cycloalkynyl, cycloarylenyll, alkarylenyl or aralkylenyl); acylamino; acylsulfo; acylphospho; alkylamino; alkylsulfo; alkylphospho; and the like; halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, orthocarbonate ester, organic acid anhydride, silyl, alkylthio, sulfonamide, trifluoromethyl, thiocyanato, mercaptocarbonyl, hydroxylthiocarbonyl, thiolester, thionoester, carbodithioc acid, carbodithio, carboyl, formyl, haloformyl, (alkoxycarbonyl)oxy, carboxy, methoxy, alkoxy, alkoxy-ol dialkoxy, trialkoxy, methylenedioxy, tetralkoxy, carboxylic anhydride, carbamoyl, imino, cyanato, isocyanato, nitroxy, nitrosooxy, nitro, nitroso, oxime, pyridyl, carbamate, phosphanyl, phosphono, phosphonoxy, [(alkoxy)hydroxyphosphoryl]oxy, etc.

Other pendant group(s) may include, but are not limited to, halogen, azide, cycloalkyl, amino, nitro, sulfhydryl, imino, amido, phophonate, phophinate, carbonyl, carboxyl, silyl, ether, alkylthio, sulfonyl, sulfonamide, ketone, aldehyde, ester, heterocyclyl, aromatic, heteroaromatic, cyano, trifluoromethyl, etc. Yet other pendant group(s) may include alcohol, ketone, aldehyde, acyl halide, carbonate, carboxylate, carboxylic acid, methoxy, ether, hemiacetal, hemiketal, acetal, orthoester, heterocycle, orthocarbonate ester, organic acid anhydride, sulfide, disulfide, thiol, sulfoxide, sulfone, sulfate, sulfinyl, cyano, thiocarboxylic acid, thioester, dithiocarboxylic acid, dithiocarboxylic acid ester, amide, amine, imine, imide, azide, azo, cyanate, isocyanate, nitrate, nitrile, nitro, oxime, pyridine, carbamate ester, phosphine, phosphoric acid, phosphate, phosphodiester, etc.

In embodiments, each n of the above formula independently ranges inclusively from 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000.

Polyvinylsulfide polymers can be prepared by any process known in the art. It is intended that the definition of polyvinylsulfide polymer(s) includes any copolymer. The polyvinyl sulfide may be prepared in the presence of other chemicals or polymers (e.g. surface-active agents, initiators, chain termination agents, polymers, solvents, catalysts, etc.). The polyvinylsulfide may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

A variety of polyvinylsulfide may be utilized in compostions of this disclosure. Some types of polyvinylsulfide polymers include, but are not limited to, poly(vinyl butyl sulfide), poly(vinyl ethyl sulfide), poly(vinyl methyl sulfide), poly(vinyl phenyl sulfide), poly(vinyl propyl sulfide), etc.

Carbohydrates

The compositions of the present disclosure may include a carrier selected from the group consisting of any type of carbohydrate including those selected from sugars, polyols, oligosaccharides, polysaccharides and/or fibers. Examples of saccharides and oligosaccharides include, but are not limited to, fructose, glucose, isomaltose, sucrose, trehalose, xylose, galactose, isomaltulose, lactose, mannose, tagatose, trehalulose, lactulose, maltose, turanose, cellobiose, melezitose, maltriose, acarbose, stachyose, ribose, arabinose, lyxose, deoxyribose, psicose, sorbose, tagatose, allose, altrose, gulose, idose, talose, fucose, fuculose, rhamnose, sedohepulose, octose, nonose, erythrose, theose, amylose, fructo-oligosaccharide, galacto-oligosaccharide, human milk-oligosaccharide, isomalto-oligosaccharide, maltotriose-oligosaccharide, mannan-oligosaccharide, raffinose, stachyose, varbascose, etc. Examples of polyols include, but are not limited to erythritol, hydrogenated starch, hydrolysates, maltitol, sorbitol, glycerol, inositol, isomalt, lactitol, mannitol, xylitol, etc. Examples of polysaccharides include, but are not limited to, starches and modified starches, such as potato starch, modified potato starch, corn starch, modified corn starch, wheat starch, modified wheat starch, rice starch; dextrins; chondroitin sulfate; maltodextrins. Examples of fibers include, but are not limited to, acacia (arabic gum), agar-agar, algin-alginate, arabinoxylan, beta-glucan, beta-mannan, carrageenan gum, carob locust bean gum, fenugreek gum, galactomannans, gallan gum, glucomannan or konjac gum, guar gum, hemicellulose, inulin, karayn gum, pectin, polydextrose, psyllium husk mucilage, resistant starches, tara gum, tragacanta gum, xanthuan gum, cellulose, chitin, chitosan, etc; Other carbohydrates include dextrins and cyclodextrins, galactonammans, alginates, carragenates, amylopectin, modified inulin, cellulose and modified cellulose, dextran, maltodextrin, cyclodextrin, oligofructose, sodium carboxymethylcellulose, linear sulfonated ∝-(1,4)-linked D-glucose polymer, γ-cyclodextrin and the like. In general, any material that includes carbohydrates or carbohydrates that have been derivatized can be used in the compositions herein. Carbohydrates include natural carbohydrates and chemically modified carbohydrates.

Carbohydrates can be harvested by any process known in the art. Derivatized carbohydrates can be prepared by any process known in the art. It is intended that the definition of carbohydrate or derivatized carbohydrate includes any copolymer. The carbohydrate and/or derivatized carbohydrate may be prepared in the presence of other chemicals or polymers (e.g. surface-active agents, initiators, chain termination agents, polymers, solvents, catalysts, etc.). The carbohydrate and/or derivatized carbohydrate may be treated post polymerization (e.g. grafting, crosslinking, other modifications or functionalization, etc.). Protecting groups may be used to protect reactive functional group moieties (e.g. —NH, —OH, acidic groups, basic groups, etc.) that would have an adverse effect on the polymerization process. They may also be used to protect reactive functional group moieties that effect any derivatization and/or substitution reaction following polymerization. The moieties may be converted into noninterfering groups using any conventional protection group chemistry and later deprotected at a desired time. Processes for protecting and deprotecting chemistry is known to one skilled in the art.

Ceramics

The compositions of the present disclosure may include, but is not limited to, a carrier selected from the group consisting of any type of ceramic and/or composite material containing a ceramic, including those selected from any advanced composite material or advanced polymer matrix composite, alumina, alumino-silicate, aluminum magnesium boride, aluminum nitride, aluminum oxynitride, barium titanate, beryllium oxide, biscuit porcelain, bismuth strontium, boron nitride, calcium copper oxide, bismuth titanate, bone china, boron nitride, briquetage, calcium aluminates, cenosphere, ceramic building materials, ceramic colorants, ceramic matrix composites, ceramic nanoparticles, ceramic impregnated fabric cerium hexaboride, coade stone, cordierite, ceramic powders, dysprosium titanate, earthenware, electroceramics, expanded clay aggregates, ferrite, ferroelectric ceramics, fire clay, fumed silica, geopolymers, geopolymer concretes, germanium dioxide, glass, glass-ceramics, green body, grog or clay, hafnium diboride, hard-paste porcelain, hydroxyapatite, jesmonite, lanthanum gallium silicate, lanthanum hexaboride, lanthanum strontium cobalt ferrite, lanthanum strontium manganite, lanthanum ytterbium oxide, lava, lead scandium tantalite, lead zirconate titanate, lithophane, lumicera, magnesium diboride, magnesium oxide, martensite, metal clay, molybdenum disilicide, mud, mullite, nanophase ceramic, nile silt, ninja rocks, niobium diboride, paper clay, petuntse, phanolith, porcelain, quartz, sapphire, silica, silicon, silicon carbide, sagger clay, sialon, SiC—SiC matrix composite, silica, silicon boride, silicon carbide, silicon dioxide, silicon nitride, silicon oxynitride, soapstone, sodium bismuth titanate, soft-paste porcelain, strontium titanate, terracotta, tetragonal polycrystalline zirconia, titanium carbide, tungsten disilicide, tungsten nitride, yttralox, yttrium barium copper oxide, zinc oxide, zirconia toughened alumina, zirconium dioxide, etc.

Additives

Surfactant

The compositions of the disclosure optionally include a surfactant. Any form of surfactant appropriate for the application of the composition can be employed. The surfactant may be anionic, cationic, zwitterionic and/or non-ionic, hydrophilic and/or hydrophobic. The surfactants used in the compositions may advantageously be added to improve solubility of the MME-transport moiety blend and/or blend components in the carrier of the solid, to improve de-adsorption or outgassing from the solid composition, to improve stability of the MME-transport moiety blend and/or blend components, enhance solubilization of the MME-transport moiety blend and/or blend components if dispersed in a solvent (e.g. water, etc.). A single surfactant or combination of surfactants may be used for the solid composition of the disclosure. “Hydrophilic” are defined as those that have a higher hydrophilic-lipophilic balance (HLB value) (>10) whereas “hydrophobic” surfactants are those with a lower HLB balance (<10). Hydrophilic surfactants have a greater solubility in aqueous or more polar solutions. Hydrophobic surfactants have a greater solubility in oil or more non-polar solutions.

A number of different surfactants may be used for compositions of this disclosure. Examples of surfactants include, but are not limited to, any polyethylene glycol fatty acid mono-ester, polyethylene glycol fatty acid di-ester, any polyethylene glycol fatty acid mono- and di-ester mixture, any polyethylene glycol fatty acid ester, any alcohol-oil transesterification products, any polyglycerized fatty acids, any propylene glycol fatty acid esters, any propylene glycol ester-glycerol fatty acid ester mixture, any mono- and di-glyceride, any sterol and sterol derivatives, any polyethylene glycol sorbitan fatty acid ester, any polyethylene glycol alkyl ether, any sugar ester, any polyethylene glycol alkyl phenol, any polyoxyethylene-polyoxypropylene block copolymer, any sorbitan fatty acid ester, any lower alcohol fatty acid ester, any ionic surfactant, triglyceride, etc.

The examples listed below illustrate the types of surfactants that may be used for this composition, but are intended to be limiting or exhaustive.

Examples of polyethylene glycol fatty acid mono-esters include, but are not limited to, PEG 4-100 monolaurate, PEG 4-100 monooleate, PEG 4-100 monostearate, PEG 400 distearate, PEG 100 monolaurate, PEG 200 monolaurate, PEG 300 monolaurate, PEG 100 monolaurate, PEG 200 monolaurate, PEG 300 monolaurate, PEG 400 dioleate, PEG 400 through 1000 monostearate, PEG-1 stearate, PEG-2 stearate, PEG-2 oleate, PEG-4 laurate, PEG-4 oleate, PEG-4 stearate, PEG-5 stearate, PEG-5 oleate, PEG-6 oleate, PEG-7 oleate, PEG-6 laurate, PEG-7 laurate, PEG-6 stearate, PEG-8 laurate, PEG-8 oleate, PEG-8 stearate, PEG-9 oleate, PEG-9 stearate, PEG-10 laurate, PEG-10 laurate, PEG-10 oleate, PEG-10 stearate, PEG-12 laurate, PEG-12 oleate, PEG-12 ricinoleate, PEG-12 stearate, PEG-15 stearate, PEG-15 oleate, PEG-20 laurate, PEG-20 oleate, PEG-20 stearate, PEG-25 stearate, PEG-32 laurate, PEG-32 oleate, PEG-32 stearate, PEG-30 stearate, PEG-40 laurate, PEG-40 oleate, PEG-40 stearate, PEG-45 stearate, PEG-50 stearate, PEG-55 stearate, PEG-100 oleate, PEG-100 stearate, PEG-200 oleate, PEG-400 oleate, PEG-600 oleate, etc.

Examples of polyethylene glycol fatty acid di-ester include, but are not limited to, PEG-4 dilaurate, PEG-4 dioleate, PEG-4 distearate, PEG-6 dilaurate, PEG 6-dioleate, PEG-6 distearate, PEG-8 dilaurate, PEG-8 dioleate, PEG-8 distearate, PEG-10 dipalmitate, PEG-12 dilaurate, PEG-12 distearate, PEG-12 dioleate, PEG-20 dilaureate, PEG-20 dioleate, PEG-20 distearate, PEG-32 dilaurate, PEG-32 dioleate, PEG-32 distearate, PEG-400 dioleate, PEG-400 distearate, etc.

Examples of polyethylene glycol fatty acid mono- and di-ester mixtures include, but are not limited to, PEG 4-150 mono-/di-laurate, PEG 4-150 mono-/di-oleate, PEG 4-150 mono-/di-stearate, etc.

Examples of polyethylene glycol glycerol fatty acid ester include, but are not limited to, PEG-20 glyceryl laurate, PEG-30 glyceryl laurate, PEG-15 glyceryl laurate, PEG-40 glyceryl laurate, PEG-20 glyceryl stearate, PEG-20 glyceryl oleate, PEG-30 glyceryl oleate, etc.

Examples of alcohol-oil transesterification products include, but are not limited to, PEG-3 castor oil, PEG-5 castor oil, PEG-9 castor oil, PEG-16 castor oil, PEG-20 castor oil, PEG-23 castor oil, PEG-30 castor oil, PEG-35 castor oil, PEG-38 castor oil, PEG-40 castor oil, PEG-50 castor oil, PEG-56 castor oil, PEG-60 castor oil, PEG-100 castor oil, PEG-200 castor oil, PEG-5 hydrogenated castor oil, PEG-7 hydrogenated castor oil, PEG-10 hydrogenated castor oil, PEG-20 hydrogenated castor oil, PEG-25 hydrogenated castor oil, PEG-30 hydrogenated castor oil, PEG-40 hydrogenated castor oil, PEG-45 hydrogenated castor oil, PEG-50 hydrogenated castor oil, PEG-60 hydrogenated castor oil, PEG-80 hydrogenated castor oil, PEG-100 hydrogenated castor oil, PEG-6 corn oil, PEG-6 almond oil, PEG-6 apricot kernel oil, PEG-6 oilve oil, PEG-6 peanut oil, PEG-6 hydrogenated palm kernel oil, PEG-6 triolein, PEG-8 corn oil, PEG-20 corn glycerides, PEG-20 almond glycerides, PEG-25 trioleate, PEG-40 palm kernel oil, PEG-60 corn glycerides, PEG-60 almond glycerides, PEG-4 caprylic/capric triglycerides, PEG-8 caprylic/capric glycerides, PEG-6 caprylic/capric glycerides, lauroyl macrogol-32 glyceride, stearoyl macrogol glyceride, mono-, di-, tri-, tetra-esters of vegetable oils and sorbitol, pentaerythrityl tetraisostearate, pentaerythrityl distearate, pentaerythrityl tetraoleate, pentaerythrityl tetrastearate, pentaerythrityl tetracaprylate/tetracaprate, pentaerythrityl tetraoctanoate, etc.

Examples of polyglycerized fatty acids include, but are not limited to, polyglyceryl-2 stearate, polyglyceryl-2 oleate, polyglyceryl-2 isostearate, polyglyceryl-3 oleate, polyglyceryl-4 oleate, polyglyceryl-4 stearate, polyglyceryl-6 oleate, polyglyceryl-10 laurate, polyglyceryl-10 oleate, polyglyceryl-10 stearate, polyglyceryl-6 ricinoleate, polyglyceryl-10 linoleate, polyglyceryl-6 pentaoleate, polyglyceryl-3 dioleate, polyglyceryl-3 distearate, polyglyceryl-4 pentaoleate, polyglyceryl-6 dioleate, polyglyceryl-2 dioleate, polyglyceryl-10 trioleate, polyglyceryl-10-pentaoleate, polyglyceryl-10 septaoleate, polyglyceryl-10 tetraoleate, polyglyceryl-10 decaisostearate, polyglyceryl-101 decaoleate, polyglyceryl-10 mono/dioleate, polyglyceryl polyricinoleate, etc.

Examples of propylene glycol fatty acid esters include, but are not limited to, propylene glycol monocaprylate, propylene glycol monolaurate, propylene glycol oleate, propylene glycol myristate, propylene glycol monostearate, propylene glycol hydroxy stearate, propylene glycol ricinoleate, propylene glycol isostearate, propylene glycol monooleate, propylene glycol dicaprylate/dicaprate, propylene glycol dioctanoate, propylene glycol caprylate/caprate, propylene glycol dilaurate, propylene glycol distearate, propylene glycol dicaprylate, propylene glycol dicaprate, etc.

Examples of propylene glycol ester-glycerol ester mixtures include, but are not limited to, oleic, stearic, etc.

Examples of mono- and di-glycerides include, but are not limited to, monopalmitolein, monoelaidin, monocaproin, monocaprylin, monocaprin, monolaurin, glyceryl monomyristate, glyceryl monooleate, glycerol monooleate/linoleate, glycerol monolinoleate, glyceryl ricinoleate, glyceryl monolaurate, glycerol monopalmitate, glycerol monostearate, glyceryl mono- and di-oleate, glyceryl palmitic/stearic, glyceryl acetate, glyceryl laurate, glyceryl citrate/lactate/oleate/linoleate, glyceryl caprylate, glyceryl caprylate/caprate, caprylic acid mono/diglycerides, caprylic/capric glycerides, mono- and diacetylated monoglycerides, glyceryl monostearate, lactic acid esters of mono- and di-glycerides, dicaproin, dicaprin, dioctanoin, dimyristin, dipalmitin, distearin, glyceryl dilaurate, glyceryl dioleate, glycerol esters of fatty acids, dipalmitolein, 1,2- and 1,3-diolein, dielaidin, dilinolein, etc.

Examples of sterol and sterol derivatives include, but are not limited to, cholesterol, sitosterol, lanosterol, PEG-24 cholesterol ether, PEG-30 cholestanol, Phytosterol, PEG-25 phyto sterol, PEG-5 soya sterol, PEG-10 soya sterol, PEG-20 soya sterol, PEG-30 soya sterol, etc.

Examples of polyethylene glycol sorbitan fatty acid esters include, but are not limited to, PEG-10 sorbitan laurate, PEG-20 sorbitan monolaurate, PEG-4 sorbitan monolaurate, PEG-80 sorbitan monolaurate, PEG-6 sorbitan monolaurate, PEG-20 sorbitan monopalmitate, PEG-20 sorbitan monostearate, PEG-4 sorbitan monostearate, PEG-8 sorbitan monostearate, PEG-6 sorbitan monostearate, PEG-20 sorbitan tristearate, PEG-6 sorbitan tetrastearate, PEG-60 sorbitan tetrastearate, PEG-5 sorbitan monooleate, PEG-6 sorbitan monooleate, PEG-20 sorbitan monooleate, PEG-40 sorbitan oleate, PEG-20 sorbitan trioleate, PEG-6 sorbitan tetraoleate, PEG-30 sorbitan tetraoleate, PEG-40 sorbitan tetraoleate, PEG-20 sorbitan monoisostearate, PEG sorbital hexaleate, PEG-6 sorbitol hexastearate, etc.

Examples of polyethylene glycol alkyl ethers include, but are not limited to, PEG-2 oleyl ether/oleth-2, PEG-3 oleyl ether/oleth-3, PEG-5 oleyl ether/oleth-5, PEG-10 oleyl ether/oleth-10, PEG-20 oleyl ether/oleth-20, PEG-4 lauryl ether/laureth-4, PEG-9 lauryl ether, PEG-23 lauryl ether/laureth-23, PEG-2 cetyl ether, PEG-10 cetyl ether, PEG-20 cetyl ether, PEG-2 stearyl ether, PEG-10 stearyl ether, PEG-20 stearyl ether, PEG-100 stearyl ether, etc.

Examples of sugar esters include, but are not limited to, sucrose distearate, sucrose distearate/monostearate, sucrose dipalmitate, sucrose monostearate, sucrose monopalmitate, sucrose monolaurate, etc.

Examples of polyethylene glycol alkyl phenols include, but are not limited to, PEG 100 nonyl phenol, PEG-15-100 octyl phenol ether, etc.

Examples of polyoxyethylene-polyoxypropylene block copolymers include, but are not limited to, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, etc.

Examples of sorbitan fatty acid esters include, but are not limited to, sorbitan monolaurate, sorbitan monopalmitate, sorbitan monooleate, sorbitan monostearate, sorbitan trioleate, sorbitan sesquioleate, sorbitan tristearate, sodium monoisostearate, sorbitan sesquistearate, etc.

Examples of lower alcohol fatty acid esters include, but are not limited to, ethyl oleate, isopropyl myristate, isopropyl palmitate, ethyl linoleate, isopropyl linoleate, etc.

Examples of ionic surfactants include, but are not limited to, any fatty acid and/or bile salts, phospholipids, phosphoric acid esters, carboxylates including acyl lactylates, sulfates and sulfonates, cationic surfactants, betaines, and/or ethyoxylated amines, such as sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate (dodecyl), sodium tetradecyl sulfate, sodium lauryl sarcosinate, sodium dioctyl sulfosuccinate (sodium docusate), sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glycol chenodeoxycholate, sodium cholylsarcosinate, sodium N-methyl taurocholate, egg/soy lecithin, cardiolipin, sphingomyelin, phosphatidylcholine, phosphatidyl ethanolamine, phosphatidic acid, phosphatidic glycerol, phosphatidyl serine, diethanolammonium polyoxyethylene-10 oleyl ether phosphate, esterification products of fatty alcohols or fatty alcohols, ethoxylates with phosphoric acid or anhydride, ether carboxylates, succinylated monoglycerides, sodium stearyl fumarate, stearoyl propylene glycol hydrogen succinate, mono/diacetylated tartaric acid esters of mono- and diglycerides, citric acid esters of mono- or di-glycerides, glyceryl-lacto esters of fatty acids, lactylic esters of fatty acids, calcium/sodium stearoyl-2-lactylate, calcium/sodium stearoyl lactylate, alginate salts, propylene glycol alginate, ethoxylated alkyl sulfates, alkyl benzene sulfones, α-olefin sulfonates, acyl isethionates, acyl taurates, alkyl glyceryl ether sulfonates, octyl sulfosuccinate disodium, disodium undecylenamideo-MEA-sulfosuccinate, hexadecyl triammonium bromide, dodecyl ammonium chloride, alkyl benzyldimethylammonium salts, diisobutyl phenoxyethyoxydimethyl benzylammonium salts, lauryl betain, polyoxyethylene-15 coconut amine, etc.

Examples of triglycerides include, but are not limited to, aceituno oil, almond oil, arachis oil, babassu oil, black current seed oil, borage oil, buffalo ground oil, candlenut oil, canola oil, castor oil, Chinese vegetable tallow oil, cocoa butter, coconut oil, coffee seed oil, corn oil, cottonseed oil, crambe oil, cuphea species oil, evening primrose oil, grapeseed oil, groundnut oil, hemp seed oil, illipe butter, kapok seed oil, linseed oil, menhaden oil, mowrah butter, mustard seed oil, oiticica oil, olive oil, palm oil, palm kernel oil, peanut oil, poppy seed oil, rapeseed oil, rice bran oil, safflower oil, sal fat, sesame oil, shark liver oil, shea nut oil, soybean oil, stillingia oil, sunflower oil, tall oil, tea seed oil, tobacco seed oil, tung oil, ucuhuba, veronica oil, wheat germ oil, hydrogenated castor oil, hydrogenated coconut oil, hydrogenated cottonseed oil, hydrogenated palm oil, hydrogenated soybean oil, hydrogenated vegetable oil, hydrogenated cottonseed/castor oil, partially hydrogenated soybean oil, partially hydrogenated soy and cottonseed oil, glyceryl tributyrate, glyceryl tricaproate, glyceryl tricaprylate, glyceryl tricaprate, glyceryl trundecanoate, glyceryl trilaurate, glyceryl trimyristate, glyceryl tripalmitate, glyceryl tristearate, glyceryl triarcidate, glyceryl trimyristoleate, glyceryl tripalmitoleate, glyceryl trioleate, glyceryl trilinoleate, glyceryl trilinolenate, glyceryl tricaprylate/caprate, glyceryl tricaprylate/caprate/laurate, glyceryl tricaprylate/caprate/linoleate, glyceryl tricaprylate/caprate/stearate, glyceryl tricaprylate/laurate/stearate, glyceryl 1,2-caprylate-3-linoleate, glyceryl 1,2-caprate-3-stearate, glyceryl 1,2-laurate-3-myristate, glyceryl 1,2-myristate-3-laurate, glyceryl 1,3-palmitate-2-butyrate, glyceryl 1,3-stearate-2-caprate, glyceryl 1,2-linoleate-3-caprylate, etc.

Other surfactant compositions that may be used for the purpose of this disclosure are known to those skilled in the art and it should be appreciated that such compositions may be suitable to provide all of part of the surfactant component of the composition.

Binder

The compositions of the invention optionally include a binder or agent that provides cohesive properties to materials that otherwise do not adhere together such as powders, particles, etc. The binder provides a mechanism by which particles are bound to one another and may be selected based on the carrier material. Binders well known in the art can be selected for use in this disclosure according to the parameters of the solid composition such that the composition fulfills the desired properties and intended functions of the disclosure. Examples of binders may include, but are not limited to, those that bind matrix materials (e.g. dry starch, dry sugars, etc.), films (e.g. PVP, starch paste, celluloses, bentonite, sucrose, etc.) and/or chemical (e.g. derivatized cellulose such as carboxy methyl cellulose, hydroxy propyl cellulose hydroxy propyl methyl cellulose; sugar syrups; corn syrup; polysaccharides such as acacia, tragacanth, guar and alginates; gelatin; gelatin hydrolysate; agar; sucrose; dextrose, and/or others (e.g. PVP, PEG, vinyl pyrrolidone copolymers, pregelatinized starch, sorbitol and glucose.

Other binder compositions that may be used for the purpose of this disclsoure are known to those skilled in the art and it should be appreciated that such compositions may be suitable to provide all of part of the buffer component of the composition.

Solubilizer

The solid form of the MME-transport blend moiety of this disclosure optionally includes one or more solublilizers (e.g. ingredient(s) added to increase the solubility of the MME-transport moiety blend and/or other ingredients of the compositions within the carrier. Some non-limiting examples of solubilizers include alcohols and polyols (e.g. ethanol, isopropanol, butanol, benzyl alcohol, ethylene glycol, propylene glycol, butanediols, glycerol, pentaerythritol, sorbitol, mannitol, transcutol, dimethyl isosorbide, polyethylene glycol, polypropylene glycol, polyvinyl alcohol, etc.), celluloses and derivatized celluloses (e.g. hydroxypropylmethyl cellulose, etc.), cyclodextrins and derivatized cyclodextrins, ethers of polyethylene glycols (e.g. tetrahydrofurfuryl alcohol PEG ether, methoxy PEG, etc.), amides (e.g. 2-pyrrollidone, 2-piperidone, ε-caprolactam, N-alkylpyrrolidone, N-hydroxyalkylpyrrollidone, N-alkylpiperidone, N-alkylcaprolactam, dimethylacetamide, polyvinylpyrrolidone, etc.), esters (e.g. ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone, O-valerolactone, β-butyrolactone, etc.), dimethyl acetamide, dimethyl isosorbide, N-methylpyrrolidones, monooctanoin, diethylene glycol monoethyl ether, water, etc. Mixtures of the described solubilizers are also considered.

Other solubilizer compositions that may be used for the purpose of this disclosure are known to those skilled in the art and it should be appreciated that such compositions may be suitable to provide all of part of the solubilizer component of the composition.

Buffering Agent

The compositions of this disclosure optionally include one or more buffering agents (e.g. ingredient(s) added to alter and maintain pH of the contents of the solid composition). Some non-limiting examples of acids include hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, parabromophenylsulfonic acid, propionic acid, p-toluenesulfonic acid, salicylic acid, stearic acid, succinic acid, tannic acid, tartaric acid, thioglycolic acid, toluenesulfonic acid, uric acid, or mixtures thereof; and bases that include, but are not limited to, amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, or mixtures thereof; or a salt of a cation and acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, parabromophenylsulfonic acid, priopionic acid, p-toluenesulfonic acid, salicyclic acid, stearic acid, succinic acid, tannic acid, tartaric acid, thioglycolic acid, toluenesulfonic acid, uric acid, or combinations thereof, etc; phosphates, carbonates, tartrates, borates, citrates, acetates, maleates, or combinations thereof, etc.

Other buffering agent compositions that may be used for the purpose of this disclosure are known to those skilled in the art and it should be appreciated that such compositions may be suitable to provide all of part of the buffering agent component of the composition.

Thickener

The compositions of this disclosure optionally include a thickening agent, if needed, that may be added to modify the viscosity of the carrier composition and optimize mechanical properties. The release-modulating power of the carrier may be manipulated by altering the mechanical properties of the solid. Thickeners well known in the art can be selected for use in the compositions of this disclsoure according to the parameters of the solid composition such that the composition fulfills the desired properties and intended functions of the disclosure. Examples of thickeners, include, but are not limited to, any sugar, polyvinylpyrrolidone, cellulosics, polymers, alginates, other viscosity modifiers (e.g. silica, bentonite, magnesium aluminum silicate, etc.), etc.

Other thickening agent compositions that may be used for the purpose of this disclosure are known to those skilled in the art and it should be appreciated that such compositions may be suitable to provide all or part of the thickening agent component of the composition.

Other Additives

Other additives conventionally used in formulating solid compositions can optionally be included in compositions of this disclosure. These additives are well known in the art and may include colorant, flavoring, solvents, co-solvents, dilutent, disintegrant, glidant, lubricant, opacifying agent, humectant, granulating agent, gelling agent, polishing agent, suspending agent, sweetener, anti-adherent, preservative, emulsifier, antioxidant, levigating agent, plasticizer, tonicity agent, viscosity agent, controlled-release agent, coating, wax, wetting agent, thickening agent, stiffening agent, stabilizing agent, solubilizing agent, sequestering agent, film forming agent, essential oil, emollient, dissolution enhancer, dispersing agent, cryoprotectant stabilizers, chelating agents, sequestering agents, disintegrates, antioxidants, acidifying agents, alkalizing agents, emulsifiers or solubilizers, perfumes, humectants or similar. The additives may be added the composition in combinations.

Other additive compositions that may be used for the purpose of this disclosure are known to those skilled in the art and it should be appreciated that such compositions may be suitable to provide all or part of the other additive component of the composition.

It should be appreciated that there is substantial overlap between the listed components and their functional usage in the composition, since a single chemical may be classified differently depending on the user. For example, polyethylene glycol may be classified as a carrier material as well as a binder. As such, the examples listed above are for illustrative purposes and should not be limiting in any manner. The amounts of described components can be readily determined by someone skilled in the art.

It should be appreciated that types and amounts of additives are selected such that their addition to the compositions herein do not substantially negatively affect the desired properties of the composition for generation of a vapor comprising MME or a transport moiety and MME. Choice and amount of additives, as is well understood in the art, is made in view of the applications for which the composition is intended. One or ordinary skill in the art can make such choices in view of the application based on disclosure herein and what is known in the art without resort to undue experimentation.

In embodiments, the solid compositions herein comprise 0.5 to 50% by weight of the MME-transport moiety blend. In more specific embodiments, the solid compositions herein comprise 1 to 50% by weight of the MME-transport moiety blend. In additional embodiments, the compositions herein comprise 1-20%, 1-15%, 1-10%, 2-15%, 3-15%, 4-15% or 5-15% by weight of the MME-transport moiety blend. In preferred embodiments, the compositions herein comprise 1-12%, 2-12%, 3-12%, 4-12%, 5-12%, 6-12%, 7-12%, 8-12%, 5-10% or 7-10% by weight of the MME-transport moiety blend. In embodiments, the compositions herein comprise 50% by weight or more of a carrier or mixture of carriers. In embodiments, the compositions herein comprise 80% by weight or more of a carrier or mixture of carriers. In embodiments, the compositions herein comprise 90% or more by weight of a carrier or mixture of carriers. In embodiments, compositions herein comprise 2% or less by weight of surfactant. In embodiments, compositions herein comprise 1% or less by weight of surfactant. In embodiments, compositions herein comprise 0.5% or less by weight of surfactant. In embodiments, compositions herein comprise 0.1% or less by weight of surfactant. In embodiments, total additives including surfactant in compositions herein represent 5% by weight or less of the composition. In embodiments, total additives including surfactant in compositions herein represent 2% by weight or less of the composition. In embodiments, total additives including surfactant in compositions herein represent 1% by weight or less of the composition.

In embodiments, the weight ratio of MME to transport moiety in compositions herein ranges from 0.01 to 1. In more specific embodiments, the weight ratio of MME to transport moiety in compositions herein ranges from 0.01 to 0.8, or 0.01 to 0.5 or 0.01 to 0.25, or 0.01 to 0.2 or 0.01 to 0.1, or 0.01 to 0.05, or 0.05 to 1 or 0.05 to 0.8 or 0.05 to 0.5 or 0.05 to 0.25 or 0.05 to 0.2 or 0.05 to 0.1, or 0.1 to 1, or 0.1 to 0.8 or 0.1 to 0.5, or 0.1 to 0.25 or 0.1 to 0.2.

Preparation of Solid MME Compositions

In some implementations the transport moiety and/or MME is included within the carrier. The MME-transport moiety blend or mixture may be mixed directly into the carrier composition or compounded into the carrier composition as liquid filled particles, micro-particles, microcapsules, nanotubes, and/or other encapsulants that are compounded within the carrier. The MME-transport moiety blend or mixture may be a liquid, slurry or solid, put preferably is a liquid. Not only may the MME-transport moiety blend be contained within the inner core of the encapsulant(s) it may also be diffused throughout or located on the surface of any particle(s), micro-particle(s), microcapsule(s), nanotube(s), etc. In some implementations, the MME-transport moiety may be reversibly bound to the backbone of the carrier material. In other implementations, the MME-transport moiety blend may be included within other components of the composition before mixing with the carrier. For example, the MME-transport moiety blend may be mixed directly into the surfactant and/or other additive or compounded into the surfactant and/or other additive as liquid filled particles, micro-particles, microcapsules, nano-tubes, and/or other encapsulants that are compounded within the surfactant and/or other additive. Selected amounts of the MME and transport moiety may be included in the compositions herein as a mixture or blend or each component may be separated included in the compositions.

The compositions described herein can be prepared in a variety of different forms, those including, but not limited to, a capsule/mini- or microcapsule, a tablet, a temporary or permanent suspension, a wafer, a dissolving tablet, a solid, a granule, a film, a pellet, a bead, a powder, a triturate, a platelet, a strip, a sachet, etc. Compositions can also be provided for use in the sub-atmospheric system as a dry powder, solid, capsule, etc. that is placed in contact with a liquid (e.g. solvent, water, etc.) that converts the solid into a liquid form. The forms described herein are known in the art and are packaged accordingly.

Processes for generating the solid may include agglomeration, air suspension chilling, air suspension drying, balling, coacervation, comminution, compression, palletization, cryopellitization, extraction, granulation, homogenization, inclusion complexation, lyophilization, nanoencasulation, melting, mixing, molding, pan coating, solvent dehydration, sonication, spheronization, spray chilling, spray congealing, spray drying, or other processes known to one skilled in the art. Other process for generating the solid may be used for the present disclosure those including compressing solids and/or waxes into tablet form, wet or dry granulation, co-melting, blending dry powders, molding, spray-congealing, layering, encapsulating and microencapsulating, spray-drying, spherionization, titurating, lyophilization, freeze-drying, torching, pelletizing, aerosilization, any liquid or semi-solid preparation (e.g. dispersions, etc.). Yet other processes for generating the solid may include balling, spheronization, extrusion, etc.

In some implementations, direct powder or wax blends may be used to comprise the solid form of the MME-transport moiety. Direct blends may be prepared by blending weighted doses of the composition components followed by encapsulating (e.g. via coating, etc.), compressing into tablet, etc. In other implementations, the solid composition or solid dispersions comprising the MME-transport moiety may be prepared using any suitable process for preparing solid compositions known within the art. Solid dispersions may be prepared in using different methods (e.g. using organic solvents or by dispersing or dissolving the MME-transport moiety in another medium suitable for the disclosure. Wet granulation solids may be prepared by dissolving the MME-transport moiety and the carrier and/or other formulation components in a common organic solvent followed by removing said solvent via evaporation.

The type of solvent may vary depending on the type of MME-transport moiety, carrier, etc. More than one type of compatible, miscible, etc. organic solvent(s) may be used for generating dispersions. Some examples of organic solvents include but are not limited to methanol, ethanol, isopropanol, methylene chloride, chloroform, ethyl acetate, acetone, mixtures thereof, etc. Separate from the organic solvent method described above a solid may be prepared by dispersing and/or dissolving the MME-transport moiety in the carrier composition by blending, compounding, agglomeration, etc. In yet other implementations a co-melting process may be used by melting a carrier or other formulation component and using that component in its melted state as a solvent to dissolve or disperse the MME-transport moiety. Following cooling and solidification of the formulation further processing may occur such as compression.

Any component described above may be added as part of the co-melting composition. The process of freeze-drying may be used in some implementations by dissolving the MME-transport moiety and any co-component (e.g. carrier, surfactant, etc.) in water. The component mixture may then be frozen and place in vacuum at which point the water of the mixture is removed via sublimation resulting in a powder which can be further processed (e.g. compressing, etc.). Spray drying may be used in some implementations. During this process the MME-transport moiety, carrier and/or other component is dispersed in a solvent. The dispersed solution is sprayed into a chamber at which point solvent is evaporated off of droplets of MME solid droplets forming a powder consisting of the MME-transport moiety and the solid. Further processing of the powder may occur after spray drying.

There are a number of different processes that may be used for generating the final solid form of the MME-transport moiety composition. The process selected will depend on the type of MME-transport moiety, carrier, other ingredients, solvent compatibility and the final dosage concentration and/or form required for release, storage, etc. of the MME-transport moiety from the solid. For example, the solid MME-transport moiety composition may be formulated such that the solid provides immediate and quick release of a single dose of MME-transport moiety. A slower more delayed release of multiple doses of the MME-transport moiety from the solid may be provided by altering the ratio and/or formulation components of the solid. The additives may be added the composition in combinations. Other processes for generating the final solid form of the MME-transport moiety are known to those skilled in the art and it should be appreciated that such processes may be suitable to provide all or part of the solid composition.

The present disclosure also provides methods of using the solid MME-transport moiety composition described above.

Any chamber capable of achieving reduced pressures can cause an MME-transport moiety to outgas from a solid and as such can be used in applications of this disclosure. In some implementations, heat can be supplied by a conductive heating assembly to facilitate MME-transport moiety outgassing from the solid.

A representative apparatus for carrying out the method of the present disclosure comprises a processing chamber and a chamber extension. The chamber extension may be connected to the process chamber externally or internally. The chamber can include any suitable MME-transport moiety solid composition delivery mechanism. The MME-transport moiety solid composition may be supplied to the process chamber and/or the chamber extension in solid form. For example, implementations of the delivery mechanism can include bulk sources of solid that are stored internal to the chamber. Implementations of the delivery mechanism can also include MME-transport moiety solid packages (e.g. solid or gel-form) that are disposed directly within the process chamber and/or the chamber extension. The container or wrapping containing the MME-transport moiety solid or gel may be removed from the solid prior to placing the MME-transport moiety solid in the chamber and/or chamber extension. The container or wrapping containing the MME-transport moiety solid or gel may be provided in a gas permeable packet or cartridge that is capable of allowing transport of MME-transport moiety vapor during the outgassing process out of the packet or cartridge while tarping other gel or solid material within the container or wrapping.

A number of vapor gas porous membrane materials suitable for use with the present are known in the art. Some examples include, but are not limited to, polytetrafluoroethylene (PTFE), polyethersulfone (PES), or high-density polyethylene (HDPE). The exact gas-permeable membrane (e.g. material type, porosity, etc.) may be selected based on the type, form, components, etc. of the MME-transport moiety solid composition. In some implementations, the solid or gel form MME-transport moiety composition may be disposed within a sealed gas-permeable membrane and the sealed within a non-permeable wrapper. Prior to use, the non-permeable wrapper may be removed and the gel- or solid-form MME-transport moiety and the gel- or solid-form MME-transport moiety contained within the gas-permeable membrane may be inserted into the drying system. MME-transport moiety solid or gel packages may be disposed within a separate chamber that is in fluid communication with the process chamber and/or chamber extension. In other implementations, the MME-transport moiety solid or gel may be dissolved in a compatible solvent within the process chamber and/or chamber extension forming a liquid prior to heating and/or reduced pressure.

MME-transport moiety vapor that is outgassed from a solid in a negative pressure environment, with or without heat, is useful in a variety of applications. Such vapor can assist in the process of cleaning, sterilization, sanitization, coating, etc.

Components of the process chamber and chamber extension are manufactured in any suitable manner and in any suitable size and shape and/or material so that the system can withstand sub-atmospheric pressures and can accompany the MME-transport moiety solid. For example, the process chamber and chamber extension can be made of metal or durable plastic and may include seals, made of any suitable material, where necessary to maintain sub-atmospheric pressures within the system. Some implementations may include multiple process and/or chamber extensions for concurrent but segregated vaporization of the MME-transport moiety from the solid. MME-transport moiety vapor may be generated in one chamber extension and transported to one or more process chambers.

One or more MME-transport moiety vapors may be generated in one or more chamber extensions and may flow to one or more process chambers. One or more MME-transport moiety vapor(s) may be generated in the process chamber. Some vacuum systems that utilize MME-transport moiety solid(s) are designed to facilitate use within the context of a larger assembly (e.g. a wall-mounted or case-integrated vacuum system). In one implementation, multiple process chambers are stacked in a configuration that allows access like a drawer, chest, etc. Other implementations further include windows, internal lighting (such as UV light) and/or other features to allow users to view the inside environment. In another implementation, the vacuum system may be connected to other processing modules. Some examples include a vacuum system that is connected in-line with one or more wet benches or solvent benches or with one or more ultrasonic cleaners, washer-decontaminators, washer disinfectors, washer-sterilizers, a sanitizer or sterilizer of any type. The vacuum system may also be connected in-line with any type of apparatus that processes the MME-transport moiety solid and/or MME-transport moiety vapor such as filters, dryers, humidifiers, catalytic materials, etc.

The vacuum chamber is depressurized by a depressurizing subsystem such as a vacuum pump or the like that is capable of achieving a reduced pressure environment in the process chamber and/or chamber extension. The specifications of the depressurizing system are selected to produce a desired vacuum level within a desired amount of time, given the air-space within the process chamber and/or chamber extension, the quality of the process chamber and chamber extension seals, etc. An exemplary depressurizing subsystem includes a one-half-horsepower, two-stage vacuum pump configured to produce a vacuum level within the process chamber and/or chamber extension of approximately 0.4 inches of mercury (“inHg”) within seconds and to maintain substantially that level of pressure throughout the process of outgassing the MME-transport moiety from the solid. Different depressurizing subsystem specifications can be used to support multiple vacuum systems, vacuum systems of different sizes, use in portable versus hard-mounted implementations, etc.

The depressurizing subsystem is in fluid communication with the vacuum system (or multiple vacuum systems) via one or more fluid paths. The fluid path can include one or more components including release valves, hoses, fittings, seals, etc. and components may be made of any suitable material. Release valve components may be controlled manually or electronically for the purpose of bringing the negative pressure environment to atmosphere in order to allow the vacuum system to be opening after a process has completed or at any other desirable time. A filter may be included as part of the release valve or as part of any other fluid path component so as to prevent contaminants (e.g. dirt/dust, moisture, etc.) from being allowed to reenter the vacuum system. The release valve may also be connected to a source of purified non-reactive gas such as argon or nitrogen, or a combination of non-reactive gas and air, which may also be bled into the drying system to bring the negative pressure environment to atmosphere. In some implementations the non-reactive gas and/or non-reactive gas and air combination may serve as a carrier gas, further enhancing mobility of the outgassed MME-transport moiety vapor throughout the process chamber and/or chamber extension. In implementations in which multiple vacuum systems are utilized, multiple fluid path components such as multiple release valves or other techniques may be used to fluidly couple the pressurizing subsystem with the vacuum system.

Depressurization of the process chamber and/or the chamber extension causes the MME-transport moiety within the solid to gasify (e.g. evaporate, vaporize, etc.) triggering the release of the MME-transport moiety vapor from the solid into the process chamber or from the chamber extension into the process chamber. Depressurization of the process chamber(s) and the chamber extension(s) may occur simultaneously or in sequence and the sequence may be repeated. MME-transport moiety vapor may also be carried from the chamber extension to the process chamber via a non-reactive carrier gas such as air, argon or nitrogen. In other implementations, heat may be provided to the process chamber and/or the chamber extension and the amount and pattern of heating may vary over time for various purposes. For example, the amount of heat (e.g. and/or a profile of changes in temperature and/or pressure over time) can be tailored to particular solid phase MME-transport moieties and corresponding vapor pressures for vaporization of those MME-transport moieties from the solid. In yet other implementations, heat may be provided to the MME-transport moiety vapor.

In some implementations, the process chamber and/or chamber extension may contain porous tray(s), box(es), envelopes(s), etc. that the user may place the solid MME-transport moiety article into for further processing. The porous tray(s), box(es), envelope(s), etc. may be configured in such a way that they may be heated, if required. Any tray, box, envelope, etc. configuration is conceivable. For example, each tray, box, envelope, etc. may contain any size pores on one or more sides of the tray, box, envelope, etc. The tray, box, envelope, etc. may be sufficiently porous to allow fluid transport of the MME-transport moiety vapor through the tray, box, envelope, etc. into the process chamber and/or chamber extension.

Conductive heat may be used to provide heating to the process chamber and/or chamber extension and/or the porous tray(s), box(es), envelope(s), etc. within the process chamber and/or chamber extension. A heating subsystem heats the process chamber and/or chamber extension or the porous tray(s), box(es), envelope(s), etc. which may be in contact with the solid. Implementations of the tray(s), box(es), envelope(s), etc. may at least partially conform to the external shape of the solid so as to partially surround the solid. For example, the tray(s), box(es), envelope(s) are designed to gently immerse or blanket the solid in such a way as the tray(s), box(es), envelope(s), etc. has full or partial conformal contact with the solid. For example, the porous tray(s), box(es), envelope(s), etc. may contain conductive beads, conductive mesh, heat packs, etc. that can be assembled in a manner that dynamically conforms to the geometry of the solid when the solid comes into contact with the beads, mesh, heat packs, etc.

The heating subsystem can heat the process chamber and/or chamber extension from the outside (e.g., from the bottom and/or side of the process chamber and/or chamber extension). The applied heat from the heating subsystem (e.g. resistive electrical or radiant heater) is conducted toward the solid and/or tray(s), box(es), envelope(s) and/or conductive beads, conductive mesh, heat packs, etc. permitting the heat to evenly be distributed.

Other subsystems can be used to provide additional functionality. These may include a monitoring subsystem that can provide feedback control, environmental monitoring within the process chamber and/or chamber extension, etc. Implementations of the monitoring subsystem may include one or more probes, sensors, cameras and/or any other suitable device. The monitoring subsystem may include one or more sensors situated inside the vacuum system and configured to monitor internal pressure (vacuum level), humidity, temperature, etc. within the vacuum system.

The monitoring subsystem can communicate its measurements through wired and/or wireless communications links to a controller located outside the vacuum system. For example, the controller includes memory (e.g. non-transient, computer-readable memory) and a processor (e.g. implemented as one or more physical processors, one or more processor cores, etc.). The memory has instructions stored thereon, which, when executed, cause the processor to perform various functions. The functions can be informed by (e.g. directed by, modified according to, etc.) feedback from the monitoring subsystem. For example, the measurements from the monitoring subsystem can be used to determine when to end the drying process and release a pressure release valve of the process chamber, when and how to modify the heat being delivered to the conductive thermal assembly, etc. The controller can also direct operation of other subsystems, such as the conveyor assembly, pressurizing subsystem, etc.

The MME-transport moiety vapors are produced by the preferential vaporization of MME-transport moiety from a solid in a reduced pressure, e.g. sub-atmospheric environment. The purity and concentration of MME-transport moiety loaded into the solid can be adjusted according to the intended use. It may be seen that an MME-transport moiety solid that contains a more concentrated form of the MME-transport moiety may be used for multiple process runs. The vacuum system can be configured for a user to easily and safely place the MME-transport moiety solid into the vacuum system before pressurizing the system.

In various implementations, it may be desirable to heat the MME-transport moiety solid to a temperature that is elevated compared to a temperature that is required in a separate process chamber. Accordingly, by providing separate heaters and/or chambers (e.g. chamber extension), the temperature of the process chambers and the temperature of the solid in the chamber extension can be individually heated. The separation of process parameters by use of an isolated chamber and/or temperature may provide improved performance.

The following description of methods are not meant to be limiting.

The MME-transport moiety composition is placed, manually or automatically, into the process chamber of the vacuum system after which point the door(s) to the system are closed and sealed and the system is pressurized by the pressurizing subsystem. The heating subsystem optionally provides heat to the conductive thermal assembly of the process chamber. As the pressure in the chamber is reduced the MME-transport moiety vapor from the solid is outgassed. In an embodiment, the pressure in the process chamber is reduced to below 1 torr. In an embodiment, the pressure in the process chamber is reduced to below 0.1 torr. In an embodiment, the pressure in the process chamber is reduced to 1×10−3 torr or less. Heated or non-heated, non-reactive carrier gas may be introduced into the process chamber enhancing flow of the MME-transport moiety vapor throughout the process chamber.

Different portions of the system may be operated at different temperatures. For example, the process chamber may be operated at one temperature and other portions of the system may be operated at temperature(s) different from the processing chamber. The process chamber which contains vapor comprising the transport moiety or a mixture of MME and transport moiety may be operated at ambient room temperature or heated (by any known means) to a higher temperature up to 100° C. Preferably, the process chamber is operated at a temperature ranging from ambient room temperature to 60° C.

The pressure in the process chamber is preferably maintained at a selected pressure for a selected time to initiate and complete on outgassing and conversion of the MME and/or transport moiety into vapor. One skilled in the art would understand that the holding vapor chamber pressure and the vapor pressure holding time can be adapted to a given application of the MME-transport moiety blend vapor. In embodiments, the selected pressure maintained in the apparatus ranges from 0.1 torr to 200 torr. More specifically, the selected pressure maintained in the apparatus ranges from 0.5 torr to 10 torr. More specifically, the selected holding pressure maintained in the apparatus is about 0.5 to 3 torr. Pressure is generally maintained at the selected value+/−10%. In embodiments, the selected holding pressure time is maintained for 1 minute to 24 hours. In embodiments, the selected holding pressure time is maintained for 5 minutes to 1 hour. In embodiments, the selected holding pressure time is 5 minutes to 30 minutes. In a specific embodiment, the selected holding pressure time is 15 minutes+/−10%.

In another description, the vacuum system is essentially the same as the one described above except that a separate process chamber and chamber extension are utilized as part of the vacuum system. In this regard, the process chamber is configured with a conductive thermal assembly. The chamber extension is configured with a separately controlled conductive thermal assembly and is designed to receive an MME-transport moiety solid. The chamber extension may also include a door or doors that include gaskets or other seals to allow the chamber extension to be sufficiently sealed when the door is closed and the chamber extension is pressurized. The process chamber and the chamber extension are in fluid communication with one another via path or conduit. The conduit may use valves (first valves) that may be actuated/controlled by the controller or user. Both vacuum system descriptions include a second valve (second valves) between the first chamber and the pressurizing system (e.g. vacuum pump). This second valve may be controlled by the user.

In operation, both valves may be opened to permit the pressurizing subsystem to evacuate each chamber. In various operations, upon achieving a desired pressure level (e.g. vacuum level) the first valve may be opened and closed to permit off-gassing and transport of the MME-transport moiety vapor from the chamber extension to the process chamber. The second valve may remain open to permit removal of unused vapor from the system. When desired the first valve may be opened to permit adiabatic expansion and transport of the gasified MME-transport moiety from the chamber extension into the process chamber. The second valve may be closed to allow the MME-transport moiety vapor to enter into the process chamber. Heated or non-heated, non-reactive carrier gas may be introduced into the process chamber enhancing flow of the MME-transport moiety vapor. Heated or non-heated, non-reactive carrier gas may be introduced into the chamber extension enhancing flow of the MME-transport moiety vapor from the chamber extension to the process chamber.

Such an arrangement permits adiabatic expansion of the gasified MME-transport moiety into the process chamber such that the gasified MME-transport moiety is able to expand into all areas of the system. Such a configuration may allow for reduced use of MME-transport moiety solid or MME-transport moiety vapor compared to a system that continually draws a vacuum. Though described as utilizing a single pressurizing system for both chambers, it will be appreciated that the process chamber and the chamber extension have unique depressuring systems. Though discussed in relation to fluidly isolating the chambers, it will be appreciated that is some implementations, the chambers may remain in fluid communication throughout the process. In this implementation, the chamber extension may primarily be used to control the separate heating of the solid. In embodiments, the chamber extension is heated to obtain a desired level of vaporization from the solid. In an embodiment, the chamber extension is heated to a temperature of 100° C. or less.

The term “acetal” and/or “dialkoxy” is recognized in the art and refers to compounds of the formula RCH(OR′)(OR″) in which R, R′ and/or R″ are independently selected from any alkyl, alkenyl, alkynyl, cycloalkyl, cycloalkenyl, cycloalkynyl, aralkyl, alkaryl, aryl, heterocyclyl, etc.

The term “acid anhydride” and/or “carboxylic anhydride” is recognized in the art and refers to compounds of the formula R(CO)O(CO)R′ in which R and R′ are independently selected from any alkyl, alkenyl, alkynyl, cycloalkyl, cycloalkenyl, cycloalkynyl, aralkyl, alkaryl, aryl, heterocyclyl.

As used herein, the term “acyl” is recognized in the art and refers to the entity represented in Formula 1.

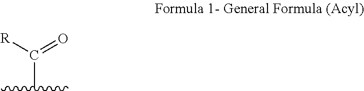

In formula 1, R is independently selected from a hydrogen, an alkyl, and alkenyl, alkynyl, aryl, cycloalkyl, cycloalkenyl, cycloalkynyl, aryl, aralkyl, alkaryl, heterocyclyl and/or a —(CH₂)ₘ—R′, where R′ is independently selected from a hydrogen, an alkyl, and alkenyl, alkynyl, aryl, cycloalkyl, cycloalkenyl, cycloalkynyl, aryl, aralkyl, alkaryl, heterocyclyl, etc., where m is zero or an integer between 1 and 200, inclusive.

As used herein, the term “acylamino” is recognized in the art and refers to the entity represented in Formula 2.

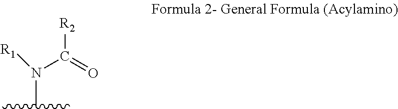

In formula 2, R₁ and/or R₂ are independently selected from a hydrogen, an alkyl, and alkenyl, alkynyl, aryl, cycloalkyl, cycloalkenyl, cycloalkynyl, aryl, aralkyl, alkaryl, heterocyclyl and/or a —(CH₂)ₘ—R′, where R′ may be independently selected from a hydrogen, an alkyl, and alkenyl, alkynyl, aryl, cycloalkyl, cycloalkenyl, cycloalkynyl, aryl, aralkyl, alkaryl, heterocyclyl, etc., where m is zero or an integer between 1 and 200, inclusive.

The term “acyl halide” is recognized in the art and refers to the entity of formula RCOX in which R is selected from any alkyl, alkenyl, alkynyl, cycloalkyl, heterocyclyl, aralkyl, alkaryl or aryl and X is selected from any halogen. The term haloformyl refers to the monovalent radical of formula X—CO—. An acyl halide group is a monovalent radical formed formally by removal of a hydrogen from the R group of an acyl halide.

The terms “acylimino-(alkanediyl, alkenediyl, alkynediyl, cycloalkanediyl, cycloalkenediyl, cycloalkynediyl, aryldiyl, aralkdiyl or alkaryldiyl) are recognized in the art and refer to moieties of the formula Rₐ—R_{b}—R_{c} where Rₐ is an acyl group, R_{b} is a divalent imino group and R_{c} is an alkanediyl, alkenediyl, alkynediyl, cycloalkanediyl, cycloalkenediyl, cycloalkynediyl, aryldiyl, aralkdiyl, or alkaryldiyl, respectively.

The term “acylphospho” is recognized in the art and refers to the entity represented in Formula 3.

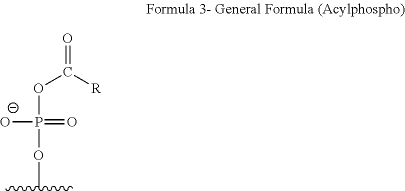

In formula 3, R is independently selected from a hydrogen, an alkyl, alkenyl, alkynyl, cycloalkyl, cycloalkenyl, cycloalkynyl, aryl, aralkyl, alkaryl, heterocyclyl and/or a —(CH₂)ₘ—R′, where R′ is independently selected from a hydrogen, an alkyl, an alkenyl, alkynyl, aryl, cycloalkyl, cycloalkenyl, cycloalkynyl, aryl, aralkyl, alkaryl, heterocyclyl, etc. where m is zero or is an integer between 1 and 200.

The term “acylsulfo” is recognized in the art and refers to the entity represented in Formula 4.

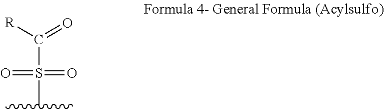

In formula 4, R is independently selected from a hydrogen, an alkyl, alkenyl, alkynyl, cycloalkyl, cycloalkenyl, cycloalkynyl, aryl, aralkyl, alkaryl and/or a —(CH₂)ₘ—R′, where R′ is independently selected from a hydrogen, an alkyl, and alkenyl, alkynyl, aryl, cycloalkyl, cycloalkenyl, cycloalkynyl, aryl, aralkyl, alkaryl, heterocyclyl, etc., where m is zero or is an integer between 1 and 200, inclusive.

The term “alcohol” is recognized in the art and refers to compounds carrying one or more hydroxyl groups —OH. Mono-alcohols include those of formula ROH in which R is selected from any alkyl, alkenyl, alkynyl, cycloalkyl, cycloalkenyl, cycloalkynyl, aryl, aralkyl, alkaryl or heterocyclyl, etc. Alcohols include those carrying more than one hydroxyl group, e.g., diols, triols, etc. The monovalent radical of formula R—O— is an alkoxyl.

The term “aldehyde” is recognized in the art and refers to compounds of carrying a —CHO group and includes compounds of formula RCHO in which R is selected from any alkyl, alkenyl, alkynyl, cycloalkyl, heterocyclyl, aralkyl, alkaryl or aryl, etc.

The term “aliphatic” is recognized in the art and refers to a linear, branched or cyclic alkane, alkene or alkyne. Linear or branched aliphatic groups referred to in compositions of the disclosure have between 1 and 20 carbon atoms. Cycloaliphatic groups may have one or more unsaturation sites (i.e. double or triple bonds) but are not aromatic.

The term “alkanediyl” is recognized in the art and refers to a linear or branched saturated divalent hydrocarbon radical.

The term “alkaryl” (also alkylaryl) is recognized in the art and refers to any monovalent aryl group or any heteroaryl (e.g. aromatic or heteroaromatic) group substituted with one or more alkyl groups. If required, other substitutions to the “alkaryl” can be made and are similar to those described for alkyls herein. The term “alkaryl” may also refer to the monovalent radical moiety of the above described. Some examples include, but are not limited to, o-tolyl, m-tolyl, p-tolyl, 2,3-dimethylphenyl, 3-butylphenyl, 2-(hexan-3-yl-)naphthalen-4-yl, 2-(1-ethoxypropyl)naphthalen-4-yl, etc.

The term “alkaryldiyl” is recognized in the art and refers to any divalent aryl group or heteroaryl (e.g. aromatic or heteroaromatic) group substituted with one or more alkyl groups.

The term “alkenediyl” is recognized in the art and refers to a linear or branched unsaturated divalent hydrocarbon radical. An “alkenediyl” is characterized by a double bond.

The term “alkenyl” is recognized in the art and refers to any monovalent linear hydrocarbon radical or branched monovalent hydrocarbon radical containing at least one double bond. The “alkenyl” may be similar in length and substitutions to the alkyls described herein. The linear or branched alkyl groups referred to in composition of the disclosure have between 3 and 200 carbon atoms and the cycloalkyl groups have between 3 and 200 carbon atoms. Examples of alkenyls include, but are not limited to, ethenyl, propenyl, and the likr.

The term “alkoxy” is recognized in the art and is defined as including an alkyl group attached to an oxygen radical. Examples of alkoxy groups include, but are not limited to methoxy, ethoxy, propyloxy, tert-butoxy, etc. Similarly, an “ether” can be defined as a molecule having two hydrocarbons covalently bound to an oxygen atom. Representative alkyl substituents that may convert said alkyl into an ether may include an “alkoxy” of the form —O-alkyl, —O-alkynyl, —O—(CH₂)m-R, where R can be an alkyl, alkenyl, aryl, cycloalkyl, cycloalkenyl, heterocyclyl or polycyclyl. Similarly, the term “alkenyloxy” is defined as including an alkenyl group attached to an oxygen radical. Examples of alkenyloxy groups include, but are not limited to, allyoxy, crytyloxy, 2-pentenyloxy, 3-hexenyloxy. The term “alkynyloxy” is defined as including an alkynyl group attached to an oxygen radical. The term “cycloalkoxy” is define as including a cycloalkyl group attached to an oxygen radical. The term “cycloalkenyloxy” is defined as including a cycloalkenyl group attached to an oxygen radical. The term “cycloalkynyloxy” is defined as including a cycloalkynyl group attached to and oxygen radical.

The term “alkoxyalkanediyl”, “alkoxyalkenediyl”, alkoxyalkynediyl″, “alkoxycycloalkanediyl”, “alkoxycycloalkenediyl”, “alkoxycycloalkynediyl”, “alkoxyaryldiyl”, “alkoxyaralkdiyl” and “alkoxyalkaryldiyl” are recognized in the art and refer to moieties of the formula Rₐ—O—R_{b}— where Rₐ is an alkyl group and R_{b} is a alkanediyl, alkenediyl, alkynediyl, cycloalkanediyl, cycloalkenediyl, cycloalkynediyl, aryldiyl, aralkdiyl, or alkaryldiyl, respectively.

The term “alkyl” is recognized in the art and refers to any monovalent saturated aliphatic group that is linear and/or branched, and further includes cycloalkyl groups, cycloalkyl groups with alkyl substitutions and/or alkyl groups with cycloalkyl substitutions. The linear or branched alkyl groups referred to in composition of the disclosure have between 1 and 200 carbon atoms and the cycloalkyl groups have between 3 and 200 carbon atoms. “Alkyl” groups are optionally substituted such that one or more hydrogen atoms in the alkyl group are substituted with an alternative substituent. Alternative substituents include, but are not limited to, a halogen, a hydroxy, a carboxyl, an alkoxycarbonyl, a formyl, an acyl, or other carbonyl, a thioester, a thioacetate, a thioformate, or other thiocarbonyl, an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aryl, an aralkyl, an aromatic or heteroaromatic group, etc. It will be understood by one skilled in the art that the alternative substituents may themselves be substituted. Substituted forms of the alternative substituents may include, but are not limited to, any form of an amino, azido, imino, amido, phophoryl (such as phosphonate, phosphinate, etc.), sulfonyl (such as sulfate, sulfonamide, sulfamoyl, sulfonate, etc.), silyl, ether, alkylthios, carbonyls (such as ketones, aldehydes, carboxylates, esters, etc.), cyano, etc. Examples of alkyls include, but are not limited to, methyl, ethyl, propyl, isopropyl, isobutyl, sec-butyl, pentyl, n-hexyl, octyl, dodecyl, and the like.

The term “alkylamino” is recognized in the art and refers to moieties of the formula (Rₐ)ₙ—R_{b} where Rₐ is any alkyl and R_{b} is any divalent radical nitrogen containing group (e.g. amide, amine, imine, imide, azide, azo, cyanate, nitrate, nitrile, nitro, nitrite, nitroso, oxime, pyridine, carbamate ester, etc.) where n is an integer between 1 and 200, inclusive.

The terms “alkylaminoalkanediyl”, “alkylaminoalkenediyl” “alkylaminoalkynediyl”, “alkylaminocycloalkanediyl”, “alkylaminocycloalkenediyl”, “alkylaminocycloalkynediyl”, “alkylaminoaryldiyl”, “alkylaminoaralkdiyl”, and “alkylaminoalkaryldiyl” are recognized in the art and refer to moieties of the formula (Rₐ)ₙ—R_{b}—R_{c} where Rₐ is any alkyl, R_{b} is any divalent radical nitrogen containing group (e.g. amide, amine, imine, imide, azide, azo, cyanate, nitrate, nitrile, nitro, nitrite, nitroso, oxime, pyridine, carbamate ester, etc.) and R_{c} is an alkanediyl, alkenediyl, alkynediyl, cycloalkanediyl, cycloalkenediyl, cycloalkynediyl, aryldiyl, aralkdiyl, and alkaryldiyl, respectively where n is an integer between 1 and 200, inclusive.

The term “alkylphospho” is recognized in the art and refers to moieties of the formula (Rₐ)ₙ—R_{b} where Rₐ is any alkyl and R_{b} is any divalent radical phosphorus containing group e.g. phosphine, phosphoric acid, phosphate, phosphodiester, etc.). n is an integer between 1 and 200, inclusive.

The terms “alkylphosphoalkanediyl”, “alkylphosphoalkenediyl” “alkylphosphoalkynediyl”, “alkylphosphocycloalkanediyl”, “alkylphosphocycloalkenediyl”, “alkylphosphocycloalkynediyl”, “alkylphosphoaryldiyl”, “alkylphosphoaralkdiyl”, and “alkylphosphoalkaryldiyl” are recognized in the art and refers to moieties of the formula (Rₐ)ₙ—R_{b}—R_{c} where Rₐ is any alkyl, R_{b} is any divalent radical phosphorus containing group (e.g. phosphine, phosphoric acid, phosphate, phosphodiester, etc.) and R_{c} is an alkanediyl, alkenediyl, alkynediyl, cycloalkanediyl, cycloalkenediyl, cycloalkynediyl, aryldiyl, aralkdiyl, and alkaryldiyl, respectively, where n is an integer between 1 and 200, inclusive.

The term “alkylsulfo” is recognized in the art and refers to moieties of the formula (Rₐ)ₙ—R_{b} where Rₐ is any alkyl and R_{b} is any divalent radical sulfur containing group (e.g., thiol, sulfide, disulfide, sulfoxide, sulfone, sulfinic acid, sulfonic acid, sulfonate ester, thiocyanate, thioketone, thiocarboxylic acid, thioester, dithiocarboxylic acid, dithiocarboxylic acid ester, etc.) where n is an integer between 1 and 200, inclusive.

The terms “alkylsulfoalkanediyl”, “alkylsulfoalkenediyl” “alkylsulfoalkynediyl”, “alkylsulfocycloalkanediyl”, “alkylsulfocycloalkenediyl”, “alkylsulfocycloalkynediyl”, “alkylsulfoaryldiyl”, “alkylsulfoaralkdiyl”, and “alkylsulfoalkaryldiyl” are recognized in the art and refer to moieties of the formula (Rₐ)ₙ—R_{b}—R_{c} where Rₐ is any alkyl, R_{b} is any divalent radical sulfur containing group (e.g. thiol, sulfide, disulfide, sulfoxide, sulfone, sulfinic acid, sulfonic acid, sulfonate ester, thiocyanate, thioketone, thiocarboxylic acid, thioester, dithiocarboxylic acid, dithiocarboxylic acid ester, etc.) and R_{c} is an alkanediyl, alkenediyl, alkynediyl, cycloalkanediyl, cycloalkenediyl, cycloalkynediyl, aryldiyl, aralkdiyl, and alkaryldiyl, respectively where n is an integer between 1 and 200, inclusive.

The term “alkylthio” is recognized in the art and is defined as having a sulfur attached to an alkyl group. In some implementations the term “alkylthio” may refer to an —S-alkyl-, —S-alkenyl, —S-alkynyl and a —S—(CH₂)ₙ—R where R is independently selected from a hydrogen, an alkyl, an alkenyl, an aryl, cycloalkyl, cycloalkenyl, heterocyclyl, polycyclyl, etc., where m is zero or an integer between 1 and 200, inclusive. Examples of alkylthio groups may include, but are not limited to, methylthio, ethylthio, etc.

The term “alkynediyl” is recognized in the art and refers to a linear or branched unsaturated divalent hydrocarbon radical. An “alkynediyl” is characterized by a triple bond.

The term “alkynyl” is recognized in the art and refers to any monovalent linear monovalent hydrocarbon radical or branched monovalent hydrocarbon radical containing at least one triple bond. The “alkynyl” may be similar in length and/or substitutions to the alkyls described herein. The linear or branched alkynyl groups referred to in compositions of the disclosure have between 3 and 200 carbon atoms and the cycloalkynyl groups have between 3 and 300 carbon atoms. Examples of alkynyls include, but are not limited to, ethynyl, propynyl, and the like.

The term “amine” and “amino” are recognized in the art and refer to both unsubstituted and substituted entities that are represented in Formula 5.

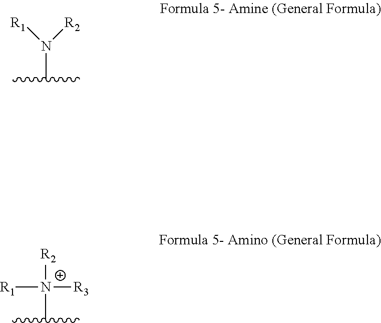

In formula 5, R₁, R₂ and/or R₃ are independently selected from a hydrogen, an alkyl, an alkenyl, alkynyl, cycloalkyl, cycloalkenyl, cycloalkynyl, aryl, aralkyl, alkaryl, heterocyclyl, —(CH₂)ₘ—R′, where R′ is independently selected from a hydrogen, an alkyl, an alkenyl, an alkynyl, an aryl, cycloalkyl, cycloalkenyl, cycloalkynyl, aralkyl, alkaryl, heterocyclyl, polycyclyl, etc., where m is zero or an integer between 1 and 200, inclusive. In some implementations the R₁, R₂ and N atom may be attached to one another in the form of a heterocyclic ring structure. In other implementations, R₁ or R₂ may be a carbonyl. As such, the term “alkylamine” which is recognized in the art, refers to an entity that includes an amine group, as detailed above, which may contain a substituted or unsubstituted alkyl group which is attached to the N. In other words, R₁, R₂ and/or R₃ is an unsubstituted or substituted alkyl group.

The term “aminoacyl” is recognized in the art and refers to the entity represented in Formula 6.

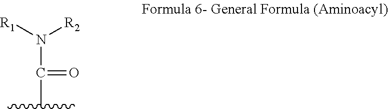

In formula 6, R₁ and/or R₂ are independently selected from a hydrogen, an alkyl, and alkenyl, alkynyl, cycloalkyl, cycloalkenyl, cycloalkynyl, aryl, aralkyl, alkaryl, heterocyclyl and/or a —(CH₂)ₘ—R′, where R′ is independently selected from a hydrogen, an alkyl, an alkenyl, an aryl, cycloalkyl, cycloalkenyl, cycloalkynyl, alkaryl, aralkyl, heterocyclyl, polycyclyl, etc., where m may be zero or may be an integer between 1 and 200, inclusive.

The terms “aminoalkanediyl”, “aminoalkenediyl”, “aminoalkynediyl”, “aminocycloalkanediyl”, “aminocycloalkenediyl”, “aminocycloalkynediyl”, “aminoaryldiyl”, “aminoaralkdiyl” and “aminoalkaryldiyl” are recognized in the art and refer to moieties of the formula Rₐ—R_{b} where Rₐ is any nitrogen containing species group (e.g. amide, amine, imine, imide, azide, azo, cyanate, nitrate, nitrile, nitrite, nitroso, oxime, pyridine, carbamate ester, etc.) and R_{b} is an alkanediyl, alkenediyl, alkynediyl, cycloalkanediyl, cycloalkenediyl, cycloalkynediyl, aryldiyl, aralkdiyl, or alkaryldiyl, respectively.

The term “amide” or “carbamoyl” is recognized in the art and refers to the entity represented in the formula RCONR′R″ in which R, R′ and R″ are independently selected from any alkyl, alkenyl, alkynyl, cycloalkyl, heterocyclyl, aralkyl, alkaryl or aryl.

The term “amido” is recognized in the art and refers to the entity represented in Formula 7.

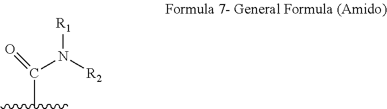

In formula 7, R₁ and/or R₂ may independently be selected from a hydrogen, an alkyl, and alkenyl, alkynyl, cycloalkyl, cycloalkenyl, cycloalkynyl, aryl, aralkyl, alkaryl and/or a —(CH₂)ₘ—R′, where R′ may be independently selected from a hydrogen, an alkyl, an alkenyl, an aryl, cycloalkyl, cycloalkenyl, cycloalkynyl, aralkyl, alkaryl, heterocycle, polycycle, etc., where m is zero or is an integer between 1 and 200, inclusive.

The term “aralkdiyl” is recognized in the art and refers to any divalent alkyl groups that have aryl group (e.g. aromatic, heteroaromatic, etc.) substitutions.

The term “aralkyl” is recognized in the art and refers to any monovalent alkyl groups that have aryl group (e.g. aromatic, heteroaromatic, etc.) substitutions.

The term “heterocyclic” refers to a ring compound or a group (mono radical), saturated or unsaturated, having one or more atoms in the ring structure that is different from carbon. The term “heterocyclyl” more specifically refers to a heterocyclic mono radical. Exemplary heteroatoms include O, N, S and P. Some examples include, but are not limited to, benzyl, phenethyl, 2-phenylbutyl, 4-phenylhexan-3-yl, 4-(pyridine-3-yl)hexan-3-yl, (benzyloxy)methyl, etc.

The term “aromatic” refers to a carbo- or heterocyclic or polycyclic moiety (e.g. carbo- or heterocyclic) that has an unsaturated conjugated electron system or (4n+2) delocalized π electrons in each aromatic ring. Some examples include, but are not limited to, phenyl, biphenyl, benzyl, xylyl, naphthyl, anthryl, phenanthryl, tetrahydro naphthyl, azulenyl, indanyl, indenyl, pyridinyl, pyrrolyl, furanyl, thiophenyl, fluorenyl, fluorenonyl, dibenzofuranyl, dibenzothienyl, furyl, thienyl, pyridyl, oxazolyl, thiazolyl, imidazolyl, pyrazolyl, 2-pyrazolinyl, pyrazolidinyl, isoxazolyl, isothizolyl, oxadiazolyl, triazolyl, thiadiazolyl, pyridazinyl, pyrimidinyl, pyrazinyl, triazinyl, trithianyl, indolinzinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, isoquinolinyl, cinnolinyl, phthalazinyl, quinazolinyl, quinoxalinyl, 1,8-naphthridinnyl, pteridinyl, acridinyl, phenazinyl, phenothiazinyl, phenoxyazinyl, pyrazolo[1,5-c]triazinyl, carbazolyl, benzo[c]cinnolinyl, 9,10-dihydrophenanthrenyl, etc. Aromatic groups are optionally substituted with at least one substituent including, but is not limited to, alkyl, halogen, haloalkyl, hydroxy, hydroxyalkyl, alkenyl, alkenyloxy, alkoxyl, alkoxyalkoxy, alkoxycarbonyl, amino, alkylamino, alkylsulfonyl, dialkylamino, aminocarbonyl, aminocarbonylalkoxy, aryl, arylalkyl, aryalkoxy, aryloxy, cyano, nitro, carboxy, cycloalkyl, cycloalkylalkyl, carboxyalkoxy, phenyl, etc. The definition of “aromatic” also included heteroaromatic moieties as well as multiple ring structures that may or may not be joined together by two carbon atoms. Aromatic compounds include those having fused rings as well as rings that are joined together by a single or double bond between atoms of different rings.

The term “aryl” is recognized in the art and refers to any monovalent radical containing an aromatic ring. Aryl groups include monovalent single-aromatic ring groups wherein the aromatic group can optionally include up to four heteroatoms. Examples include, but are not limited to, phenyl, pyrolyl, furanyl, thiophenyl, imidazolyl, oxazolyl, thiazolyl, triazolyl, tetrazolyl, pyrazolyl, pyridinyl, pyrazinyl, pyridazinyl, pyrimidinyl, etc. “Heteroaryl” groups are aryl groups that include heteroatoms in the ring structure. The aromatic ring structure may be substituted with one or more substituents such as, but not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamide, ketone, aldehyde, ester, heterocyclyl, aromatic or heteroaromatic groups, —CF3, —CN, etc. The definition of “aryl” also includes polycyclic ring structures that may have two or more cyclic rings with two or more adjoined carbon atoms. At least one of the adjoined ring structures is aromatic. Other cyclic rings of the aryl group may include cycloalkyl, cycloalkenyl, cycloalkynyl, aryl and/or heterocyclyl. Examples of “aryl” entities include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl benzopyrrolidinyl, benzomorpholinyl, methylenedioxypenyl, ethylenedioyphenyl, and the like.

The terms “arylaminoalkanediyl”, “arylaminoalkenediyl” “arylaminoalkynediyl”, “arylaminocycloalkanediyl”, “arylaminocycloalkenediyl”, “arylaminocycloalkynediyl”, “arylaminoaryldiyl”, “arylaminoaralkdiyl”, and “arylaminoalkaryldiyl” are recognized in the art and refer to moieties of the formula (Ra)n-Rb-Rc where Ra is any aryl, Rb is any divalent radical nitrogen containing group (e.g. amide, amine, imine, imide, azide, azo, cyante, isocyanate, nitrate, nitrile, nitro, nitrite, oxime, pyridine, carbamate ester, etc.) and Rc is an alkanediyl, alkenediyl, alkynediyl, cycloalkanediyl, cycloalkenediyl, cycloalkynediyl, aryldiyl, aralkdiyl, and alkaryldiyl, respectively, where n is an integer between 1 and 200, inclusive.

The terms “arylphosphoalkanediyl”, “arylphosphoalkenediyl” “arylphosphoalkynediyl”, “arylphosphocycloalkanediyl”, “arylphosphocycloalkenediyl”, “arylphosphocycloalkynediyl”, “arylphosphoaryldiyl”, “arylphosphoaralkdiyl”, and “arylphosphoalkaryldiyl” are recognized in the art and refers to moieties of the formula (Ra)n-Rb-Rc where Ra is any aryl, Rb is any divalent radical phosphorus containing group (e.g. phosphine, phosphoric acid, phosphate, phosphodiester, etc.) and Rc is an alkanediyl, alkenediyl, alkynediyl, cycloalkanediyl, cycloalkenediyl, cycloalkynediyl, aryldiyl, aralkdiyl, and alkaryldiyl, respectively, where n is an integer between 1 and 200, inclusive.

The terms “arylsulfoalkanediyl”, “arylsulfoalkenediyl” “arylsulfoalkynediyl”, “arylsulfoycloalkanediyl”, “arylsulfocycloalkenediyl”, “arylsulfoycloalkynediyl”, “arylsulfoaryldiyl”, “arylsulfoaralkdiyl”, and “arylsulfoalkaryldiyl” are recognized in the art and refers to moieties of the formula (Ra)n-Rb-Rc where Ra is any aryl, Rb is any divalent radical sulfur containing group (e.g. thiol, sulfide, disulfide, sulfoxide, sulfone, sulfinic acid, sulfonic acid, sulfonate ester, thiocyante, thioketone, thial, thiocarboxylic acid, thioester, dithiocarboxylic acid, dithiocarboxylic acid ester, etc.) and Rc is an alkanediyl, alkenediyl, alkynediyl, cycloalkanediyl, cycloalkenediyl, cycloalkynediyl, aryldiyl, aralkdiyl, and alkaryldiyl, respectively, where n is an integer between 1 and 200, inclusive.

The terms “alkylphosphoalkanediyl”, “alkylphosphoalkenediyl” “alkylphosphoalkynediyl”, “alkylphosphocycloalkanediyl”, “alkylphosphocycloalkenediyl”, “alkylphosphocycloalkynediyl”, “alkylphosphoaryldiyl”, “alkylphosphoaralkdiyl”, and “alkylphosphoalkaryldiyl” are recognized in the art and refer to moieties of the formula (Ra)n-Rb-Rc where Ra is any alkyl, Rb is any divalent radical phosphorus containing group (e.g. phosphine, phosphoric acid, phosphate, phosphodiester, etc.) and Rc is an alkanediyl, alkenediyl, alkynediyl, cycloalkanediyl, cycloalkenediyl, cycloalkynediyl, aryldiyl, aralkdiyl, and alkaryldiyl, respectively, wherein n is an integer 1 through 3.

The term “aryldiyl” is recognized in the art and refers to any divalent aromatic hydrocarbon group that may include up to four heteroatoms. Substitutions may be similar to those described for an aryl group.

The term “arylenediyl” is recognized in the art and refers to a divalent unsaturated aromatic carbocyclic radical having one or two rings.

The term “arylphosphanyl” is recognized in the art and refers to the entities that are represented in Formula 8.

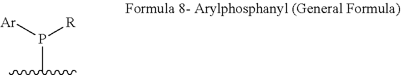

In formula 8, Ar is independently selected from any aryl group. R is selected from a hydrogen, an alkyl, an alkenyl, an aryl, cycloalkyl, cycloalkenyl, heterocycle, polycycle, etc.

The term “arylphosphono” is recognized in the art and refers to the entities that are represented in Formula 9.

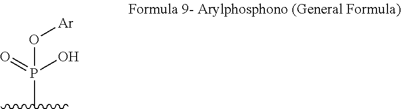

In formula 9, Ar is selected from any aryl group.

Used herein, the term “arylsulfinyl” is recognized in the art and refers to the entities that are represented in Formula 10.

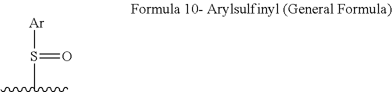

In formula 10, Ar is independently selected from any aryl group.

The term “arylsulfonyl” is recognized in the art and refers to the entities that are represented in Formula 11.

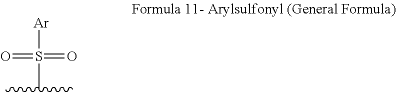

In formula 11, Ar is independently selected from any aryl group.

The term “arylthiol” is recognized in the art and refers to the entities that are represented in Formula 12

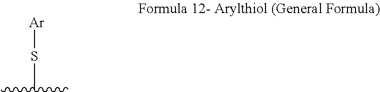

In formula 12, Ar is independently selected from any aryl group.

The term “alkarylenediyl” is recognized in the art and refers to an arylenediyl group that is substituted with at least one alkyl group.

The term “aralkylenediyl” is recognized in the art and refers to an alkylenediyl that is substituted with at least one aryl group.

The term “aryloxy” is recognized in the art and refers to a monovalent radical of formula —O—R where R is an aryl group as described herein.

The term “azide” or “azido” is recognized in the art and refers to the monovalent radical of formula —RN3 where R is selected from any alkyl, alkenyl, alkynyl, cycloalkyl, cycloalkenyl, cycloalkynyl, heterocyclyl, aralkyl, alkaryl or aryl.

The term “azo” is recognized in the art and refers to the monovalent radical of formula —RN₂R′ where R and R′ are independently selected from any alkyl, alkenyl, alkynyl, cycloalkyl, cycloalkenyl, cycloalkynyl, heterocyclyl, aralkyl, alkaryl or aryl.

The term “branch” or “branched” is recognized in the art and refers to one or more appendage off of the polymer length.

The term “carbamate ester” and/or “carbamate” is recognized in the art and refers to a monovalent radical of formula —RO(C═O)NR′₂ in which R and R′ are selected from any alkyl, alkenyl, alkynyl, cycloalkyl, cycloalkenyl, cycloalkynyl, heterocyclyl, aralkyl, alkaryl or aryl, etc.

The term “carbonate” is recognized in the art and refers to a monovalent radical of formula —ROCOOR′ in which R and R′ are independently selected from any alkyl, alkenyl, alkynyl, cycloalkyl, cycloalkenyl, cycloalkynyl, heterocyclyl, aralkyl, alkaryl or aryl, etc.

As used herein, the term “carbonyl” is recognized in the art and refers to the entity represented in Formula 13a and 13b.

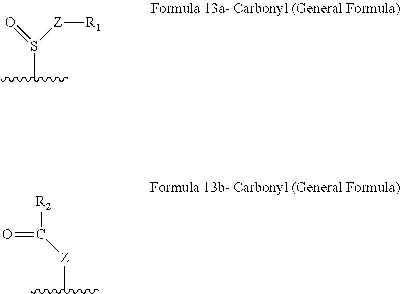

In formulas 13a and 13b, Z may be an oxygen or sulfur atom and R₁ and R₂ are independently selected from hydrogen, an alkyl, an alkenyl, a —(CH₂)ₘ—R where R is selected from an alkyl, alkenyl, alkynyl, aryl, cycloalkyl, cycloalkenyl, cycloalkynyl, aralkyl, alkaryl, heterocyclyl or polycyclyl, where m is zero or is an integer between 1 and 200, inclusive. In some implementations, R₁ and/or R₂ may be a salt.

The term “ester” is recognized in the art and may also be described by Formula 13a and 13b in implementations in which Z is an oxygen and R₁ and R₂ are not hydrogen.

The term “carboxylic acid” is recognized in the art and may also be described by Formula 13a in implementations in which Z is an oxygen and R₁ is a hydrogen.

The term “formate” is recognized in the art and may also be described by Formula 13b in implementations in which Z is an oxygen and R₂ is hydrogen.

The term “thiocarbonyl” is recognized in the art and may also be described by Formula 13a and 13b in implementations in which the oxygen atom is replaced by a sulfur atom. Similarly, in implementations where Z is a sulfur and R₁ and R₂ are not hydrogen the structure is a “thioester”. In implementations where Z is a sulfur and R₁ is a hydrogen, the formula 13a is a “thiocarboxylic acid”. In the instance of a “thioformate”, Z is a sulfur and R₂ is a hydrogen.

The term “ketone” is recognized in the art and may also be described by Formula 13a in implementations where Z is a C—C bond and R₁ is not a hydrogen.

The term “aldehyde” is recognized in the art and may also be described by Formula 13b in implementations where Z is a C—C bond and R₂ is a hydrogen.

The term “carboxylate” is recognized in the art and refers to the monovalent radical of formula —RCOO⁻ in which R may be selected from any alkyl, alkenyl, alkynyl, cycloalkyl, cycloalkenyl, cycloalkynyl, heterocyclyl, aralkyl, alkaryl or aryl, etc. A carboxylate can be negatively charged as indicated, but may also be in the form of a salt with an appropriate cation, such as an alkali metal cation (e.g., Na+).

The term “carboxylic acid” group is recognized in the art and refers to the monovalent radical of formula —RCOOH in which R may be selected from any alkyl, alkenyl, alkynyl, cycloalkyl, cycloalkenyl, cycloalkynyl, heterocyclyl, aralkyl, alkaryl or aryl, etc.

The term “cyanate” or “cyanate” is recognized in the art and refers to the monovalent radical of formula —ROCN which R may be selected from any alkyl, alkenyl, alkynyl, cycloalkyl, cycloalkenyl, cycloalkynyl, heterocyclyl, aralkyl, alkaryl or aryl, etc.

The term “cycloalkanediyl” is recognized in the art and refers to a cyclic saturated divalent hydrocarbon radical.

The term “cycloalkenediyl” is recognized in the art and refers to a cyclic unsaturated divalent hydrocarbon radical. A “cycloalkanediyl” is characterized by a double bond.

The term “cycloalkenyl” is recognized in the art and refers to any cycloalkyl as described herein with at least one double bond. Examples of cycloalkenyls include, but are not limited to, cyclohexenyl, cyclopentenyl, cyclobutenyl, and the like.

The term “cycloalkyl” is recognized in the art and refers to any monovalent saturated carbocyclic entity that comprises a mono- or bicyclic ring structure. “Cycloalkyl” entities may be unsubstituted or substituted similarly to the alkyls described above or as described herein. Examples of cycloalkyls include, but are not limited to, cyclopropyl, cyclobutyl, cyclopentyl, cyclohexyl, cycloheptyl, etc.

The term “cycloalkynediyl” is recognized in the art and refers to a cyclic unsaturated divalent hydrocarbon radical. A “cycloalkynediyl” is characterized by a triple bond.

The term “cycloalkynyl” is recognized in the art and refers to any cycloalkyl as described herein with at least one double bond. Examples of cycloalkynyls include, but are not limited to, cycloheptynyl, cyclooctynyl, cyclononyl, cyclodecynyl, etc.

The term “disulfide” is recognized in the art and refers to the entities of formula RSSR′ in which R and R′ are independently selected from any alkyl, alkenyl, alkynyl, cycloalkyl, cycloalkenyl, cycloalkynyl, heterocyclyl, aralkyl, alkaryl or aryl, etc.

The term “dithiocarboxylic acid” is recognized in the art and refers to entities of formula RC═S₂H in which R is selected from any alkyl, alkenyl, alkynyl, cycloalkyl, cycloalkenyl, cycloalkynyl, heterocyclyl, aralkyl, alkaryl or aryl, etc.

The term “dithiocarboxylic acid ester” is recognized in the art and refers to entities of formula RC═S2R′ in which R and R′ are independently selected from any alkyl, alkenyl, alkynyl, cycloalkyl, cycloalkenyl, cycloalkynyl, heterocyclyl, aralkyl, alkaryl or aryl, etc.

The term “ester” is recognized in the art and refers to the entities of formula RCOOR′ in which R and R′ may independently be selected from any alkyl, alkenyl, alkynyl, cycloalkyl, cycloalkenyl, cycloalkynyl, heterocyclyl, aralkyl, alkaryl or aryl, etc.

The term “halo” is recognized in the art and refers to a “halogen” or “halide” substituent such as fluoro, chloro, bromo, iodo. Halo-substituted groups include among others haloalkyl groups, haloalkenyl groups, haloalkynyl groups, haloaryl groups, halocylcoalkyl groups, halocyloalkenyl groups, halocycloalkynyl groups, haloalkylaryl groups, haloarylalkyl groups and the like.

The term “haloalkanediyl” is recognized in the art and refers to any alkanediyl in which one or more hydrogen atoms have been substituted with the same or different halogens.

The term “haloalkaryl” is recognized in the art and refers to an alkaryl in which one or more hydrogen atoms have been substituted with the same or different halogens.

The term “haloalkaryldiyl” is recognized in the art and refers to an alkaryldiyl in which one or more hydrogen atoms have been substituted with the same or different halogens.

The term “haloalkenediyl” is recognized in the art and refers to an alkenediyl in which one or more hydrogen atoms have been substituted with the same or different halogens.

The term “haloalkenyl” is recognized in the art and refers to an alkenyl in which one or more hydrogen atoms have been substituted with the same or different halogens.

The term “haloalkyl” is recognized in the art and refers to an alkyl in which one or more hydrogen atoms have been substituted with the same or different halogens.

The term “haloalkynediyl” is recognized in the art and refers to an alkynediyl in which one or more hydrogen atoms have been substituted with the same or different halogens.

The term “haloalkynyl” is recognized in the art and refers to an alkynyl in which one or more hydrogen atoms have been substituted with the same or different halogens.

The term “haloaralkyl” is recognized in the art and refers to an aralkyl in which one or more hydrogen atoms have been substituted with the same or different halogens.

The term “haloaralkyldiyl” is recognized in the art and refers to an araalkyldiyl in which one or more hydrogen atoms have been substituted with the same or different halogens.

The term “haloaryl” is recognized in the art and refers to an aryl in which one or more hydrogen atoms have been substituted with the same or different halogens.

The term “haloaryldiyl” is recognized in the art and refers to an aryldiyl in which one or more hydrogen atoms have been substituted with the same or different halogens.

The term “halocycloalkanediyl” is recognized in the art and refers to a cycloalkanediyl in which one or more hydrogen atoms have been substituted with the same or different halogens.

The term “halocycloalkenediyl” is recognized in the art and refers to a cycloalkenediyl in which one or more hydrogen atoms have been substituted with the same or different halogens.

The term “halocycloalkenyl” is recognized in the art and refers to a cycloalkenyl in which one or more hydrogen atoms have been substituted with the same or different halogens.

The term “halocycloalkyl” is recognized in the art and refers to a cycloalkyl in which one or more hydrogen atoms have been substituted with the same or different halogens.

The term “halocycloalkynediyl” is recognized in the art and refers to a cycloalkynediyl in which one or more hydrogen atoms have been substituted with the same or different halogens.

The term “halocycloalkynyl” is recognized in the art and refers to a cycloalkynyl in which one or more hydrogen atoms have been substituted with the same or different halogens.

The term “hemiacetal” is recognized in the art and refers to entities of formula RCH(OR′)(OH) in which R and R′ are independently selected from any alkyl, alkenyl, alkynyl, cycloalkyl, cycloalkenyl, cycloalkynyl, heterocyclyl, aralkyl, alkaryl or aryl, etc.

The term “hemiketal” and/or “alkoxy-ol” is recognized in the art and refers to entities of formula RC(OR″)(OH)R′ in which R, R′ and R″ are independently selected from any alkyl, alkenyl, alkynyl, cycloalkyl, cycloalkenyl, cycloalkynyl, heterocyclyl, aralkyl, alkaryl or aryl, etc.

The term “hetero-”, such as in “heterosubstitution” or “heteroatom” is recognized in the art and refers to an atom of any element other than carbon, such as, but not limited to, N, O, P, B, S, Si, Sb, Al, Sn, As, Se, Ge, etc. that replaces any one or more hydrogen atoms to any carbon and/or any one or more carbon atom within a chemical group. Some examples include, but are not limited to, methoxy, ethoxy, propoxy, isopropoxy, methylthio, ethylthio, propylthio, isopropylthio, quinolinyl, pyridyl, pyrazinyl, indolyl, carbazoyl, furyl, pyrrolyl, thienyl, thiazolyl, pyrazolyl, oxazolyl, etc.

The terms “heterocyclyl” and “heterocyclic” are recognized in the art and refers to a ring structure that includes one or more heteroatoms. A “heterocyclic” compound or a heterocyclyl group may be polycyclic. “Heterocyclyl” and “heterocyclic” groups refer to monovalent radicals. Heterocyclic compounds include, but are not limited to thiophene, thianthrene, furan, pyran, isobenzofuran, chromene, xanthene, phenoxathiin, pyrrole, imidazole, pyrazole, isothiazole, isoxaole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams, pyrrolidinones, sultams, sultones, etc. Heterocyclyl or heterocyclic groups are monovalent radical of heterocyclic compounds, including monovalent radicals of the heterocyclic compounds listed herein. The ring(s) of a heterocyclic compound or monovalent radical may be substituted at one or more positions on the structure with a substituent that includes, but is not limited to, a halogen, a alkyl, a aralkyl, a alkenyl, a alkynyl, a cycloalkyl, a hydroxyl, an amino, a nitro, a sulfhydryl, an imino, an amido, a phosphonate, a phosphinate, a carbonyl, a carboxyl, a silyl, an ether, an alkylthio, a sulfonyl, a ketone, an aldehyde, an ester, a heterocyclyl, and other aromatic or heteroaromatic group, a trifluoromethyl, a cyano group, etc.

The term “hydrocarbon” is recognized in the art and refers to compounds with at least one hydrogen and one carbon atom. More broadly, the definition of hydrocarbon includes, among others, acyclic, cyclic, branched, unbranched (straight-chain), carbocyclic, heterocyclic, aromatic, nonaromatic compounds each of which may be substituted or unsubstituted. Optional substituents for hydrocarbons include, but are not limited to, those substituents noted herein for alkyl groups, aryl groups or heterocyclic groups.

The term “imide” or “imido” is recognized in the art and refers to monovalent radicals of formula —(RCO₂)NR′ in which R and R′ are independently selected from any alkyl, alkenyl, alkynyl, cycloalkyl, cycloalkenyl, cycloalkynyl, heterocyclyl, aralkyl, alkaryl or aryl, etc.

The term “imine” or “imino” is recognized in the art and refers to monovalent radicals of formulas —RC(═NH)R′, —RC(═NR″)R′, —RC(═NH)H, and/or —RC(═NR′)H in which R, R′ and R″ are independently selected from any alkyl, alkenyl, alkynyl, cycloalkyl, cycloalkenyl, cycloalkynyl, heterocyclyl, aralkyl, alkaryl or aryl, etc.

The term “isocyanate” is recognized in the art and refers to entities of formula RNCO in which R may be selected from any alkyl, alkenyl, alkynyl, cycloalkyl, cycloalkenyl, cycloalkynyl, heterocyclyl, aralkyl, alkaryl or aryl, etc. Isocyanate groups include monovalent radicals in which a hydrogen is formally removed from the R group.

The term “ketone” is recognized in the art and refers to entities of formula RCOR′ in which R and R″ are independently selected from any alkyl, alkenyl, alkynyl, cycloalkyl, cycloalkenyl, cycloalkynyl, heterocyclyl, aralkyl, alkaryl or aryl, etc. A ketone group refers to a monoradical in which a hydrogen is formally removed from an R or R′ of a ketone. A ketone group includes a carbonyl group —CO—, which may be substituted with any alkyl, alkenyl, alkynyl, cycloalkyl, cycloalkenyl, cycloalkynyl, heterocyclyl, aralkyl, alkaryl or aryl, etc.

The term “linear” is recognized in the art and refers to a polymer in which the monomers are joined together in one continuous length along the chain of the polymer. At no point along the length of the polymer are there branches.

The term “nitrate” or “nitroxy” is recognized in the art and refers to entities of formula RONO₂ in which R may be selected from any alkyl, alkenyl, alkynyl, cycloalkyl, cycloalkenyl, cycloalkynyl, heterocyclyl, aralkyl, alkaryl or aryl.

The term “nitrile” is recognized in the art and refers to entities of formula RCN and/or —RNC in which R may be selected from any alkyl, alkenyl, alkynyl, cycloalkyl, cycloalkenyl, cycloalkynyl, heterocyclyl, aralkyl, alkaryl or aryl, etc. A nitrile group is the group of formula —CN. An isonitrile group is the group of formula —NC.

The term “nitrite” is recognized in the art and refers to entities of formula RONO in which R may be selected from any alkyl, alkenyl, alkynyl, cycloalkyl, cycloalkenyl cycloalkyyl, heterocyclyl, aralkyl, alkaryl or aryl, etc. A nitrite group is the groups of formula —ONO.

The term “nitro” is recognized in the art and refers to entities of formula RNO₂ in which R may be selected from any alkyl, alkenyl, alkynyl, cycloalkyl, cycloalkenyl, cycloalkynyl, heterocyclyl, aralkyl, alkaryl or aryl, etc. A nitro group is the group of formula —NO₂.

The term “nitroso” is recognized in the art and refers to entities of formula RNO in which R may be selected from any alkyl, alkenyl, alkynyl, cycloalkyl, cycloalkenyl, cycloalkynyl, heterocyclyl, aralkyl, alkaryl or aryl, etc. A nitroso group is the group of formula —NO.

The term “orthocarbonate ester” is recognized in the art and refers to compounds of formula C(OR)(OR′)(OR″)(OR′″) in which R, R′, R″ and/or R′″ are independently selected from any alkyl, alkenyl, alkynyl, cycloalkyl, cycloalkenyl, cycloalkynyl, heterocyclyl, aralkyl, alkaryl or aryl, etc. An orthocarbonate ester group is a monoradical group of an orthocarbonate ester group.

The term “orthoester” is recognized in the art and refers to compounds of formula RC(OR′)(OR″)(OR″) in which R, R′ and/or R″ are independently selected from any alkyl, alkenyl, alkynyl, cycloalkyl, cycloalkenyl, cycloalkynyl, heterocyclyl, aralkyl, alkaryl or aryl, etc.

The term “oxime” is recognized in the art and refers to entities of formula RCH═NOH in which R may be selected from any alkyl, alkenyl, alkynyl, cycloalkyl, cycloalkenyl, cycloalkynyl, heterocyclyl, aralkyl, alkaryl or aryl, etc.

The term “phosphine” and/or “phosphanyl” is recognized in the art and refers to entities of formula R₃P in which R may be selected from any alkyl, alkenyl, alkynyl, cycloalkyl, cycloalkenyl, cycloalkynyl, heterocyclyl, aralkyl, alkaryl or aryl, etc.

The term “phosphoacyl” is recognized in the art and refers to the entity represented in Formula 14.

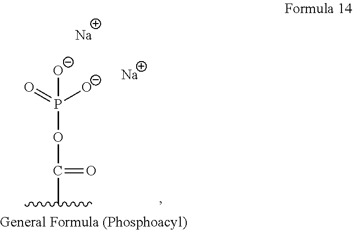

where Na+ is an exemplary cation.

The terms “phosphoalkanediyl”, “phosphpalkenediyl”, “phosphoalkynediyl”, “phosphocycloalkanediyl”, “phosphocycloalkenediyl”, “phosphocycloalkynediyl”, “phosphoaryldiyl”, “phosphoaralkdiyl” and “phosphoalkaryldiyl” are recognized in the art and refer to moieties of the formula Rₐ—R_{b} where Rₐ is any nitrogen containing species group (e.g. phosphine, phosphonic acid, phosphate, phosphodiester, etc.) and R_{b} is an alkanediyl, alkenediyl, alkynediyl, cycloalkanediyl, cycloalkenediyl, cycloalkynediyl, aryldiyl, aralkdiyl, or alkaryldiyl, respectively.

The term “phosphate” and/or “phosphonoxy” is recognized in the art and refers to entities of formula ROP(═O)(OH)₂ in which R may be selected from any alkyl, alkenyl, alkynyl, cycloalkyl, cycloalkenyl, cycloalkynyl, heterocyclyl, aralkyl, alkaryl or aryl, etc.

The term “phosphodiester” and/or “(alkoxy)hydroxyphosphoryloxyis recognized in the art and refers to entities of formula HOPO(OR)₂ in which R may be selected from any alkyl, alkenyl, alkynyl, cycloalkyl, cycloalkenyl, cycloalkynyl, heterocyclyl, aralkyl, alkaryl or aryl, etc. A “(alkoxy)hydroxyphosphoryloxy” relates to a monovalent radical of a phosphodiester.

The term “phosphoric acid” is recognized in the art and refers to entities of formula RP(═O)(OH)₂ in which R may be selected from any alkyl, alkenyl, alkynyl, cycloalkyl, cycloalkenyl, cycloalkynyl, heterocyclyl, aralkyl, alkaryl or aryl, etc. “Phosphono” relates to monovalent radicals of phosphoric acid.

The term “phosphoryl” is recognized in the art and may be represented in general by Formulas 15a-15c:

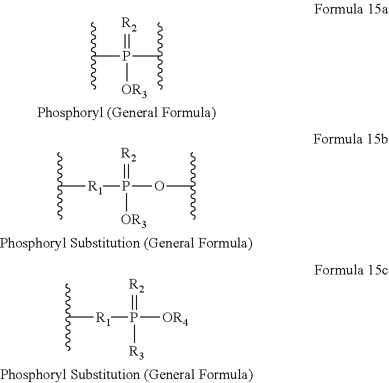

In formulas 15a-c, R₂ is S or O, R₃ and R₄ are independently selected from a hydrogen, an alkyl, alkenyl, alkynyl, cycloalkyl, cycloalkenyl, cycloalkynyl, heterocyclyl, aralkyl, alkaryl or an aryl, etc. and R₁ is S, O or NH. The phosphoryl group becomes a “phosphorothioate” when R₂ is an S. In embodiments of formulas 15a-c, R₁ represents S or O and R₃ and R₄ are independently selected from hydrogen, alkyl, alkenyl, alkynyl, cycloalkyl, cycloalkenyl, cycloalkynyl, heterocyclyl, aralkyl, alkaryl or aryl. In some implementations, when a phosphoryl is used in substitution, for example with an alkyl, the phosphoryl moiety of phosphorylalkyl may by represented by the general formulas 15b or c.

The term “polycyclic” is recognized in the art and refers to with two or more ring structures in which two or more adjacent carbons of the ring structures are adjoined together (e.g. cycloalkyls, cycloalkenyls, cycloalkynyl, aryls and/or heterocyclyl). The rings may also be adjoined by carbons that are non-adjacent. Rings in the “polycyclic” structure may be substituted as described above. Substituents include, but are not limited to, one or more of halogen, alkyl, aralky, alkenyl, alkynyl, cycloalkyl, hydroxyl, amino, nitro, sulfydryl, imino, amido, phosphonate, phophinate, carbonyl, carboxyl, silyl, ether, alkylthio, sulfonyl, ketone, aldehyde, ester, heterocyclyl, aromatic or heteroaromatic, trifluoromethyl, cyano, etc. group(s). A “polycyclic” compound with more than one substituent may contain the same type substituent or different types of substituents.

The term “polymer” is recognized in the art and is used to describe a substance that are macromolecules that are composed of monomers that have been polymerized. The polymer may contain monomers that are all the same or a mixture of monomers. The macromolecule has a relatively high molecular mass and has a chain-like structure that comprises repeating units. The chain-like structure optionally has pendant atoms or groups bonded to the chain-like structure. The term “polymer” includes any homopolymer, copolymer, terpolymer, interpolymer, etc. A homopolymer is a macromolecule generated from one species of monomer, in other words it has one type of repeating unit. A copolymer is a polymer generated from multiple species of compatible monomers or comonomers. The term copolymer may be used interchangeable with interpolymer. A terpolymer refers to a polymer generated by three different species of monomer, and the like.

The term “protecting group” is recognized in the art and is used to describe substituents that are temporary and are used to protect a reactive functional group from reacting undesirably during chemical reactions. Some examples of protecting groups include, but are not limited to, esters of carboxylic acids, silyl ethers of alcohols, acetals of aldehydes, ketals of ketones, etc. As is known in the art there may be different protecting groups for different reactive functional groups and different protective groups may be needed for different reaction conditions.

The term “pyridine” is recognized in the art and refers to the heterocyclic aromatic compound of formula C₅H₄N. Pyridine may be substituted as described herein for other heterocyclic compounds or groups. Substituted pyridines can include among others those of formula R—O₅H₄N in which R may be selected from any alkyl, alkenyl, alkynyl, cycloalkyl, cycloalkenyl, cycloalkynyl, heterocyclyl, aralkyl, alkaryl or aryl, etc. The term “pyridyl” refers to a monovalent radical generated formally by removal of a H from a pyridine or substituted pyridine. As is known in the art, pyridyl groups include isomeric groups, pyrid-1-yl, pyrid-2-yl and pyrid-3-yl dependent upon the ring position of the H removed from pyridine.

The term “substitution” is understood in the art and may include all acceptable organic, inorganic, organic-inorganic, metallic, organometallic, etc. compounds that may substitute an atom(s) in a compound without spontaneously transforming (e.g. rearranging, cyclizing, eliminating, etc.) the said compound. Recognized substituents include acyclic, cyclic, branched, unbranched, carbocyclic, heterocyclic, aromatic and/or nonaromatic compounds. Additional substituents include various functional groups including among others, hydroxyl, halo, nitro, cyano, isocyano, carboxyl, carboxylate, amino, amido, acyl, alkoxy, alkyl, cycloalkyl, aryl, arylalkyl, alkylaryl, heterocyclyl and heteroaryl. The substituents may be one or more of the same or different moieties that are appropriate for the particular application. This disclosure is not intended to be limited in any manner by the substituents permitted.

The term “silyl” is recognized in the art and refers to the monovalent radical of formula —SiRR′R″ in which R, R′ and/or R″ are independently selected from hydrogen or any alkyl, alkenyl, alkynyl, cycloalkyl, cycloalkenyl, cycloalkynyl, heterocyclyl, aralkyl, alkaryl or aryl, etc. In an embodiment, at least one of R, R′ and/or R″ is a group other than hydrogen.

As used herein, the term “sulfate” is recognized in the art and refers to the entity represented in Formula 16.

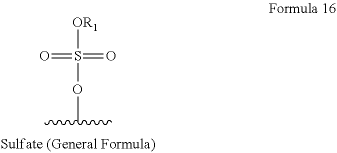

In formula 16, R₁ represent a pair of electrons, a hydrogen, an alkyl, alkenyl, alkynyl, a cycloalkyl, cycloalkenyl, cycloalkynyl, alkaryl, aralkyl or an aryl.

As used herein, the term “sulfamoyl” is recognized in the art and refers to the entity represented in Formula 17.

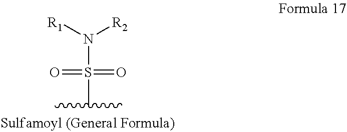

In formula 17, R₁ and R₂ independently are selected from a hydrogen, an alkyl, an alkenyl, alkynyl, cycloalkyl, cycloalkenyl, cycloalkynyl, aralky, alkaryl, aryl, heterocyclyl, —(CH₂)ₘ—R—, where R represents an alkyl, alkenyl, alkynyl, aryl, cycloalkyl, cycloalkenyl, cycloalkynyl, heterocycle or polycycle where m is zero or is an integer between 1 and 200, inclusive.

The terms “sulfoalkanediyl”, “sulfoalkenediyl”, “sulfoalkynediyl”, “sulfocycloalkanediyl”, “sulfocycloalkenediyl”, “sulfocycloalkynediyl”, “sulfoaryldiyl”, “sulfoaralkdiyl” and “sulfoalkaryldiyl” are recognized in the art and refer to moieties of the formula Rₐ—R_{b} where Rₐ is any sulfur containing species group (e.g. thiol, sulfide, disulfide, sulfoxide, sulfone, sulfinic acid, sulfonic acid, sulfonate ester, thiocyanate, thioketone, thial, thiocarboxylic acid, thioester, dithiocarboxylic acid, dithiocarboxylic acid ester, etc.) and R_{b} is an alkanediyl, alkenediyl, alkynediyl, cycloalkanediyl, cycloalkenediyl, cycloalkynediyl, aryldiyl, aralkdiyl, or alkaryldiyl.

The term “sulfonamido” is recognized in the art and refers to the entity represented in Formula 18.

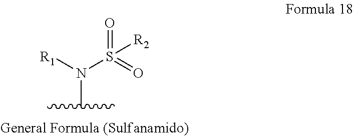

In formula 18, R₁ and R₂ independently represent a hydrogen, an alkyl, an alkenyl, alkynyl, cycloalkyl, cycloalkenyl, cycloalkynyl, aralkyl, alkaryl, aryl heterocyclyl, where R′ represents an alkyl, alkenyl, alkynyl, aryl, cycloalkyl, cycloalkenyl, cycloalkynyl, aralkyl, alkaryl, heterocycle or polycycle and m may be zero or may be an integer between 1 and 200, inclusive.

The term “sulfoxide” is recognized in the art and refers to the entity of formula RSOR′ which R and R′ may independently be selected from any alkyl, alkenyl, alkynyl, cycloalkyl, cycloalkenyl, cycloalkynyl, heterocyclyl, aralkyl, alkaryl or aryl, etc.

The term “sulfide” is recognized in the art and refers to the entity of formula RSR′ in which R and R′ is independently selected from any alkyl, alkenyl, alkynyl, cycloalkyl, cycloalkenyl, cycloalkynyl, heterocyclyl, aralkyl, alkaryl or aryl, etc. A “thiol” or “sulfhydryl group” is a monovalent radical of formula —SR, where R is hydrogen or any alkyl, alkenyl, alkynyl, cycloalkyl, cycloalkenyl, cycloalkynyl, heterocyclyl, aralkyl, alkaryl or aryl, etc.

The term “sulfinic acid” is recognized in the art and refers to the entity of formula RSO₂H in which R may be selected from any alkyl, alkenyl, alkynyl, cycloalkyl, cycloalkenyl, cycloalkynyl, heterocyclyl, aralkyl, alkaryl or aryl, etc.

The term “sulfoacyl” is recognized in the art and refers to the entity represented in Formula 19.

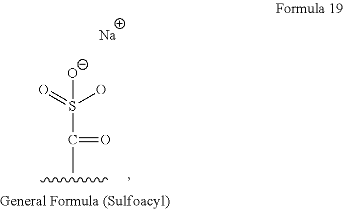

where Na⁺ is an exemplary cation.

As used herein, the term “sulfonate” is recognized in the art and refers to the entity represented in Formula 20.

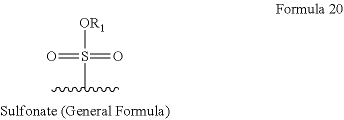

In formula 20, R₁ represents a pair of electrons, a hydrogen, an alkyl, alkenyl, alkynyl, a cycloalkyl, cycloalkenyl, cycloalkynyl, aralkyl, alkaryl, heterocyclyl or an aryl, etc.

The term “sulfonate ester” is recognized in the art and refers to the entity of formula RSO₃R′ in which R and R′ may be independently selected from any alkyl, alkenyl, alkynyl, cycloalkyl, cycloalkenyl, cycloalkynyl, heterocyclyl, aralkyl, alkaryl or aryl, etc. A sulfonate ester group is a monovalent radical formed formally by removal of a hydrogen from a sulfonate ester.

The term “sulfone” is recognized in the art and refers to the entity of formula RSO₂R′ which R and R′ may independently be selected from any alkyl, alkenyl, alkynyl, cycloalkyl, cycloalkenyl, cycloalkynyl, heterocyclyl, aralkyl, alkaryl or aryl, etc. A sulfone group is a monovalent radical formed formally by removal of a hydrogen from a sulfone.

The term “sulfonic acid” is recognized in the art and refers to the entity of formula RSO₃H in which R may be selected from any alkyl, alkenyl, alkynyl, cycloalkyl, cycloalkenyl, cycloalkynyl, heterocyclyl, aralkyl, alkaryl or aryl, etc. A sulfonic acid group is a monovalent radical formally formed by removal of a hydrogen from the R group of a sulfonic acid.

The term “sulfoxido” or “sulfinyl” is recognized in the art and is represented in general by Formula 21.

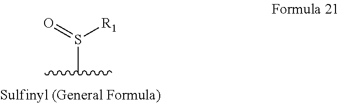

In formula 21, R₁ is independently selected from hydrogen, alkyl, alkenyl, alkynyl, cycloalkyl, cycloalkenyl, cycloalkynyl, heterocyclyl, aralkyl, alkaryl or aryl, etc.

The term “thial” is recognized in the art and refers to the formula RC(═S)H in which R may be selected from any alkyl, alkenyl, alkynyl, cycloalkyl, cycloalkenyl, cycloalkynyl, heterocyclyl, aralkyl, alkaryl or aryl, etc. A thial group is a monovalent radical group formally formed by removal of hydrogen from the R group of a thial.

The term “thiocarboxylic acid” is recognized in the art and refers to an entity of formulas RC═OSH and/or RC═SOH in which R are selected from any alkyl, alkenyl, alkynyl, cycloalkyl, cycloalkenyl, cycloalkynyl, heterocyclyl, aralkyl, alkaryl or aryl etc. A thiocarboxylic acid group is a monovalent radical formally formed by removal of a hydrogen from an R group of a thiocarboxylic acid.

The term “thiocyanate” is recognized in the art and refers to the entity of formula RSCN in which R are selected from any alkyl, alkenyl, alkynyl, cycloalkyl, cycloalkenyl, cycloalkynyl, heterocyclyl, aralkyl, alkaryl or aryl, etc. A thiocyanate group is a monovalent radical formed formally by removal of a hydrogen from the R group of a thiocyanate. The functional group —SCN is the thiocyanate functional group.

The term “thioester” is recognized in the art and refers to the entity of formulas RC═OSR′ and/or RC═SOR′ in which R and R′ are independently selected from any alkyl, alkenyl, alkynyl, cycloalkyl, cycloalkenyl, cycloalkynyl, heterocyclyl, aralkyl, alkaryl or aryl, etc. A thioester group is a monovalent radical formed formally by removal of a hydrogen from an R or R′ group of a thioester.

The term “thioketone” is recognized in the art and refers to the entity of formula RCSR′ in which R and R′ are independently selected from any alkyl, alkenyl, alkynyl, cycloalkyl, cycloalkenyl, cycloalkynyl, heterocyclyl, aralkyl, alkaryl or aryl, etc.

The term “thiol” is recognized in the art and refers to the formula RSH where R may be any alkyl, alkenyl, alkynyl, cycloalkyl, cycloalkenyl, cycloalkynyl, heterocyclyl, aralkyl, alkaryl or aryl.

The term “ester” is recognized in the art and refers to a —O(C═O)— or —C(═O)O— linkage. The term “thioester” refers to an —SC(═O)— or —C(═O)S— linkage. The term “amide” refers to an —N(R)C(═O) or —C(═O)N(R)— linkage, where R is hydrogen, any C1 to C100 alkyl, any C3 to C100 cycloalkyl, C3 to C100 cycloalkyl-C1 to C100 alkyl, aryl, heteroaryl, aryl(C1 to 100)alkyl, heteroaryl(C1 to C100)alkyl, etc.

The term “protected” is recognized in the art and refers to a moiety that is added to a reactive functional group (e.g. oxygen, nitrogen, etc.) to prevent it from reacting during the course of polymerization, derivatization, etc. The reactive functional group is later deprotected. A variety of protecting groups are available for each reactive functional group and are known to one skilled in the art.

In the definitions herein which refer to the number of carbon atoms in a given chemical group, such groups can contain 1-20, 2-20, 3-20, 1-12, 2-12, 3-12, 1-10, 2-10, 3-10, 1-6, 2-6, 3-6, 1-4, 2-4, or 3-4 carbon atoms. In those groups carrying one or more ring structures such groups can contain, 3-20, 3-12, 3-10, 3-8, 3-6, 4-20, 4-12, 4-10, 4-8, 4-6, 5 or 6 ring members. In specific embodiments, rings contain 5 or 6 ring members. In heterocyclic rings, the heterocyclic ring may contain 1, 2, 3 or 4 heteroatoms. In specific embodiments, heterocyclic rings have 5 or 6 ring members of which 1-4 ring members are heteroatoms. In specific embodiments, heterocyclic rings contain 1 or 2 heteroatoms.

Comparable substitutions may be made to other groups within the definitions such as alkenyl, alkynyl, aryl, etc. moieties. Some examples include, but are not limited to, aminoalkenyls, aminoalkynyls, amidoalkenyls, amidoalkenyls, amidoalkynyls, iminoalkenyls, iminoalkynyls, thioalkenyls, thioalkynyls, carbonyl-substituted alkenyls or alkynyls.

All geometric and stereoisomeric forms of compounds listed are meant to fall within the scope of the present disclosure. Compounds of such include cis- and trans-isomers, R- and S-enantiomers, diastereomers, (D)-isomers, (L)-isomers, racemic mixtures and/or other mixtures thereof.

Apparatus and Method Employing MME

The invention describes an apparatus and method for generating, providing diffusion limited transport and evacuating gaseous phase material in a sub-atmospheric environment. The apparatus and method described herein, “diffusion system”, is extremely adaptable to use with various processing methods. For example, the diffusion system may be used as part of a rinsing/cleaning system, drying system, etc. and/or as part of a sterilization/sanitization or sanitization system. The diffusion system may be used alone or may be used in conjunction with other systems. For example, the diffusion system may have pre-treatment process such as a cleaning/rinsing and/or drying.

Any vacuum chamber that can achieve reduced pressures can be used to cause the MME-transport moiety blend to vaporize. In some implementations, heat can be supplied by a conductive heating assembly to facilitate MME-transport moiety outgassing from the solid.

The MME-transport moiety composition is placed, manually or automatically, into the process chamber of the vacuum system after which point the door(s) to the system are closed and sealed and the system is pressurized by the pressurizing subsystem. The heating subsystem optionally provides heat to the conductive thermal assembly of the process chamber. As the pressure in the chamber is reduced the MME-transport moiety vapor from the solid is outgassed. In an embodiment, the pressure in the process chamber is reduced to below 1 torr. In an embodiment, the pressure in the process chamber is reduced to below 0.1 torr. In an embodiment, the pressure in the process chamber is reduced to 1×10⁻³ torr or less. Heated or non-heated, non-reactive carrier gas may be introduced into the process chamber enhancing flow of the MME-transport moiety vapor throughout the process chamber.

Different portions of the system may be operated at different temperatures. For example, the process chamber may be operated at one temperature and other portions of the system may be operated at temperature(s) different from the processing chamber. The process chamber which contains vapor comprising the MME, the transport moiety or a mixture of MME and transport moiety may be operated at ambient room temperature or heated (by any known means) to a higher temperature 100° C. Preferably, the process chamber is operated at a temperature ranging from ambient room temperature to 60° C.

The pressure in the process chamber is preferably maintained at a selected pressure for a selected time to initiate and complete outgassing and conversion of the MME and/or transport moiety liquid into vapor. One skilled in the art understands that the holding vapor chamber pressure and the vapor chamber pressure holding time can be adapted to a given application of the MME-transport moiety blend vapor. In embodiments, the selected pressure maintained in the apparatus ranges from 0.1 torr to 200 torr. More specifically, the selected pressure maintained in the apparatus ranges from 0.5 torr to 10 torr. More specifically, the selected pressure maintained in the apparatus ranges from 0.5 torr to 3 torr. More specifically, the selected holding pressure and holding pressure time of the apparatus is maintained within approximately +/−10%. In embodiments, the selected holding pressure time is maintained for 1 minute to 24 hours. In embodiments, the selected holding pressure time is maintained for 5 minutes to 1 hour. In embodiments, the selected holding pressure time is 5 minutes to 30 minutes. In a specific embodiment, the selected holding pressure time is 15 minutes+/−10%.

In some implementations, the MME and transport moiety are vaporized independently after which vapors are mixed to execute the described method of the invention. In some implementations, heat can be supplied by a conductive heating assembly, for example, to facilitate MME-transport moiety blend vaporization. Generating and transporting vapors into or through diffusion restricted spaces and evacuation of gaseous materials in and from a sub-atmospheric pressure environment, with or without heat, is facilitated by an MME. Heating may be used to enhance vaporization of the MME and/or transport moiety and/or it may assist molecular transport of gaseous materials in the diffusion system by increasing kinetic energy of the MME-transport moiety blend.

A representative apparatus for performing the method described herein using an MME-transport moiety blend vapor includes a diffusion system that can be used to flow MME-transport moiety blend vapor through diffusion restricted objects or the like. As described herein, and in some implementations, the diffusion system consists of a processing chamber and a chamber extension. The chamber extension may be connected to the process chamber externally or internally. The substrate(s), devices(s) and/or other instrument(s) to be treated may be placed into the process chamber for exposure to the MME-transport moiety blend vapor. The process chamber and the chamber extension may be manufactured from any suitable materials that can withstand repeated exposure to reduced pressures and MME-transport moiety blend vapor contact.

The diffusion system can include any suitable MME-transport moiety blend delivery mechanism. The MME-transport moiety blend may be supplied to the process chamber and/or chamber extension in liquid form. For example, implementations of the delivery mechanism can include bulk sources of MME-transport moiety blend liquid that are stored external or internal to the diffusion system and are delivered in aliquots to the diffusion system manually or automatically by a device that controls flow of liquid such as a mass flow controller (MFC) or a syringe pump, for example. MME-transport moiety blend vapor(s) or non-reactive gases containing quantities of MME-transport moiety blend vapor(s) (e.g. nitrogen with a methanol-hydrogen peroxide blend) may be stored external or internal to the drying system in containers such as compressed gas containers or containers that generate compressed gas on demand and may be delivered in aliquots to the diffusion system manually or automatically by a device that controls flow of gas such as an MFC, for example Implementations of the delivery mechanism can also include MME-transport moiety blend packages (e.g. liquid MME-transport moiety blend or components, ampoules and/or cartridges containing the MME-transport moiety blend or components, etc.) that are disposed directly within the process chamber and/or the chamber extension. The container or wrapping containing the MME-transport moiety blend liquid may be provided in a gas permeable packet or cartridge that is capable of allowing transport of MME-transport moiety blend vapor formed during the outgassing process out of the packet or cartridge. Examples of vapor gas porous membrane materials that may be used to manufacture the packets or cartridges that allow gaseous material transport include, but are not limited to, polytetrafluoroethylene (PTFE), polyethersulfone (PES), or high-density polyethylene (HDPE). The exact gas-permeable membrane (e.g. material, porosity, etc.) may be selected based on the type of the MME-transport moiety blend. In some implementations, the MME-transport moiety blend may be disposed within a sealed gas-permeable membrane and then sealed within a non-permeable wrapper. Prior to use, the non-permeable wrapper may be removed and the MME-transport moiety blend within the gas-permeable membrane may be inserted into the diffusion system. MME-transport moiety blend packages may be disposed within a separate chamber that is in fluid communication with the process chamber and/or the chamber extension. MME-transport moiety blend or components can be disposed or injected into the process chamber and/or chamber extension, in liquid-form, independently and then mixed within the system prior to processing. In some implementations, MME-transport moiety blend components, the MME and the transport moiety, may be vaporized and injected independently into the process chamber and or chamber extension after which point the gases are mixed prior to processing.

The process chamber can be used to diffuse gaseous material onto, into and/or through any suitable substrate, device and/or instrument, such as a medical device (e.g. lumen) or an object with 3D components (e.g. protoboards (PCBs)). Other substrates, devices and/or instruments may include electronic components such as semiconductors, integrated circuits, solar cells, microelectromechanical systems, disc drives, etc. or medical devices such as clips, forceps, scissors, cameras, pacemaker items, hooks/retractors, etc. One non-limiting exemplary medical device is an endoscope or device that contains one or more lumens. Typically, objects with diffusion restricted spaces have limitations that prevent transportation of gaseous materials through the spaces using conventional techniques. In terms of vapor sterilization/sanitization of medical devices containing one or more lumens inadequate diffusion of vapor sterilant into the inner core(s) of a lumen(s) causes device contamination ultimately leading to patient infection. For example, conventional vapor diffusion techniques are complicated, time consuming, incompatible and ineffective due to the challenges associated with transporting gaseous material through objects with diffusion restricted spaces. Accordingly, the process described herein can use MME vapor flow in conjunction with negative vacuum, with or without heat, to overcome the limitations of diffusion restricted transport of gaseous material that previous methods have failed to provide. Advantageously, the process described herein additionally provides a method by which an MME vapor in combination with negative vacuum, with or without heat, facilitates the vaporization of a desired transport moiety from liquid and expedites the evacuation unused or unwanted gaseous material from the diffusion system. In terms of medical devices and/or electronic components, many have relatively low exposure limits for temperature and/or for chemicals. Accordingly, the process described herein can use negative pressure and MME vapor under no or relatively low temperatures to facilitate vaporization of the transport moiety from a liquid, transportation of vaporized transport moiety into diffusion restricted spaces and evacuation of any unused or unwanted transport moiety vapor from the diffusion system at process termination while remaining well within the thermal exposure limits of the device. Additionally, the MME vapor when used in a reduced pressure environment acts as a conduit by which the transport moiety vapor travels. The use a conduit generated by MME vapor prevents the transport moiety vapor for stagnating, congregating or condensing in undesirable locations within the diffusion system. Without undesirable aggregation of transport moiety vapor molecules lower concentrations of the desired transport moiety, lower processing times and/or lower temperatures may be used to effectively achieve diffusion into restricted spaces.

Components of the diffusion system are manufactured in any suitable manner and in any suitable size and shape and/or material so that the system can withstand sub-atmospheric pressures and can accompany the desired number of substrates, devices and/or instruments. For example, the diffusion system can be made of metal or durable plastic and may include seals, made of any suitable material, where necessary to maintain sub-atmospheric pressures within the diffusion system. Some implementations may include multiple process chambers for synchronous but segregated diffusion of multiple substrates, devices and/or instruments. Some implementations may include multiple chamber extensions for concurrent but segregated vaporization of MMEs and/or transport moieties of different types. MME-transport moiety vapor may be generated in one chamber extension and transported to one or more process chambers. One or more MME-transport moiety vapors may be generated in one or more chamber extensions and may flow to one or more process chambers. In some implementations, the MME and transport moiety vapor may be generated independently in one chamber extension, mixed within the chamber extension, and transported to one or more process chambers. In other implementations, the MME and transport moiety may be generated independently in multiple chamber extensions and may independently flow to the process chamber where they are mixed. Some diffusion systems are designed to facilitate use within the context of a larger assembly (e.g., a wall-mounted or case-integrated diffusion chamber). In one implementation, multiple process chambers are stacked in a configuration that allows access like a drawer, chest, etc. Other implementations further include windows, internal lighting (such as UV light) and/or other features to allow users to view the inside environment. In another implementation, the diffusion system may be connected to other processing modules. Some examples include a diffusion system that is connected in-line with a drying system and/or one or more wet benches or solvent benches or with one or more ultrasonic cleaners, washer-decontaminators, washer-disinfectors, washer-sterilizers or any manual automatic unit that may expose substrates, devices and/or instruments to aqueous conditions.

The diffusion system is pressurized by a depressurizing subsystem such as a vacuum pump or the like that is capable of achieving a reduced pressure environment in the process chamber and/or chamber extension. The specifications of the depressurizing subsystem are selected to produce a desired vacuum level within a desired amount of time, given the air-space within the drying system, the quality of the process chamber and the chamber extension seals, etc. The depressurizing subsystem includes a one-half-horsepower, two-stage vacuum pump configured to produce a vacuum level within the diffusion system of approximately 0.4 inches of mercury (“inHg”) within seconds and to maintain substantially that level of pressure throughout the drying process. Different depressurizing subsystem specifications can be used to support simultaneous diffusion in multiple diffusion systems of different sizes, use in portable versus hart-mounted implementations.

The depressurizing subsystem is in fluid communication with the diffusion system (or multiple diffusion systems) via one or more fluid paths. The fluid path can include one or more components including release valves, hoses, fittings, seals, etc. and may consist of any suitable material. The fluid path components are selected to operate within the produced level of negative pressure. Release valve components may be controlled manually or electronically for the purpose of bringing the negative pressure environment to atmosphere in order to allow the diffusion system to be opened after the diffusion process has completed or at any other desirable time. A filter may be included as part of the release valve or as part of any other fluid path component so as to prevent contaminants (e.g. dirt/dust, moisture, etc.) from being allowed to reenter the diffusion system and cause contamination. The release valve may also be connected to a source of purified non-reactive gas such as argon, nitrogen, or a combination of non-reactive gas and air, which may also be bled into the drying system to bring the negative pressure environment to atmosphere. In implementations in which multiple diffusion systems are utilized, multiple fluid paths including multiple fluid path components such as multiple release valves or other techniques may be used to fluidly couple the depressurizing subsystem with the diffusion system.

Depressurization of the process chamber and/or the chamber extension causes the MME-transport moiety liquid to gasify (e.g. evaporate, vaporize, etc.) triggering the vaporization of the MME-transport moiety followed by the release of the MME-transport moiety blend vapor into the process chamber or from the chamber extension into the process chamber. Depressurization of the process chamber(s) and the chamber extension(s) may occur simultaneously or in sequence and the sequence may be repeated. MME-transport moiety blend vapor may be carried in the process chamber or from the chamber extension to the process chamber via a non-reactive carrier gas such as air, argon or nitrogen. The flow of the MME-transport moiety blend vapor follows a path onto or into the surfaces, ports, inner areas or other non-sealed portions of the substrates, devices and/or instruments. Flowing MME vapor on or into substrates, devices and/or instruments facilitates movement of transport moiety vapor through diffusion restricted spaces by negating transport moiety vapor molecule stagnation and aggregation essentially increasing velocity and laminar flow. In some implementations, heat may be added to the diffusion environment in only as much as sufficient to overcome the latent heat of vaporization. In other implementations, other amounts of heat are provided to the diffusion environment within the process chamber and/or chamber extension. For example, even though it is not required, additional heat can be added to accelerate the diffusion process, or heat can be added in varying amounts over time for various purposes. For example, the amount of heat (e.g. and/or a profile of changes in temperature and/or pressure over time) can be tailored to particular implementations of MME-transport moiety blends and corresponding vapor pressures for gasification of MMEs, transport moieties and/or MME-transport moiety blends. In another implementation, heat can be provided to the MME-transport moiety blend vapor. In yet another implementation, heat can be provided to the non-reactive carrier gas (e.g. argon, nitrogen, air, etc.).

In some implementations, the substrate(s), device(s) or instrument(s) are placed on or in an assembly inside of the process chamber. The assembly resembles a substrate holder that may be capable of exposing the faces and/or inner areas of a substrate(s), device(s) and/or instrument(s) to the flow of the MME-transport moiety blend vapor. The assembly can consist of any suitable material and can hold the intended objects in a horizontal, vertical or any custom angle position and can be interchangeable. Substrate(s), device(s) and/or instrument(s) can be individually supported so as to minimize shadowing or obstruction of the substrate(s), device(s) and/or instrument(s) surface and/or openings. In other implementations, manifolds of any configuration may be used to support a substrate(s), device(s) and/or instrument(s) in order to provide direct delivery of MME-transport moiety blend vapor to the immediate surface or inner core of an object. For example, a device containing a lumen, such as an endoscope, may be slid over a porous manifold designed to feed MME-transport moiety blend vapor to the inner area of the device. In another example, substrate(s), device(s) and/or instrument(s) may be placed in an envelope- or box-type structure machined to be a manifold. Such a design may also achieve direct flow of MME-transport moiety vapor to the objects in the envelope or box. In another implementation, isolated containers may be used to perform the functions of this invention. In yet another implementation, the assembly is configured to be part of the heating assembly such that the assembly (e.g. tray or manifold) or any extension of the assembly (e.g. thermally conductive conformable media) may be heated to assist in the process of drying.

Other implementations of the substrate holder or assembly or conductive thermal assembly may include manifolds that are in direct fluid communication with a substrate(s), device(s) and/or instrument(s). Some implementations may include vapor distribution manifolds that are used to route vapors from one entry port to multiple outlet locations that is in fluid communication with substrate(s), device(s) and/or instrument(s). The vapor distribution manifolds may advantageously direct MME-transport moiety blend vapor to diffusion restricted locations on or within a substrate(s), device(s) and/or instrument(s). For example, a manifold insert may be inserted into a device that contains a lumen (e.g. endoscope) in a longitudinal manner. The manifold insert may be of any shape and consist of any type of material that is machinable and contains any number of inlets and outlets. The inlets and outlets may have any diameter opening or diameters may vary. Any manifold configuration is conceivable. For example, there may be one or more outlets that may line the entire length of the insert and all outlets are in fluid communication with the inner area of the lumen. There may be one or more inlets and they may be located at each end of the insert. More than one manifold may exist in a drying system. Multiple manifolds may be independent from one another in which MME-transport moiety blend vapor is supplied separately to the inlets of each insert. Multiple manifolds may be connected to one another and the MME-transport blend vapor may be supplied separately to the inlets of each insert or the MME-transport blend vapor may be supplied to a multiple manifold assembly that may have one insert. The cross-sectional area of the inserts may vary along the length of a single inlet or may vary between inserts.

In another implementation, the manifolds may be heated and porous in such a capacity that MME-transport moiety blend liquid that may be delivered to the manifolds can be outgassed into the process chamber or directly onto the surface of or within the diffusion restricted spaces of substrate(s), device(s) and/or instrument(s).

The MME-transport moiety blend vapor manifolds have been described such that the MME-transport moiety blend vapor may be directed from an inlet location on the insert to outlet locations on the insert by way of a closed channel that is created by the insert. In another implementation the manifold may include closed, partially closed or porous trays, boxes, cartridges, bags or any other types of substrate(s), device(s) and/or instrument(s) holder that is attached to the insert or to protrusions of the insert. A porous tray or box is a substrate holder assembly that is configured to be attached to the insert by way of an open through-hole such that MME-transport moiety blend vapor can be routed from the insert into the closed tray or box. In some implementations, the MME-transport moiety vapor is directed into the tray or box containing substrate(s), device(s) and/or instrument(s) by way of an insert and residual vapor is exchanged through the pores on the tray or box assembly. Any insert-tray/box substrate holder assembly configuration is conceivable. For example, a single insert may be any length and contain multiple insert protrusions each containing a tray or box. Each single substrate tray or box may be attached to multiple inserts. In this manner, each tray or box can have multiple inlets. The substrate box or tray can be configured to have any size pores and one or more sides or the box or tray may contain the pores. The substrate box or tray assembly may be sufficiently porous to allow fluid transport of the MME-transport moiety vapor through the substrate assembly and to allow transport of residual vapor out of the substrate assembly. Any combination of inlet and outlet channels can be envisioned.

Conductive heat may be used to provide heating to the substrate(s), device(s) and/or instrument(s) within the drying chamber. A heating subsystem heats the substrate holder or conductive thermal assembly (e.g., and the process chamber and/or chamber extension in some implementations) which is in contact with the substrate(s), device(s) and/or instrument(s) and is configured to conduct heat to the objects. Implementations of the substrate holder or conductive thermal assembly at least partially conform to an external shape of the substrate(s), device(s) and/or instrument(s) so as to partially surround the object. For example, the substrate holder or conductive thermal assembly is designed to gently immerse or blanket an object in such a way as the substrate holder or conductive thermal assembly has full or partial conformal contact with the substrate(s), device(s) and/or instrument(s). For example, conductive beads, conductive mesh, heat packs, etc. can be assembled in a manner that dynamically conforms to the geometry of one or more types of substrate(s), device(s) and/or instrument(s) when the object(s) comes into contact with the substrate holder conductive thermal assembly. Examples of various conformal conductive thermal assemblies are set forth in co-owned U.S. Pat. No. 8,689,461, the entire contents of which are incorporated herein by reference.

The heating subsystem can heat the diffusion system process chamber and/or the chamber extension from the outside (e.g., from the bottom and/or side of the process chamber and/or the chamber extension). The applied heat from the heating subsystem (e.g. resistive electrical or radiant heater) is conducted toward the substrate(s), device(s) and/or instrument(s) via conductive beads, conductive mesh, heat packs, etc. and/or the manifold(s) and/or the tray(s), box(es), cartridge(s) insert assembly, permitting the heat to evenly be distributed to the inner and outer surfaces of the substrate(s), device(s) and/or instrument(s). In order to minimize cooling from latent heat of vaporization and maximize gasification from MME-transport vapor flow and heating, materials for machining substrate holders may be machined from material(s) that has relatively high thermal capacity (e.g. storage) which can provide a steady flow of heat to the substrate(s), device(s) and/or instrument(s) without exceeding temperature limits.

Other subsystems can be used to provide additional functionality. These may include a monitoring subsystem that can provide feedback control, environmental monitoring within the diffusion system, monitoring of substrate(s), device(s) and/or instrument(s), etc. Implementations of the monitoring subsystem may include one or more sensors situated inside the diffusion system and configured to monitor internal pressure (vacuum level), humidity, temperature, etc. within the diffusion system.

The monitoring subsystem can communicate its measurements through wired and/or wireless communications links to a controller located outside the drying system. For example, the controller includes memory (e.g., non-transient, computer-readable memory) and a processor (e.g., implemented as one or more physical processors, one or more processor cores, etc.). The memory has instructions stored thereon, which, when executed, cause the processor to perform various functions. The functions can be informed by (e.g. directed by, modified according to, etc.) feedback from the monitoring subsystem. For example, the measurements from the monitoring subsystem can be used to determine when to end the diffusion process and release a pressure valve of the process chamber, when and how to modify the heat being delivered to the conductive thermal assembly, etc. The controller can also direct operation of other subsystems, such as the conveyor assembly, pressurizing subsystem, etc.

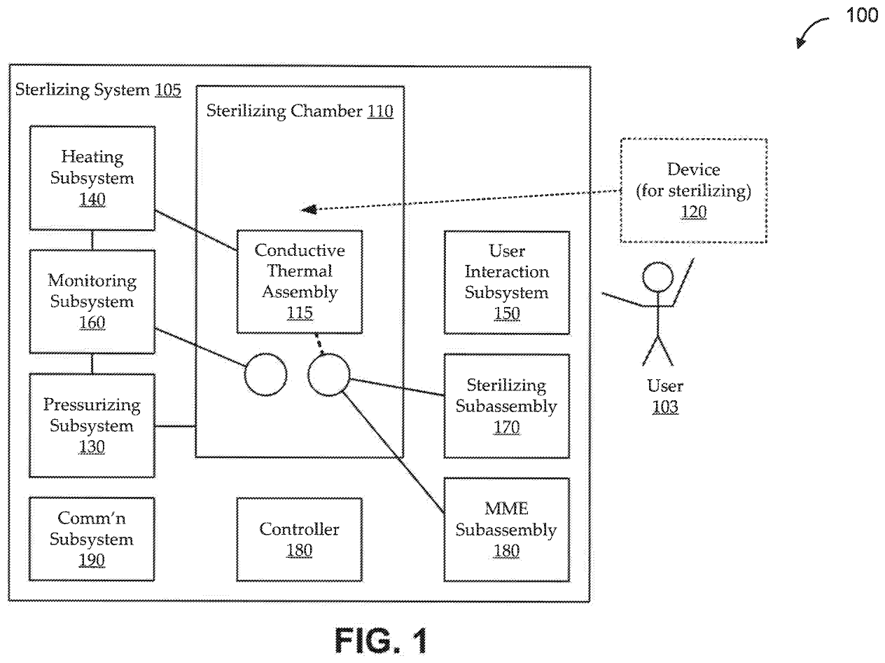

An exemplary system for practice of the invention, particularly with respect to sterilization, is described in FIGS. 1-3. Details of systems useful in the practice of this invention are provided in PCT application WO2018/175455, published Sep. 27, 2018, U.S. published application US2018/0289846, U.S. provisional applications 62/473,543, filed Mar. 20, 2017 and 62/598,004, filed Dec. 13, 2017, each of which is incorporated by reference herein in its entirety. U.S. provisional application 62/797,789, filed Jan. 28, 2019 and PCT application WO2020/160027 published Aug. 6, 2020 also provide additional details particularly for sterilization applications. Each of the listed patent references is incorporated by reference herein in its entirety.

The invention presents several advantages, such as 1) expedited rate of vaporization of liquids in a reduced pressure environment at low temperatures or no heat; 2) accelerated movement of gaseous material through diffusion restricted spaces in a reduced pressure environment at low temperatures or no heat; 3) rapid evacuation of unused or unwanted gaseous material from a reduced pressure environment; 4) the use of high temperatures is avoided; 4) the use of concentrated liquids is avoided; 5) exposure of substrate(s), device(s) and/or instrument(s) surface(s) to liquid is avoided; 6) selective heating and/or outgassing of substrate(s), devices(s) and/or instrument(s) and MME-transport moiety blend liquid or component liquids can occur via chamber isolation.

A general operation of an apparatus and/or method of the invention, which is useful for transporting gaseous material through diffusion restricted object such as a lumen, is as follows:

The lumen to be treated is placed within the process chamber;

The MME-transport blend liquid is placed inside of the process chamber. (The MME-transport blend liquid or liquid components, that can also be mixed within the process chamber, can also be delivered to the process chamber from an external supply that is injected into the process chamber);

The process chamber is heated to a temperature that is sufficient for the process;

The diffusion system is sealed and evacuated;

The MME-transport blend liquid is outgassed;

The lumen is exposed to the MME-transport blend vapor and vacuum for a given time at a temperature that is sufficient for the process; and

The diffusion system is brought to atmosphere and the lumen is removed from the process chamber.

The general operation of an alternative apparatus and/or method of the invention, which is useful for transporting gaseous material through diffusion restricted objects such as a lumen, is as follows:

The lumen to be treated is placed within the process chamber;

The MME-transport blend liquid is placed inside of the chamber extension. (The MME-transport blend liquid or liquid components, that can also be mixed within the chamber extension, can also be delivered to the chamber extension from an external supply that is injected into the chamber extension);

The process chamber and the chamber extension are heated to a temperature that is sufficient for the process;

The diffusion system is sealed and evacuated;

The MME-transport blend liquid is outgassed and transported to the process chamber;

The lumen is exposed to the MME-transport blend vapor and vacuum for a given time at a temperature that is sufficient for the process; and

The diffusion system is brought to atmosphere and the lumen is removed from the process chamber.

The general operation of an alternative apparatus and/or method of the invention, which is useful for transporting gaseous material through diffusion restricted objects such as a lumen, is as follows:

The lumen to be treated is placed within the process chamber;

The MME and the transport liquid are placed independently inside of the process chamber. (The MME-transport blend liquid components can also be delivered to the process chamber from an external supply that is injected into the process chamber);

The process chamber is heated to a temperature that is sufficient for the process;

The diffusion system is sealed and evacuated;

The MME and transport liquid are independently outgassed;

The MME and transport liquid vapors are mixed within the process chamber;

The lumen is exposed to the MME-transport blend vapor and vacuum for a given time at a temperature that is sufficient for the process; and

The diffusion system is brought to atmosphere and the lumen is removed from the process chamber.

The general operation of an alternative apparatus and/or method of the invention, which is useful for transporting gaseous material through diffusion restricted objects such as a lumen, is as follows:

The lumen to be treated is placed within the process chamber;

The MME and the transport liquid are placed independently inside of the chamber extension. (The MME-transport blend liquid components can also be delivered to the chamber extension from an external supply that is injected into the chamber extension);

The process chamber and chamber extension are heated to a temperature that is sufficient for the process;

The diffusion system is sealed and evacuated;

The MME and transport liquid are independently outgassed;

The MME and transport liquid vapors are mixed within the chamber extension and transported to the process chamber;

The lumen is exposed to the MME-transport blend vapor and vacuum for a given time at a temperature that is sufficient for the process; and

The diffusion system is brought to atmosphere and the lumen is removed from the process chamber.

Methods, Apparatus and Compositions for Drying Employing MDE

Compositions of the present invention for generating MDE vapors used for carrying out a drying process in vacuum comprise of one or more volatile hydrocarbon, alcohol, ketone, nitrile, carboxylic acid, ester, ether, glycol ether, polysiloxane, aldehyde, etc. The above list of MDE groups is a subset, not an exhaustive list of groups of volatile compounds that can be used in the present invention. The MDE improves the drying characteristics of the vacuum process, particulary the drying of surfaces or objects that contain small openings or crevices (e.g. lumens, 3D objects, etc.). In addition to the components described above, the composition may contain additional agents. These agents, may include, for example, volatile surfactants, stabilizers, catalyst, etc. One skilled in the art will recognize that a number of different additional agents may be used in the present invention depending upon the type(s) of MDE(s), properties required of the final MDE composition and application or type of drying process and/or device(s) to be dried by the MDE. One skilled in the art will also recognize that the quantities of the additional agents are within the teachings of the art.

In general, the MDE is included in compositions of the present invention in amounts effective to provide enhanced drying of surfaces or objects in a vacuum process chamber. In general, the amount and type of MDE is selected so that the MDE has a vapor pressure that is between 1 and 650 mmHg, more preferably between 100 and 500 mmHg and most preferably between 250 and 450 mmHg, at room temperature, and that interacts with liquid to be removed from the surfaces or objects in in such a way as to essentially displace unwanted liquid on surfaces or objects. This interaction results in an expedited and complete drying process. The MDE drying process as well as the type and amount of MDE using during the process will vary depending on the temperature and the humidity of the vacuum process chamber.

Furthermore, the MDEs as described above demonstrate excellent transport or penetrating properties in small openings and crevices, such as those that may exist on various articles, substrates or device, particularly those made of metal or plastic, including electronic device or electronic boards or related devices or semiconductor surfaces, including those in or one devices. As such, unwanted liquid trapped in small spaces on or within a device, such as a narrow lumen following a preparatory rinse step, can easily be accessed by the MDE and effectively removed by vaporization in the vacuum process chamber. The use of MDE compounds of the present invention enables expeditious drying of complicated surfaces following exposure of MDE vapor to the moist surface.

In some implementations, the use of a volatile surfactant in combination with an MDE may be a means for removing fluids from surfaces or objects. The role of the surfactant is to aid in displacing or repelling residual fluid from the surface or object to be dried. Any surfactant that is compatible may be used. The type and amount of surfactant that may be used in combination with the MDE is determined by the chemistry of the MDE, the surface chemistry (e.g. hydrophobicity, etc.) and of the surface(s) or object(s) to be dried and amount of liquid to be dried off of the surface(s) or object(s). Surfactants used in the present invention may be anionic, nonionic, cationic and/or amphoteric. More than one surfactant may be used. One skilled in the art will also recognize that the quantities of the additional agents are within the teachings of the art.

A stabilizer, sequestering agent, preservative, antioxidant or the like is optionally added to the composition to extend the shelf-life of the MDE and/or MDE composition. Any stabilizer, sequestering agent, preservative, antioxidant or the like can be used, so long as it does not negatively interfere with the action or mechanism of the MDE and/or the MDE composition. One skilled in the art will also recognize that the quantities of the additional agents are within the teachings of the art.

In some implementations, the method of this invention can be combined with methods for cleaning, sanitizing and sterilizing articles, particularly as applied to medical devices and electronic equipment. PCT application WO2018/175455, published Sep. 27, 2018, U.S. published application US2018/0289846 and U.S. provisional applications 62/473,543, filed Mar. 20, 2017 and 62/598,004, filed Dec. 13, 2017, each of which is incorporated by reference herein in its entirety, describe apparatus, methods and material for sterilizing articles, including medical implements, medical devices and medical instruments. U.S. provisional application 62/797,789, filed Jan. 28, 2019 and PCT application WO2020/160027 published Aug. 6, 2020 also provide additional details particularly for sterilization applications and provide additional examples of compositions, particularly for use in sanitation and sterilization application which employ sub-atmospheric pressure. Each of these applications is also incorporated by reference herein in its entirety. The methods of the present invention can be employed in combination with the methods of cleaning, sanitation and/or sterilization as described in the listed patent preferences as one or more steps prior to a final sterilization process. For example, the methods herein can be employed to dry articles that have been cleaned or sanitized by any convention method or by a sub-atmospheric pressure method as described in the forgoing patent applications. Articles dried by the method herein can then be subjected to sterilization under sub-atmospheric pressure as described in the forgoing patent application or in fact any conventional sterilization methods. These patent application describe an apparatus comprising a process chamber capable of achieving reduced pressure with accompanying vacuum equipment valve and ancillary device elements for carrying out sub-atmospheric treatment with a vapor generated by reducing the pressure in the system form certain compositions. The apparatus described therein can in embodiments be employed for the practice of the method of the present invention. The apparatus can be used in an embodiment to generate a vapor comprising the MDE herein and to provide for contact of the MDE vapor with an article form which undesired liquid is to be removed.

U.S. Pat. No. 8,689,461, the entire contents of which are incorporated herein by reference, provides descriptions of apparatus and system for drying articles under vacuum. The materials and methods of this invention can be practiced in such apparatus and systems.

### Definitions with Respect to MDE and Compositions Containing MDE

Used herein, the term “hydroxyl” is recognized in the art and refers to formula —OH.

Used herein, the term “alkane” is recognized in the art and refers to any saturated aliphatic group that includes linear and/or branched alkane groups, cycloalkane groups, cycloalkane groups with alkane substitutions and/or alkane groups with cycloalkane substitutions. As described herein, the term “alkane” is subject to valence tolerance and may be used interchangeably in place of a monovalent or alkyl, divalent or alkdiyl, trivalent or alktriyl, tetravalent or alkquaternyl, and so on. The linear or branched alkane groups referred to in composition of the invention have between 1 and 200 carbon atoms and the cycloalkane groups have between 3 and 200 carbon atoms. “Alkane” may also refer to a group that is halosubstituted. “Alkane” may also refer to alkanes that have been substituted or alkane moieties in which one or more hydrogen atoms have been substituted with an alternative substituent. Alternative substituents may include, but are not limited to, an alkane, an alkene, an alkyne, a cycloalkane, a cycloalkene, a cycloalkyne, an aryl, a halogen, a hydroxy, a carboxyl, an alkoxycarbonyl, a formyl, an acyl, or other carbonyl, a thioester, a thioacetate, a thioformate, or other thiocarbonyl, an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic or heteroaromatic, or others described herein, etc. It will be understood by one skilled in the art that the alternative substituents may themselves be substituted. Substituted forms of the alternative substituents may include, but are not limited to, any form of an amino, azido, imino, amido, (such as phosphonate, phosphinate, etc.), sulfonyl (such as sulfate, sulfonamide, sulfamoyl, sulfonate, etc.), silyl, ether, alkylthios, carbonyls (such as ketones, aldehydes, carboxylates, esters, etc.), cyano, etc. Examples of alkanes include, but are not limited to, methyl, ethyl, propyl, isopropyl, isobutyl, sec-butyl, pentyl, n-hexyl, octyl, dodecyl, and the like. The term “alkyl” refers to any monovalent form of the term “alkane” described above. The term “alkdiyl” refers to any divalent form of the term “alkane” described above. The term “alktriyl” refers to any trivalent form of the term “alkane” described above. The term “alkquaternyl” refers to any tetravalent form of the term “alkane” described above. “Alkyl”, “alkdiyl”, “alktriyl” and “alkquaternyl” moieties may also undergo substitutions as described above for “alkanes”.

Used herein, the term “alkene” is recognized in the art and refers to any unsaturated aliphatic group that includes linear and/or branched alkene groups, cycloalkane groups, cycloalkane groups with alkene substitutions and/or alkene groups with cycloalkane substitutions; linear hydrocarbon or branched hydrocarbon containing at least one double bond. As described herein, the term “alkene” is subject to valence tolerance and may be used interchangeably in place of a monovalent or alkenyl, divalent or alkendiyl, trivalent or alkentriyl, tetravalent or alkenquaternyl, and so on. The “alkene” may be similar in length and substitutions to the alkanes described herein. The linear or branched alkene groups referred to in composition of the invention have between 3 and 200 carbon atoms and the cycloalkene groups have between 3 and 200 carbon atoms. “Alkene” may also refer to alkenes that have been substituted or alkene moieties in which one or more hydrogen atoms have been substituted with an alternative substituent(s). Alternative substituent(s) may include, but are not limited to, an alkane, an alkene, an alkyne, a cycloalkane, a cycloalkene, a cycloalkyne, an aryl, a halogen, a hydroxy, a carboxyl, an alkoxycarbonyl, a formyl, an acyl, or other carbonyl, a thioester, a thioacetate, a thioformate, or other thiocarbonyl, an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic or heteroaromatic, or others described herein, etc. It may be understood by one skilled in the art that the alternative substituent(s) may be themselves be substituted. Substituted forms of the alternative substituent(s) may include, but are not limited to, any form of an amino, azido, imino, amido, phophoryl (such as phosphonate, phosphinate, etc.), sulfonyl (such as sulfate, sulfonamide, sulfamoyl, sulfonate, etc.), silyl, ether, alkylthios, carbonyls (such as ketones, aldehydes, carboxylates, esters, etc.), cyano, etc. Examples of alkenes include, but are not limited to, ethenyl, propenyl, and the like. The term “alkenyl” refers to any monovalent form of the term “alkene” described above. The term “alkendiyl” refers to any divalent form of the term “alkene” described above. The term “alkentriyl” refers to any trivalent form of the term “alkene” described above. The term “alkenquaternyl” refers to any tetravalent form of the term “alkene” described above. “Alkenyl”, “alkendiyl”, “alkentriyl” and “alkenquaternyl” moieties may also undergo substitution as described above for “alkenes”.

Used herein, the term “alkoxy” or “alkyloxy” is recognized in the art and can be defined as including an alkane group attached to an oxygen radical. Examples of alkoxy groups include, but are not limited to methoxy, ethoxy, propyloxy, tert-butoxy, etc. Similarly, an “ether” can be defined as a molecule having two hydrocarbons covalently bound to an oxygen atom. Representative alkane substituents that may convert said alkanes into an ether my include an “alkoxy” of the form —O-alkane, —O-alkene, —O-alkyne, —O-cycloalkane, —O-cycloalkene, —O-cycloalkyne, —O-aryl, —O—(CH2)m-Z, where Z may be an alkane, alkene, alkyne, aryl, cycloalkane, cycloalkene, cycloalkyne, heterocycle or polycycle. Similarly, the term “alkenyloxy” can be defined as including an alkenyl group attached to an oxygen radical. Examples of alkenyloxy groups include, but are not limited to, allyoxy, crytyloxy, 2-pentenyloxy, 3-hexenyloxy. The term “alkynyloxy” can be defined as including an alkynyl group attached to an oxygen radical. The term “cycloalkaneoxy” can be define as including a cycloalkane group attached to an oxygen radical. The term “cycloalkeneoxy” can be defined as including a cycloalkene group attached to an oxygen radical. The term “cycloalkyneloxy” can be defined as including a cycloalkyne group attached to and oxygen radical. The term “aryloxy” can be defined as including an aryl group attached to an oxygen radical. Similarly, the term “dialkoxy” or “dialkaneoxy” refers to two alkoxy or alkyloxy groups, described above, attached to a central atom. The term “trialkoxy” or “trialkaneoxy” refers to three alkoxy or alkaneoxy groups, described above, attached to a central atom. The term “dialkenoxy” or “dialkeneoxy” refers to two alkenoxy groups, described above, attached to a central atom. The term “trialkenoxy” or “trialkeneoxy” refers to three alkenoxy or alkeneoxy groups, described above, attached to a central atom. The term “dialkynoxy” or “dialkyneoxy” refers to two alkynoxy or alkyneoxy groups, described above, attached to a central atom. The term “trialkynoxy” or “trialkyneoxy” refers to three alkynoxy or alkyneoxy groups, described above, attached to a central atom. The term “dicycloalkoxy” or “dicycloalkaneoxy” refers to two cycloalkoxy or cycloalkaneoxy groups, described above, attached to a central atom. The term “tricycloalkoxy” or “tricycloalkaneoxy” refers to three cycloalkoxy or cycloalkaneoxy groups, described above, attached to a central atom. The term “dicycloalkenoxy” or “dicycloalkeneoxy” refers to two cycloalkenoxy or cycloalkeneoxy groups, described above, attached to a central atom. The term “tricycloalkenoxy” or “tricycloalkeneoxy” refers to three cycloalkenoxy or cycloalkeneoxy groups, described above, attached to a central atom. The term “dicycloalkynoxy” or “dicycloalkyneoxy” refers to two cycloalkynoxy or cycloalkyneoxy groups, described above, attached to a central atom. The term “tricycloalkynoxy” or “tricycloalkyneoxy” refers to three cycloalkynoxy or cycloalkyneoxy groups, described above, attached to a central atom. The term “diaryloxy” refers to two aryloxy groups, described above, attached to a central atom. The term “triaryloxy” refers to three aryloxy groups, described above, attached to a central atom.

Used herein, the term “alkoxycarbonyl” or “alkaneoxycarbonyl” is recognized in the art and refers to the formula —C(═O)(alkoxy/alkaneoxy). Similarly, “alkenoxycarbonyl” or “alkeneoxycarbonyl” refers to the formula —C(═O)(alkenoxy/alkeneoxy). The term “alkynoxycarbonyl” or “alkyneoxycarbonyl” refers to the formula —C(═O)(alkynoxy/alkyneoxy). The term “cycloalkoxycarbonyl” or “cycloalkaneoxycarbonyl” refers to the formula —C(═O)(cycloalkoxy/cycloalkaneoxy). The term “cycloalkenoxycarbonyl” or “cycloalkeneoxycarbonyl” refers to the formula —C(═O)(cycloalkenoxy/cycloalkeneoxy). The term “cycloalkynoxycarbonyl” or “cycloalkyneoxycarbonyl” refers to the formula —C(═O)(cycloalkynoxy/cycloalkyneoxy). The term “aryloxycarbonyl” refers to the formula —C(═O)(aryloxy).

Used herein, the term “alkoxycarbonyloxy” is recognized in the art and refers to the formula (alkoxy/alkaneoxy)C(═O)O—. Similarly, the term “alkenoxycarbonyloxy” refers to the group (alkenoxy/alkeneoxy)C(═O)O—; the term “alkynoxycarbonyloxy” refers to the group (alkynoxy/alkyneoxy)C(═O)O—; the term “cycloalkoxycarbonyloxy” refers to the group (cycloalkoxy/cycloalkaneoxy)C(═O)O—; the term “cycloalkenoxycarbonyloxy” refers to the group (cycloalkenoxy/cycloalkane)C(═O)O—; the term “cycloalkynoxycarbonyloxy” refers to the group (cycloalkynoxy/cycloalkyne)C(═O)O—; the term “aryloxycarbonyloxy” refers to the group (aryl)C(═O)O—.

Used herein, the term “alkoxycarbonylamino” or “alkanecarbonylamino” is recognized in the art and refers to the formula (alkoxy/alkaneoxy)C(═O)NZ— where Z may independently be selected from hydrogen, alkane, alkene, alkyne, cycloalkane, cycloalkene, cycloalkyne, aryl or a substituent(s) described herein. The Group representing Z may be substituted or include heteroatom(s) or their heteroatoms may be substituted. Groups on the amino or carbonyl moiety may be protected by conventional protecting groups known to one skilled in the art. Substitutions of the alkoxycarbonylamino moiety may be substituted by groups described herein. Similarly, “alkenoxycarbonylamino” refers to the formula (alkenoxy/alkeneoxy)C(═O)NZ—; “alkynoxycarbonylamino” refers to the formula (alkynoxy/alkyneoxy)C(═O)NZ—; “cycloalkoxycarbonylamino” refers to the formula (cycloalkoxy/cycloalkane)C(═O)NZ—; “cycloalkenoxycarbonylamino” refers to the formula (cycloalkenoxy/cycloalkane)C(═O)NZ—; “cycloalkynoxycarbonylamino” refers to the formula (cycloalkynoxy/cycloalkyne)C(═O)NZ—; “aryloxycarbonylamino” refers to the group (aryl)C(═O)NZ— where Z is defined above for alkoxycarbonylamino.

Used herein, the term “alkyne” is recognized in the art and refers to any unsaturated aliphatic group that includes linear and/or branched alkyne groups, cycloalkyne groups, cycloalkyne groups with alkyne substitutions and/or alkyne groups with cycloalkyne substitutions; hydrocarbon or branched hydrocarbon containing at least one triple bond. As described herein, the term “alkyne” is subject to valence tolerance and may be used interchangeably in place of a monovalent or alkynyl, divalent or alkyndiyl, trivalent or alkyntriyl, tetravalent or alkynquaternyl, and so on. The “alkyne” may be similar in length and/or substitutions to the alkanes described herein. The linear or branched alkyne groups referred to in compositions of the invention have between 3 and 200 carbon atoms and the cycloalkynyl groups have between 3 and 300 carbon atoms. “Alkyne” may also refer to alkynes that have been substituted or alkyne moieties in which one or more hydrogen atoms have been substituted with an alternative substituent. Alternative substituents may include, but are not limited to, an alkane, an alkene, an alkyne, a cycloalkane, a cycloalkene, a cycloalkyne, an aryl, a halogen, a hydroxy, a carboxyl, an alkoxycarbonyl, a formyl, an acyl, or other carbonyl, a thioester, a thioacetate, a thioformate, or other thiocarbonyl, an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic or heteroaromatic, or others described herein, etc. It may be understood by one skilled in the art that the alternative substituents may be themselves be substituted. Substituted form of the alternative substituents may include, but are not limited to, any form of an amino, azido, imino, amido, phophoryl (such as phosphonate, phosphinate, etc.), sulfonyl (such as sulfate, sulfonamide, sulfamoyl, sulfonate, etc.), silyl, ether, alkylthios, carbonyls (such as ketones, aldehydes, carboxylates, esters, etc.), cyano, etc. Examples of alkynes include, but are not limited to, ethynyl, propynyl, and the like. The term “alkynyl” refers to any monovalent form of the term “alkyne” described above. The term “alkyndiyl” refers to any divalent form of the term “alkyne” described above. The term “alkyntriyl” refers to any trivalent form of the term “alkyne” described above. The term “alkynquaternyl” refers to any quadrivalent form of the term “alkyne” described above. “Allkynyl”, “alkyndiyl”, “alkyntriyl” and “alkynquaternyl” moieties may also undergo substitution as described above for “alkane” moieties.

Used herein, the term “amide” is recognized in the art and refers to the group —NZC(═O)— or —C(═O)N(Z′)— where Z and Z′ may independently be selected from hydrogen, alkane, alkene, alkyne, cycloalkane, cycloalkene, cycloalkyne, aryl or a substituent(s) described herein. Groups representing Z and/or Z′ may be substituted or include heteroatom(s) or their heteroatoms may be substituted. Groups on the amide moiety may be protected by conventional protecting groups known to one skilled in the art. Substitutions of the amide moiety may be substituted by groups described herein.

Used herein, the term “amidino” is recognized in the art and refers to the group —ZNC(═NZ′)— where Z and Z′ may independently be selected from hydrogen, alkane, alkene, alkyne, cycloalkane, cycloalkene, cycloalkyne, aryl or a substituent(s) described herein. Groups representing Z and/or Z′ may be substituted or include heteroatom(s) or their heteroatoms may be substituted. Groups on the amidino moiety may be protected by conventional protecting groups known to one skilled in the art. Z and/or Z′ may be joined with the nitrogen atom to form a cyclic structure. Substitutions of the amidino structure may be substituted by groups described herein.

Used herein, the term “amino” and/or “amine” is recognized in the art and refers to the group —NZ—, where Z may be selected from hydrogen, alkane, alkene, alkyne, cycloalkane, cycloalkene, cycloalkyne, aryl or a substituent(s) described herein. Groups representing Z may be substituted or include heteroatom(s) or their heteroatoms may be substituted. Groups on the amino moiety may be protected by conventional protecting groups known to one skilled in the art. Substitutions of the amino moiety as well as the cyclic amine structure may be substituted by groups described herein.

Used herein, the term “aminocarbonyl” is recognized in the art and refers to the group —C(═O)NZ— where Z may be selected from hydrogen, alkane, alkene, alkyne, cycloalkane, cycloalkene, cycloalkyne, aryl or a substituent(s) described herein. Groups representing Z may be substituted or include heteroatom(s) or their heteroatoms may be substituted. Groups on the aminocarbonyl moiety may be protected by conventional protecting groups known to one skilled in the art. Substitutions of the aminocarbonyl moiety may be substituted by groups described herein.

Used herein, the term “aminocarbonylamino” is recognized in the art and refers to the group —NZC(═O)NZ′— where Z and Z″ may be selected from hydrogen, alkane, alkene, alkyne, cycloalkane, cycloalkene, cycloalkyne, aryl or a substituent(s) described herein. Groups representing Z and Z′ may be substituted or include heteroatom(s) or their heteroatoms may be substituted. Groups on the aminocarbonyl moiety may be protected by conventional protecting groups known to one skilled in the art. Substitutions of the aminocarbonylamino moiety may be substituted by groups described herein.

Used herein, the term “aminocarbonyloxy” is recognized in the art and refers to the group —CC(═O)NZ— where Z may independently be selected from hydrogen, alkane, alkene, alkyne, cycloalkane, cycloalkene, cycloalkyne, aryl or a substituent(s) described herein. Groups representing Z may be substituted or include heteroatom(s) or their heteroatoms may be substituted. Groups on the aminocarbonyl moiety may be protected by conventional protecting groups known to one skilled in the art. Substitutions of the aminocarbonyloxy moiety may be substituted by groups described herein.

Used herein, the term “aminosulfonyl” is recognized in the art and refers to the group —S(═O)2NZ— where Z may independently be selected from hydrogen, alkane, alkene, alkyne, cycloalkane, cycloalkene, cycloalkyne, aryl or a substituent(s) described herein. Groups representing Z may be substituted or include heteroatom(s) or their heteroatoms may be substituted. Groups on the aminocarbonyl moiety may be protected by conventional protecting groups known to one skilled in the art. Substitutions of the aminosulfonyl moiety may be substituted by groups described herein.

Used herein, the term “aminosulfonylamino” is recognized in the art and refers to the group —NZSO2NZ′— where Z and Z′ may independently be selected from hydrogen, alkane, alkene, alkyne, cycloalkane, cycloalkene, cycloalkyne, aryl or a substituent(s) described herein. Groups representing Z and Z′ may be substituted or include heteroatom(s) or their heteroatoms may be substituted. Groups on the aminosulfonylamino moiety may be protected by conventional protecting groups known to one skilled in the art. Substitutions of the aminosulfonylamino moiety may be substituted by groups described herein. The Z and/or Z′ may be joined with the nitrogen atom(s) to form a cyclic structure. Substitutions of the as Cyclic aminosulfonylamino structure may be substituted by groups described herein.

Used herein, the term “aminosulfonyloxy” is recognized in the art and refers to the group —OS(═O)₂NZ— where Z may independently be selected from hydrogen, alkane, alkene, alkyne, cycloalkane, cycloalkene, cycloalkyne, aryl or a substituent(s) described herein. Groups representing Z may be substituted or include heteroatom(s) or their heteroatoms may be substituted. Groups on the aminocarbonyl moiety may be protected by conventional protecting groups known to one skilled in the art. Substitutions of the aminosulfonyloxy moiety may be substituted by groups described herein.

Used herein, the term “aminothiocarbonyl” or “aminosulfinyl” is recognized in the art and refers to the group —C(═S)NZ— where Z may independently be selected from hydrogen, alkane, alkene, alkyne, cycloalkane, cycloalkene, cycloalkyne, aryl or a substituent(s) described herein. Groups representing Z may be substituted or include heteroatom(s) or their heteroatoms may be substituted. Groups on the aminocarbonyl moiety may be protected by conventional protecting groups known to one skilled in the art. Substitutions of the aminothiocarbonyl moiety may be substituted by groups described herein.

Used herein, the term “aminothiocarbonylamino” is recognized in the art and refers to the group —NZC(═S)NZ′— where Z and Z′ may be selected from hydrogen, alkane, alkene, alkyne, cycloalkane, cycloalkene, cycloalkyne, aryl or a substituent(s) described herein. Groups representing Z and Z′ may be substituted or include heteroatom(s) or their heteroatoms may be substituted. Groups on the aminocarbonyl moiety may be protected by conventional protecting groups known to one skilled in the art. Substitutions of the aminothiocarbonylamino moiety may be substituted by groups described herein.

Used herein, the term “ammonio” or “ammonium” is recognized in the art and refers to the group —NZZ′-+ where Z and Z′ may independently be selected from hydrogen, alkane, alkene, alkyne, cycloalkane, cycloalkene, cycloalkyne, aryl or a substituent(s) described herein. Groups representing Z and Z′ may be substituted or include heteroatom(s) or their heteroatoms may be substituted. Groups on the ammonio or ammonium moiety may be protected by conventional protecting groups known to one skilled in the art. Substitutions of the ammonio or ammonium moiety may be substituted by groups described herein.

Used herein, the term “anhydride” is recognized in the art and refers to the group —C(═O)OC(═O)—.

Used herein, the term “aryl” is recognized in the art and refers to any single-ring aromatic group that may include up to four heteroatoms. As described herein, the term “aryl” is subject to valence tolerance and may be used interchangeably in place of a monovalent or arylyl, divalent or aryldiyl, trivalent or aryltriyl, tetravalent of arylquaternyl, and so on. Examples include, but are not limited to, benzene, pyrole, furan, thiophene, imidazole, oxazole, thiazole, triazole, tetrazole, pyrazole, pyridine, pyrazine, pyridazine, pyrimidine, etc. “Heteraromatics” are aryl groups that include heteroatoms in the ring structure. The aromatic ring structure may be substituted with one or more substituents such as, but not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamide, ketone, aldehyde, ester, heterocyclyl, aromatic or heteroaromatic groups, —CF₃, —CN, etc. The definition of “aryl” also includes polycyclic ring structures that may have two or more cyclic rings with two or more adjoined carbon atoms. At least one of the adjoined ring structures is aromatic. Other cyclic rings may include cycloalkyls, cycloalkenyls, cycloalkynyl, aryls and/or heterocyclyls. Examples of “aryl” entities include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl benzopyrrolidinyl, benzomorpholinyl, methylenedioxypenyl, ethylenedioypheny, and the like. The term “arylyl” refers to any monovalent form of the term “aryl” described above. The term “aryldiyl” refers to any divalent form of the term “aryl” described above. The term “aryltriyl” refers to any trivalent form of the term “aryl” described above. The term “arylquaternyl” refers to any quadrivalent form of the term “aryl” described above. “Arylyl”, “aryldiyl”, “aryltriyl” and “arylquaternyl” moieties may also undergo substitution as described above for “aryl” moieties.

Used herein, the term “azido” is recognized in the art and refers to the group —N₃.

Used herein, the term “azo” or “diazine” is recognized in the art and refers to the group —N═N—.

Used herein, the term “carbamoyl” is recognized in the art and refers to the group —C(═O)NZ— where Z may be selected from hydrogen, alkane, alkene, alkyne, cycloalkane, cycloalkene, cycloalkyne, aryl or a substituent(s) described herein. The group representing Z may be substituted or include heteroatom(s) or their heteroatoms may be substituted. Groups on the carbamoyl moiety may be protected by conventional protecting groups known to one skilled in the art. Substitutions of the carbamoyl moiety may be substituted by groups described herein.

Used herein, the term “carbamoyloxy” is recognized in the art and refers to the group —OC(═O)NZ— where Z may independently be selected from hydrogen, alkane, alkene, alkyne, cycloalkane, cycloalkene, cycloalkyne, aryl or a substituent(s) described herein. The Group representing Z may be substituted or include heteroatom(s) or their heteroatoms may be substituted. Groups on the amino or carbonyl moiety may be protected by conventional protecting groups known to one skilled in the art. Substitutions of the carbonylamino moiety may be substituted by groups described herein.

Used herein, the term “carbonate” is recognized in the art and refers to the group —O(C═O)O—.

Used herein, the term “carbonothioyl” is recognized in the art and refers to the group —C(═S)—.

Used herein, the term “carbonyl” or “acyl” is recognized in the art and refers to the group —C(═O)—.

Used herein, the term “carbonylamino” is recognized in the art and refers to the group —NZC(═O)— where Z may independently be selected from hydrogen, alkane, alkene, alkyne, cycloalkane, cycloalkene, cycloalkyne, aryl or a substituent(s) described herein. The Group representing Z may be substituted or include heteroatom(s) or their heteroatoms may be substituted. Groups on the amino or carbonyl moiety may be protected by conventional protecting groups known to one skilled in the art. Substitutions of the carbonylamino moiety may be substituted by groups described herein.

Used herein, the term “carbonyloxy” is recognized in the art and refers to the group —C(═O)O—.

Used herein, the term “carbothioic acid” is recognized in the art and refers to the groups —C(═O)SH and/or —C(═S)OH.

Used herein, the term “carboxy” or carboxyl” is recognized in the art and refers to the group —COOH.

Used herein, the term “cyanate” is recognized in the art and refers to the group —OCN.

Used herein, the term “cyano” or “nitrile” is recognized in the art and refers to the group —CN.

Used herein, the term “cycloalkane” is recognized in the art and refers to any saturated carbocyclic entity that consists of a mono- or bicyclic ring structure. As described herein, the term “cycloalkane” is subject to valence tolerance and may be used interchangeably in place of a monovalent of cycloalkyl, divalent or cycloalkdiyl, trivalent or cycloalktriyl, tetravalent or cycloalkquaternyl, and so on. “Cycloalkane” entities may be unsubstituted or substituted similarly to the alkanes described above or as described herein. “Cycloalkane” may also refer to cycloalkanes that have been substituted or cycloalkane moieties in which one or more hydrogen atoms have been substituted with an alternative substituent(s). Alternative substituent(s) may include, but are not limited to, an alkane, an alkene, an alkyne, a cycloalkane, a cycloalkene, a cycloalkyne, an aryl, a halogen, a hydroxy, a carboxyl, an alkoxycarbonyl, a formyl, an acyl, or other carbonyl, a thioester, a thioacetate, a thioformate, or other thiocarbonyl, an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic or heteroaromatic, or others described herein, etc. It may be understood by one skilled in the art that the alternative substituent(s) may be themselves be substituted. Substituted forms of the alternative substituent(s) may include, but are not limited to, any form of an amino, azido, imino, amido, phophoryl (such as phosphonate, phosphinate, etc.), sulfonyl (such as sulfate, sulfonamide, sulfamoyl, sulfonate, etc.), silyl, ether, alkylthios, carbonyls (such as ketones, aldehydes, carboxylates, esters, etc.), cyano, etc. The term “cycloalkane” refers to any monovalent form of the term “cycloalkane” described above. The term “cycloalkdiyl” refers to any divalent form of the term “cycloalkane” described above. The term “cycloalktriyl” refers to any trivalent form of the term “cycloalkane” cycloalkane” described above. The term “cycloalkquaternyl” refers to any quadrivalent form of the term “cycloalkane” described above. “Cycloalkyl”, “cycloalkdiyl”, “cycloalktriyl” and “cycloalkquaternyl” moieties may also undergo substitution as described above for “cycloalkane” moieties.

Used herein, the term “cycloalkene” is recognized in the art and refers to any cycloalkane as described hererin with at least one double bond. As described herein, the term “cycloalkene” cycloalkene” is subject to valence tolerance and may be used interchangeably in place of a monovalent or cycloalkenyl, divalent or cycloalkendiyl, trivalent or cycloalkentriyl, tetravalent or cycloalkenquaternyl, and so on. “Cycloalkene” may also refer to cycloalkenes that have been substituted or cycloalkene moieties in which one or more hydrogen atoms have been substituted with an alternative substituent. Alternative substituent(s) may include, but are not limited to, an alkane, an alkene, an alkyne, a cycloalkane, a cycloalkene, a cycloalkyne, an aryl, a halogen, a hydroxy, a carboxyl, an alkoxycarbonyl, a formyl, an acyl, or other carbonyl, a thioester, a thioacetate, a thioformate, or other thiocarbonyl, an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic or heteroaromatic, or others described herein, etc. It may be understood by one skilled in the art that the alternative substituent(s) may be themselves be substituted. Substituted forms of the alternative substituent(s) may include, but are not limited to, any form of an amino, azido, imino, amido, phophoryl (such as phosphonate, phosphinate, etc.), sulfonyl (such as sulfate, sulfonamide, sulfamoyl, sulfonate, etc.), silyl, ether, alkylthios, carbonyls (such as ketones, aldehydes, carboxylates, esters, etc.), cyano, etc. Examples of cycloalkenes include, but are not limited to, cyclohexene, cyclopentene, cyclobutene, and the like. The term “cycloalkenyl” refers to any monovalent form of the term “cycloalkene” described above. The term “cycloalkendiyl” refers to any divalent form of the term “cycloalkene” described above. The term “cycloalkentriyl” refers to any trivalent form of the term “cycloalkene” described above. The term “cycloalkenquaternyl” refers to any quadrivalent form of the term “cycloalkene” described above. “Cycloalkenyl”, “cycloalkendiyl”, “cycloalkentriyl” and “cycloalkenquaternyl” moieties may also undergo substitution as described above for “cycloalkane” moieties.

Used herein, the term “cycloalkyne” is recognized in the art and refers to any cycloalkane as described herein with at least one triple bond. As described herein, the term “cycloalkane” cycloalkane” is subject to valence tolerance and may be used interchangeably in place of a monovalent or cycloalkynyl, divalent or cycloalkyndiyl, trivalent or cycloalkyntriyl, tetravalent or cycloalkynquaternyl, and so on. “Cycloalkyne” may also refer to cycloalkynes that have been substituted or cycloalkyne moieties in which one or more hydrogen atoms have been substituted with an alternative substituent. Alternative substituents may include, but are not limited to, an alkane, an alkene, an alkyne, a cycloalkane, a cycloalkene, a cycloalkyne, an aryl, a halogen, a hydroxy, a carboxyl, an alkoxycarbonyl, a formyl, an acyl, or other carbonyl, a thioester, a thioacetate, a thioformate, or other thiocarbonyl, an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic or heteroaromatic, or others described herein, etc. It may be understood by one skilled in the art that the alternative substituents may themselves be substituted. Substituted forms of the alternative substituents may include, but are not limited to, any form of an amino, azido, imino, amido, phophoryl (such as phosphonate, phosphinate, etc.), sulfonyl (such as sulfate, sulfonamide, sulfamoyl, sulfonate, etc.), silyl, ether, alkylthios, carbonyls (such as ketones, aldehydes, carboxylates, esters, etc.), cyano, etc. Examples of cycloalkynes include, but are not limited to, cycloheptynyl, cyclooctynyl, cyclononyl, cyclodecynyl, etc. The term “cycloalkynyl” refers to any monovalent form of the term “cycloalkyne” described above. The term “cycloalkyndiyl” refers to any divalent form of the term “cycloalkyne” described above. The term “cycloalkyntriyl” refers to any trivalent form of the term “cycloalkyne” described above. The term “cycloalkynquaternyl” refers to any tetravalent form of the term “cycloalkyne” described above. “Cycloalkynyl”, “cycloalkyndiyl”, “cycloalkyntriyl” and “cycloalkynquaternyl” moieties may also undergo substitution as described above for “cycloalkyne” moieties.

Used herein, the term “disulfide” is recognized is recognized in the art and refers to the group —SS—.

Used herein, the term “dithioate” is recognized in the art and refers to the group —C(═S)S—.

Used herein, the term “dithiocarboxy” is recognized in the art and refers to the group —C(═S)SH.

Used herein, the term “dithioic acid” is recognized in the art and refers to the group —C(═S)SH.

Used herein, the term “formyl” is recognized in the art and refers to the formula —CHO.

Used herein, the term “guanidino” is recognized in the art and refers to the formula —NZC(═NZ′)NZ″—, where Z, Z′ and Z″ may be independently selected from hydrogen, alkane, alkene, alkane, cycloalkane, cycloalkene, cycloalkyne, aryl or a substituent(s) described herein. Groups representing Z, Z′ and Z″ may be substituted or include heteroatom(s) or their heteroatoms may be substituted. Groups on the imido or imide moiety may be protected by conventional protecting groups known to one skilled in the art.

Used herein, the term “guanidinosulfone” is recognized in the art and refers to the group —NZC(═NZ′)NZ″ SO2-, where Z, Z′ and Z″ may independently selected from hydrogen, alkyl, substituted alkyl, alkenyl, substituted alkenyl, alkynyl, substituted alkynyl, cycloalkyl, substituted cycloalkyl, cycloalkenyl, substituted cycloalkenyl, alkynyl, substituted cycloalkynyl, aryl, substituted aryl, alkaryl, substituted alkaryl, aralkyl, substituted aralkyl. Groups representing R may include heteroatom(s). Group(s) on the guanidinosulfone molecule (e g amino group) may be protected by conventional protecting groups known to one skilled in the art.

Use herein, the term “halogen” or “halide” is recognized in the art and refers to a substituent such as fluoro, chloro, bromo, iodo.

Used herein, the term “hetero-”, such as in “heterosubstitution” or “heteroatom” is recognized in the art and refers to an atom of any element other than carbon, such as, but not limited to, N, O, P, B, S, Si, Sb, Al, Sn, As, Se, Ge, etc. that replaces any one or more hydrogen atoms to any carbon and/or any one or more carbon atom within a chemical group.

Used herein, the term “heteroaliphatic” is recognized in the art and refers to aliphatic moieties containing one or more atom such as, but not limited to, N, O, P, B, S, Si, etc. that replaces any carbon atom. Heteroaliphatic moieites may be cyclic or acyclic, linear or branched, saturated or unsaturated. The carbon atoms of the heteroaliphatic moiety may optionally be substituted, as described herein, or the heteroatoms themselves may optionally be oxidized or substituted, as described herein.

Used herein, the term “heteroaryl” is recognized in the art and refers to an aromatic group that contains one or more atoms such as, but not limited to, N, O, P, B, S, Si, etc. that replaces any carbon atom. The heteroaryl moiety may contain a one or more rings that are condensed. The carbon atoms of the heteroaryl moiety may optionally be substituted, as described herein, or the heteroatoms themselves may optionally be oxidized or substituted, as described hererin.

Used herein, the term “heterocyclyl”, “heterocyclic” or “heterocycle” is recognized in the art and refers to cyclic heteroaliphatic groups with a ring system that is non-aromatic, partially unsaturated or fully saturated. The heterocycle moiety may be polycyclic (e.g. monocyclic, bicyclic, tricyclic, etc.) system that may include aryl or heteroaryl moieties that are fused to the cyclic structure(s). The heterocycles contain one or more atom(s) such as, but not limited to, N, O, P, B, S, Si, etc. that replaces any carbon atom. The atoms may be quaternized. The carbon atoms of the heterocycle moiety may optionally be substituted as described herein, or the heteroatoms themselves may optionally be oxidized or substituted, as described herein.

Used herein, the term “hydroxy(thiocarbonyl)” is recognized in the art and refers to the group —C(═S)OH.

Used herein, the term “imido” or “imide” is recognized in the art and refers to the formula —C(═O)NZC(═O)— where Z may be selected from hydrogen, alkane, alkene, alkyne, cycloalkane, cycloalkene, cycloalkyne, aryl or a substituent(s) described herein. Groups representing Z may be substituted or include heteroatom(s) or their heteroatoms may be substituted. Groups on the imido or imide moiety may be protected by conventional protecting groups known to one skilled in the art.

Used herein, the term “imino” or “imine” is recognized in the art and refers to the formula —C(═NZ)— where Z may be selected from hydrogen, alkane, alkene, alkyne, cycloalkane, cycloalkene, cycloalkyne, aryl or a substituent(s) described herein. Groups representing Z may be substituted or include heteroatom(s) or their heteroatoms may be substituted. Groups on the amino moiety may be protected by conventional protecting groups known to one skilled in the art.

Used herein, the term “isocyanate” is recognized in the art and refers to the formula —NCO.

Used herein, the term “isocyano” or “isonitrile” is recognized in the art and refers to the group —NC.

Used herein, the term “isothiocyanate” is recognized in the art and refers to the group —NCS.

Used herein, the term “methylenedioxy” is recognized in the art and refers to the group —OCZZ′O— where Z and Z′ may independently be selected from hydrogen, alkane, alkene, alkyne, cycloalkane, cycloalkene, cycloalkyne, aryl or a substituent(s) described herein. Groups representing Z and/or Z′ may be substituted or include heteroatom(s) or their heteroatoms may be substituted. Groups on the amino moiety may be protected by conventional protecting groups known to one skilled in the art.

Used herein, the term “mercaptocarbonyl” is recognized in the art and refers to the group —C(═O)SH.

Used herein, the term “nitrooxy” is recognized in the art and refers to the group —ON(═O).

Used herein, the term “nitrate” is recognized in the art and refers to the group —ONO2.

Used herein, the term “nitrite” is recognized in the art and refers to the group —ON(═O).

Used herein, the term “nitro” is recognized in the art and refers to the group —NO2.

Used herein, the term “nitroso” is recognized in the art and refers to the group —NO.

Used herein, the term “oxime” is recognized in the art and refers to the group —C(═N—OZ)— where Z may be selected from hydrogen, alkane, alkene, alkyne, cycloalkane, cycloalkene, cycloalkyne, aryl or a substituent(s) described herein. Groups representing Z may be substituted or include heteroatom(s) or their heteroatoms may be substituted. Groups on the oxime moiety may be protected by conventional protecting groups known to one skilled in the art

Used herein, the term “oxo” is recognized in the art and refers to the group ═O.

Used herein, the term “oxy” is recognized in the art and refers to the group —O—.

Used herein, the term “phosphate” is recognized in the art and refers to the group —OP(═O)OZ— where Z may be selected from hydrogen, alkane, alkene, alkyne, cycloalkane, cycloalkene, cycloalkyne, aryl or a substituent(s) described herein. Groups representing Z may be substituted or include heteroatom(s) or their heteroatoms may be substituted. Groups on the phosphate moiety may be protected by conventional protecting groups known to one skilled in the art.

Used herein, the term “phosphino” is recognized in the art and refers to the group —PZ— where Z may be selected from hydrogen, alkane, alkene, alkyne, cycloalkane, cycloalkene, cycloalkyne, aryl or a substituent(s) described herein. Groups representing Z may be substituted or include heteroatom(s) or their heteroatoms may be substituted. Groups on the phosphino moiety may be protected by conventional protecting groups known to one skilled in the art.

Used herein, the term “phosphono” is recognized in the art and refers to the group —PO3Z— where Z may be selected from hydrogen, alkane, alkene, alkyne, cycloalkane, cycloalkene, cycloalkyne, aryl or a substituent(s) described herein. Groups representing Z may be substituted or include heteroatom(s) or their heteroatoms may be substituted. Groups on the phosphono moiety may be protected by conventional protecting groups known to one skilled in the art.

Used herein, the term “pyridyl” is recognized in the art and refers to an aryl group in which one CH unit is replaced by a N and has the general formula —C5Z2N—.

Used herein, the term “sulfhydryl” is recognized in the art and refer to a —SH group.

Used herein, the term “sulfide” is recognized in the art and refers to a —S— group.

Used herein, the term “sulfino” is recognized in the art and refers to a —S(═O)OH group.

Used herein, the term “sulfinyl” is recognized in the art and refers to a —S(═O)— group.

Used herein, the term “sulfo” is recognized in the art and refers to a —S(═O)2O— group.

Used herein, the term “sulfonyl” is recognized in the art and refers to a —S(═O)2- group.

Used herein, the term “thiocyanate” is recognized in the art and refers to a —SCN group.

Used herein, the term “thiolester” is recognized in the art and refers to the group —C(═O)S—.

Used herein, the term “thionoester” is recognized in the art and refers to the group —C(═S)O—.

Used herein, the term “thioyl” is recognized in the art and refers to a —C(═S)— group.

It is appreciated that the chemical moieties described herein of the present invention may be substituted with one or more substituent of functional groups. Generally, the term “substituted” or “substitution” is recognized in the art and refers to the hydrogen radical(s) of a chemical moiety being replaced with a specific substituent radical. One or more substitutions of a chemical moiety may occur with one or more substituents selected from a specific functional group and the substituents may be the same or they may be different. The terms substituted or substitution is contemplated to include all permissible substituents of organic compounds, any of the substituents described herein and/or any combinations thereof. Substituents are subject to valence tolerance as described herein and include, where appropriate, any monovalent, divalent, trivalent, tetravalent, and so on form of halogen, aliphatic; heteroaliphatic, alkane; alkene; alkyne; cyclic; heterocyclic; cycloalkane; cycloalkene; cycloalkyne; heteroaryl; aryl; hydroxyl; carbonyl or acyl; carbonyloxy; alkoxycarbonyl; alkenoxycarbonyl; alkynoxycarbonyl; cycloalkoxycarbonyl; cycloalkenoxycarbonyl; cycloalkynoxycarbonyl; aryloxycarbonyl; carbonylamino; alkoxycarbonylamino; alkenoxycarbonylamino; alkynoxycarbonylamino; cycloalkoxycarbonylamino; cycloalkenoxycarbonylamino; cycloalkynoxycarbonylamino; aryloxycarbonylamino; formyl; oxo; oxy; formyl; alkoxycarbonyloxy, alkenoxycarbonyloxy, alkynoxycarbonyloxy; cycloalkoxycarbonyloxy, cycloalkenoxycarbonyloxy, cycloalkynoxycarbonyloxy, aryloxycarbonyloxy, carbonate, carboxy, methylenedioxy; anhydride; carbamoyl; amide; amino; aminocarbonyl; amine; ammonio; imino; imido; azido; azo; cyanate; isocyanate; nitrooxy; nitrate; cyano; isocyano; nitrite; nitro; nitroso; guanidino; guanidinosulfone; oxime; pyridyl; carbamoyloxy; aminothiocarbonyl; aminocarbonylamino; aminothiocarbonylamino; aminocarbonyloxy; aminosulfonyl; aminosulfonyloxy; aminosulfonylamino; amidino; sulfhydryl; sulfide; disulfide; sulfinyl; sulfonyl; sulfino; sulfo; thiocyanate; isothiocyanate; carbonothioyl; marcapto; mercaptocarbonyl; hydroxythiocarbonyl; thiolester; thionoester; dithiocarboxy; dithioate; phosphino; phosphono; phosphate; thiooxo; carbonyloxyamino; silyl. It is understood that any group or substituent used in substitution may themselves also be substituted. Any group or substituent that is used as part of a substitution may include one or more heteroatoms. Any of the substituents used for substitution may be protected by conventional methods, known to one skilled in the art.

MDE of the invention may include any type of hydrocarbon (e.g. saturated/unsaturated, acyclic/cyclic, etc.) represented by the general structure in Formula 101a through 101e.

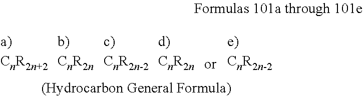

The present invention may include any type of alcohol represented by the general structure in Formulas 102a through 102f.

Formulas 102a through 102e (Alcohol General Formulas):

a) R₁(OH)n, where in embodiments n is an integer which in embodiments is 1-12 or 1-6 or 1, 2 or 3.

b) Ar(OH)n, where in embodiments n is an integer which in embodiments is 1-12 or 1-6 or 1, 2, 3 or 4;

c) Ar₂(R₂OH)n, where in embodiments n is an integer which in embodiments is 1-12 or 1-6 or 1, 2, 3 or 4;

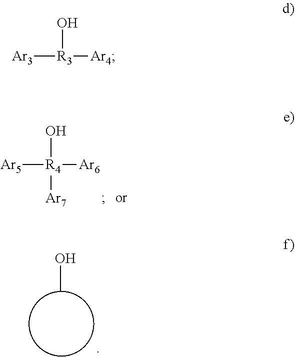

The present invention may include any type of ketone represented by the general structure in Formulas 103a through 103e.

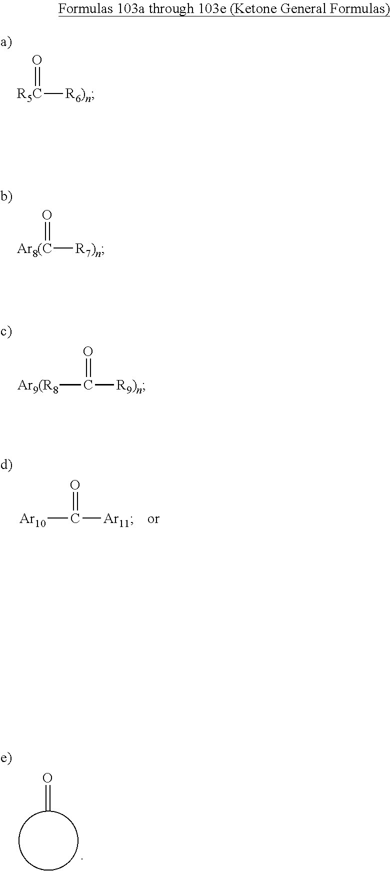

The present invention may include any type of nitrile represented by the general structure in Formulas 104a through 104f.

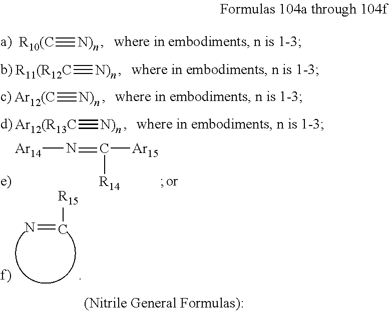

The present invention may include any type of carboxylic acid represented by the general structure in Formulas 105a through 105e.

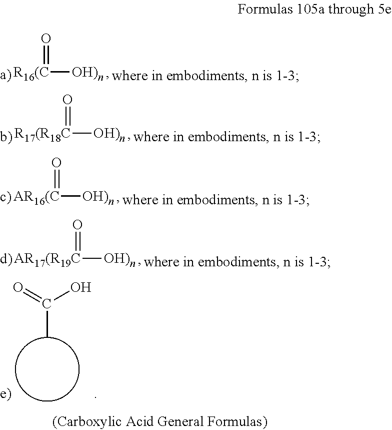

The present invention may include any type of ester represented by the general structure in Formulas 106a though 106f.

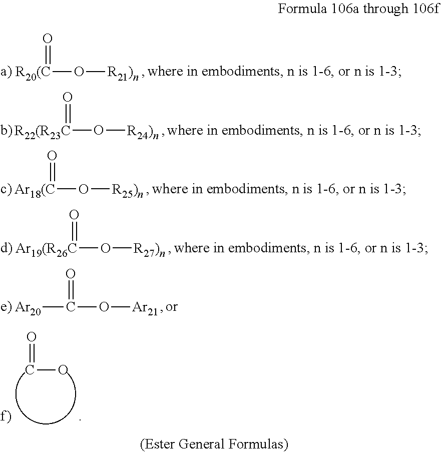

The present invention may include any type of ether represented by the general structure in Formulas 107a though 107f.

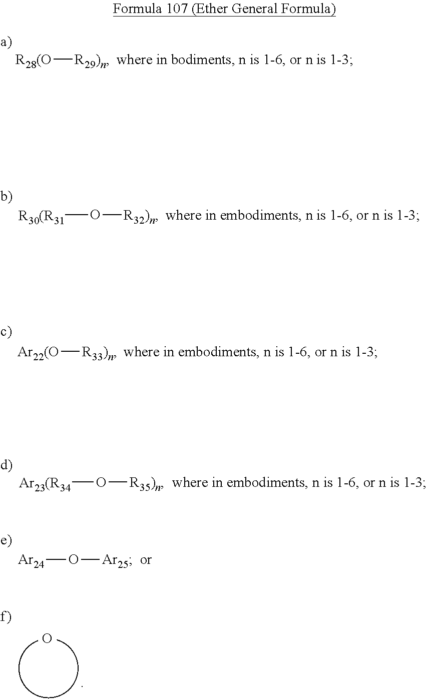

The present invention may include any type of ether represented by the general structure in Formulas 108a though 108f.

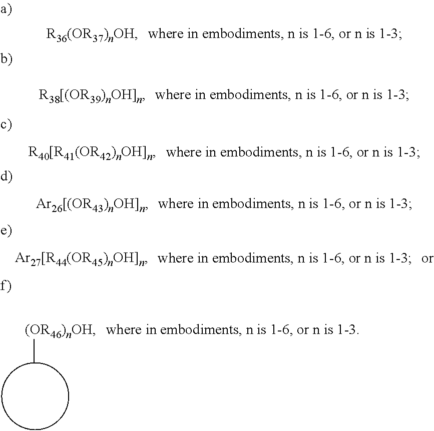

The present invention may include any type of siloxane represented by the general structure in Formulas 109a and 109b.

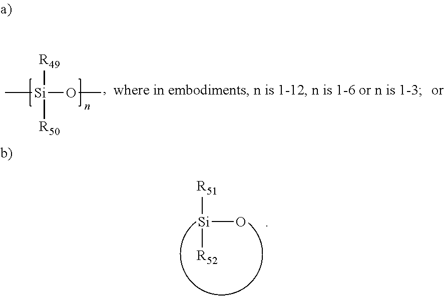

The present invention may include any type of aldehyde represented by the general structure in Formula 110a through 110e.

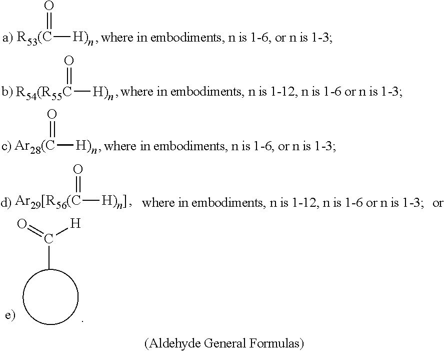

R through R₅₆ and Ar through Ar₂₉, inclusive, are optionally substituted or unsubstituted. R through R₅₆ and Ar through Ar₂₉, inclusive, and/or their substituent(s) or combination of substitutent(s), described herein, are subject to valence tolerance. It is understood that R through R₅₆ and Ar through Ar₂₉, inclusive, and/or their substituent(s) and/or combination of substitutent(s) are consistent with the moiety that is being chemically bonded to and/or being substituted, even though it may not be identified explicitly. R through R₅₆ and Ar through Ar₂₉, inclusive, and/or substitutent(s) and/or combination of substitutent(s) may be monovalent, divalent, trivalent, tetravalent, pentavalent and so on, by inclusion of one, two, three, four, five and so on sites of attachment, respectively, even though they are not explicitly stated. For example, the term “alkane” may interchangeably refer to an example monovalent or alkyl, divalent or alkdiyl, trivalent or alktriyl, tetravalent or alkquaternyl group, and so on. The term “alkene” may refer to an example monovalent or alkenyl, divalent or alkendiyl, trivalent or alkentriyl, tetravalent or alkenquaternyl group, and so on. The term “alkyne” may refer to an example monovalent or alkynyl, divalent or alkyndiyl, trivalent or alkyntriyl, tetravalent or alkynquaternyl, and so on. The term “cycloalkane” may refer to an example monovalent or cycloalkyl, divalent cycloalkdiyl, trivalent cycloalktriyl or tetravalent or cycloalkquaternyl. The term “cycloalkene” may refer to an example monovalent or cycloalkenyl, divalent or cycloalkendiyl, trivalent or cycloalkentriyl, tetravalent or cycloalkenquaternyl, and so on. The term “cycloalkyne” may refer to an example monovalent or cycloalkynyl, divalent or cycloalkyndiyl, trivalent or cycloalkyntriyl, tetravalent or cycloalkynquaternyl, and so on. For example, an alkane used as a pendant group may be included as a monovalent group whereas the same alkane used within a carbon backbone substitution may be included as a divalent, trivalent, tetravalent group. This inclusion applies to all groups, substitutions, pendant groups, etc. referred to in the present invention.

R through R₅₆ and/or Ar through Ar₂₉, inclusive, may include, but are not limited to, any suitable valent form (e.g. monovalent, divalent, trivalent, tetravalent, etc.) of hydrogen; halogen, alkane; alkene; alkyne; cycloalkane; cycloalkene; cycloalkyne; aryl; hydroxyl; hydroxylalkane; hydroxylalkene; hydroxylalkyne; hydroxylcycloalkane; hydroxylcycloalkene; hydroxylcycloalkyne; hydroxylaryl; carbonyl; carbonylalkane; carbonylalkene; carbonylalkyne; carbonylcycloalkane; carbonylcycloalkene; carbonylcycloalkyne; carbonylaryl; alkanecarbonyl; alkenecarbonyl; alkynecarbonyl; cycloalkanecarbonyl; cycloalkenecarbonyl; cycloalkynecarbonyl; arylcarbonyl; carbonyloxy; carbonyloxyalkane; carbonyloxyalkene; carbonyloxyalkyne; carbonyloxycycloalkane; carbonyloxycycloalkene; carbonyloxycycloalkyne; carbonyloxyaryl; alkanecarbonyloxy; alkenecarbonyloxy; alkynecarbonyloxy; cycloalkanecarbonyloxy; cycloalkenecarbonyloxy; cycloalkynecarbonyloxy; arylcarbonyloxy; alkoxycarbonyl; alkoxycarbonylalkane; alkoxycarbonylalkene; alkoxycarbonylalkyne; alkoxycarbonylcycloalkane; alkoxycarbonylcycloalkene; alkoxycarbonylcycloalkyne; alkoxycarbonylaryl; alkenoxycarbonyl; alkenoxycarbonylalkane; alkenoxycarbonylalkene; alkenoxycarbonylalkyne; alkenoxycarbonylcycloalkane; alkenoxycarbonylcycloalkene; alkenoxycarbonylcycloalkyne; alkenoxycarbonylaryl; alkynoxycarbonylalkane; alkynoxycarbonylalkene; alkynoxycarbonylalkyne; alkynoxycarbonylcycloalkane; alkynoxycarbonylcycloalkene; alkynoxycarbonylcycloalkyne; alkynoxycarbonylaryl; cycloalkoxycarbonylalkane; cycloalkoxycarbonylalkene; cycloalkoxycarbonylalkyne; cycloalkoxycarbonylcycloalkane; cycloalkoxycarbonylcycloalkene; cycloalkoxycarbonylcycloalkyne; cycloalkoxycarbonylaryl; cycloalkenoxycarbonylalkane; cycloalkenoxycarbonylalkene; cycloalkenoxycarbonylalkyne; cycloalkenoxycarbonylcycloalkane; cycloalkenoxycarbonylcycloalkene; cycloalkenoxycarbonylcycloalkyne; cycloalkenoxycarbonylaryl; cycloalkynoxycarbonylalkane; cycloalkynoxycarbonylalkene; cycloalkynoxycarbonylalkyne; cycloalkynoxycarbonylcycloalkane; cycloalkynoxycarbonylcycloalkene; cycloalkynoxycarbonylcycloalkyne; cycloalkynoxycarbonylaryl; aryloxycarbonylalkane; aryloxycarbonylalkene; aryloxycarbonylalkyne; aryloxycarbonylcycloalkane; aryloxycarbonylcycloalkene; aryloxycarbonylcycloalkyne; aryloxycarbonylaryl; carbonylamino; carbonylaminoalkane; carbonylaminoalkene; carbonylaminoalkyne; carbonylaminocycloalkane; carbonylaminocycloalkene; carbonylaminocycloalkyne; carbonylaminoaryl; alkanecarbonylamino; alkenecarbonylamino; alkynecarbonylamino; cycloalkanecarbonylamino; cycloalkenecarbonylamino; cycloalkynecarbonylamino; arylcarbonylamino; alkoxycarbonylamino; alkoxycarbonylaminoalkane; alkoxycarbonylaminoalkene; alkoxycarbonylaminoalkyne; alkoxycarbonylaminocycloalkane; alkoxycarbonylaminocycloalkene; alkoxycarbonylaminocycloalkyne; alkoxycarbonylaminoaryl; alkenoxycarbonylamino; alkenoxycarbonylaminoalkane; alkenoxycarbonylaminoalkene; alkenoxycarbonylaminoalkyne; alkenoxycarbonylaminocycloalkane; alkenoxycarbonylaminocycloalkene; alkenoxycarbonylaminocycloalkyne; alkenoxycarbonylaminoaryl; alkynoxycarbonylamino; alkynoxycarbonylaminoalkane; alkynoxycarbonylaminoalkene; alkynoxycarbonylaminoalkyne; alkynoxycarbonylaminocycloalkane; alkynoxycarbonylaminocycloalkene; alkynoxycarbonylaminocycloalkyne; alkynoxycarbonylaminoaryl; cycloalkoxycarbonylamino; cycloalkoxycarbonylaminoalkane; cycloalkoxycarbonylaminoalkene; cycloalkoxycarbonylaminoalkyne; cycloalkoxycarbonylaminocycloalkane; cycloalkoxycarbonylaminocycloalkene; cycloalkoxycarbonylaminocycloalkyne; cycloalkoxycarbonylaminoaryl; cycloalkenoxycarbonylamino; cycloalkenoxycarbonylaminoalkane; cycloalkenoxycarbonylaminoalkene; cycloalkenoxycarbonylaminoalkyne; cycloalkenoxycarbonylaminocycloalkane; cycloalkenoxycarbonylaminocycloalkene; cycloalkenoxycarbonylaminocycloalkyne; cycloalkenoxycarbonylaminoaryl; cycloalkynoxycarbonylamino; cycloalkynoxycarbonylaminoalkane; cycloalkynoxycarbonylaminoalkene; cycloalkynoxycarbonylaminoalkyne; cycloalkynoxycarbonylaminocycloalkane; cycloalkynoxycarbonylaminocycloalkene; cycloalkynoxycarbonylaminocycloalkyne; cycloalkynoxycarbonylaminoaryl; aryloxycarbonylamino; aryloxycarbonylaminoalkane; aryloxycarbonylaminoalkene; aryloxycarbonylaminoalkyne; aryloxycarbonylaminocycloalkane; aryloxycarbonylaminocycloalkene; aryloxycarbonylaminocycloalkyne; aryloxycarbonylaminoaryl; formyl; formylalkane; formylalkene; formylalkyne; formylcycloalkane; formylcycloalkene; formylcycloalkyne; formylaryl; oxy; oxyalkane; oxyalkene; oxyalkyne; oxycycloalkane; oxycycloalkene; oxycycloalkyne; oxyaryl; alkaneoxy; alkeneoxy; alkyneoxy; cycloalkaneoxy; cycloalkeneoxy; cycloalkyneoxy; aryloxy; alkoxycarbonyloxy, alkoxycarbonyloxyalkane; alkoxycarbonyloxyalkene; alkoxycarbonyloxyalkyne; alkoxycarbonyloxycycloalkane; alkoxycarbonyloxycycloalkene; alkoxycarbonyloxycycloalkyne; alkoxycarbonyloxyaryl; alkenoxycarbonyloxy, alkenoxycarbonyloxyalkane; alkenoxycarbonyloxyalkene; alkenoxycarbonyloxyalkyne; alkenoxycarbonyloxycycloalkane; alkenoxycarbonyloxycycloalkene; alkenoxycarbonyloxycycloalkyne; alkenoxycarbonyloxyaryl; alkynoxycarbonyloxy, alkynoxycarbonyloxyalkane; alkynoxycarbonyloxyalkene; alkynoxycarbonyloxyalkyne; alkynoxycarbonyloxycycloalkane; alkynoxycarbonyloxycycloalkene; alkynoxycarbonyloxycycloalkyne; alkynoxycarbonyloxyaryl; cycloalkoxycarbonyloxy, cycloalkoxycarbonyloxyalkane; cycloalkoxycarbonyloxyalkene; cycloalkoxycarbonyloxyalkyne; cycloalkoxycarbonyloxycycloalkane; cycloalkoxycarbonyloxycycloalkene; cycloalkoxycarbonyloxycycloalkyne; cycloalkoxycarbonyloxyaryl; cycloalkenoxycarbonyloxy, cycloalkenoxycarbonyloxyalkane; cycloalkenoxycarbonyloxyalkene; cycloalkenoxycarbonyloxyalkyne; cycloalkenoxycarbonyloxycycloalkane; cycloalkenoxycarbonyloxycycloalkene; cycloalkenoxycarbonyloxycycloalkyne; cycloalkenoxycarbonyloxyaryl; cycloalkynoxycarbonyloxy, cycloalkynoxycarbonyloxyalkane; cycloalkynoxycarbonyloxyalkene; cycloalkynoxycarbonyloxyalkyne; cycloalkynoxycarbonyloxycycloalkane; cycloalkynoxycarbonyloxycycloalkene; cycloalkynoxycarbonyloxycycloalkyne; cycloalkynoxycarbonyloxyaryl; aryloxycarbonyloxy, aryloxycarbonyloxyalkane; aryloxycarbonyloxyalkene; aryloxycarbonyloxyalkyne; aryloxycarbonyloxycycloalkane; aryloxycarbonyloxycycloalkene; aryloxycarbonyloxycycloalkyne; aryloxycarbonyloxyaryl; carbonate; carbonatealkane; carbonatealkene; carbonatealkyne; carbonatecycloalkane; carbonatecycloalkene; carbonatecycloalkyne; carbonatearyl; alkanecarbonate; alkenecarbonate; alkynecarbonate; cycloalkanecarbonate; cycloalkenecarbonate; cycloalkynecarbonate; arylcarbonate; carboxy; carboxyalkane; carboxyalkene; carboxyalkyne; carboxycycloalkane; carboxycycloalkene; carboxycycloalkyne; carboxyaryl; alkoxy/dialkoxy/trialkoxy; (alkoxy/dialkoxy/trialkoxy)alkane; (alkoxy/dialkoxy/trialkoxy)alkene; (alkoxy/dialkoxy/trialkoxy)alkyne; (alkoxy/dialkoxy/trialkoxy)cycloalkane; (alkoxy/dialkoxy/trialkoxy)cycloalkene; (alkoxy/dialkoxy/trialkoxy)cycloalkyne; (alkoxy/dialkoxy/trialkoxy)aryl; alkenoxy/dialkenoxy/trialkenoxy; (alkenoxy/dialkenoxy/trialkenoxy)alkane; (alkenoxy/dialkenoxy/trialkenoxy)alkene; (alkenoxy/dialkenoxy/trialkenoxy)alkyne; (alkenoxy/dialkenoxy/trialkenoxy)cycloalkane; (alkenoxy/dialkenoxy/trialkenoxy)cycloalkene; (alkenoxy/dialkenoxy/trialkenoxy)cycloalkyne; (alkenoxy/dialkenoxy/trialkenoxy)aryl; alkynoxy/dialkynoxy/trialkynoxy; (alkynoxy/dialkynoxy/trialkynoxy)alkane; (alkynoxy/dialkynoxy/trialkynoxy)alkene; (alkynoxy/dialkynoxy/trialkynoxy)alkyne; (alkynoxy/dialkynoxy/trialkynoxy)cycloalkane; (alkynoxy/dialkynoxy/trialkynoxy)cycloalkene; (alkynoxy/dialkynoxy/trialkynoxy)cycloalkyne; (alkynoxy/dialkynoxy/trialkynoxy)aryl; cycloalkoxy/dicycloalkoxy/tricycloalkoxy; (cycloalkoxy/dicycloalkoxy/trialkoxy)alkane; (cycloalkoxy/dicycloalkoxy/tricycloalkoxy)alkene; (cycloalkoxy/dicycloalkoxy/tricycloalkoxy)alkyne; (cycloalkoxy/dicycloalkoxy/tricycloalkoxy)cycloalkane; (cycloalkoxy/dicycloalkoxy/tricycloalkoxy)cycloalkene; (cycloalkoxy/dicycloalkoxy/tricycloalkoxy)cycloalkyne; (cycloalkoxy/dicycloalkoxy/tricycloalkoxy)aryl; cycloalkenoxy/dicycloalkenoxy/tricycloalkenoxy; (cycloalkenoxy/dicycloalkenoxy/tricycloalkenoxy)alkane; (cycloalkenoxy/dicycloalkenoxy/tricycloalkenoxy)alkene; (cycloalkenoxy/dicycloalkenoxy/tricycloalkenoxy)alkyne; (cycloalkoxy/dicycloalkenoxy/tricycloalkenoxy)cycloalkane; (cycloalkenoxy/dicycloalkenoxy/tricycloalkenoxy)cycloalkene; (cycloalkenoxy/dicycloalkenoxy/tricycloalkenoxy)cycloalkyne; (cycloalkenoxy/dicycloalkenoxy/tricycloalkenoxy)aryl; cycloalkynoxy/dicycloalkynoxy/tricycloalkynoxy; (cycloalkynoxy/dicycloalkynoxy/tricycloalkynoxy)alkane; (cycloalkynoxy/dicycloalkynoxy/tricycloalkynoxy)alkene; (cycloalkynoxy/dicycloalkynoxy/tricycloalkynoxy)alkyne; (cycloalkynoxy/dicycloalkynoxy/tricycloalkynoxy)cycloalkane; (cycloalkynoxy/dicycloalkynoxy/tricycloalkynoxy)cycloalkene; (alkynoxy/dicycloalkynoxy/tricycloalkynoxy)cycloalkyne; (cycloalkynoxy/dicycloalkynoxy/tricycloalkynoxy)aryl; aryloxy/diaryloxy/triaryloxy; (aryloxy/diaryloxy/triaryloxy)alkane; (aryloxy/diaryloxy/triaryloxy)alkene; (aryloxy/diaryloxy/triaryloxy)alkyne; (aryloxy/diaryloxy/triaryloxy)cycloalkane; (aryloxy/diaryloxy/triaryloxy)cycloalkene; (aryloxy/diaryloxy/triaryloxy)cycloalkyne; (aryloxy/diaryloxy/triaryloxy)aryl; methylenedioxy; methylenedioxyalkane; methylenedioxyalkene; methylenedioxyalkyne; methylenedioxycycloalkane; methylenedioxycycloalkene; methylenedioxycycloalkyne; methylenedioxyaryl; alkanemethylenedioxy; alkenemethylenedioxy; alkynemethylenedioxy; cycloalkanemethylenedioxy; cycloalkenemethylenedioxy; cycloalkynemethylenedioxy; arylmethylenedioxy; anhydride; anhydridealkane; anhydridealkene; anhydridealkyne; anhydridecycloalkane; anhydridecycloalkene; anhydridecycloalkyne; anhydridearyl; alkaneanhydride; alkeneanhydride; alkyneanhydride; cycloalkaneanhydride; cycloalkaneanhydride; cycloalkyneanhydride; arylanhydride; carbamoyl; carbamoylalkane; carbamoylalkene; carbamoylalkyne; carbamoylcycloalkane; carbamoylcycloalkene; carbamoylcycloalkyne; carbamoylaryl; alkanecarbamoyl; alkenecarbamoyl; alkynecarbamoyl; cycloalkanecarbamoyl; cycloalkenecarbamoyl; cycloalkynecarbamoyl; arylcarbamoyl; amide; amidealkane; amidealkene; amidealkyne; amidecycloalkane; amidecycloalkene; amidecycloalkyne; amidearyl; alkaneamide; alkeneamide; alkyneamide; cycloalkaneamide; cycloalkeneamide; cycloalkyneamide; arylamide; amino; aminoalkane; aminoalkene; aminoalkane; aminocycloalkane; aminocycloalkene; aminocycloalkyne; aminoaryl; alkaneamino; alkeneamino; alkyneamino; cycloalkaneamino; cycloalkeneamino; cycloalkyneamino; arylamino; aminocarbonyl; aminocarbonylalkane; aminocarbonylalkene; aminocarbonylalkyne; aminocarbonylcycloalkane; aminocarbonylcycloalkene; aminocarbonylcycloalkyne; aminocarbonylaryl; alkaneaminocarbonyl; alkeneaminocarbonyl; alkyneaminocarbonyl; cycloalkaneaminocarbonyl; cycloalkeneaminocarbonyl; cycloalkyneaminocarbonyl; arylaminocarbonyl; amine; aminealkane; aminealkene; aminealkyne; aminecycloalkane; aminecycloalkene; aminecycloalkyne; aminearyl; alkaneamine; alkeneamine; alkyneamine; cycloalkaneamine; cycloalkeneamine; cycloalkyneamine; ammonio; ammonioalkane; ammonioalkane; ammonioalkene; ammonioalkyne; ammoniocycloalkane; ammoniocycloalkene; ammoniocycloalkyne; ammonioaryl; alkaneammonio; alkeneammonio; alkyneammonio; cycloalkaneammonio; cycloalkeneammonio; cycloalkyneammonio; arylammonio; imino; iminoalkane; iminoalkene; iminoalkyne; iminocycloalkane; iminocycloalkene; iminocycloalkyne; iminoaryl; alkaneimino; alkeneimino; alkyneimino; cycloalkaneimino; cycloalkeneimino; cycloalkyneimido; arylimino; imido; imidoalkane; imidoalkene; imidoalkyne; imidocycloalkane; imidocycloalkene; imidocycloalkyne; imidoaryl; alkaneimido; alkeneimido; alkyneimido; cycloalkaneimido; cycloalkeneimido; cycloalkyneimido; arylimido; azido; azidoalkane; azidoalkene; azidoalkyne; azidocycloalkane; azidocycloalkene; azidocycloalkyne; azidoaryl; azo; azoalkane; azoalkene; azoalkyne; azocycloalkane; azocycloalkene; azocycloalkyne; azoaryl; alkaneazo; alkeneazo; alkyneazo; cycloalkaneazo; cycloalkeneazo; cycloalkyneazo; arylazo; cyanate; cyanatealkane; cyanatealkene; cyanatealkyne; cyanatecycloalkane; cyanatecycloalkene; cyanatecycloalkyne; cyanatearyl; isocyanate; isocyanatealkane; isocyanatealkene; isocyanatealkyne; isocyanatecycloalkane; isocyanatecycloalkene; isocyanatecycloalkyne; isocyanatearyl; nitrooxy; nitrooxyalkane; nitrooxyalkene; nitrooxyalkyne; nitrooxycycloalkane; nitrooxycycloalkene; nitrooxycycloalkyne; nitrooxycycloaryl; nitrate; nitratealkane; nitratealkene; nitratealkyne; nitratecycloalkane; nitratecycloalkene; nitratecycloalkyne; nitratearyl; cyano; cyanoalkane; cyanoalkene; cyanoalkyne; cyanocycloalkane; cyanocycloalkene; cyanocycloalkyne; cyanoaryl; isocyano; isocyanoalkane; isocyanoalkene; isocyanoalkyne; isocyanocycloalkane; isocyanocycloalkene; isocyanocycloalkyne; isocyanoaryl; nitrite; nitritealkane; nitritealkene; nitritealkyne; nitritecycloalkane; nitritecycloalkene; nitritecycloalkyne; nitritearyl; nitro; nitroalkane; nitroalkene; nitroalkyne; nitrocycloalkane; nitrocycloalkene; nitrocycloalkyne; nitroaryl; nitroso; nitrosoalkane; nitrosoalkene; nitrosoalkyne; nitrosocycloalkane; nitrosocycloalkene; nitrosocycloalkyne; nitrosoaryl; guanidino; guanidinoalkane; guanidinoalkene; guanidinoalkyne; guanidinocycloalkane; guanidinocycloalkene; guanidinocycloalkyne; guanidinoaryl; alkaneguanidino; alkeneguanidino; alkyneguanidino; cycloalkaneguanidino; cycloalkeneguanidino; cycloalkyneguanidino; arylguanidino; guanidinosulfone; guanidinosulfonealkane; guanidinosulfonealkene; guanidinosulfonealkyne; guanidinosulfonecycloalkane; guanidinosulfonecycloalkene; guanidinosulfonecycloalkyne; guanidinosulfonearyl; alkane guanidinosulfone; alkeneguanidinosulfone; alkyneguanidinosulfone; cycloalkaneguanidinosulfone; cycloalkeneguanidinosulfone; cycloalkyneguanidinosulfone; arylguanidinosulfone; oxime; oximealkane; oximealkene; oximealkyne; oximecycloalkane; oximecycloalkene; oximecycloalkyne; oximearyl; alkaneoxime; alkeneoxime; alkyneoxime; cycloalkaneoxime; cycloalkeneoxime; cycloalkyneoxime; aryloxime; pyridyl; pyridylalkane; pyridylalkene; pyridylalkyne; pyridylcycloalkane; pyridylcycloalkene; pyridylcycloalkyne; pyridylaryl; alkanepyridyl; alkenepyridyl; alkynepyridyl; cycloalkanepyridyl; cycloalkenepyridyl; cycloalkynepyridyl; arylpyridyl; carbamoyloxy; carbamoyloxyalkane; carbamoyloxyalkene; carbamoyloxyalkane; carbamoyloxycycloalkane; carbamoyloxycycloalkene; carbamoyloxycycloalkyne; carbamoyloxyaryl; alkanecarbamoyloxy; alkenecarbamoyloxy; alkynecarbamoyloxy; cycloalkanecarbamoyloxy; cycloalkenecarbamoyloxy; cycloalkynecarbamoyloxy; arylcarbamoyloxy; aminothiocarbonyl; alkaneaminiothiocarbonyl; alkeneaminothiocarbonyl; alkyneaminothiocarbonyl; cycloalkaneaminothiocarbonyl; cycloalkeneaminothiocarbonyl; cycloalkyneaminothiocarbonyl; arylaminothiocarbonyl; aminothiocarbonylalkane; aminothiocarbonylalkene; aminothiocarbonylalkyne; aminothiocarbonylcycloalkane; aminothiocarbonylcycloalkene; aminothiocarbonylcycloalkyne; aminothiocarbonylaryl; aminocarbonylamino; aminocarbonylaminoalkane; aminocarbonylaminoalkene; aminocarbonylaminoalkyne; aminocarbonylaminocycloalkane; aminocarbonylaminocycloalkene; aminocarbonylaminocycloalkyne; aminocarbonylaminoaryl; alkaneaminocarbonylamino; alkeneaminocarbonylamino; alkyneaminocarbonylamino; cycloalkaneaminocarbonylamino; cycloalkeneaminocarbonylamino; cycloalkyneaminocarbonylamino; arylaminocarbonylamino; aminothiocarbonylamino; aminothiocarbonylaminoalkane; aminothiocarbonylaminoalkene; aminothiocarbonylaminoalkyne; aminothiocarbonylaminocycloalkane; aminothiocarbonylaminocycloalkene; aminothiocarbonylaminocycloalkyne; aminothiocarbonylaminoaryl; alkaneaminothiocarbonylamino; alkeneaminothiocarbonylamino; alkyneaminothiocarbonylamino; cycloalkaneaminothiocarbonylamino; cycloalkeneaminothiocarbonylamino; cycloalkyneaminothiocarbonylamino; arylaminothiocarbonylamino; aminocarbonyloxy; aminocarbonyloxyalkane; aminocarbonyloxyalkene; aminocarbonyloxyalkyne; aminocarbonyloxycycloalkane; aminocarbonyloxycycloalkene; aminocarbonyloxycycloalkyne; aminocarbonyloxyaryl; alkaneaminocarbonyloxy; alkeneaminocarbonyloxy; alkyneaminocarbonyloxy; cycloalkaneaminocarbonyloxy; cycloalkeneaminocarbonyloxy; cycloalkyneaminocarbonyloxy; arylaminocarbonyloxy; aminosulfonyl; aminosulfonylalkane; aminosulfonylalkene; aminosulfonylalkyne; aminosulfonylcycloalkane; aminosulfonylcycloalkene; aminosulfonylcycloalkyne; aminosulfonylaryl; alkaneaminosulfonyl; alkeneaminosulfonyl; alkyneaminosulfonyl; cycloalkaneaminosulfonyl; cycloalkeneaminosulfonyl; cycloalkyneaminosulfonyl; arylaminosulfonyl; aminosulfonyloxy; aminosulfonyloxyalkane; aminosulfonyloxyalkene; aminosulfonyloxyalkyne; aminosulfonyloxycycloalkane; aminosulfonyloxycycloalkene; aminosulfonyloxycycloalkyne; aminosulfonyloxyaryl; alkaneaminosulfonyloxy; alkeneaminosulfonyloxy; alkyneaminosulfonyloxy; cycloalkaneaminosulfonyloxy; cycloalkeneaminosulfonyloxy; cycloalkyneaminosulfonyloxy; arylaminosulfonyloxy; aminosulfonylamino; aminosulfonylaminoalkane; aminosulfonylaminoalkene; aminosulfonylaminoalkyne; aminosulfonylaminocycloalkane; aminosulfonylaminocycloalkene; aminosulfonylaminocycloalkyne; aminosulfonylaminoaryl; alkaneaminosulfonylamino; alkeneaminosulfonylamino; alkyneaminosulfonylamino; cycloalkaneaminosulfonylamino; cycloalkeneaminosulfonylamino; cycloalkyneaminosulfonylamino; arylaminosulfonylamino; amidino; amidinoalkane; amidinoalkene; amidinoalkyne; amidinocycloalkane; amidinocycloalkene; amidinocycloalkyne; amidinoaryl; alkaneamidino; alkeneamidino; alkyneamidino; cycloalkaneamidino; cycloalkeneamidino; cycloalkyneamidino; arylamidino; sulfhydryl; sulfhydrylalkane; sulfhydrylalkene; sulfhydrylalkyne; sulfhydrylcycloalkane; sulfhydrylcycloalkene; sulfhydrylcycloalkyne; sulfhydrylaryl; sulfide; sulfidealkane; sulfidealkene; sulfidealkyne; sulfidecycloalkane; sulfidecycloalkene; sulfidecycloalkyne; sulfidearyl; alkanesulfide; alkenesulfide; alkynesulfide; cycloalkanesulfide; cycloalkenesulfide; cycloalkynesulfide; arylsulfide; disulfide; disulfidealkane; disulfidealkene; disulfidealkyne; disulfidecycloalkane; disulfidecycloalkene; disulfidecycloalkyne; disulfidearyl; alkanedisulfide; alkenedisulfide; alkynedisulfide; cycloalkanedisulfide; cycloalkenedisulfide; cycloalkynedisulfide; aryldisulfide; sulfinyl; sulfinylalkane; sulfinylalkene; sulfinylalkyne; sulfinylcycloalkane; sulfinylcycloalkene; sulfinylcycloalkyne; sulfinylaryl; alkanesulfinyl; alkenesulfinyl; alkynesulfinyl; cycloalkanesulfinyl; cycloalkenesulfinyl; cycloalkynesulfinyl; arylsulfinyl; sulfonyl; sulfonylalkane; sulfonylalkene; sulfonylalkyne; sulfonylcycloalkane; sulfonylcycloalkene; sulfonylcycloalkyne; sulfonylaryl; alkanesulfonyl; alkenesulfonyl; alkynesulfonyl; cycloalkanesulfonyl; cycloalkenesulfonyl; cycloalkynesulfonyl; arylsulfonyl; sulfino; sulfinoalkane; sulfinoalkene; sulfinoalkyne; sulfinocycloalkane; sulfinocycloalkene; sulfinocycloalkyne; sulfinoaryl; sulfo; sulfoalkane; sulfoalkene; sulfoalkyne; sulfocycloalkane; sulfocycloalkene; sulfocycloalkyne; sulfoaryl; alkanesulfo; alkenesulfo; alkynesulfo; cycloalkanesulfo; cycloalkenesulfo; cycloalkynesulfo; arylsulfo; thiocyanate; thiocyanatealkane; thiocyanatealkene; thiocyanatealkyne; thiocyanatecycloalkane; thiocyanatecycloalkene; thiocyanatecycloalkyne; thiocyanatearyl; isothiocyanate; isothiocyanatealkane; isothiocyanatealkene; isothiocyanatealkyne; isothiocyanatecycloalkane; isothiocyanatecycloalkene; isothiocyanatecycloalkyne; isothiocyantearyl; carbonothioyl; carbonothioylalkane; carbonothioylalkene; carbonothioylalkyne; carbonothioylcycloalkane; carbonothioylcycloalkene; carbonothioylcycloalkyne; carbonothioylaryl; alkanecarbonothioyl; alkenecarbonothioyl; alkynecarbonothioyl; cycloalkanecarbonothioyl; cycloalkenecarbonothioyl; cycloalkynecarbonothioyl; arylcarbonothioyl; mercaptocarbonyl; mercaptocarbonylalkane; mercaptocarbonylalkene; mercaptocarbonylalkyne; mercaptocarbonylcycloalkane; mercaptocarbonylcycloalkene; mercaptocarbonylcycloalkyne; mercaptocarbonylaryl; hydroxythiocarbonyl; hydroxythiocarbonylalkane; hydroxythiocarbonylalkene; hydroxythiocarbonylalkyne; hydroxythiocarbonylcycloalkane; hydroxythiocarbonylcycloalkene; hydroxythiocarbonylcycloalkyne; hydroxythiocarbonylaryl; thiolester, thiolesteralkane; thiolesteralkene; thiolesteralkyne; thiolestercycloalkane; thiolestercycloalkene; thiolestercycloalkyne; thiolesteraryl; alkanethiolester; alkenethiolester; alkynethiolester; cycloalkanethiolester; cycloalkenethiolester; cycloalkynethiolester; arylthiolester; thionoester; thionoesteralkane; thionoesteralkene; thionoesteralkyne; thionoestercycloalkane; thionoestercycloalkene; thionoestercycloalkyne; thionoesteraryl; alkanethionoester; alkenethionoester; alkynethionoester; cycloalkanethionoester; cycloalkenethionoester; cycloalkynethionoester; arylthionoester; dithiocarboxy; dithiocarboxyalkane; dithiocarboxyalkene; dithiocarboxyalkyne; dithiocarboxycycloalkane; dithiocarboxycycloalkene; dithiocarboxycycloalkyne; dithiocarboxyaryl; dithioate; dithioatealkane; dithioatealkene; dithioatealkyne; dithioatecycloalkane; dithioatecycloalkene; dithioatecycloalkyne; dithioatearyl; alkanedithioate; alkenedithioate; alkynedithioate; cycloalkanedithioate; cycloalkenedithioate; cycloalkynedithioate; aryldithioate; phosphino; phosphinoalkane; phosphinoalkene; phosphinoalkyne; phosphinocycloalkane; phophinocycloalkene; phosphinocycloalkyne; phosphinoaryl; alkanephosphino; alkenephosphino; alkynephosphino; cycloalkanephosphino; cycloalkenephosphino; cycloalkynephosphino; arylphosphino; phosphono; phosphonoalkane; phosphonoalkene; phosphonoalkyne; phosphonocycloalkane; phosphonocycloalkene; phosphonocycloalkyne; phosphonoaryl; alkanephosphono; alkenephosphono; alkynephosphono; cycloalkanephosphono; cycloalkenephosphono; cycloalkynephosphono; arylphosphono; phosphate; phosphatealkane; phosphatealkene; phosphatealkyne; phosphatecycloalkane; phosphatecycloalkene; phosphatecycloalkyne; phosphatearyl; alkanephosphate; alkenephosphate; alkynephosphate; cycloalkanephosphate; cycloalkenephosphate; cycloalkynephosphate; or arylphosphate.

R through R₅₆ and/or Ar through Ar₂₉, inclusive, may also be independently selected from any unsubstituted or substitued, suitable valent form of a carboxyl, carboxylester, carboxylesteramino, carboxylesteroxy, haloalkoxy, halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, cycloalkoxy, orthocarbonate ester, organic acid anhydride, silyl, alkylthio, sulfonamide, trifluoromethyl, thiocyano, mercaptocarbonyl, hydroxythiocarbonyl, thiolester, thionoester, carbodithioic acid, carbodithiol, carboyl, formyl, haloformyl, (alkoxycarbonyl)oxy, carboxy, methoxy, alkoxy, alkoxyol dialkoxy, trialkoxy methylenedioxy; tetralkoxy; carboxylic anhydride; carbamoyl; imino; cyanate; isocyanato; nitroxy; nitrosoxy; nitro; nitroso; oxime; pyridyl; carbamate; phosphanyl; phosphono; phosphonoxy; [(alkoxy)hydroxyphosphoryl]oxy, or acyl, etc. Groups defined by R through R₅₆ and/or Ar through Ar₂₉, inclusive, may include heteroatoms (e.g. heteroalkyl, heteroaryl, heterocyclyl, etc.). Those groups with heteroatoms may also be substituted.

In certain implementations, R through R₅₆, inclusive, or Ar through Ar₂₉, inclusive, may be a group haing at least one ring structure. In formulas with more than one ring structure, ring structure radicals may be fused to one another or bonded to one another by way of a C—C bond or by other bridging groups. The bridging groups may be other ring structures. Ring structures may include any aromatic ring, heterocyclic, etc. and is not limited by the type of element used in the backbone of the ring. The single ring structures may include, but is not limited to, between 5 and 7 members. Other non-limiting bridging group examples may include, but are not limited to, —(CH₂)ₓ—, —C(═O)—, —O—, —S(═O)₂—, —C(CF₃)₂—, —Si(CH₃)₂—, —C(CH₃)₂—, —(CH₂O)ₓ—, etc. where x is an integer between 1 and 20,000. In embodiments, x is an integer ranging inclusively from 1-10, 1-20, 1-40, 1-50, 1-100, 1-1,000, 1-5,000, 1-10,000, 10-50, 10-100, 10-1,000, 10-5,000, 10-10,000, 10-20,000, 100-1,000, 100-5,000, 100-10,000 or 100-20,000.

In the formulas above, n is an integer between 1 and 20,000.

In embodiments, n of the above formulas independently ranges inclusively from 1-3, or 1-6, or 1-10, or 1-12, or 1-20, or 1-50, 1-25, or 100 to 20,000 or from 500 to 20,000 or from 1,000 to 20,000 or from 5,000 to 20,000 or from 100 to 10,000 or from 500 to 10,000 or from 1,000 to 10,000 or from 5,000 to 10,000, or from 100 to 5,000 or from 500 to 5,000 or from 1,000 to 5,000. In some embodiments of the above formulas n is 1, 2 or 3. In some embodiments of the above formulas, n is 4, 5, 6, 7, 8, 9, 10, 11 or 12.

In some implmentations, the carbon backbone of the MDE may be substituted within the backbone of the MDE. More than one backbone substitution may be selected and is subject to valence tolerance. The group may also be substituted or unsubstituted and may include, but are not limited to, any suitable valent form (e.g. monovalent, divalent, trivalent, tetravalent, etc.) of halogen, alkane; alkene; alkyne; cycloalkane; cycloalkene; cycloalkyne; aryl; hydroxyl; hydroxylalkane; hydroxylalkene; hydroxylalkyne; hydroxylcycloalkane; hydroxylcycloalkene; hydroxylcycloalkyne; hydroxylaryl; carbonyl; carbonylalkane; carbonylalkene; carbonylalkyne; carbonylcycloalkane; carbonylcycloalkene; carbonylcycloalkyne; carbonylaryl; alkanecarbonyl; alkenecarbonyl; alkynecarbonyl; cycloalkanecarbonyl; cycloalkenecarbonyl; cycloalkynecarbonyl; arylcarbonyl; carbonyloxy; carbonyloxyalkane; carbonyloxyalkene; carbonyloxyalkyne; carbonyloxycycloalkane; carbonyloxycycloalkene; carbonyloxycycloalkyne; carbonyloxyaryl; alkanecarbonyloxy; alkenecarbonyloxy; alkynecarbonyloxy; cycloalkanecarbonyloxy; cycloalkenecarbonyloxy; cycloalkynecarbonyloxy; arylcarbonyloxy; alkoxycarbonyl; alkoxycarbonylalkane; alkoxycarbonylalkene; alkoxycarbonylalkyne; alkoxycarbonylcycloalkane; alkoxycarbonylcycloalkene; alkoxycarbonylcycloalkyne; alkoxycarbonylaryl; alkenoxycarbonyl; alkenoxycarbonylalkane; alkenoxycarbonylalkene; alkenoxycarbonylalkyne; alkenoxycarbonylcycloalkane; alkenoxycarbonylcycloalkene; alkenoxycarbonylcycloalkyne; alkenoxycarbonylaryl; alkynoxycarbonylalkane; alkynoxycarbonylalkene; alkynoxycarbonylalkyne; alkynoxycarbonylcycloalkane; alkynoxycarbonylcycloalkene; alkynoxycarbonylcycloalkyne; alkynoxycarbonylaryl; cycloalkoxycarbonylalkane; cycloalkoxycarbonylalkene; cycloalkoxycarbonylalkyne; cycloalkoxycarbonylcycloalkane; cycloalkoxycarbonylcycloalkene; cycloalkoxycarbonylcycloalkyne; cycloalkoxycarbonylaryl; cycloalkenoxycarbonylalkane; cycloalkenoxycarbonylalkene; cycloalkenoxycarbonylalkyne; cycloalkenoxycarbonylcycloalkane; cycloalkenoxycarbonylcycloalkene; cycloalkenoxycarbonylcycloalkyne; cycloalkenoxycarbonylaryl; cycloalkynoxycarbonylalkane; cycloalkynoxycarbonylalkene; cycloalkynoxycarbonylalkyne; cycloalkynoxycarbonylcycloalkane; cycloalkynoxycarbonylcycloalkene; cycloalkynoxycarbonylcycloalkyne; cycloalkynoxycarbonylaryl; aryloxycarbonylalkane; aryloxycarbonylalkene; aryloxycarbonylalkyne; aryloxycarbonylcycloalkane; aryloxycarbonylcycloalkene; aryloxycarbonylcycloalkyne; aryloxycarbonylaryl; carbonylamino; carbonylaminoalkane; carbonylaminoalkene; carbonylaminoalkyne; carbonylaminocycloalkane; carbonylaminocycloalkene; carbonylaminocycloalkyne; carbonylaminoaryl; alkanecarbonylamino; alkenecarbonylamino; alkynecarbonylamino; cycloalkanecarbonylamino; cycloalkenecarbonylamino; cycloalkynecarbonylamino; arylcarbonylamino; alkoxycarbonylamino; alkoxycarbonylaminoalkane; alkoxycarbonylaminoalkene; alkoxycarbonylaminoalkyne; alkoxycarbonylaminocycloalkane; alkoxycarbonylaminocycloalkene; alkoxycarbonylaminocycloalkyne; alkoxycarbonylaminoaryl; alkenoxycarbonylamino; alkenoxycarbonylaminoalkane; alkenoxycarbonylaminoalkene; alkenoxycarbonylaminoalkyne; alkenoxycarbonylaminocycloalkane; alkenoxycarbonylaminocycloalkene; alkenoxycarbonylaminocycloalkyne; alkenoxycarbonylaminoaryl; alkynoxycarbonylamino; alkynoxycarbonylaminoalkane; alkynoxycarbonylaminoalkene; alkynoxycarbonylaminoalkyne; alkynoxycarbonylaminocycloalkane; alkynoxycarbonylaminocycloalkene; alkynoxycarbonylaminocycloalkyne; alkynoxycarbonylaminoaryl; cycloalkoxycarbonylamino; cycloalkoxycarbonylaminoalkane; cycloalkoxycarbonylaminoalkene; cycloalkoxycarbonylaminoalkyne; cycloalkoxycarbonylaminocycloalkane; cycloalkoxycarbonylaminocycloalkene; cycloalkoxycarbonylaminocycloalkyne; cycloalkoxycarbonylaminoaryl; cycloalkenoxycarbonylamino; cycloalkenoxycarbonylaminoalkane; cycloalkenoxycarbonylaminoalkene; cycloalkenoxycarbonylaminoalkyne; cycloalkenoxycarbonylaminocycloalkane; cycloalkenoxycarbonylaminocycloalkene; cycloalkenoxycarbonylaminocycloalkyne; cycloalkenoxycarbonylaminoaryl; cycloalkynoxycarbonylamino; cycloalkynoxycarbonylaminoalkane; cycloalkynoxycarbonylaminoalkene; cycloalkynoxycarbonylaminoalkyne; cycloalkynoxycarbonylaminocycloalkane; cycloalkynoxycarbonylaminocycloalkene; cycloalkynoxycarbonylaminocycloalkyne; cycloalkynoxycarbonylaminoaryl; aryloxycarbonylamino; aryloxycarbonylaminoalkane; aryloxycarbonylaminoalkene; aryloxycarbonylaminoalkyne; aryloxycarbonylaminocycloalkane; aryloxycarbonylaminocycloalkene; aryloxycarbonylaminocycloalkyne; aryloxycarbonylaminoaryl; formyl; formylalkane; formylalkene; formylalkyne; formylcycloalkane; formylcycloalkene; formylcycloalkyne; formylaryl; oxy; oxyalkane; oxyalkene; oxyalkyne; oxycycloalkane; oxycycloalkene; oxycycloalkyne; oxyaryl; alkaneoxy; alkeneoxy; alkyneoxy; cycloalkaneoxy; cycloalkeneoxy; cycloalkyneoxy; aryloxy; alkoxycarbonyloxy, alkoxycarbonyloxyalkane; alkoxycarbonyloxyalkene; alkoxycarbonyloxyalkyne; alkoxycarbonyloxycycloalkane; alkoxycarbonyloxycycloalkene; alkoxycarbonyloxycycloalkyne; alkoxycarbonyloxyaryl; alkenoxycarbonyloxy, alkenoxycarbonyloxyalkane; alkenoxycarbonyloxyalkene; alkenoxycarbonyloxyalkyne; alkenoxycarbonyloxycycloalkane; alkenoxycarbonyloxycycloalkene; alkenoxycarbonyloxycycloalkyne; alkenoxycarbonyloxyaryl; alkynoxycarbonyloxy, alkynoxycarbonyloxyalkane; alkynoxycarbonyloxyalkene; alkynoxycarbonyloxyalkyne; alkynoxycarbonyloxycycloalkane; alkynoxycarbonyloxycycloalkene; alkynoxycarbonyloxycycloalkyne; alkynoxycarbonyloxyaryl; cycloalkoxycarbonyloxy, cycloalkoxycarbonyloxyalkane; cycloalkoxycarbonyloxyalkene; cycloalkoxycarbonyloxyalkyne; cycloalkoxycarbonyloxycycloalkane; cycloalkoxycarbonyloxycycloalkene; cycloalkoxycarbonyloxycycloalkyne; cycloalkoxycarbonyloxyaryl; cycloalkenoxycarbonyloxy, cycloalkenoxycarbonyloxyalkane; cycloalkenoxycarbonyloxyalkene; cycloalkenoxycarbonyloxyalkyne; cycloalkenoxycarbonyloxycycloalkane; cycloalkenoxycarbonyloxycycloalkene; cycloalkenoxycarbonyloxycycloalkyne; cycloalkenoxycarbonyloxyaryl; cycloalkynoxycarbonyloxy, cycloalkynoxycarbonyloxyalkane; cycloalkynoxycarbonyloxyalkene; cycloalkynoxycarbonyloxyalkyne; cycloalkynoxycarbonyloxycycloalkane; cycloalkynoxycarbonyloxycycloalkene; cycloalkynoxycarbonyloxycycloalkyne; cycloalkynoxycarbonyloxyaryl; aryloxycarbonyloxy, aryloxycarbonyloxyalkane; aryloxycarbonyloxyalkene; aryloxycarbonyloxyalkyne; aryloxycarbonyloxycycloalkane; aryloxycarbonyloxycycloalkene; aryloxycarbonyloxycycloalkyne; aryloxycarbonyloxyaryl; carbonate; carbonatealkane; carbonatealkene; carbonatealkyne; carbonatecycloalkane; carbonatecycloalkene; carbonatecycloalkyne; carbonatearyl; alkanecarbonate; alkenecarbonate; alkynecarbonate; cycloalkanecarbonate; cycloalkenecarbonate; cycloalkynecarbonate; arylcarbonate; carboxy; carboxyalkane; carboxyalkene; carboxyalkyne; carboxycycloalkane; carboxycycloalkene; carboxycycloalkyne; carboxyaryl; alkoxy/dialkoxy/trialkoxy; (alkoxy/dialkoxy/trialkoxy)alkane; (alkoxy/dialkoxy/trialkoxy)alkene; (alkoxy/dialkoxy/trialkoxy)alkyne; (alkoxy/dialkoxy/trialkoxy)cycloalkane; (alkoxy/dialkoxy/trialkoxy)cycloalkene; (alkoxy/dialkoxy/trialkoxy)cycloalkyne; (alkoxy/dialkoxy/trialkoxy)aryl; alkenoxy/dialkenoxy/trialkenoxy; (alkenoxy/dialkenoxy/trialkenoxy)alkane; (alkenoxy/dialkenoxy/trialkenoxy)alkene; (alkenoxy/dialkenoxy/trialkenoxy)alkyne; (alkenoxy/dialkenoxy/trialkenoxy)cycloalkane; (alkenoxy/dialkenoxy/trialkenoxy)cycloalkene; (alkenoxy/dialkenoxy/trialkenoxy)cycloalkyne; (alkenoxy/dialkenoxy/trialkenoxy)aryl; alkynoxy/dialkynoxy/trialkynoxy; (alkynoxy/dialkynoxy/trialkynoxy)alkane; (alkynoxy/dialkynoxy/trialkynoxy)alkene; (alkynoxy/dialkynoxy/trialkynoxy)alkyne; (alkynoxy/dialkynoxy/trialkynoxy)cycloalkane; (alkynoxy/dialkynoxy/trialkynoxy)cycloalkene; (alkynoxy/dialkynoxy/trialkynoxy)cycloalkyne; (alkynoxy/dialkynoxy/trialkynoxy)aryl; cycloalkoxy/dicycloalkoxy/tricycloalkoxy; (cycloalkoxy/dicycloalkoxy/trialkoxy)alkane; (cycloalkoxy/dicycloalkoxy/tricycloalkoxy)alkene; (cycloalkoxy/dicycloalkoxy/tricycloalkoxy)alkyne; (cycloalkoxy/dicycloalkoxy/tricycloalkoxy)cycloalkane; (cycloalkoxy/dicycloalkoxy/tricycloalkoxy)cycloalkene; (cycloalkoxy/dicycloalkoxy/tricycloalkoxy)cycloalkyne; (cycloalkoxy/dicycloalkoxy/tricycloalkoxy)aryl; cycloalkenoxy/dicycloalkenoxy/tricycloalkenoxy; (cycloalkenoxy/dicycloalkenoxy/tricycloalkenoxy)alkane; (cycloalkenoxy/dicycloalkenoxy/tricycloalkenoxy)alkene; (cycloalkenoxy/dicycloalkenoxy/tricycloalkenoxy)alkyne; (cycloalkoxy/dicycloalkenoxy/tricycloalkenoxy)cycloalkane; (cycloalkenoxy/dicycloalkenoxy/tricycloalkenoxy)cycloalkene; (cycloalkenoxy/dicycloalkenoxy/tricycloalkenoxy)cycloalkyne; (cycloalkenoxy/dicycloalkenoxy/tricycloalkenoxy)aryl; cycloalkynoxy/dicycloalkynoxy/tricycloalkynoxy; (cycloalkynoxy/dicycloalkynoxy/tricycloalkynoxy)alkane; (cycloalkynoxy/dicycloalkynoxy/tricycloalkynoxy)alkene; (cycloalkynoxy/dicycloalkynoxy/tricycloalkynoxy)alkyne; (cycloalkynoxy/dicycloalkynoxy/tricycloalkynoxy)cycloalkane; (cycloalkynoxy/dicycloalkynoxy/tricycloalkynoxy)cycloalkene; (alkynoxy/dicycloalkynoxy/tricycloalkynoxy)cycloalkyne; (cycloalkynoxy/dicycloalkynoxy/tricycloalkynoxy)aryl; aryloxy/diaryloxy/triaryloxy; (aryloxy/diaryloxy/triaryloxy)alkane; (aryloxy/diaryloxy/triaryloxy)alkene; (aryloxy/diaryloxy/triaryloxy)alkyne; (aryloxy/diaryloxy/triaryloxy)cycloalkane; (aryloxy/diaryloxy/triaryloxy)cycloalkene; (aryloxy/diaryloxy/triaryloxy)cycloalkyne; (aryloxy/diaryloxy/triaryloxy)aryl; methylenedioxy; methylenedioxyalkane; methylenedioxyalkene; methylenedioxyalkyne; methylenedioxycycloalkane; methylenedioxycycloalkene; methylenedioxycycloalkyne; methylenedioxyaryl; alkanemethylenedioxy; alkenemethylenedioxy; alkynemethylenedioxy; cycloalkanemethylenedioxy; cycloalkenemethylenedioxy; cycloalkynemethylenedioxy; arylmethylenedioxy; anhydride; anhydridealkane; anhydridealkene; anhydridealkyne; anhydridecycloalkane; anhydridecycloalkene; anhydridecycloalkyne; anhydridearyl; alkaneanhydride; alkeneanhydride; alkyneanhydride; cycloalkaneanhydride; cycloalkaneanhydride; cycloalkyneanhydride; arylanhydride; carbamoyl; carbamoylalkane; carbamoylalkene; carbamoylalkyne; carbamoylcycloalkane; carbamoylcycloalkene; carbamoylcycloalkyne; carbamoylaryl; alkanecarbamoyl; alkenecarbamoyl; alkynecarbamoyl; cycloalkanecarbamoyl; cycloalkenecarbamoyl; cycloalkynecarbamoyl; arylcarbamoyl; amide; amidealkane; amidealkene; amidealkyne; amidecycloalkane; amidecycloalkene; amidecycloalkyne; amidearyl; alkaneamide; alkeneamide; alkyneamide; cycloalkaneamide; cycloalkeneamide; cycloalkyneamide; arylamide; amino; aminoalkane; aminoalkene; aminoalkane; aminocycloalkane; aminocycloalkene; aminocycloalkyne; aminoaryl; alkaneamino; alkeneamino; alkyneamino; cycloalkaneamino; cycloalkeneamino; cycloalkyneamino; arylamino; aminocarbonyl; aminocarbonylalkane; aminocarbonylalkene; aminocarbonylalkyne; aminocarbonylcycloalkane; aminocarbonylcycloalkene; aminocarbonylcycloalkyne; aminocarbonylaryl; alkaneaminocarbonyl; alkeneaminocarbonyl; alkyneaminocarbonyl; cycloalkaneaminocarbonyl; cycloalkeneaminocarbonyl; cycloalkyneaminocarbonyl; arylaminocarbonyl; amine; aminealkane; aminealkene; aminealkyne; aminecycloalkane; aminecycloalkene; aminecycloalkyne; aminearyl; alkaneamine; alkeneamine; alkyneamine; cycloalkaneamine; cycloalkeneamine; cycloalkyneamine; ammonio; ammonioalkane; ammonioalkane; ammonioalkene; ammonioalkyne; ammoniocycloalkane; ammoniocycloalkene; ammoniocycloalkyne; ammonioaryl; alkaneammonio; alkeneammonio; alkyneammonio; cycloalkaneammonio; cycloalkeneammonio; cycloalkyneammonio; arylammonio; imino; iminoalkane; iminoalkene; iminoalkyne; iminocycloalkane; iminocycloalkene; iminocycloalkyne; iminoaryl; alkaneimino; alkeneimino; alkyneimino; cycloalkaneimino; cycloalkeneimino; cycloalkyneimino; arylimino; imido; imidoalkane; imidoalkene; imidoalkyne; imidocycloalkane; imidocycloalkene; imidocycloalkyne; imidoaryl; alkaneimido; alkeneimido; alkyneimido; cycloalkaneimido; cycloalkeneimido; cycloalkyneimido; arylimido; azido; azidoalkane; azidoalkene; azidoalkyne; azidocycloalkane; azidocycloalkene; azidocycloalkyne; azidoaryl; azo; azoalkane; azoalkene; azoalkyne; azocycloalkane; azocycloalkene; azocycloalkyne; azoaryl; alkaneazo; alkeneazo; alkyneazo; cycloalkaneazo; cycloalkeneazo; cycloalkyneazo; arylazo; cyanate; cyanatealkane; cyanatealkene; cyanatealkyne; cyanatecycloalkane; cyanatecycloalkene; cyanatecycloalkyne; cyanatearyl; isocyanate; isocyanatealkane; isocyanatealkene; isocyanatealkyne; isocyanatecycloalkane; isocyanatecycloalkene; isocyanatecycloalkyne; isocyanatearyl; nitrooxy; nitrooxyalkane; nitrooxyalkene; nitrooxyalkyne; nitrooxycycloalkane; nitrooxycycloalkene; nitrooxycycloalkyne; nitrooxycycloaryl; nitrate; nitratealkane; nitratealkene; nitratealkyne; nitratecycloalkane; nitratecycloalkene; nitratecycloalkyne; nitratearyl; cyano; cyanoalkane; cyanoalkene; cyanoalkyne; cyanocycloalkane; cyanocycloalkene; cyanocycloalkyne; cyanoaryl; isocyano; isocyanoalkane; isocyanoalkene; isocyanoalkyne; isocyanocycloalkane; isocyanocycloalkene; isocyanocycloalkyne; isocyanoaryl; nitrite; nitritealkane; nitritealkene; nitritealkyne; nitritecycloalkane; nitritecycloalkene; nitritecycloalkyne; nitritearyl; nitro; nitroalkane; nitroalkene; nitroalkyne; nitrocycloalkane; nitrocycloalkene; nitrocycloalkyne; nitroaryl; nitroso; nitrosoalkane; nitrosoalkene; nitrosoalkyne; nitrosocycloalkane; nitrosocycloalkene; nitrosocycloalkyne; nitrosoaryl; guanidino; guanidinoalkane; guanidinoalkene; guanidinoalkyne; guanidinocycloalkane; guanidinocycloalkene; guanidinocycloalkyne; guanidinoaryl; alkaneguanidino; alkeneguanidino; alkyneguanidino; cycloalkaneguanidino; cycloalkeneguanidino; cycloalkyneguanidino; arylguanidino; guanidinosulfone; guanidinosulfonealkane; guanidinosulfonealkene; guanidinosulfonealkyne; guanidinosulfonecycloalkane; guanidinosulfonecycloalkene; guanidinosulfonecycloalkyne; guanidinosulfonearyl; alkane guanidinosulfone; alkeneguanidinosulfone; alkyneguanidinosulfone; cycloalkaneguanidinosulfone; cycloalkeneguanidinosulfone; cycloalkyneguanidinosulfone; arylguanidinosulfone; oxime; oximealkane; oximealkene; oximealkyne; oximecycloalkane; oximecycloalkene; oximecycloalkyne; oximearyl; alkaneoxime; alkeneoxime; alkyneoxime; cycloalkaneoxime; cycloalkeneoxime; cycloalkyneoxime; aryloxime; pyridyl; pyridylalkane; pyridylalkene; pyridylalkyne; pyridylcycloalkane; pyridylcycloalkene; pyridylcycloalkyne; pyridylaryl; alkanepyridyl; alkenepyridyl; alkynepyridyl; cycloalkanepyridyl; cycloalkenepyridyl; cycloalkynepyridyl; arylpyridyl; carbamoyloxy; carbamoyloxyalkane; carbamoyloxyalkene; carbamoyloxyalkyne; carbamoyloxycycloalkane; carbamoyloxycycloalkene; carbamoyloxycycloalkyne; carbamoyloxyaryl; alkanecarbamoyloxy; alkenecarbamoyloxy; alkynecarbamoyloxy; cycloalkanecarbamoyloxy; cycloalkenecarbamoyloxy; cycloalkynecarbamoyloxy; arylcarbamoyloxy; aminothiocarbonyl; alkaneaminiothiocarbonyl; alkeneaminothiocarbonyl; alkyneaminothiocarbonyl; cycloalkaneaminothiocarbonyl; cycloalkeneaminothiocarbonyl; cycloalkyneaminothiocarbonyl; arylaminothiocarbonyl; aminothiocarbonylalkane; aminothiocarbonylalkene; aminothiocarbonylalkyne; aminothiocarbonylcycloalkane; aminothiocarbonylcycloalkene; aminothiocarbonylcycloalkyne; aminothiocarbonylaryl; aminocarbonylamino; aminocarbonylaminoalkane; aminocarbonylaminoalkene; aminocarbonylaminoalkyne; aminocarbonylaminocycloalkane; aminocarbonylaminocycloalkene; aminocarbonylaminocycloalkyne; aminocarbonylaminoaryl; alkaneaminocarbonylamino; alkeneaminocarbonylamino; alkyneaminocarbonylamino; cycloalkaneaminocarbonylamino; cycloalkeneaminocarbonylamino; cycloalkyneaminocarbonylamino; arylaminocarbonylamino; aminothiocarbonylamino; aminothiocarbonylaminoalkane; aminothiocarbonylaminoalkene; aminothiocarbonylaminoalkyne; aminothiocarbonylaminocycloalkane; aminothiocarbonylaminocycloalkene; aminothiocarbonylaminocycloalkyne; aminothiocarbonylaminoaryl; alkaneaminothiocarbonylamino; alkeneaminothiocarbonylamino; alkyneaminothiocarbonylamino; cycloalkaneaminothiocarbonylamino; cycloalkeneaminothiocarbonylamino; cycloalkyneaminothiocarbonylamino; arylaminothiocarbonylamino; aminocarbonyloxy; aminocarbonyloxyalkane; aminocarbonyloxyalkene; aminocarbonyloxyalkyne; aminocarbonyloxycycloalkane; aminocarbonyloxycycloalkene; aminocarbonyloxycycloalkyne; aminocarbonyloxyaryl; alkaneaminocarbonyloxy; alkeneaminocarbonyloxy; alkyneaminocarbonyloxy; cycloalkaneaminocarbonyloxy; cycloalkeneaminocarbonyloxy; cycloalkyneaminocarbonyloxy; arylaminocarbonyloxy; aminosulfonyl; aminosulfonylalkane; aminosulfonylalkene; aminosulfonylalkyne; aminosulfonylcycloalkane; aminosulfonylcycloalkene; aminosulfonylcycloalkyne; aminosulfonylaryl; alkaneaminosulfonyl; alkeneaminosulfonyl; alkyneaminosulfonyl; cycloalkaneaminosulfonyl; cycloalkeneaminosulfonyl; cycloalkyneaminosulfonyl; arylaminosulfonyl; aminosulfonyloxy; aminosulfonyloxyalkane; aminosulfonyloxyalkene; aminosulfonyloxyalkyne; aminosulfonyloxycycloalkane; aminosulfonyloxycycloalkene; aminosulfonyloxycycloalkyne; aminosulfonyloxyaryl; alkaneaminosulfonyloxy; alkeneaminosulfonyloxy; alkyneaminosulfonyloxy; cycloalkaneaminosulfonyloxy; cycloalkeneaminosulfonyloxy; cycloalkyneaminosulfonyloxy; arylaminosulfonyloxy; aminosulfonylamino; aminosulfonylaminoalkane; aminosulfonylaminoalkene; aminosulfonylaminoalkyne; aminosulfonylaminocycloalkane; aminosulfonylaminocycloalkene; aminosulfonylaminocycloalkyne; aminosulfonylaminoaryl; alkaneaminosulfonylamino; alkeneaminosulfonylamino; alkyneaminosulfonylamino; cycloalkaneaminosulfonylamino; cycloalkeneaminosulfonylamino; cycloalkyneaminosulfonylamino; arylaminosulfonylamino; amidino; amidinoalkane; amidinoalkene; amidinoalkyne; amidinocycloalkane; amidinocycloalkene; amidinocycloalkyne; amidinoaryl; alkaneamidino; alkeneamidino; alkyneamidino; cycloalkaneamidino; cycloalkeneamidino; cycloalkyneamidino; arylamidino; sulfhydryl; sulfhydrylalkane; sulfhydrylalkene; sulfhydrylalkyne; sulfhydrylcycloalkane; sulfhydrylcycloalkene; sulfhydrylcycloalkyne; sulfhydrylaryl; sulfide; sulfidealkane; sulfidealkene; sulfidealkyne; sulfidecycloalkane; sulfidecycloalkene; sulfidecycloalkyne; sulfidearyl; alkanesulfide; alkenesulfide; alkynesulfide; cycloalkanesulfide; cycloalkenesulfide; cycloalkynesulfide; arylsulfide; disulfide; disulfidealkane; disulfidealkene; disulfidealkyne; disulfidecycloalkane; disulfidecycloalkene; disulfidecycloalkyne; disulfidearyl; alkanedisulfide; alkenedisulfide; alkynedisulfide; cycloalkanedisulfide; cycloalkenedisulfide; cycloalkynedisulfide; aryldisulfide; sulfinyl; sulfinylalkane; sulfinylalkene; sulfinylalkyne; sulfinylcycloalkane; sulfinylcycloalkene; sulfinylcycloalkyne; sulfinylaryl; alkanesulfinyl; alkenesulfinyl; alkynesulfinyl; cycloalkanesulfinyl; cycloalkenesulfinyl; cycloalkynesulfinyl; arylsulfinyl; sulfonyl; sulfonylalkane; sulfonylalkene; sulfonylalkyne; sulfonylcycloalkane; sulfonylcycloalkene; sulfonylcycloalkyne; sulfonylaryl; alkanesulfonyl; alkenesulfonyl; alkynesulfonyl; cycloalkanesulfonyl; cycloalkenesulfonyl; cycloalkynesulfonyl; arylsulfonyl; sulfino; sulfinoalkane; sulfinoalkene; sulfinoalkyne; sulfinocycloalkane; sulfinocycloalkene; sulfinocycloalkyne; sulfinoaryl; sulfo; sulfoalkane; sulfoalkene; sulfoalkyne; sulfocycloalkane; sulfocycloalkene; sulfocycloalkyne; sulfoaryl; alkanesulfo; alkenesulfo; alkynesulfo; cycloalkanesulfo; cycloalkenesulfo; cycloalkynesulfo; arylsulfo; thiocyanate; thiocyanatealkane; thiocyanatealkene; thiocyanatealkyne; thiocyanatecycloalkane; thiocyanatecycloalkene; thiocyanatecycloalkyne; thiocyanatearyl; isothiocyanate; isothiocyanatealkane; isothiocyanatealkene; isothiocyanatealkyne; isothiocyanatecycloalkane; isothiocyanatecycloalkene; isothiocyanatecycloalkyne; isothiocyantearyl; carbonothioyl; carbonothioylalkane; carbonothioylalkene; carbonothioylalkyne; carbonothioylcycloalkane; carbonothioylcycloalkene; carbonothioylcycloalkyne; carbonothioylaryl; alkanecarbonothioyl; alkenecarbonothioyl; alkynecarbonothioyl; cycloalkanecarbonothioyl; cycloalkenecarbonothioyl; cycloalkynecarbonothioyl; arylcarbonothioyl; mercaptocarbonyl; mercaptocarbonylalkane; mercaptocarbonylalkene; mercaptocarbonylalkyne; mercaptocarbonylcycloalkane; mercaptocarbonylcycloalkene; mercaptocarbonylcycloalkyne; mercaptocarbonylaryl; hydroxythiocarbonyl; hydroxythiocarbonylalkane; hydroxythiocarbonylalkene; hydroxythiocarbonylalkyne; hydroxythiocarbonylcycloalkane; hydroxythiocarbonylcycloalkene; hydroxythiocarbonylcycloalkyne; hydroxythiocarbonylaryl; thiolester, thiolesteralkane; thiolesteralkene; thiolesteralkyne; thiolestercycloalkane; thiolestercycloalkene; thiolestercycloalkyne; thiolesteraryl; alkanethiolester; alkenethiolester; alkynethiolester; cycloalkanethiolester; cycloalkenethiolester; cycloalkynethiolester; arylthiolester; thionoester; thionoesteralkane; thionoesteralkene; thionoesteralkyne; thionoestercycloalkane; thionoestercycloalkene; thionoestercycloalkyne; thionoesteraryl; alkanethionoester; alkenethionoester; alkynethionoester; cycloalkanethionoester; cycloalkenethionoester; cycloalkynethionoester; arylthionoester; dithiocarboxy; dithiocarboxyalkane; dithiocarboxyalkene; dithiocarboxyalkyne; dithiocarboxycycloalkane; dithiocarboxycycloalkene; dithiocarboxycycloalkyne; dithiocarboxyaryl; dithioate; dithioatealkane; dithioatealkene; dithioatealkyne; dithioatecycloalkane; dithioatecycloalkene; dithioatecycloalkyne; dithioatearyl; alkanedithioate; alkenedithioate; alkynedithioate; cycloalkanedithioate; cycloalkenedithioate; cycloalkynedithioate; aryldithioate; phosphino; phosphinoalkane; phosphinoalkene; phosphinoalkyne; phosphinocycloalkane; phophinocycloalkene; phosphinocycloalkyne; phosphinoaryl; alkanephosphino; alkenephosphino; alkynephosphino; cycloalkanephosphino; cycloalkenephosphino; cycloalkynephosphino; arylphosphino; phosphono; phosphonoalkane; phosphonoalkene; phosphonoalkyne; phosphonocycloalkane; phosphonocycloalkene; phosphonocycloalkyne; phosphonoaryl; alkanephosphono; alkenephosphono; alkynephosphono; cycloalkanephosphono; cycloalkenephosphono; cycloalkynephosphono; arylphosphono; phosphate; phosphatealkane; phosphatealkene; phosphatealkyne; phosphatecycloalkane; phosphatecycloalkene; phosphatecycloalkyne; phosphatearyl; alkanephosphate; alkenephosphate; alkynephosphate; cycloalkanephosphate; cycloalkenephosphate; cycloalkynephosphate; arylphosphate. Any suitable valent form of a carboxyl, carboxylester, carboxylesteramino, carboxylesteroxy, haloalkoxy, halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, cycloalkoxy, orthocarbonate ester, organic acid anhydride, silyl, alkylthio, sulfonamide, trifluoromethyl, thiocyano, mercaptocarbonyl, hydroxythiocarbonyl, thiolester, thionoester, carbodithioic acid, carbodithiol, carboyl, formyl, haloformyl, (alkoxycarbonyl)oxy, carboxy, methoxy, alkoxy, alkoxyol dialkoxy, trialkoxy methylenedioxy; tetralkoxy; carboxylic anhydride; carbamoyl; imino; cyanate; isocyanato; nitroxy; nitrosoxy; nitro; nitroso; oxime; pyridyl; carbamate; phosphanyl; phosphono; phosphonoxy; [(alkoxy)hydroxyphosphoryl]oxy, or acyl, etc.

In certain implementations, the backbone of the MDE may be substituted with one or more ring structure(s). In formulas with more than one ring structure, ring structure radicals may be fused to one another or bonded to one another by way of a C—C bond or by other bridging groups. The bridging groups may be other ring structures. Ring structures may include any aromatic ring, aliphatic ring, heterocyclic, etc. and is not limited by the type of element used in the backbone of the ring. The single ring structures may include, but is not limited to, between 5 and 7 members. Other non-limiting bridging group examples may include, but are not limited to, —(CH₂)ₓ—, —C(═O)—, S(═O)₂—, —C(CF₃)₂—, —Si(CH₃)₂—, —C(CH₃)₂—, —(CH₂O)ₓ—, etc. where X is an integer between 1 and 20,000.

In some implementations, the backbone of the MDE may be configured with pendant group(s). More than one pendant group may be selected and is subject to valence tolerance. The group(s) may also be substituted or unsubstituted and may include, but are not limited to, any suitable valent form of hydrogen; halogen, alkane; alkene; alkyne; cycloalkane; cycloalkene; cycloalkyne; aryl; hydroxyl; hydroxylalkane; hydroxylalkene; hydroxylalkyne; hydroxylcycloalkane; hydroxylcycloalkene; hydroxylcycloalkyne; hydroxylaryl; carbonyl; carbonylalkane; carbonylalkene; carbonylalkyne; carbonylcycloalkane; carbonylcycloalkene; carbonylcycloalkyne; carbonylaryl; alkanecarbonyl; alkenecarbonyl; alkynecarbonyl; cycloalkanecarbonyl; cycloalkenecarbonyl; cycloalkynecarbonyl; arylcarbonyl; carbonyloxy; carbonyloxyalkane; carbonyloxyalkene; carbonyloxyalkyne; carbonyloxycycloalkane; carbonyloxycycloalkene; carbonyloxycycloalkyne; carbonyloxyaryl; alkanecarbonyloxy; alkenecarbonyloxy; alkynecarbonyloxy; cycloalkanecarbonyloxy; cycloalkenecarbonyloxy; cycloalkynecarbonyloxy; arylcarbonyloxy; alkoxycarbonyl; alkoxycarbonylalkane; alkoxycarbonylalkene; alkoxycarbonylalkyne; alkoxycarbonylcycloalkane; alkoxycarbonylcycloalkene; alkoxycarbonylcycloalkyne; alkoxycarbonylaryl; alkenoxycarbonyl; alkenoxycarbonylalkane; alkenoxycarbonylalkene; alkenoxycarbonylalkyne; alkenoxycarbonylcycloalkane; alkenoxycarbonylcycloalkene; alkenoxycarbonylcycloalkyne; alkenoxycarbonylaryl; alkynoxycarbonylalkane; alkynoxycarbonylalkene; alkynoxycarbonylalkyne; alkynoxycarbonylcycloalkane; alkynoxycarbonylcycloalkene; alkynoxycarbonylcycloalkyne; alkynoxycarbonylaryl; cycloalkoxycarbonylalkane; cycloalkoxycarbonylalkene; cycloalkoxycarbonylalkyne; cycloalkoxycarbonylcycloalkane; cycloalkoxycarbonylcycloalkene; cycloalkoxycarbonylcycloalkyne; cycloalkoxycarbonylaryl; cycloalkenoxycarbonylalkane; cycloalkenoxycarbonylalkene; cycloalkenoxycarbonylalkyne; cycloalkenoxycarbonylcycloalkane; cycloalkenoxycarbonylcycloalkene; cycloalkenoxycarbonylcycloalkyne; cycloalkenoxycarbonylaryl; cycloalkynoxycarbonylalkane; cycloalkynoxycarbonylalkene; cycloalkynoxycarbonylalkyne; cycloalkynoxycarbonylcycloalkane; cycloalkynoxycarbonylcycloalkene; cycloalkynoxycarbonylcycloalkyne; cycloalkynoxycarbonylaryl; aryloxycarbonylalkane; aryloxycarbonylalkene; aryloxycarbonylalkyne; aryloxycarbonylcycloalkane; aryloxycarbonylcycloalkene; aryloxycarbonylcycloalkyne; aryloxycarbonylaryl; carbonylamino; carbonylaminoalkane; carbonylaminoalkene; carbonylaminoalkyne; carbonylaminocycloalkane; carbonylaminocycloalkene; carbonylaminocycloalkyne; carbonylaminoaryl; alkanecarbonylamino; alkenecarbonylamino; alkynecarbonylamino; cycloalkanecarbonylamino; cycloalkenecarbonylamino; cycloalkynecarbonylamino; arylcarbonylamino; alkoxycarbonylamino; alkoxycarbonylaminoalkane; alkoxycarbonylaminoalkene; alkoxycarbonylaminoalkyne; alkoxycarbonylaminocycloalkane; alkoxycarbonylaminocycloalkene; alkoxycarbonylaminocycloalkyne; alkoxycarbonylaminoaryl; alkenoxycarbonylamino; alkenoxycarbonylaminoalkane; alkenoxycarbonylaminoalkene; alkenoxycarbonylaminoalkyne; alkenoxycarbonylaminocycloalkane; alkenoxycarbonylaminocycloalkene; alkenoxycarbonylaminocycloalkyne; alkenoxycarbonylaminoaryl; alkynoxycarbonylamino; alkynoxycarbonylaminoalkane; alkynoxycarbonylaminoalkene; alkynoxycarbonylaminoalkyne; alkynoxycarbonylaminocycloalkane; alkynoxycarbonylaminocycloalkene; alkynoxycarbonylaminocycloalkyne; alkynoxycarbonylaminoaryl; cycloalkoxycarbonylamino; cycloalkoxycarbonylaminoalkane; cycloalkoxycarbonylaminoalkene; cycloalkoxycarbonylaminoalkyne; cycloalkoxycarbonylaminocycloalkane; cycloalkoxycarbonylaminocycloalkene; cycloalkoxycarbonylaminocycloalkyne; cycloalkoxycarbonylaminoaryl; cycloalkenoxycarbonylamino; cycloalkenoxycarbonylaminoalkane; cycloalkenoxycarbonylaminoalkene; cycloalkenoxycarbonylaminoalkyne; cycloalkenoxycarbonylaminocycloalkane; cycloalkenoxycarbonylaminocycloalkene; cycloalkenoxycarbonylaminocycloalkyne; cycloalkenoxycarbonylaminoaryl; cycloalkynoxycarbonylamino; cycloalkynoxycarbonylaminoalkane; cycloalkynoxycarbonylaminoalkene; cycloalkynoxycarbonylaminoalkyne; cycloalkynoxycarbonylaminocycloalkane; cycloalkynoxycarbonylaminocycloalkene; cycloalkynoxycarbonylaminocycloalkyne; cycloalkynoxycarbonylaminoaryl; aryloxycarbonylamino; aryloxycarbonylaminoalkane; aryloxycarbonylaminoalkene; aryloxycarbonylaminoalkyne; aryloxycarbonylaminocycloalkane; aryloxycarbonylaminocycloalkene; aryloxycarbonylaminocycloalkyne; aryloxycarbonylaminoaryl; formyl; formylalkane; formylalkene; formylalkyne; formylcycloalkane; formylcycloalkene; formylcycloalkyne; formylaryl; oxy; oxyalkane; oxyalkene; oxyalkyne; oxycycloalkane; oxycycloalkene; oxycycloalkyne; oxyaryl; alkaneoxy; alkeneoxy; alkyneoxy; cycloalkaneoxy; cycloalkeneoxy; cycloalkyneoxy; aryloxy; alkoxycarbonyloxy, alkoxycarbonyloxyalkane; alkoxycarbonyloxyalkene; alkoxycarbonyloxyalkyne; alkoxycarbonyloxycycloalkane; alkoxycarbonyloxycycloalkene; alkoxycarbonyloxycycloalkyne; alkoxycarbonyloxyaryl; alkenoxycarbonyloxy, alkenoxycarbonyloxyalkane; alkenoxycarbonyloxyalkene; alkenoxycarbonyloxyalkyne; alkenoxycarbonyloxycycloalkane; alkenoxycarbonyloxycycloalkene; alkenoxycarbonyloxycycloalkyne; alkenoxycarbonyloxyaryl; alkynoxycarbonyloxy, alkynoxycarbonyloxyalkane; alkynoxycarbonyloxyalkene; alkynoxycarbonyloxyalkyne; alkynoxycarbonyloxycycloalkane; alkynoxycarbonyloxycycloalkene; alkynoxycarbonyloxycycloalkyne; alkynoxycarbonyloxyaryl; cycloalkoxycarbonyloxy, cycloalkoxycarbonyloxyalkane; cycloalkoxycarbonyloxyalkene; cycloalkoxycarbonyloxyalkyne; cycloalkoxycarbonyloxycycloalkane; cycloalkoxycarbonyloxycycloalkene; cycloalkoxycarbonyloxycycloalkyne; cycloalkoxycarbonyloxyaryl; cycloalkenoxycarbonyloxy, cycloalkenoxycarbonyloxyalkane; cycloalkenoxycarbonyloxyalkene; cycloalkenoxycarbonyloxyalkyne; cycloalkenoxycarbonyloxycycloalkane; cycloalkenoxycarbonyloxycycloalkene; cycloalkenoxycarbonyloxycycloalkyne; cycloalkenoxycarbonyloxyaryl; cycloalkynoxycarbonyloxy, cycloalkynoxycarbonyloxyalkane; cycloalkynoxycarbonyloxyalkene; cycloalkynoxycarbonyloxyalkyne; cycloalkynoxycarbonyloxycycloalkane; cycloalkynoxycarbonyloxycycloalkene; cycloalkynoxycarbonyloxycycloalkyne; cycloalkynoxycarbonyloxyaryl; aryloxycarbonyloxy, aryloxycarbonyloxyalkane; aryloxycarbonyloxyalkene; aryloxycarbonyloxyalkyne; aryloxycarbonyloxycycloalkane; aryloxycarbonyloxycycloalkene; aryloxycarbonyloxycycloalkyne; aryloxycarbonyloxyaryl; carbonate; carbonatealkane; carbonatealkene; carbonatealkyne; carbonatecycloalkane; carbonatecycloalkene; carbonatecycloalkyne; carbonatearyl; alkanecarbonate; alkenecarbonate; alkynecarbonate; cycloalkanecarbonate; cycloalkenecarbonate; cycloalkynecarbonate; arylcarbonate; carboxy; carboxyalkane; carboxyalkene; carboxyalkyne; carboxycycloalkane; carboxycycloalkene; carboxycycloalkyne; carboxyaryl; alkoxy/dialkoxy/trialkoxy; (alkoxy/dialkoxy/trialkoxy)alkane; (alkoxy/dialkoxy/trialkoxy)alkene; (alkoxy/dialkoxy/trialkoxy)alkyne; (alkoxy/dialkoxy/trialkoxy)cycloalkane; (alkoxy/dialkoxy/trialkoxy)cycloalkene; (alkoxy/dialkoxy/trialkoxy)cycloalkyne; (alkoxy/dialkoxy/trialkoxy)aryl; alkenoxy/dialkenoxy/trialkenoxy; (alkenoxy/dialkenoxy/trialkenoxy)alkane; (alkenoxy/dialkenoxy/trialkenoxy)alkene; (alkenoxy/dialkenoxy/trialkenoxy)alkyne; (alkenoxy/dialkenoxy/trialkenoxy)cycloalkane; (alkenoxy/dialkenoxy/trialkenoxy)cycloalkene; (alkenoxy/dialkenoxy/trialkenoxy)cycloalkyne; (alkenoxy/dialkenoxy/trialkenoxy)aryl; alkynoxy/dialkynoxy/trialkynoxy; (alkynoxy/dialkynoxy/trialkynoxy)alkane; (alkynoxy/dialkynoxy/trialkynoxy)alkene; (alkynoxy/dialkynoxy/trialkynoxy)alkyne; (alkynoxy/dialkynoxy/trialkynoxy)cycloalkane; (alkynoxy/dialkynoxy/trialkynoxy)cycloalkene; (alkynoxy/dialkynoxy/trialkynoxy)cycloalkyne; (alkynoxy/dialkynoxy/trialkynoxy)aryl; cycloalkoxy/dicycloalkoxy/tricycloalkoxy; (cycloalkoxy/dicycloalkoxy/trialkoxy)alkane; (cycloalkoxy/dicycloalkoxy/tricycloalkoxy)alkene; (cycloalkoxy/dicycloalkoxy/tricycloalkoxy)alkyne; (cycloalkoxy/dicycloalkoxy/tricycloalkoxy)cycloalkane; (cycloalkoxy/dicycloalkoxy/tricycloalkoxy)cycloalkene; (cycloalkoxy/dicycloalkoxy/tricycloalkoxy)cycloalkyne; (cycloalkoxy/dicycloalkoxy/tricycloalkoxy)aryl; cycloalkenoxy/dicycloalkenoxy/tricycloalkenoxy; (cycloalkenoxy/dicycloalkenoxy/tricycloalkenoxy)alkane; (cycloalkenoxy/dicycloalkenoxy/tricycloalkenoxy)alkene; (cycloalkenoxy/dicycloalkenoxy/tricycloalkenoxy)alkyne; (cycloalkoxy/dicycloalkenoxy/tricycloalkenoxy)cycloalkane; (cycloalkenoxy/dicycloalkenoxy/tricycloalkenoxy)cycloalkene; (cycloalkenoxy/dicycloalkenoxy/tricycloalkenoxy)cycloalkyne; (cycloalkenoxy/dicycloalkenoxy/tricycloalkenoxy)aryl; cycloalkynoxy/dicycloalkynoxy/tricycloalkynoxy; (cycloalkynoxy/dicycloalkynoxy/tricycloalkynoxy)alkane; (cycloalkynoxy/dicycloalkynoxy/tricycloalkynoxy)alkene; (cycloalkynoxy/dicycloalkynoxy/tricycloalkynoxy)alkyne; (cycloalkynoxy/dicycloalkynoxy/tricycloalkynoxy)cycloalkane; (cycloalkynoxy/dicycloalkynoxy/tricycloalkynoxy)cycloalkene; (alkynoxy/dicycloalkynoxy/tricycloalkynoxy)cycloalkyne; (cycloalkynoxy/dicycloalkynoxy/tricycloalkynoxy)aryl; aryloxy/diaryloxy/triaryloxy; (aryloxy/diaryloxy/triaryloxy)alkane; (aryloxy/diaryloxy/triaryloxy)alkene; (aryloxy/diaryloxy/triaryloxy)alkyne; (aryloxy/diaryloxy/triaryloxy)cycloalkane; (aryloxy/diaryloxy/triaryloxy)cycloalkene; (aryloxy/diaryloxy/triaryloxy)cycloalkyne; (aryloxy/diaryloxy/triaryloxy)aryl; methylenedioxy; methylenedioxyalkane; methylenedioxyalkene; methylenedioxyalkyne; methylenedioxycycloalkane; methylenedioxycycloalkene; methylenedioxycycloalkyne; methylenedioxyaryl; alkanemethylenedioxy; alkenemethylenedioxy; alkynemethylenedioxy; cycloalkanemethylenedioxy; cycloalkenemethylenedioxy; cycloalkynemethylenedioxy; arylmethylenedioxy; anhydride; anhydridealkane; anhydridealkene; anhydridealkyne; anhydridecycloalkane; anhydridecycloalkene; anhydridecycloalkyne; anhydridearyl; alkaneanhydride; alkeneanhydride; alkyneanhydride; cycloalkaneanhydride; cycloalkaneanhydride; cycloalkyneanhydride; arylanhydride; carbamoyl; carbamoylalkane; carbamoylalkene; carbamoylalkyne; carbamoylcycloalkane; carbamoylcycloalkene; carbamoylcycloalkyne; carbamoylaryl; alkanecarbamoyl; alkenecarbamoyl; alkynecarbamoyl; cycloalkanecarbamoyl; cycloalkenecarbamoyl; cycloalkynecarbamoyl; arylcarbamoyl; amide; amidealkane; amidealkene; amidealkyne; amidecycloalkane; amidecycloalkene; amidecycloalkyne; amidearyl; alkaneamide; alkeneamide; alkyneamide; cycloalkaneamide; cycloalkeneamide; cycloalkyneamide; arylamide; amino; aminoalkane; aminoalkene; aminoalkane; aminocycloalkane; aminocycloalkene; aminocycloalkyne; aminoaryl; alkaneamino; alkeneamino; alkyneamino; cycloalkaneamino; cycloalkeneamino; cycloalkyneamino; arylamino; aminocarbonyl; aminocarbonylalkane; aminocarbonylalkene; aminocarbonylalkyne; aminocarbonylcycloalkane; aminocarbonylcycloalkene; aminocarbonylcycloalkyne; aminocarbonylaryl; alkaneaminocarbonyl; alkeneaminocarbonyl; alkyneaminocarbonyl; cycloalkaneaminocarbonyl; cycloalkeneaminocarbonyl; cycloalkyneaminocarbonyl; arylaminocarbonyl; amine; aminealkane; aminealkene; aminealkyne; aminecycloalkane; aminecycloalkene; aminecycloalkyne; aminearyl; alkaneamine; alkeneamine; alkyneamine; cycloalkaneamine; cycloalkeneamine; cycloalkyneamine; ammonio; ammonioalkane; ammonioalkane; ammonioalkene; ammonioalkyne; ammoniocycloalkane; ammoniocycloalkene; ammoniocycloalkyne; ammonioaryl; alkaneammonio; alkeneammonio; alkyneammonio; cycloalkaneammonio; cycloalkeneammonio; cycloalkyneammonio; arylammonio; imino; iminoalkane; iminoalkene; iminoalkyne; iminocycloalkane; iminocycloalkene; iminocycloalkyne; iminoaryl; alkaneimino; alkeneimino; alkyneimino; cycloalkaneimino; cycloalkeneimino; cycloalkyneimino; arylimino; imido; imidoalkane; imidoalkene; imidoalkyne; imidocycloalkane; imidocycloalkene; imidocycloalkyne; imidoaryl; alkaneimido; alkeneimido; alkyneimido; cycloalkaneimido; cycloalkeneimido; cycloalkyneimido; arylimido; azido; azidoalkane; azidoalkene; azidoalkyne; azidocycloalkane; azidocycloalkene; azidocycloalkyne; azidoaryl; azo; azoalkane; azoalkene; azoalkyne; azocycloalkane; azocycloalkene; azocycloalkyne; azoaryl; alkaneazo; alkeneazo; alkyneazo; cycloalkaneazo; cycloalkeneazo; cycloalkyneazo; arylazo; cyanate; cyanatealkane; cyanatealkene; cyanatealkyne; cyanatecycloalkane; cyanatecycloalkene; cyanatecycloalkyne; cyanatearyl; isocyanate; isocyanatealkane; isocyanatealkene; isocyanatealkyne; isocyanatecycloalkane; isocyanatecycloalkene; isocyanatecycloalkyne; isocyanatearyl; nitrooxy; nitrooxyalkane; nitrooxyalkene; nitrooxyalkyne; nitrooxycycloalkane; nitrooxycycloalkene; nitrooxycycloalkyne; nitrooxycycloaryl; nitrate; nitratealkane; nitratealkene; nitratealkyne; nitratecycloalkane; nitratecycloalkene; nitratecycloalkyne; nitratearyl; cyano; cyanoalkane; cyanoalkene; cyanoalkyne; cyanocycloalkane; cyanocycloalkene; cyanocycloalkyne; cyanoaryl; isocyano; isocyanoalkane; isocyanoalkene; isocyanoalkyne; isocyanocycloalkane; isocyanocycloalkene; isocyanocycloalkyne; isocyanoaryl; nitrite; nitritealkane; nitritealkene; nitritealkyne; nitritecycloalkane; nitritecycloalkene; nitritecycloalkyne; nitritearyl; nitro; nitroalkane; nitroalkene; nitroalkyne; nitrocycloalkane; nitrocycloalkene; nitrocycloalkyne; nitroaryl; nitroso; nitrosoalkane; nitrosoalkene; nitrosoalkyne; nitrosocycloalkane; nitrosocycloalkene; nitrosocycloalkyne; nitrosoaryl; guanidino; guanidinoalkane; guanidinoalkene; guanidinoalkyne; guanidinocycloalkane; guanidinocycloalkene; guanidinocycloalkyne; guanidinoaryl; alkaneguanidino; alkeneguanidino; alkyneguanidino; cycloalkaneguanidino; cycloalkeneguanidino; cycloalkyneguanidino; arylguanidino; guanidinosulfone; guanidinosulfonealkane; guanidinosulfonealkene; guanidinosulfonealkyne; guanidinosulfonecycloalkane; guanidinosulfonecycloalkene; guanidinosulfonecycloalkyne; guanidinosulfonearyl; alkane guanidinosulfone; alkeneguanidinosulfone; alkyneguanidinosulfone; cycloalkaneguanidinosulfone; cycloalkeneguanidinosulfone; cycloalkyneguanidinosulfone; arylguanidinosulfone; oxime; oximealkane; oximealkene; oximealkyne; oximecycloalkane; oximecycloalkene; oximecycloalkyne; oximearyl; alkaneoxime; alkeneoxime; alkyneoxime; cycloalkaneoxime; cycloalkeneoxime; cycloalkyneoxime; aryloxime; pyridyl; pyridylalkane; pyridylalkene; pyridylalkyne; pyridylcycloalkane; pyridylcycloalkene; pyridylcycloalkyne; pyridylaryl; alkanepyridyl; alkenepyridyl; alkynepyridyl; cycloalkanepyridyl; cycloalkenepyridyl; cycloalkynepyridyl; arylpyridyl; carbamoyloxy; carbamoyloxyalkane; carbamoyloxyalkene; carbamoyloxyalkyne; carbamoyloxycycloalkane; carbamoyloxycycloalkene; carbamoyloxycycloalkyne; carbamoyloxyaryl; alkanecarbamoyloxy; alkenecarbamoyloxy; alkynecarbamoyloxy; cycloalkanecarbamoyloxy; cycloalkenecarbamoyloxy; cycloalkynecarbamoyloxy; arylcarbamoyloxy; aminothiocarbonyl; alkaneaminiothiocarbonyl; alkeneaminothiocarbonyl; alkyneaminothiocarbonyl; cycloalkaneaminothiocarbonyl; cycloalkeneaminothiocarbonyl; cycloalkyneaminothiocarbonyl; arylaminothiocarbonyl; aminothiocarbonylalkane; aminothiocarbonylalkene; aminothiocarbonylalkyne; aminothiocarbonylcycloalkane; aminothiocarbonylcycloalkene; aminothiocarbonylcycloalkyne; aminothiocarbonylaryl; aminocarbonylamino; aminocarbonylaminoalkane; aminocarbonylaminoalkene; aminocarbonylaminoalkyne; aminocarbonylaminocycloalkane; aminocarbonylaminocycloalkene; aminocarbonylaminocycloalkyne; aminocarbonylaminoaryl; alkaneaminocarbonylamino; alkeneaminocarbonylamino; alkyneaminocarbonylamino; cycloalkaneaminocarbonylamino; cycloalkeneaminocarbonylamino; cycloalkyneaminocarbonylamino; arylaminocarbonylamino; aminothiocarbonylamino; aminothiocarbonylaminoalkane; aminothiocarbonylaminoalkene; aminothiocarbonylaminoalkyne; aminothiocarbonylaminocycloalkane; aminothiocarbonylaminocycloalkene; aminothiocarbonylaminocycloalkyne; aminothiocarbonylaminoaryl; alkaneaminothiocarbonylamino; alkeneaminothiocarbonylamino; alkyneaminothiocarbonylamino; cycloalkaneaminothiocarbonylamino; cycloalkeneaminothiocarbonylamino; cycloalkyneaminothiocarbonylamino; arylaminothiocarbonylamino; aminocarbonyloxy; aminocarbonyloxyalkane; aminocarbonyloxyalkene; aminocarbonyloxyalkyne; aminocarbonyloxycycloalkane; aminocarbonyloxycycloalkene; aminocarbonyloxycycloalkyne; aminocarbonyloxyaryl; alkaneaminocarbonyloxy; alkeneaminocarbonyloxy; alkyneaminocarbonyloxy; cycloalkaneaminocarbonyloxy; cycloalkeneaminocarbonyloxy; cycloalkyneaminocarbonyloxy; arylaminocarbonyloxy; aminosulfonyl; aminosulfonylalkane; aminosulfonylalkene; aminosulfonylalkyne; aminosulfonylcycloalkane; aminosulfonylcycloalkene; aminosulfonylcycloalkyne; aminosulfonylaryl; alkaneaminosulfonyl; alkeneaminosulfonyl; alkyneaminosulfonyl; cycloalkaneaminosulfonyl; cycloalkeneaminosulfonyl; cycloalkyneaminosulfonyl; arylaminosulfonyl; aminosulfonyloxy; aminosulfonyloxyalkane; aminosulfonyloxyalkene; aminosulfonyloxyalkyne; aminosulfonyloxycycloalkane; aminosulfonyloxycycloalkene; aminosulfonyloxycycloalkyne; aminosulfonyloxyaryl; alkaneaminosulfonyloxy; alkeneaminosulfonyloxy; alkyneaminosulfonyloxy; cycloalkaneaminosulfonyloxy; cycloalkeneaminosulfonyloxy; cycloalkyneaminosulfonyloxy; arylaminosulfonyloxy; aminosulfonylamino; aminosulfonylaminoalkane; aminosulfonylaminoalkene; aminosulfonylaminoalkyne; aminosulfonylaminocycloalkane; aminosulfonylaminocycloalkene; aminosulfonylaminocycloalkyne; aminosulfonylaminoaryl; alkaneaminosulfonylamino; alkeneaminosulfonylamino; alkyneaminosulfonylamino; cycloalkaneaminosulfonylamino; cycloalkeneaminosulfonylamino; cycloalkyneaminosulfonylamino; arylaminosulfonylamino; amidino; amidinoalkane; amidinoalkene; amidinoalkyne; amidinocycloalkane; amidinocycloalkene; amidinocycloalkyne; amidinoaryl; alkaneamidino; alkeneamidino; alkyneamidino; cycloalkaneamidino; cycloalkeneamidino; cycloalkyneamidino; arylamidino; sulfhydryl; sulfhydrylalkane; sulfhydrylalkene; sulfhydrylalkyne; sulfhydrylcycloalkane; sulfhydrylcycloalkene; sulfhydrylcycloalkyne; sulfhydrylaryl; sulfide; sulfidealkane; sulfidealkene; sulfidealkyne; sulfidecycloalkane; sulfidecycloalkene; sulfidecycloalkyne; sulfidearyl; alkanesulfide; alkenesulfide; alkynesulfide; cycloalkanesulfide; cycloalkenesulfide; cycloalkynesulfide; arylsulfide; disulfide; disulfidealkane; disulfidealkene; disulfidealkyne; disulfidecycloalkane; disulfidecycloalkene; disulfidecycloalkyne; disulfidearyl; alkanedisulfide; alkenedisulfide; alkynedisulfide; cycloalkanedisulfide; cycloalkenedisulfide; cycloalkynedisulfide; aryldisulfide; sulfinyl; sulfinylalkane; sulfinylalkene; sulfinylalkyne; sulfinylcycloalkane; sulfinylcycloalkene; sulfinylcycloalkyne; sulfinylaryl; alkanesulfinyl; alkenesulfinyl; alkynesulfinyl; cycloalkanesulfinyl; cycloalkenesulfinyl; cycloalkynesulfinyl; arylsulfinyl; sulfonyl; sulfonylalkane; sulfonylalkene; sulfonylalkyne; sulfonylcycloalkane; sulfonylcycloalkene; sulfonylcycloalkyne; sulfonylaryl; alkanesulfonyl; alkenesulfonyl; alkynesulfonyl; cycloalkanesulfonyl; cycloalkenesulfonyl; cycloalkynesulfonyl; arylsulfonyl; sulfino; sulfinoalkane; sulfinoalkene; sulfinoalkyne; sulfinocycloalkane; sulfinocycloalkene; sulfinocycloalkyne; sulfinoaryl; sulfo; sulfoalkane; sulfoalkene; sulfoalkyne; sulfocycloalkane; sulfocycloalkene; sulfocycloalkyne; sulfoaryl; alkanesulfo; alkenesulfo; alkynesulfo; cycloalkanesulfo; cycloalkenesulfo; cycloalkynesulfo; arylsulfo; thiocyanate; thiocyanatealkane; thiocyanatealkene; thiocyanatealkyne; thiocyanatecycloalkane; thiocyanatecycloalkene; thiocyanatecycloalkyne; thiocyanatearyl; isothiocyanate; isothiocyanatealkane; isothiocyanatealkene; isothiocyanatealkyne; isothiocyanatecycloalkane; isothiocyanatecycloalkene; isothiocyanatecycloalkyne; isothiocyantearyl; carbonothioyl; carbonothioylalkane; carbonothioylalkene; carbonothioylalkyne; carbonothioylcycloalkane; carbonothioylcycloalkene; carbonothioylcycloalkyne; carbonothioylaryl; alkanecarbonothioyl; alkenecarbonothioyl; alkynecarbonothioyl; cycloalkanecarbonothioyl; cycloalkenecarbonothioyl; cycloalkynecarbonothioyl; arylcarbonothioyl; mercaptocarbonyl; mercaptocarbonylalkane; mercaptocarbonylalkene; mercaptocarbonylalkyne; mercaptocarbonylcycloalkane; mercaptocarbonylcycloalkene; mercaptocarbonylcycloalkyne; mercaptocarbonylaryl; hydroxythiocarbonyl; hydroxythiocarbonylalkane; hydroxythiocarbonylalkene; hydroxythiocarbonylalkyne; hydroxythiocarbonylcycloalkane; hydroxythiocarbonylcycloalkene; hydroxythiocarbonylcycloalkyne; hydroxythiocarbonylaryl; thiolester, thiolesteralkane; thiolesteralkene; thiolesteralkyne; thiolestercycloalkane; thiolestercycloalkene; thiolestercycloalkyne; thiolesteraryl; alkanethiolester; alkenethiolester; alkynethiolester; cycloalkanethiolester; cycloalkenethiolester; cycloalkynethiolester; arylthiolester; thionoester; thionoesteralkane; thionoesteralkene; thionoesteralkyne; thionoestercycloalkane; thionoestercycloalkene; thionoestercycloalkyne; thionoesteraryl; alkanethionoester; alkenethionoester; alkynethionoester; cycloalkanethionoester; cycloalkenethionoester; cycloalkynethionoester; arylthionoester; dithiocarboxy; dithiocarboxyalkane; dithiocarboxyalkene; dithiocarboxyalkyne; dithiocarboxycycloalkane; dithiocarboxycycloalkene; dithiocarboxycycloalkyne; dithiocarboxyaryl; dithioate; dithioatealkane; dithioatealkene; dithioatealkyne; dithioatecycloalkane; dithioatecycloalkene; dithioatecycloalkyne; dithioatearyl; alkanedithioate; alkenedithioate; alkynedithioate; cycloalkanedithioate; cycloalkenedithioate; cycloalkynedithioate; aryldithioate; phosphino; phosphinoalkane; phosphinoalkene; phosphinoalkyne; phosphinocycloalkane; phophinocycloalkene; phosphinocycloalkyne; phosphinoaryl; alkanephosphino; alkenephosphino; alkynephosphino; cycloalkanephosphino; cycloalkenephosphino; cycloalkynephosphino; arylphosphino; phosphono; phosphonoalkane; phosphonoalkene; phosphonoalkyne; phosphonocycloalkane; phosphonocycloalkene; phosphonocycloalkyne; phosphonoaryl; alkanephosphono; alkenephosphono; alkynephosphono; cycloalkanephosphono; cycloalkenephosphono; cycloalkynephosphono; arylphosphono; phosphate; phosphatealkane; phosphatealkene; phosphatealkyne; phosphatecycloalkane; phosphatecycloalkene; phosphatecycloalkyne; phosphatearyl; alkanephosphate; alkenephosphate; alkynephosphate; cycloalkanephosphate; cycloalkenephosphate; cycloalkynephosphate; or arylphosphate.

Pendant group(s) also include any suitable valent form of a carboxyl, carboxylester, carboxylesteramino, carboxylesteroxy, haloalkoxy, halogen, amide, amine, imine, imide, azide, azo, cyano, isocyano, nitrate, nitrile, nitro, oxime, pyridine, carbamate ester, sulfide, disulfide, thiol, sulfoxide, sulfone, sulfate, sulfinyl, thiocarboxylic acid, thioester, dithiocarboxylic acid, dithiocarboxylic acid ester, phosphine, phosphoric acid, phosphate, phosphodiester, alcohol, ketone, aldehyde, acyl halide, carbonate, carboxylate, carboxylic acid, ether, hemiacetal, hemiketal, acetal, orthoester, heterocycle, cycloalkoxy, orthocarbonate ester, organic acid anhydride, silyl, alkylthio, sulfonamide, trifluoromethyl, thiocyano, mercaptocarbonyl, hydroxythiocarbonyl, thiolester, thionoester, carbodithioic acid, carbodithiol, carboyl, formyl, haloformyl, (alkoxycarbonyl)oxy, carboxy, methoxy, alkoxy, alkoxyol dialkoxy, trialkoxy methylenedioxy; tetralkoxy; carboxylic anhydride; carbamoyl; imino; cyanate; isocyanato; nitroxy; nitrosoxy; nitro; nitroso; oxime; pyridyl; carbamate; phosphanyl; phosphono; phosphonoxy; [(alkoxy)hydroxyphosphoryl]oxy, or acyl, etc.

Examples of hydrocarbons used in the present invention include, but are not limited to, pentane, hexane, hexene, hexyne, isohexane, isohexyne, heptane, heptane, heptyne, isoheptane, isoheptene, octane, octene, octyne, isooctane, isooctane, nonane, nonene, nonyne, isononane, decane, isodecane, benzene, toluene, xylene(s), decalin, cyclopropane, cyclopentane, cyclohexane, methylcyclohexane, heptalene, chloroform, dichloromethane, carbon tetrachloride, trichloroethylene, tetrachloroethylene, paraffin, perfluoro-1,4-dimethylcyclohexane, nitromethane, terpene, etc. Examples may include one or mixtures of more than one hydrocarbon. Mixtures may include other group(s) of MDE(s).

Examples of alcohol used in the present invention include, but are not limited to, methanol, ethanol, 1-propanol, 2-propanol, allyl alcohol, 1-butanol, 2-butanol, 2-methyl-1-propanol, 2-methyl-2-propanol, 3-butene-2-ol, crotyl alcohol, cyclopropanemethanol, 3-butene-1-ol, 2-methyl-2-propene-1-ol, 2-butyne-1-ol, 1-pentanol, 2-pentanol, 3-pentanol, 2-methyl-2-butanol, 3-methyl butanol, 2,2-dimethyl-1-propanol, 1-cyclopropylethanol, 1-pentene-3-ol, 4-pentene-2-ol, 2-methyl butene-2-ol, cyclobutanemethanol, 2-methylcyclopropanemethanol, 2-methyl-3-butene-1-ol, 2-methyl-3-butyne-2-ol, 2-pentyne-1-ol, 4-pentyne-2-ol, 1,4-pentadiene-3-ol, 2-pentyne-1-ol, 1-hexanol, 2-hexanol, 3-hexanol, 3-methyl-3-pentanol, 4-methyl-2-pentanol, 2-methyl-3-pentanol, 2-ethyl-1-butanol, 2,3-dimethyl-2-butanol, cyclohexanol, 1-heptanol, 2-heptanol, 3-heptanol, 2-methyl-3-hexanol, 5-methyl-1-hexanol, 2,2-dimethyl-3-pentanol, 3-ethyl-3-pentanol, 2,3-dimethyl-3-pentanol, 1-octanol, 2-octanol, 3-octanol, 4-methyl-3-heptanol, 2,4,4-trimethyl-1-pentanol, 2-propyl-1-pentanol, 1-nonanol, 2-nonanol, 3-methyl-3-octanol, 2,6-dimethyl-4-heptanol, 3,5,5-trimethyl-1-hexanol, 3-ethyl-2,2-dimethyl-4-heptanol, 3-ethyl-2,2-dimethyl-3-pentanol, 1-decanol, 2-decanol, 3,7-dimethyl-3-octanol, 1-undecanol, 2-undecanol, 1-dodecanol, 2-dodecanol, 2-butyl-1-octanol, cyclododecanol, 1-tridecanol, 1-tetradecanol, 2-tetradecanol, 1-pentadecanol, 1-hexadecanol, 2-hexadecanol, 2-hexyl-1-decanol, 1-heptadecanol, 1-octadecanol, etc. Examples may include one or mixtures of more than one alcohol. Mixtures may include other group(s) of MDE(s).

The MDE is a compound that is different from the liquid to be removed from the article. When the liquid to be removed is water, the MDE is not water.

Examples of ketones used in the present invention include, but are not limited to, acetone, methyl ethyl ketone, cyclohexanone, methyl amyl ketone, dimethyl ketone, dipropyl ketone, hexafluoroacetone, etc. Examples may include one or mixtures of more than one ketone. Mixtures may include other group(s) of MDE(s).

Examples of nitriles used in the present invention include, but are not limited to, acetonitrile, propionitrile, acrylonitrile, etc. Examples may include one or mixtures of more than one nitrile. Mixtures may include other group(s) of MDE(s).

Examples of carboxylic acids used in the present invention include, but are not limited to, acetic acid, formic acid, propionic acid, butyric acid, valeric acid, etc. Examples may include one or mixtures of more than one carboxylic acid. Mixtures may include other group(s) of MDE(s).

Examples of esters used in the present invention include, but are not limited to, methyl acetate, ethyl acetate, butyl acetate, isopropyl acetate, amyl acetate, ethyl butyrate, isoamylacetate, ethyl propionate, ethyl caproate, ethyl isobutyrate, propyl acetate, isobutyl acetate, methyl propyl acetate, methyl butyl acetate, hexyl acetate, etc. Examples may include one or mixtures of more than one ester. Mixtures may include other group(s) of MDE(s).

Examples of ethers used in the present invention include, but are not limited to, diethyl ether, tetrahydrofuran, 1,4-dioxane, perfluoro-2-butyltetrahydrofuran, methoxyfuran, etc. Examples may include one or mixtures of more than one ether. Mixtures may include other group(s) of MDE(s).

Examples of glycol ethers used in the present invention include, but are not limited to, ethylene glycol monomethyl ether, ethylene glycol monoethyl ether, ethylene glycol monopropyl ether, ethylene glycol monobutyl ether, ethylene glycol monobutyl ether acetate, diethylene glycol monobutyl ether, etc. Examples may include one or mixtures of more than one glycol ether. Mixtures may include other group(s) of MDE(s).

Examples of aldehydes used in the present invention include, but are not limited to, acetaldehyde, octanal, decanal, etc. Examples may include one or mixture of more than one aldehyde. Mixtures may include other group(s) of MDE(s).

The method by MDEs are utilized in the present invention will be described, in general, for the purposes of illustration only and is not intended to be limiting. A number of different variations of the described method are apparent to one skilled in the art and is highly adaptable for use under various process conditions (e.g. time, temperature, pressure, etc.). Methods for contacting the MDE to the surface(s) or object(s) to be dried can widely vary. For example, drying using the above-mentioned MDEs in a vacuum-based drying system may be completed by first, placing the surface(s) or object(s) to be dried in a vacuum chamber and second, generating an MDE vapor and third, contacting the surface(s) or object(s), to be dried, to the MDE vapor using a vacuum drying process. In another example, the above-mentioned MDE may also contact the surface(s) or object(s) to be dried by first immersing the surface(s) or object(s) in a MDE solution and secondly placing the immersed surface(s) or object(s) in a vacuum chamber to generate MDE vapor in order to initiate drying. In some implementations, the surface(s) or object(s) may be sprayed with MDE solution instead of full immersion. Other methods of contact may be used and may vary widely depending on the type of MDE, type of surface(s) or object(s) and/or application.

In some implementations an MDE may be used in combination with a surfactant. There are a number of different methods by which a surfactant containing MDE composition of the present invention may contact the the surface(s) or object(s) to be dried. These methods can widely vary and those described herein are meant to be descriptive and not meant to be limiting. A number of different variations of the described method are apparent to one skilled in the art and is highly adaptable for use under various process conditions (e.g. time, temperature, pressure, etc.). For example, drying using the above-mentioned surfactant containing MDEs in a vacuum-based drying system may be completed by first, placing the surface(s) or object(s) to be dried in a vacuum chamber and second, generating a vapor consisting of a pre-mixed MDE-surfactant formulation and third, contacting the surface(s) or object(s), to be dried, to the MDE-surfactant vapor using a vacuum drying process. In another example, the surface(s) or object(s) to be dried may first be placed in a vacuum chamber, second, surfactant and MDE vapors may independently be generated followed by sequential exposure of surface(s) or object(s) to be dried to the surfactant vapor followed by the MDE vapor or the MDE vapor followed by the surfactant vapor.

In some implementations, it may be useful for the surface(s) or object(s) to be dried to only be exposed to the surfactant vapor. In another example, the above-mentioned MDE-surfactant may also contact the surface(s) or object(s) to be dried by first immersing the surface(s) or object(s) in a pre-mixed MDE-surfactant solution and second placing the immersed surface(s) or object(s) in a vacuum chamber to generated the MDE and surfactant vapors in order to initiate drying. In some implementations, the surface(s) or object(s) may be sprayed with a pre-mixed MDE-surfactant solution, simultaneously with an isolated MDE solution and an isolated surfactant solution, with an MDE solution followed by a surfactant solution, or a surfactant solution followed by an MDE solution. Other methods of contact may be used and may vary widely depending on the type of MDE, the type of surfactant, type of surface(s) or object(s) and/or application.

Any vacuum chamber that can achieve reduced pressures can cause an MDE to vaporize. In some implementations, heat can be supplied by a conductive heating assembly to facilitate MDE vaporization. MDE vapor flowing across a surface that is being dried in a negative pressure environment, with or without heat, assists in the process for removing liquid from substrate(s), device(s) and/or instrument(s). Heating may be used to enhance vaporization of the MDE or it may be used to facilitate removal of moisture from a substrate, device and/or other instrument surface during drying in a reduced pressure environment. The MDE vapor may be delivered directly to the items to be dried improving the drying process.

A representative apparatus for drying substrate(s), device(s) and/or other instrument(s) using MDE vapor includes a drying system that can be used to dry any one or more suitable substrate(s), device(s) and/or other instrument(s). The drying system can be usd to dry objects that have been exposed to moisture or other cleaning liquids, such as those exposed to a prewash cycle. As described herein, and in some implementations, the drying system consists of a processing chamber and a chamber extension. The chamber extension may be connected to the process chamber externally or internally. The substrate(s), device(s), and/or other instrument(s) to be dried may be placed into the process chamber for drying. The MDE(s) may be supplied to and/or processed in the process chamber and/or the chamber extension.

The drying system can include any suitable MDE delivery mechanism. The MDE(s) may be supplied to the process chamber and/or the chamber extension in liquid form. For example, implementations of the delivery mechanism can include bulk sources of MDE liquid that are stored external or internal to the drying system and are delivered in aliquots to the drying system manually or automatically by a device that controls flow of liquid such as a mass flow controller (MFC) or a syringe pump, etc. MDE vapor(s) and/or non-reactive gases containing quantities of MDE vapor(s) (e.g. nitrogen with methanol) may be stored external or internal to the drying system in containers such as compressed gas containers or containers that generate compressed gas on demand and may be delivered in aliquots to the drying system manually or automatically by a device that controls flow of gas such as an MFC.

Implementations of the delivery mechanism can also include MDE packages or packets, etc. (e.g. liquid or gel-form MDEs or components, ampoules and/or cartridges containing MDE(s) or components, etc.) that may be disposed directly within the process chamber and/or the chamber extension. The container or wrapping containing the MDE liquid, gel or solid-form MDE may be provided in a gas permeable and size-exclusion packet or cartridge that is capable of allowing vacuum in and transport of outgassed or vaporized MDE vapor molecules out of the packet or cartridge while trapping other gel or solid material within the container or wrapping. Examples of vapor gas porous membrane materials that may be used to manufacture the packets or cartridges described herein include, but are not limited to, polytetrafluoroethylene (PTFE), polyethersulfone (PES) or high-density polyethylene (HDPE). The exact vacuum and/or gas permeable membrane (e.g. material type, porosity, etc.) may be selected based on the type and form of the MDE (e.g. liquid versus gel- or solid-form). In some implementations, the liquid, gel or solid form MDE may be disposed within a sealed gas permeable membrane and then sealed within a non-permeable wrapper. Prior to use, the non-permeable wrapper may be removed and the liquid, gel or solid form MDE within the gas-permeable membrane may be inserted into the drying system.

Alternatively, the liquid, gel or solid form MDE may be removed from a non-permeable container or wrapper and placed directly within the drying system. MDE packets or cartridges may be disposed within a separate chamber that is in fluid communication with the process chamber and/or chamber extension. MDE(s), components and/or other agents, as described herein, can be disposed or injected into the process chamber and/or chamber extension independently and then mixed as described herein. In other implementations, the MDEs may be encased in a polymer or other matrix material and released (e.g. outgassed or vaporized) upon heating and/or reduced pressure. A liquid drying moiety or MDE disposed within a matrix is sometimes referred to herein, as a gel or solid form MDE.

The process chamber in conjunction with an MDE(s) can be used to dry any suitable substrate, device and/or instrument. In an electronic component context, the substrate(s) or device(s) can be any item that has been pretreated or treated (e.g. etching, ozone, deionized water rinse, etc.) and may include semiconductors, integrated circuits, solar cells, fuel cells, microelectromechanical systems, disc drives, etc. In a medical context, the device(s) and/or instrument(s) can be any item that has been prewashed and/or treated in a liquid sterilant. Some examples of device(s) and/or instrument(s) include clips, forcepts, scissors, cameras, pacemaker items, hooks/retractors, etc. One non-limiting exemplary medical device is an endoscope or device(s) that contains one or more lumens. Typically, objects, described herein, to be dried have limitations that prevent them from entirely being dried using conventional techniques and result in device and/or instrument failure. In terms of electronic components, residual contamination (e.g. streaking, spotting and/or surface residue) from ectching and/or cleaning processes can cause component failure. Additionally, conventional drying methodologies for electronic components are time consuming. In terms of medical device components, residual moisture from cleaning and/or rinsing processes can cause sterilization failure and, subsequently, potentially life-threatening illnesses from exposure to contaminated device and/or instrument surfaces. Accordingly, the process described herein can use MDE vapor flow in conjunction with negative vacuum, with or without heat, to overcome the limitations of electronic component cleaning that previous methods have failed to provide. The option for use without or minimal heat is advantageous as many device(s) and instrument(s) (e.g. endoscopes with electronics, circuit boards, etc.) have low temperature exposure limits. Accordingly, quick and effective drying of saturated surfaces can be accomplished by utilizing MDE vapor flow in the presence of negative vacuum that is remains well within the thermal exposure limits of the device(s) being dried.

In some implementations, multiple MDEs, of different types, may be used. In such a scenario, segregated or non-segregated vaporization of the MDEs may occur concurrently using multiple chamber extensions. MDE vapor may be generated in the process chamber or may be generated in a chamber extension and transported to one or more process chambers. One or more MDE vapors may be generated in one or more chamber extensions and may flow to one or more process chambers.

Depressurization of the process chamber by a depressurizing subsystem or item, such as a vacuum pump or the like that is capable of achieving a reduced pressure environment in the process chamber and/or chamber extension, causes liquid on and in the substrate(s), device(s) and/or instrument(s) to gasify (e.g. evaporate, vaporize, etc.) triggering the release of liquid vapor from the surfaces, ports or other non-sealed portion of the objects to be dried. Depressurization of the the chamber extension also causes the MDE liquid to gasify (e.g. evaporate, vaporize, etc.) triggering the MDE vapor to be delivered or injected into the process chamber or from the chamber extension into the process chamber. MDE vapor may also be carried throughout the process chamber or from the chamber extension to the process chamber via a non-reactive carrier gas such as air, argon and/or nitrogen.

The flow of the MDE vapor(s) follows a path onto or into the surface(s), port(s) and/or other non-sealed portions of substrate(s), device(s) and/or instrument(s) in which liquid escapes. The movement of MDE vapor on or into substrate(s), device(s) and/or instrument(s) that contain liquid facilitates the complete removal of iquid from the object(s) to be dried by speeding up the process of evaporation, vaporization, etc and by penetrating hard to reach crevices, opening, etc. that may house residual liquid. In some implentations, heat may be added to accelerate the drying process, or heat may be added in varying amounts over time for various purposes. For example, the amount of heat (e.g. and/or a profile of changes in temperature and/or pressure over time) can be tailored to particular implentations of liquid MDEs or gel or solid form MDEs which correspond to vapor pressures for gasification of those MDEs. In yet other implementations, heat can be provided to the MDE vapor.

In some implementations, the substrate(s), device(s) and/or other instrument(s) are placed on or in a drying assembly. In embodiments, the drying assembly resembles a substrate holder, inside of the processing chamber that exposes the faces and/or inter areas of the substrate(s), device(s) and/or other instruments to the MDE vapor. In other implementations, delivery of MDE vapor to the immediate surface or core of an object to be dried can occur via a direct conduit such as a porous manifold. The porous manifold is designed to feed MDE vapor to the inner area of the object. In another example, substrate(s), device(s) and/or other instrument(s) may be be placed in an envelope type structure or isolated containers machined to be a manifold of which design achieves directs flow of MDE vapor to the objects in the envelope. In yet another implementation, the drying assembly (e.g. tray, manifold, etc.) or any extension of the assembly (e.g. thermally conductive conformable media) may be heated to assist in the process of drying.

The MDE composition comprises one or more MDEs. THE MDE composition may exist in a solution or a solvent. Additionally, any suitable substance that when in contact with a solution of solvent generates the MDE can be used in the present method. For example, the substance can be a salt of alkoxide, e.g. a sodium salt of ethoxide, which in the presence of water generates ethanol. Or the substance may be an alkene, such as ethylene, which in the presence of water and a catalyst generates ethanol.

Description of methods that follow are not meant to be limiting.

In embodiments, the drying system is designed to receive a wet substrate(s), device(s) and/or other instrument(s) into the process chamber via a door. The term wet is often used to describe an item that contains water. Herein the term wet refers generally to the presence of any undesired fluid that is to be remove and includes water of aqueous solutions. For example, the door may be disposed on a top surface of the chamber or on the side of the chamber and includes any gaskets or other seals to allow the vacuum process chamber to be sufficiently sealed when the door is closed and the process chamber is pressurized. A similar form factor can be designed to support multiple drying chamber for concurrent drying and/or for drying substrate(s), device(s) and/or other instruments of various sizes and or shapes. The substrate(s), device(s), and/or other instruments are assembled onto or in a substrate holder(s) or drying assembly, as required. MDE liquid or gel- or solid-form is placed or injected, manually or automatically, into the drying system after which point the drying system door(s) are closed and sealed and the system is pressurized by the pressurizing subsystem. The heating subsystem provides heat to the conductive thermal assembly of the process chamber and/or the drying assembly. As the pressure in the chamber is reduced MDE vapor from the MDE source (e.g. liquid, gel- or solid-form) is simultaneously outgassed causing the moisture from the substrate(s), device(s) and/or other instrument(s) to vaporized and dry. Heated or non-heated, non-reactive carrier gas may be introduced into the process chamber enhancing flow of the MDE vapor over the surface of wet substrate(s), device(s) and/or other instrument(s).

In another embodiment, the drying system is essentially the same as the one described above except that a separate process chamber and chamber extension are utilized as part of the drying system. In this regard, the process chamber is configured with a conductive thermal assembly and is configured to receive one or more substrate(s), device(s) and/or other instrument(s) on a drying assembly or a substrate holder. The chamber extension is configured with a separately controlled conductive thermal assembly and is designed to receive the MDE liquid or gel- or solid-form. The chamber extension may also include a door or doors that include gaskets or other seals to allow the chamber extension to be sufficiently sealed when the door is closed and the chamber extension is pressurized. The chamber extension may also include an inlet from whereby the MDE liquid is injected into the chamber extension. The process chamber and the chamber extension are in fluid connection with one another via path or conduit. The conduit may include valves (first valves) that may be actuated/controlled by the controller or user. Both drying system descriptions include a second valve (second valves) between the first chamber and the pressurizing system (e.g. vacuum pump). This second valve may be controlled by the controller or the user.

In operation, both valves may be opened to permit the pressurizing subsystem to evacuate each chamber. In various operations, upon achieving a desired pressure level (e.g. vacuum level) the first valve may be opened and closed to permit off-gassing and transport of the MDE vapor from the chamber extension into the process chamber. The second valve may remain open to permit continued evaporation and removal of water or other rinsing liquids from the substrate(s), device(s) and/or other instrument(s). When desired the first valve may be opened to permit adiabatic expansion and transport of the gasified MDE from the chamber extension into the process chamber. The second valve may be closed before the water or other rinsing liquids are evaporated from the substrate(s), device(s) and/or other instrument(s) to allow the MDE vapor to enter into the process chamber and flow over the substrate(s), device(s) and/or other instrument(s). Heated or non-heated, non-reactive carrier gas may be introduced into the process chamber enhancing flow of the MDE vapor over the surface of wet substrate(s), device(s) and/or other instrument(s). Heated or non-heated, non-reactive carrier gas may be introduced into the chamber extension enhancing flow of the MDE vapor from the chamber extension to the process chamber and further over the surface of wet substrate(s), device(s) and/or other instrument(s).

Such an arrangement permits adiabatic expansion of the gasified MDE into the process chamber and onto and into the substrate(s), devices(s) and/or other instrument(s) such that gasified MDE is able to expand into all interior areas of the objects to be dried. Such a configuration may allow for reduced use of MDE or MDE vapor compared to a system that continually draws a vacuum. Though illustrated as utilizing a single pressurizing system for both chambers, it will be appreciated that the process chamber and the chamber extension may have unique depressurizing systems. Though discussed in relation to fluidly isolating the chambers, it will be appreciated that in some implementations, the chambers may remain in fluid communication throughout the process. In this implementation, the chamber extension may primarily be used to control the separate heating of the MDE agent.

MDEs selected to generate vapors using the disclosed method have excellent mass transfer properties in the form of concentration and/or temperature effects in the vacuum system described herein. Their mass transfer properties permit formation of a surface-tension gradient in the vapor-water interface of water located on the surface of a substrate. A vapor consisting of an MDE, such as methanol, when passed over the surface of a wet device is absorbed by the fluid used to rinse the substrate(s), device(s) and/or other instrument(s) (e.g. deionized water) which lower the surface tension of the rinsing fluid. A decrease in the surface tension of the fluid on the surface being washed induced the Marangoni effect and causes the rinsing fluid to flow away from areas on the substrate thereby drying the substrate(s), device(s) and/or other instrument(s).

There are a number of advantages to drying using MDE vapors generated in the vacuum system described herein. Vaporization of MDEs is facilitated and can be achieved quickly by using an outgassing process unique to the vacuum system described. Advantageously, using MDE vapors in a reduced pressure process as described facilitates the work of introducing drying vapor and assisting with removal of moisture from substrate surfaces. The concentration of MDE vapor introduced into the system during a drying process may be adjusted to fit the amount of moisture present at the start of the process. In an instance where more moisture is detected a greater amount of MDE vapor can be introduced. Increasing the amount of MDE vapor may be advantageous for increasing the surface-tension gradient via concentration and subsequently enhancing moisture removal, as described above. However, the concentration of MDE vapor has to be controlled to flow in the appropriate concentration range for the type and number of substrate(s), device(s) and/or other instruments being dried. When the concentration of the MDE vapor is too low, sufficient drying is not achieved whereas on the other hand when the concentration is too high, the chances of MDE particle deposition and surface contamination occurring are higher.

Additionally, vaporization of the MDEs can be expedited by using elevated temperatures. Even though the use of heat may increase the vaporization time of the MDEs in a sub-atmospheric environment it is not required as part of this process. However, heating the MDE vapor may be advantageous for improving heat transfer, generating a surface-tension gradient via temperature and subsequently enhancing moisture removal, as described above. Equally important, the drying action of the process can be enhanced by heating the devices or instruments in combination with exposing them to MDE vapor flow. The devices or instruments may be exposed to the ambient heat from the vacuum chamber or heat delivered to the substrate trays or holder.

Advantageously, permeation of the objects to be dried by the MDE vapor and delivery of the MDE vapor into otherwise hard to reach apertures, e.g. inner area of a lumen, can be enhanced by adding an inert carrier gas or applying heat to the object to be dried. Some examples of a non-reactive carrier gas include air, carbon dioxide, nitrogen, and argon. Increasing the pressure of the vacuum system by introducing a carrier gas may promoted transport of the MDE vapor into hard to reach pores and small openings of devices that are wet. The carrier gas may also be heated to improve heat transfer subsequently generating a surface-tension gradient via temperature, as described above.

Another advantage of the invention is that the MDE vapor can be vaporized in a separate chamber, the chamber extension, from the vacuum chamber where the objects to be dried are located. The operation of independent chambers allows for the use of different process conditions. The chamber extension can be independently controlled from the vacuum chamber. This may be advantageous when a higher temperature is required to vaporize the MDE liquid than what is desirable to expose heat sensitive devices to be dried. It will be appreciated that there may be more than one vacuum chamber in which MDE vapors from an extension chamber may generate and deliver MDE vapor into one or more vacuum chambers simultaneously. Additionally, there may be more than one extension chamber connected to a vacuum chamber in which the same MDE or different types of MDEs can be vaporized and delivered into the vacuum chamber.

While the compositions, methods and apparatus of the invention have been particularly shown and described with reference to the formulas, apparatus and methods herein, it will be understood by those skilled in the art that various changes in the form and details may be made therein without departing from the spirit and scope of the invention. While the method has been specifically described for treatment of a lumen, it will be readily apparent to one of ordinary skill in the art that a variety of articles including substrates, devices including medical devices, and electronic devices (e.g., computers) can be treated using the methods as described herein.

When a Markush group or other grouping is used herein, all individual members of the group and all combinations and subcombinations possible of the group are intended to be individually included in the invention. Every formulation or combination of components described or exemplified can be used to practice the invention, unless otherwise stated. Specific names of compounds are intended to be exemplary, as it is known that one of ordinary skill in the art can name the same compounds differently.

One of ordinary skill in the art will appreciate that methods, including experimental procedure, preparation methods and analytical methods, materials and device elements other than those specifically exemplified can be employed in the practice of the invention without resort to undue experimentation. All art-known functional equivalents of any such methods or materials are intended to be included in this invention.

Whenever a range is given in the specification, for example, a composition range, a range of process conditions, a range of pressures or temperatures or the like, all intermediate ranges and subranges, as well as all individual values included in the ranges given are intended to be included in the invention.

The invention illustratively described herein suitably may be practiced in the absence of any element or elements, limitation or limitations which is not specifically disclosed herein.

Without wishing to be bound by any particular theory, there can be discussion herein of beliefs or understandings of underlying principles or mechanisms of action relating to the invention. It is recognized that regardless of the ultimate correctness of any mechanistic explanation or hypothesis, an embodiment of the invention can nonetheless be operative and useful.

All references throughout this application, for example patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

### THE EXAMPLES

The following examples are for illustrative purposes and are not intended to limit the scope of the invention.

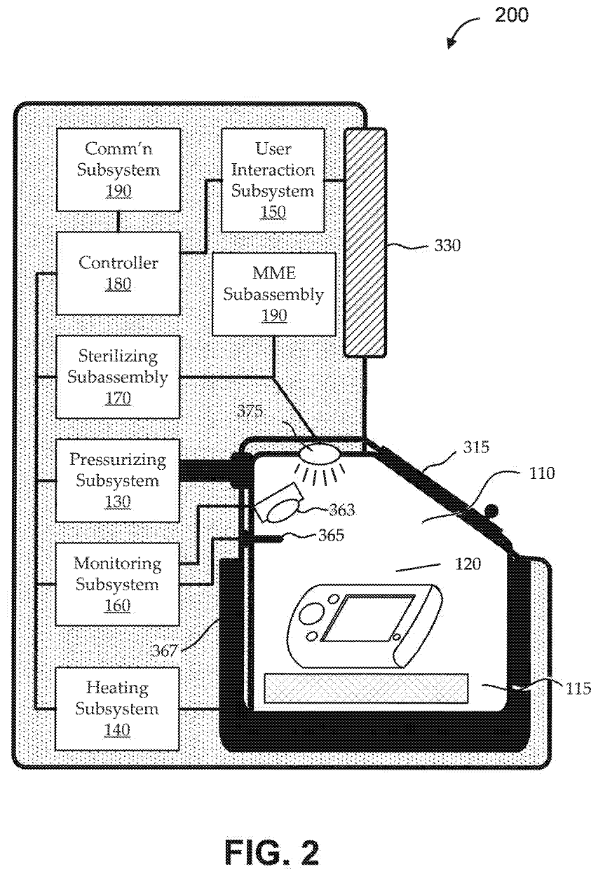

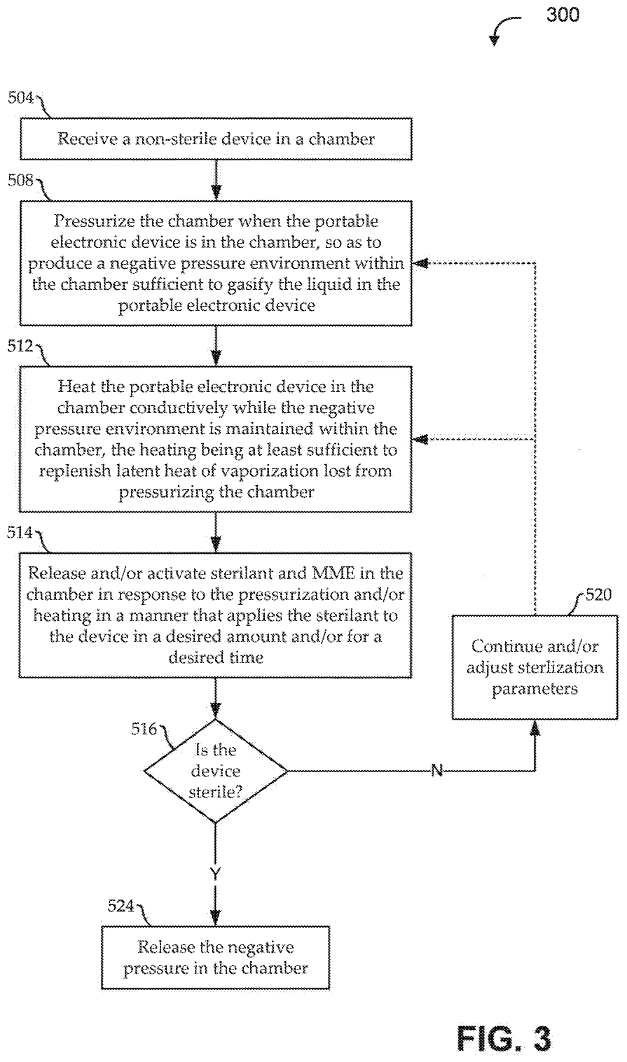

A typical diffusion apparatus for conducting these evaluations includes a stainless-steel pressure chamber which is connected to a smaller stainless-steel extension chamber. Connection is made by means of a stainless-steel tube that contains a valve for isolating vapor flow between the vacuum chamber and the chamber extension. The chamber extension contains isolated compartments that admitted molecular mobility enhancer (MME) and/or liquid or solid agent components. The vacuum chamber and the chamber extension are independently heated. The methods herein can be practiced without heating. The isolated compartments within the chamber extension were independently heated. The heated compartments of the system can be altered when needed and monitored. The chamber extension as a whole as well as the isolated compartments within the chamber extension are connected to MME and transport moiety sources via a valve and a conduit connected to a flow controller. The chamber extension is also outfitted with a door that can be used for placing solid agent sources within the extension. The entire system (vacuum chamber and chamber extension) is connected to a vacuum system e.g., (comprising a vacuum pump) capable of reducing the pressure and vaporizing the molecular mobility enhancer and liquid or solid transport moieties. FIG. 1 illustrates the sterilization/sanitization process 100 in an exemplary sterilizing system 105. FIG. 2 shows a diagram of an exemplary sterilizer and how the subsystems connect to the process chamber 110. The object to be sterilized 120 is placed in the vacuum sterilization chamber 110 on a radiative/conductive heater 115. FIG. 3 shows a low chart 300 of the generic process for sterilization of a device using the MME. Note: this is the same generic process for sanitization, with an adjustment of the specific process parameters for the sanitization versus sterilization.

### Evaluation of Vaporization Rate

### Example 1 (Process Chamber, Hydrogen Peroxide with and without MME Methanol)

A transport moiety solution of aqueous hydrogen peroxide (30 wt %), 5 mL, was placed inside of the process chamber of the diffusion system. In a separate, but similar experiment, an MME-transport moiety blend solution containing 5% (v/v) methanol in 5 mL of aqueous hydrogen peroxide (30 wt %) was placed in the process chamber of the diffusion system. In both tests the diffusion system was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles over time for each process were analyzed to show that the downward inflection point, or the time at which all liquid has been converted to vapor, of the MME-transport moiety blend pressure profile occurred at an earlier time point in the process than did the downward inflection point of the transport moiety only.

### Example 2 (Chamber Extension, Hydrogen Peroxide with and without MME Methanol)

A transport moiety solution of aqueous hydrogen peroxide (30 wt %), 5 mL, was placed inside of the chamber extension of the diffusion system. In a separate, but similar experiment, an MME-transport moiety blend solution containing 5% (v/v) methanol in 5 mL of aqueous hydrogen peroxide (30 wt %) was placed in the chamber extension of the diffusion system. In both tests the diffusion system was heated to 50° C. The door to the diffusion system for each was closed and sealed. The diffusion system including the chamber extension was opened to vacuum and the pressure of the system was monitored over the course of each 30-minute time period. At the end of each process the diffusion system was brought to atmosphere. The pressure profiles over time for each process were analyzed to show that the downward inflection point, or the time at which all liquid has been converted to vapor, of the MME-transport moiety blend pressure profile occurred at an earlier time point in the process than did the downward inflection point of the transport moiety only.

### Example 3 (Process Chamber, Performic Acid with and without MME Diethyl Ether)

A transport moiety solution without MME (5 mL of performic acid solution) was placed inside of the process chamber of the diffusion system. The performic acid solution was prepared by mixing 3.50 g formic acid (98%) with 1.48 g of 30 wt % hydrogen peroxide. The performic acid solution contains 8.9% by mass performic acid in solution. In a separate, but similar experiment, an MME-transport moiety blend solution containing 0.5% (v/v) diethyl ether in 5 mL of performic acid solution was placed in the process chamber of the diffusion system. In both tests the diffusion system was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles over time for each process were analyzed to show that the downward inflection point, or the time at which all liquid has been outgassed, of the MME-transport moiety blend pressure profile occurred at an earlier time point in the process than did the downward inflection point of the performic acid transport moiety only.

### Example 4 (Chamber Extension, Performic Acid with and without MME Diethyl Ether)

A transport moiety solution of performic acid, 5 mL, was placed inside of the chamber extension of the diffusion system. In a separate, but similar experiment, an MME-transport moiety blend solution containing 0.5% (v/v) diethyl ether in 5 mL of performic acid solution (as described in Example 3) was placed in the chamber extension of the diffusion system. In both tests the diffusion system was heated to 50° C. The door to the diffusion system for each was closed and sealed. The diffusion system including the chamber extension was opened to vacuum and the pressure of the system was monitored over the course of each 30-minute time period. At the end of each process the diffusion system was brought to atmosphere. The pressure profiles over time for each process were analyzed to show that the downward inflection point, or the time at which all liquid has been outgassed, of the MME-transport moiety blend pressure profile occurred at an earlier time point in the process than did the downward inflection point of the transport moiety only.

### Example 5 (Process Chamber, Peracetic Acid with and without MME Pentane)

A transport moiety solution of peracetic acid, 5 mL (32 wt % peracetic acid in acetic acid (40-45%), was placed inside of the process chamber of the diffusion system. In a separate, but similar experiment, an MME-transport moiety blend solution containing 1% (v/v) pentane in 5 mL of peracetic acid solution was placed in the process chamber of the diffusion system. In both tests the diffusion system was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles over time for each process were analyzed to show that the downward inflection point, or the time at which all liquid has been outgassed, of the MME-transport moiety blend pressure profile occurred at an earlier time point in the process than did the downward inflection point of the transport moiety only.

### Example 6 (Chamber Extension, Peracetic Acid with and without MME Pentane)

A transport moiety solution of peracetic acid, 5 mL (as described in Example 5), was placed inside of the chamber extension of the diffusion system. In a separate, but similar experiment, an MME-transport moiety blend solution containing 1% (v/v) pentane in 5 mL of peracetic acid solution was placed in the chamber extension of the diffusion system. In both tests the diffusion system was heated to 50° C. The door to the diffusion system for each was closed and sealed. The diffusion system including the chamber extension was opened to vacuum and the pressure of the system was monitored over the course of each 30-minute time period. At the end of each process the diffusion system was brought to atmosphere. The pressure profiles over time for each process were analyzed to show that the downward inflection point, or the time at which all liquid has been outgassed, of the MME-transport moiety blend pressure profile occurred at an earlier time point in the process than did the downward inflection point of the transport moiety only.

### Example 7 (Process Chamber, Isopropanol with and without MME Formic Acid)

A transport moiety solution of isopropanol, 5 mL, was placed inside of the process chamber of the diffusion system. In a separate, but similar experiment, an MME-transport moiety blend solution containing 4% (v/v) formic acid in 5 mL of isopropanol was placed in the process chamber of the diffusion system. In both tests the diffusion system was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles over time for each process were analyzed to show that the downward inflection point, or the time at which all liquid has been outgassed, of the MME-transport moiety blend pressure profile occurred at an earlier time point in the process than did the downward inflection point of the transport moiety only.

### Example 8 (Chamber Extension, Isopropanol with and without MME Formic Acid)

A transport moiety solution of isopropanol, 5 mL, was placed inside of the chamber extension of the diffusion system. In a separate, but similar experiment, an MME-transport moiety blend solution containing 4% (v/v) formic acid in 5 mL of isopropanol was placed in the chamber extension of the diffusion system. In both tests the diffusion system was heated to 50° C. The door to the diffusion system for each was closed and sealed. The diffusion system including the chamber extension was opened to vacuum and the pressure of the system was monitored over the course of each 30-minute time period. At the end of each process the diffusion system was brought to atmosphere. The pressure profiles over time for each process were analyzed to show that the downward inflection point, or the time at which all liquid has been outgassed, of the MME-transport moiety blend pressure profile occurred at an earlier time point in the process than did the downward inflection point of the transport moiety only.

### Example 9 (Process Chamber, Hydrogen Peroxide with and without MME Ethyl Acetate)

A transport moiety solution of aqueous hydrogen peroxide (30 wt %), 5 mL, was placed inside of the process chamber of the diffusion system. In a separate, but similar experiment, an MME-transport moiety blend solution containing 2% (v/v) ethyl acetate in 5 mL of aqueous hydrogen peroxide (30 wt %) was placed in the process chamber of the diffusion system. In both tests the diffusion system was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles over time for each process were analyzed to show that the downward inflection point, or the time at which all liquid has been outgassed, of the MME-transport moiety blend pressure profile occurred at an earlier time point in the process than did the downward inflection point of the transport moiety only.

### Example 10 (Chamber Extension, Hydrogen Peroxide with and without MME Ethyl Acetate)

A transport moiety solution of aqueous hydrogen peroxide (30 wt %), 5 mL, was placed inside of the chamber extension of the diffusion system. In a separate, but similar experiment, an MME-transport moiety blend solution containing 2% (v/v) ethyl acetate in 5 mL of aqueous hydrogen peroxide (30 wt %) was placed in the chamber extension of the diffusion system. In both tests the diffusion system was heated to 50° C. The door to the diffusion system for each was closed and sealed. The diffusion system including the chamber extension was opened to vacuum and the pressure of the system was monitored over the course of each 30-minute time period. At the end of each process the diffusion system was brought to atmosphere. The pressure profiles over time for each process were analyzed to show that the downward inflection point, or the time at which all liquid has been outgassed, of the MME-transport moiety blend pressure profile occurred at an earlier time point in the process than did the downward inflection point of the transport moiety only.

### Example 11 (Process Chamber, Isopropanol with and without MME Acetone)

A transport moiety solution of isopropanol, 5 mL, was placed inside of the process chamber of the diffusion system. In a separate, but similar experiment, an MME-transport moiety blend solution containing 1% (v/v) acetone in 5 mL of isopropanol was placed in the process chamber of the diffusion system. In both tests the diffusion system was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles over time for each process were analyzed to show that the downward inflection point, or the time at which all liquid has been outgassed, of the MME-transport moiety blend pressure profile occurred at an earlier time point in the process than did the downward inflection point of the transport moiety only.

### Example 12 (Process Chamber, Isopropanol with and without MME Acetone)

A transport moiety solution of isopropanol, 5 mL, was placed inside of the chamber extension of the diffusion system. In a separate, but similar experiment, an MME-transport moiety blend solution containing 1% (v/v) acetone in 5 mL of isopropanol was placed in the chamber extension of the diffusion system. In both tests the diffusion system was heated to 50° C. The door to the diffusion system for each was closed and sealed. The diffusion system including the chamber extension was opened to vacuum and the pressure of the system was monitored over the course of each 30-minute time period. At the end of each process the diffusion system was brought to atmosphere. The pressure profiles over time for each process were analyzed to show that the downward inflection point, or the time at which all liquid has been outgassed, of the MME-transport moiety blend pressure profile occurred at an earlier time point in the process than did the downward inflection point of the transport moiety only.

### Evaluation of Vapor Diffusion

### Example 13 (Process Chamber, Hydrogen Peroxide without MME Methanol)

Vapor hydrogen peroxide chemical indicators were placed along the entire inner length of a 2-meter long 4-mm inner diameter polytetrafluoroethylene tube. The tube and its contents were placed in the process chamber of the diffusion system. In this test the process chamber was heated to 50° C. A transport moiety liquid solution containing aqueous hydrogen peroxide (30 wt %) was introduced into the process chamber. The door to the diffusion system was closed and sealed. The diffusion system was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had not been exposed to the transport moiety vapor.

### Example 14 (Process Chamber, Hydrogen Peroxide with Methanol MME)

Vapor hydrogen peroxide chemical indicators were placed along the entire inner length of a 2-meter long 4-mm inner diameter polytetrafluoroethylene tube. The tube and its contents were placed in the process chamber of the diffusion system. In this test the process chamber was heated to 50° C. An MME-transport moiety blend solution containing 5% (v/v) methanol in aqueous hydrogen peroxide (30 wt %) was introduced into the process chamber. The door to the diffusion system was closed and sealed. The diffusion system was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had been exposed to the transport moiety vapor.

### Example 15 (Chamber Extension, Hydrogen Peroxide without Methanol MME)

Vapor hydrogen peroxide chemical indicators were placed along the entire inner length of a 2-meter long 4-mm inner diameter polytetrafluoroethylene tube. The tube and its contents were placed in the process chamber of the diffusion system. In this test the diffusion system was heated to 50° C. A transport moiety blend solution containing aqueous hydrogen peroxide (30 wt %) was introduced into the chamber extension. The door to the diffusion system was closed and sealed. The diffusion system including the chamber extension was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had not been exposed to the transport moiety vapor.

### Example 16 (Chamber Extension, Hydrogen Peroxide with MME Methanol)

Vapor hydrogen peroxide chemical indicators were placed along the entire inner length of a 2-meter long 4-mm inner diameter polytetrafluoroethylene tube. The tube and its contents were placed in the process chamber of the diffusion system. In this test the diffusion system was heated to 50° C. An MME-transport moiety blend solution containing 5% (v/v) methanol in aqueous hydrogen peroxide (30 wt %) was introduced into the chamber extension. The door to the diffusion system was closed and sealed. The diffusion system including the chamber extension was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had been exposed to the transport moiety vapor.

### Example 17 (Process Chamber, Performic Acid without MME Diethyl Ether)

Vapor performic acid chemical indicators were placed along the entire inner length of a 2-meter long 4-mm inner diameter polytetrafluoroethylene tube. The tube and its contents were placed in the process chamber of the diffusion system. In this test the process chamber was heated to 50° C. A transport moiety liquid solution containing performic acid solution (as described above) was introduced into the process chamber. The door to the diffusion system was closed and sealed. The diffusion system was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had not been exposed to the transport moiety vapor.

### Example 18 (Process Chamber, Performic Acid with MME Diethyl Ether

Vapor performic acid chemical indicators were placed along the entire inner length of a 2-meter long 4-mm inner diameter polytetrafluoroethylene tube. The tube and its contents were placed in the process chamber of the diffusion system. In this test the process chamber was heated to 50° C. An MME-transport moiety blend solution containing 0.5% (v/v) diethyl ether in performic acid solution (as described above) was introduced into the process chamber. The door to the diffusion system was closed and sealed. The diffusion system was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had been exposed to the transport moiety vapor.

### Example 19 (Chamber Extension, Performic Acid without MME Diethyl Ether)

Vapor performic acid chemical indicators were placed along the entire inner length of a 2-meter long 4-mm inner diameter polytetrafluoroethylene tube. The tube and its contents were placed in the process chamber of the diffusion system. In this test the diffusion system was heated to 50° C. A transport moiety liquid solution containing performic acid solution (as described above) was introduced into the chamber extension. The door to the diffusion system was closed and sealed. The diffusion system including the chamber extension was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had not been exposed to the transport moiety vapor

### Example 20 (Chamber Extension, Performic Acid with MME Diethyl Ether)

Vapor performic acid chemical indicators were placed along the entire inner length of a 2-meter long 4-mm inner diameter polytetrafluoroethylene tube. The tube and its contents were placed in the process chamber of the diffusion system. In this test the diffusion system was heated to 50° C. An MME-transport moiety blend solution containing 0.5% (v/v) diethyl ether in performic acid solution (as described above) was introduced into the chamber extension. The door to the diffusion system was closed and sealed. The diffusion system including the chamber extension was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had been exposed to the transport moiety vapor.

### Example 21 (Process Chamber, Peracetic Acid without MME Pentane)

Vapor peracetic acid chemical indicators were placed along the entire inner length of a 2-meter long 4-mm inner diameter polytetrafluoroethylene tube. The tube and its contents were placed in the process chamber of the diffusion system. In this test the process chamber was heated to 50° C. A transport moiety liquid solution containing peracetic acid solution (as described above) was introduced into the process chamber. The door to the diffusion system was closed and sealed. The diffusion system was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had not been exposed to the transport moiety vapor.

### Example 22 (Process Chamber, Peracetic Acid with MME Pentane)

Vapor peracetic acid chemical indicators were placed along the entire inner length of a 2-meter long 4-mm inner diameter polytetrafluoroethylene tube. The tube and its contents were placed in the process chamber of the diffusion system. In this test the process chamber was heated to 50° C. An MME-transport moiety blend solution containing 2% pentane in peracetic acid solution (as described above) was introduced into the process chamber. The door to the diffusion system was closed and sealed. The diffusion system was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had been exposed to the transport moiety vapor.

### Example 23 (Chamber Extension, Peracetic Acid without MME Pentane)

Vapor peracetic acid chemical indicators were placed along the entire inner length of a 2-meter long 4-mm inner diameter polytetrafluoroethylene tube. The tube and its contents were placed in the process chamber of the diffusion system. In this test the diffusion system was heated to 50° C. A transport moiety liquid solution containing peracetic acid solution (as described above) was introduced into the chamber extension. The door to the diffusion system was closed and sealed. The diffusion system including the chamber extension was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had not been exposed to the transport moiety vapor.

### Example 24 (Chamber Extension, Peracetic Acid with MME Pentane

Vapor peracetic acid chemical indicators were placed along the entire inner length of a 2-meter long 4-mm inner diameter polytetrafluoroethylene tube. The tube and its contents were placed in the process chamber of the diffusion system. In this test the diffusion system was heated to 50° C. An MME-transport moiety liquid solution containing 2% (v/v) pentane in peracetic acid (as described above) was introduced into the chamber extension. The door to the diffusion system was closed and sealed. The diffusion system including the chamber extension was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had been exposed to the transport moiety vapor.

### Example 25 (Process Chamber, Isopropanol without MME Formic Acid)

Vapor alcohol chemical indicators were placed along the entire inner length of a 2-meter long 4-mm inner diameter polytetrafluoroethylene tube. The tube and its contents were placed in the process chamber of the diffusion system. In this test the process chamber was heated to 50° C. A transport moiety liquid solution containing isopropanol was introduced into the process chamber. The door to the diffusion system was closed and sealed. The diffusion system was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had not been exposed to the transport moiety vapor.

### Example 26 (Process Chamber, Isopropanol with MME Formic Acid)

Vapor alcohol chemical indicators were placed along the entire inner length of a 2-meter long 4-mm inner diameter polytetrafluoroethylene tube. The tube and its contents were placed in the process chamber of the diffusion system. In this test the process chamber was heated to 50° C. An MME-transport moiety blend solution containing 5% (v/v) formic acid in isopropanol was introduced into the process chamber. The door to the diffusion system was closed and sealed. The diffusion system was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had been exposed to the transport moiety vapor.

### Example 27 (Chamber Extension, Isopropanol without MME Formic Acid)

Vapor alcohol chemical indicators were placed along the entire inner length of a 2-meter long 4-mm inner diameter polytetrafluoroethylene tube. The tube and its contents were placed in the process chamber of the diffusion system. In this test the diffusion system was heated to 50° C. A transport moiety liquid solution containing isopropanol was introduced into the chamber extension. The door to the diffusion system was closed and sealed. The diffusion system including the chamber extension was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had not been exposed to the transport moiety vapor.

### Example 28 (Chamber Extension, Isopropanol with MME Formic Acid)

Vapor alcohol chemical indicators were placed along the entire inner length of a 2-meter long 4-mm inner diameter polytetrafluoroethylene tube. The tube and its contents were placed in the process chamber of the diffusion system. In this test the diffusion system were heated to 50° C. An MME-transport moiety blend solution containing 5% (v/v) formic acid in isopropanol was introduced into the chamber extension. The door to the diffusion system was closed and sealed. The diffusion system including the chamber extension was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had been exposed to the transport moiety vapor.

### Example 29 (Process Chamber, Hydrogen Peroxide without MME Ethyl Acetate)

Vapor hydrogen peroxide chemical indicators were placed along the entire inner length of a 2-meter long 4-mm inner diameter polytetrafluoroethylene tube. The tube and its contents were placed in the process chamber of the diffusion system. In this test the process chamber was heated to 50° C. A transport moiety liquid solution containing aqueous hydrogen peroxide (30 wt %) was introduced into the process chamber. The door to the diffusion system was closed and sealed. The diffusion system was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had not been exposed to the transport moiety vapor.

### Example 30 (Process Chamber, Hydrogen peroxide with MME Ethyl Acetate)

Vapor hydrogen peroxide chemical indicators were placed along the entire inner length of a 2-meter long 4-mm inner diameter polytetrafluoroethylene tube. The tube and its contents were placed in the process chamber of the diffusion system. In this test the process chamber was heated to 50° C. An MME-transport moiety blend solution containing 3% (v/v) ethyl acetate in aqueous hydrogen peroxide (30 wt %) was introduced into the process chamber. The door to the diffusion system was closed and sealed. The diffusion system was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had been exposed to the transport moiety vapor.

### Example 31 (Chamber Extension, Hydrogen Peroxide without MME Ethyl Acetate)

Vapor hydrogen peroxide chemical indicators were placed along the entire inner length of a 2-meter long 4-mm inner diameter polytetrafluoroethylene tube. The tube and its contents were placed in the process chamber of the diffusion system. In this test the diffusion system was heated to 50° C. A transport moiety solution containing aqueous hydrogen peroxide (30 wt %) was introduced into the chamber extension. The door to the diffusion system was closed and sealed. The diffusion system including the chamber extension was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had not been exposed to the transport moiety vapor.

### Example 32 (Chamber Extension, Hydrogen Peroxide with MME Ethyl Acetate)

Vapor hydrogen peroxide chemical indicators were placed along the entire inner length of a 2-meter long 4-mm inner diameter polytetrafluoroethylene tube. The tube and its contents were placed in the process chamber of the diffusion system. In this test the diffusion system was heated to 50° C. An MME-transport moiety blend solution containing 3% (v/v) ethyl acetate in aqueous hydrogen peroxide (30 wt %) was introduced into the chamber extension. The door to the diffusion system was closed and sealed. The diffusion system including the chamber extension was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had been exposed to the transport moiety vapor.

### Example 33. (Process Chamber, Isopropanol without MME Acetone)

Vapor alcohol chemical indicators were placed along the entire inner length of a 2-meter long 4-mm inner diameter polytetrafluoroethylene tube. The tube and its contents were placed in the process chamber of the diffusion system. In this test the process chamber was heated to 50° C. A transport moiety liquid solution containing isopropanol was introduced into the process chamber. The door to the diffusion system was closed and sealed. The diffusion system was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had not been exposed to the transport moiety vapor.

### Example 34 (Process Chamber, Isopropanol with MME Acetone)

Vapor alcohol chemical indicators were placed along the entire inner length of a 2-meter long 4-mm inner diameter polytetrafluoroethylene tube. The tube and its contents were placed in the process chamber of the diffusion system. In this test the process chamber was heated to 50° C. An MME-transport moiety blend solution containing 0.5% (v/v) acetone in isopropanol was introduced into the process chamber. The door to the diffusion system was closed and sealed. The diffusion system was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had been exposed to the transport moiety vapor.

### Example 35 (Chamber Extension, Isopropanol without MME Acetone)

Vapor alcohol chemical indicators were placed along the entire inner length of a 2-meter long 4-mm inner diameter polytetrafluoroethylene tube. The tube and its contents were placed in the process chamber of the diffusion system. In this test the diffusion system was heated to 50° C. A transport moiety solution containing isopropanol was introduced into the chamber extension. The door to the diffusion system was closed and sealed. The diffusion system including the chamber extension was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had not been exposed to the transport moiety vapor.

### Example 36 (Chamber Extension, Isopropanol with MME Acetone)

Vapor alcohol chemical indicators were placed along the entire inner length of a 2-meter long 4-mm inner diameter polytetrafluoroethylene tube. The tube and its contents were placed in the process chamber of the diffusion system. In this test the diffusion system was heated to 50° C. An MME-transport moiety blend solution containing 0.5% (v/v) acetone in isopropanol was introduced into the chamber extension. The door to the diffusion system was closed and sealed. The diffusion system including the chamber extension was evacuated to a pressure less than 10 torr. Multiple vaporization/diffusion cycles were performed over the course of a 15-minute time period. At the end of the process the diffusion system was brought to atmosphere and the tube and its contents were removed from the process chamber. Visual observation of color change on the chemical indicators was made indicating the entire inner length of the tube had been exposed to the transport moiety vapor.

### Evaluation of Vapor Evacuation Rate

### Example 37 (Process Chamber, Hydrogen Peroxide with and without MME Methanol)

A transport moiety solution of aqueous hydrogen peroxide (30 wt %), 5 mL, was placed inside of the process chamber of the diffusion system. In a separate, but similar experiment, an MME-transport moiety blend solution containing 5% (v/v) methanol in 5 mL of aqueous hydrogen peroxide (30 wt %) was placed in the process chamber of the diffusion system. In both tests the process chamber was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles over time for each process were analyzed to show that the negative slope, or the rate of vapor evacuation, of aqueous hydrogen peroxide vapor in the presence of MME vapor, methanol, was greater than the rate of evacuation of aqueous hydrogen peroxide vapor without MME.

### Example 38 (Chamber Extension, Hydrogen Peroxide with and without MME Methanol)

A transport moiety solution of aqueous hydrogen peroxide (30 wt %), 5 mL, was placed inside of the chamber extension of the diffusion system. In a separate, but similar experiment, an MME-transport moiety blend solution containing 5% (v/v) methanol in 5 mL of aqueous hydrogen peroxide (30 wt %) was placed in the chamber extension of the diffusion system. In both tests the diffusion system was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles over time for each process were analyzed to show that the negative slope, or the rate of vapor evacuation, of aqueous hydrogen peroxide vapor in the presence of MME vapor, methanol, was greater than the rate of evacuation of aqueous hydrogen peroxide vapor without MME.

### Example 39 (Process Chamber, Performic Acid with and without MME Diethyl Ether)

A transport moiety solution of performic acid (as described above), 5 mL, was placed inside the process chamber of the diffusion system. In a separate, but similar experiment, an MME-transport moiety blend solution containing 0.5% (v/v) diethyl ether in 5 mL of performic acid solution was placed in the process chamber of the diffusion system. In both tests the process chamber was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles over time for each process were analyzed to show that the negative slope, or the rate of vapor evacuation, of performic acid vapor in the presence of MME vapor, diethyl ether, was greater than the rate of evacuation of performic acid vapor without MME.

### Example 40 (Chamber Extension, Performic Acid with and without MME Diethyl Ether)

A transport moiety solution of performic acid, 5 mL (as described above), was placed inside of the chamber extension of the diffusion system. In a separate, but similar experiment, an MME-transport moiety blend solution containing 0.5% (v/v) diethyl ether in 5 mL of performic acid solution was placed in the chamber extension of the diffusion system. In both tests the diffusion system was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles over time for each process were analyzed to show that the negative slope, or the rate of vapor evacuation, of performic acid vapor in the presence of MME vapor, diethyl ether, was greater than the rate of evacuation of performic acid vapor without MME.

### Example 41. (Process Chamber, Peracetic Acid with and without MME Pentane)

A transport moiety solution of peracetic acid, 5 mL (as described above), was placed inside of the process chamber of the diffusion system. In a separate, but similar experiment, an MME-transport moiety blend solution containing 1% (v/v) pentane in 5 mL of peracetic acid solution was placed in the process chamber of the diffusion system. In both tests the process chamber was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles for each process were analyzed to show that the negative slope, or rate of vapor evacuation, of peracetic acid in the presence of MME vapor, pentane, was greater than the rate of evacuation of peracetic acid vapor without MME.

### Example 42 (Chamber Extension, Peracetic Acid with and without MME Pentane)

A transport moiety solution of peracetic acid, 5 mL (as described above), was placed inside of the chamber extension of the diffusion system. In a separate, but similar experiment, an MME-transport moiety blend solution containing 1% (v/v) pentane in 5 mL of peracetic acid solution was placed in the chamber extension of the diffusion system. In both tests the diffusion system was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles for each process were analyzed to show that the negative slope, or rate of vapor evacuation, of peracetic acid in the presence of MME vapor, pentane, was greater than the rate of evacuation of peracetic acid vapor without MME.

### Example 43 (Process Chamber, Isopropanol with and without MME Formic Acid)

A transport moiety solution of isopropanol, 5 mL, was placed inside of the process chamber of the diffusion system. In a separate, but similar experiment, an MME-transport moiety blend solution containing 4% (v/v) formic acid in 5 mL of isopropanol was placed in the process chamber of the diffusion system. In both tests the process chamber was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles for each process were analyzed to show that the negative slope, or rate of vapor evacuation, of peracetic acid in the presence of MME vapor, formic acid, was greater than the rate of evacuation of isopropanol vapor without MME.

### Example 44 (Chamber Extension, Isopropanol with and without MME Formic Acid)

A transport moiety solution of isopropanol, 5 mL, was placed inside of the chamber extension of the diffusion system. In a separate, but similar experiment, an MME-transport moiety blend solution containing 4% (v/v) formic acid in 5 mL of isopropanol was placed in the chamber extension of the diffusion system. In both tests the diffusion chamber was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles for each process were analyzed to show that the negative slope, or rate of vapor evacuation, of peracetic acid in the presence of MME vapor, formic acid, was greater than the rate of evacuation of isopropanol vapor without MME.

### Example 45 (Process Chamber, Hydrogen Peroxide with and without MME Ethyl Acetate)

A transport moiety solution of aqueous hydrogen peroxide (30 wt %), 5 mL, was placed inside of the process chamber of the diffusion system. In a separate, but similar experiment, an MME-transport moiety blend solution containing 2% (v/v) ethyl acetate in 5 mL of aqueous hydrogen peroxide (30 wt %) was placed in the process chamber of the diffusion system. In both tests the process chamber was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles for each process were analyzed to show that the negative slope, or rate of vapor evacuation, of hydrogen peroxide in the presence of MME vapor, ethyl acetate, was greater than the rate of evacuation of hydrogen peroxide vapor without MME.

### Example 46 (Chamber Extension, Hydrogen Peroxide with and without MME Ethyl Acetate)

A transport moiety solution of aqueous hydrogen peroxide (30 wt %), 5 mL, was placed inside of the chamber extension of the diffusion system. In a separate, but similar experiment, an MME-transport moiety blend solution containing 2% (v/v) ethyl acetate in 5 mL of aqueous hydrogen peroxide (30 wt %) was placed in the chamber extension of the diffusion system. In both tests the diffusion system was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles for each process were analyzed to show that the negative slope, or rate of vapor evacuation, of hydrogen peroxide in the presence of MME vapor, ethyl acetate, was greater than the rate of evacuation of hydrogen peroxide vapor without MME.

### Example 47 (Process Chamber, Isopropanol with and without MME Acetone)

A transport moiety solution of isopropanol, 5 mL, was placed inside of the process chamber of the diffusion system. In a separate, but similar experiment, an MME-transport moiety blend solution containing 1% (v/v) acetone in 5 mL of isopropanol was placed in the process chamber of the diffusion system. In both tests the process chamber was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles for each process were analyzed to show that the negative slope, or rate of vapor evacuation, of isopropanol in the presence of MME vapor, acetone, was greater than the rate of evacuation of isopropanol vapor without MME.

### Example 48 (Chamber Extension, Isopropanol with and without MME Acetone)

A transport moiety solution of isopropanol, 5 mL, was placed inside of the chamber extension of the diffusion system. In a separate, but similar experiment, an MME-transport moiety blend solution containing 1% (v/v) acetone in 5 mL of isopropanol was placed in the chamber extension of the diffusion system. In both tests the diffusion system was heated to 50° C. The door to the diffusion system for each was closed and sealed. The diffusion system was opened to vacuum and the pressure of the system was monitored over the course of each 30-minute time period. At the end of each process the diffusion system was brought to atmosphere. The pressure profiles for each process were analyzed to show that the negative slope, or rate of vapor evacuation, of isopropanol in the presence of MME vapor, acetone, was greater than the rate of evacuation of isopropanol vapor without MME.

### Example 49: Preparation of Microparticles of MME-Transport Moiety (Methanol (MeOH)-Hydrogen Peroxide (H₂O₂))

35 g of a 1 wt % MeOH in H₂O₂ (50 wt % aqueous solution) (Sigma Aldrich, St. Louis, Mo.) is used to dissolve 35 g of polyethylene glycol Mₙ 400 (Sigma Aldrich, St. Louis, Mo.). The prepared solution is mixed with 600 g of a 4:1 solution of a Nonidet P-40 surfactant: n-Butanol (Sigma Aldrich, St. Louis, Mo.). 120 g of water is added to the solution. The mixture is agitated while adding water to obtain a microemulsion. 120 g of a 1 wt % bovine serum albumin (BSA) (Sigma Aldrich, St. Louis, Mo.) is added during agitation. A sucrose (2 wt %) (Sigma Aldrich, St. Louis, Mo.) solution is added to the dispersion of microparticles. The dispersion of microparticles are lyophilized to obtain a solid material.

The microparticle material containing the MME-transport moiety, MeOH—H₂O₂, a scalpel and a self-contained biological indicator ampoule (MesaLabs, Bozeman, Mont.) containing a sterility assurance level (SAL) strip with a Geobacillus stearothermophilus population of 10⁶ are placed inside of the process chamber of a vacuum sterilization chamber. The process chamber is then evacuated to 1×10⁻³ Torr while heat of 50° C. is evenly applied to the vacuum system. Once the base pressure is reached; the system is allowed to remain at a selected steady state pressure of about 1 Torr for 15 minutes. The selected steady state pressure is maintained in the process chamber by modulation of pumping speed. At the end of the 15 minutes, the chamber is vented with argon gas until it reaches atmospheric pressure. The scalpel and biological indicator ampoule are removed using well-established sterile techniques. The SAL strip is tested following manufacturer's instructions within the biological indicator by gently crushing the ampoule followed by incubating the ampoule for 24 hours at 60° C. On completion of the assay, the SAL is found to be acceptably sterile at a level of 10⁻⁶.

An exemplary vacuum sterilization system is described in PCT application WO2018/175455, published Sep. 27, 2018, U.S. published application US2018/0289846 and U.S. provisional applications 62/473,543, filed Mar. 20, 2017 and 62/598,004, filed Dec. 13, 2017.

### Example 50: Preparation of Polyacrylamide Gel(s), Polyacrylic acid Gel(s), and Poly(acrylamide-co-methacrylic acid) Gel(s) of MME-Transport Moiety (Diethyl Ether (DEE)-Hydrogen Peroxide (H₂O₂)

Polyacrylamide (Sigma Aldrich, St. Louis, Mo.), polyacrylic acid (Sigma Aldrich, St. Louis, Mo.) and poly(acrylamide-co-acrylic acid) (Sigma Aldrich, St. Louis, Mo.) gels containing 3 wt % (DEE (diethyl ether) in H₂O₂) (50 wt % aqueous solution) (Sigma Aldrich, St. Louis, Mo.) are produced with the polymer formulas set forth in Table 1 (below).

The DEE-H2O2 gels prepared in Example 50, formulations A through F, are prepared via blending by mixing the ingredients listed above. In independent experiments, each of the gel materials containing the MME-transport moiety, a scalpel and a self-contained biological indicator ampoule (MesaLabs, Bozeman, Mont.) containing a sterility assurance level (SAL) strip with a Geobacillus stearothermophilus population of 106 are placed inside of the process chamber of a vacuum sterilization chamber. The process chamber is then evacuated to 1×10−3 Torr while heat of 50° C. is evenly applied to the vacuum system. Once the base pressure is reached; the system is allowed to remain in a steady state of about 1 Torr for 15 minutes. At the end of the 15 minutes, the chamber is vented with argon gas until it reaches atmospheric pressure. The scalpel and biological indicator ampoule are removed using well-established sterile techniques. The SAL strip is tested within the biological indicator by gently crushing the ampoule followed by incubating the ampoule for 24 hours at 60° C. After the assay is completed, the SAL is found to be acceptably sterile at a level of 10⁻⁶.

TABLE 1

| | % Concentration By Weight | | | | | |
|---|---|---|---|---|---|---|
| Ingredient | A | B | C | D | E | F |
| Polyacrylamide (Mn 150,000) | 5 | — | — | — | 5 | — |
| Polyisopropylacrylamide | — | 5 | — | — | — | — |
| (Mn 85,000) | | | | | | |
| Polymethacrylic acid | — | — | 5 | — | — | 5 |
| (Mn 75,100) | | | | | | |
| Poly(N-isopropylacrylamide- | — | — | — | 5 | — | — |
| co-methacrylic acid) | | | | | | |
| Sodium Chloride | 0.5 | 0.5 | 0.5 | 0.5 | 0.5 | 0.5 |
| Calcium Chloride | 0.5 | 0.5 | 0.5 | 0.5 | 0.5 | 0.5 |
| PEG100 Stearate | 1 | 1 | 1 | 1 | 3 | 3 |
| Water | 93 | 93 | 93 | 93 | 91 | 91 |

### Example 51: Preparation of Polyamide, Polysiloxane and Polyamide/Polysiloxane Blend(s) Gel(s) and Solid(s) of MME-Transport Moiety (Isopropanol (IPA)-Hydrogen Peroxide (H₂O₂))

Polyamide (Sigma Aldrich, St. Louis, Mo.), polysiloxane (Sigma Aldrich, St. Louis, Mo.) and polyamide/polysiloxane blend(s) gel(s) and solid(s) containing 5 wt % (IPA in H₂O₂ (50 wt % aqueous solution))(Sigma Aldrich, St. Louis, Mo.) are produced with the polymer formulas set forth in Table 2 below.

The IPA-H₂O₂ solids and gels prepared in Example 51, formulations A through F, were prepared via blending by mixing the ingredients listed above.

In independent experiments, each of the gel materials containing the MME-transport moiety, scalpel and a self-contained biological indicator ampoule (MesaLabs, Bozeman, Mont.) containing a sterility assurance level (SAL) strip with a Geobacillus stearothermophilus population of 106 are placed inside of the process chamber of a vacuum sterilization chamber. The process chamber is then evacuated to 1×10−3 Torr while heat of 50 □C is evenly applied to the vacuum system. One the base pressure is reached; the system is allowed to remain in a steady state of about 1 Torr for 15 minutes. At the end of the 15 minutes, the chamber is vented with argon gas until it reaches atmospheric pressure. The scalpel and biological indicator ampoule are removed using well-established sterile techniques. The SAL strip is tested within the biological indicator by gently crushing the ampoule followed by incubating the ampoule for 24 hours at 60□C. After the assay is completed, the SAL is found to be acceptably sterile at a level of 10-6.

### Example 52: Preparation of Polyethylene Glycol Solid(s) of MME-Transport Moiety (MeOH-Performic acid (PFA))

Polyethylene glycol (Sigma Aldrich, St. Louis, Mo.) solid(s) containing 5 wt % (MeOH in Performic Acid) are produced with the polymer formulas set forth in Table 3 below:

TABLE 2

| | % Concentration By Weight | | | | | |
|---|---|---|---|---|---|---|
| Ingredient | A | B | C | D | E | F |
| ATPA Resin | 32 | 47 | 74 | — | — | — |
| Silicone Oil | — | — | 15 | 35 | 69 | 85 |
| Canola Oil | 42 | 19 | — | — | 5 | — |
| Sorbitan Monolaurate | 26 | | 11 | 65 | — | 15 |
| Sodium Caproate | — | 34 | — | — | 26 | — |

TABLE 3

| | % Concentration By Weight | | | | | |
|---|---|---|---|---|---|---|
| Ingredient | A | B | C | D | E | F |
| Polyethylene glycol Mn 400 | 30 | — | — | 5 | — | 34 |
| Polyethylene glycol Mn 600 | — | 64 | — | — | — | 48 |
| Polyethylene glycol Mn 1000 | — | — | 86 | — | 77 | — |
| Polyethylene glycol Mn 3350 | 65 | — | — | 45 | 12 | — |
| Povidone | — | 10 | 13 | — | 10 | 8 |
| Hydroxypropylmethylcellulose | 5 | 16 | 1 | — | 1 | 10 |
| Cellulose Acetate | — | 10 | — | 50 | — | — |

The MeOH-PFA solids prepared in Example 52, formulations A through F, were prepared via blending by mixing the ingredients listed above.

In independent experiments, each of the solid materials containing the MME-transport moiety solid, a scalpel and a self-contained biological indicator ampoule (MesaLabs, Bozeman, Mont.) containing a sterility assurance level (SAL) strip with a Geobacillus stearothermophilus population of 106 are placed inside of the process chamber of a vacuum sterilization chamber. The process chamber is then evacuated to 1×10−3 Torr while heat of 50° C. is evenly applied to the vacuum system. Once the base pressure is reached; the system is allowed to remain at a steady state pressure of about 1 Torr for 15 minutes. At the end of the 15 minutes, the chamber is vented with argon gas until it reaches atmospheric pressure. The scalpel and biological indicator ampoule are removed using well-established sterile techniques. The SAL strip is tested within the biological indicator by gently crushing the ampoule followed by incubating the ampoule for 24 hours at 60° C. After the assay is completed, the SAL is found to be acceptably sterile at a level of 10-6.

### Example 53: Preparation of Poly(DL-lactide-co-glycolide) and Poly(DL-lactide-co-glycolide)-co-Polyethylene glycol Solid(s) of MME-Transport Moiety (DEE-Peracetic acid (PAA))

Poly(DL-lactide-co-glycolide) (Polysciences, Warrington, Pa.) and Poly(DL-lactide-co-glycolide)-co-Polyethylene glycol Mₙ 5000 copolymer (Sigma Aldrich, St. Louis, Mo.) solid(s) containing 10 wt % (DEE-PAA) are produced with the polymer formulas set forth in Table 4 (below).

The DEE-PAA solids prepared in Example 53, formulations A through J, were prepared via blending by mixing the ingredients listed above.

In independent experiments, each of the solid materials containing the MME-transport moiety, a scalpel and a self-contained biological indicator ampoule (MesaLabs, Bozeman, Mont.) containing a sterility assurance level (SAL) strip with a Geobacillus stearothermophilus population of 106 are placed inside of the process chamber of a vacuum sterilization chamber. The process chamber is then evacuated to 1×10−3 Torr while heat of 50 □C is evenly applied to the vacuum system. Once the base pressure is reached; the system is allowed to remain in a steady state of about 1 Torr for 15 minutes. At the end of the 15 minutes, the chamber is vented with argon gas until it reaches atmospheric pressure. The scalpel and biological indicator ampoule are removed using well-established sterile techniques. The SAL strip is tested within the biological indicator by gently crushing the ampoule followed by incubating the ampoule for 24 hours at 60° C. After this assay, the SAL is found to be acceptably sterile at a level of 10⁻⁶.

TABLE 4

| | % Concentration By Weight | | | | | | | | | |
|---|---|---|---|---|---|---|---|---|---|---|
| Ingredient | A | B | C | D | E | F | G | H | I | J |
| 50:50 PLGA-COOH | 85 | 95 | — | — | — | — | — | — | — | — |
| 75:25 PLGA-COOH | — | — | 80 | 90 | — | — | — | — | — | — |
| 65:35 PLGA-COOH | — | — | — | — | 86 | 94 | — | — | — | — |
| 85:15 PLGA-COOH | — | — | — | — | — | — | 75 | 95 | — | — |
| 70:30 PLG:PEG5000 | — | — | — | — | — | — | — | — | 90 | 97 |
| Polyvinypyrrolidone | 15 | — | 20 | — | 14 | — | 15 | — | 10 | — |
| Hydroxypropylmethylcellulose | — | 5 | — | 10 | — | 6 | — | 5 | — | 3 |

PLGA-COOH Poly(DL-lactide-co-glycolide) with Carboxylic Acid End Group
PLG:PEG5000 Poly(DL-lactide-co-glycolide)-Polyethylene glycol Mn 5000 Copolymer

### Example 54: Preparation of Maltodextrin and Sorbitol Solid(s) of MME-Transport Moiety (IPA-Performic acid (PFA))

Maltodextrin (Sigma Aldrich, St. Louis, Mo.) and Sorbitol (Sigma Aldrich, St. Louis, Mo.) solid(s) containing 7 wt % IPA-PFA are produced with the polymer formulas set forth in Table 5 below.

The IPA-PFA gels prepared in Example 5, formulations A through F, were prepared via blending by mixing the ingredients listed above.

In independent experiments, each of the solid materials containing the MME-transport moiety solids and a scalpel and a self-contained biological indicator ampoule (MesaLabs, Bozeman, Mont.) containing a sterility assurance level (SAL) strip with a Geobacillus stearothermophilus population of 106 are placed inside of the process chamber of a vacuum sterilization chamber. The process chamber is then evacuated to 1×10−3 Torr while heat of 50□C is evenly applied to the vacuum system. Once the base pressure is reached; the system is allowed to remain at a steady state pressure of about 1 Torr for 15 minutes. At the end of the 15 minutes, the chamber is vented with argon gas until it reaches atmospheric pressure. The scalpel and biological indicator ampoule are removed using well-established sterile techniques. The SAL strip is tested within the biological indicator by gently crushing the ampoule followed by incubating the ampoule for 24 hours at 60° C. After completion of the assay, the SAL is found to be acceptably sterile at a level of 10⁻⁶.

TABLE 5

| | % Concentration By Weight | | | | | |
|---|---|---|---|---|---|---|
| Ingredient | A | B | C | D | E | F |
| Maltodextrin | 90 | — | 50 | 75 | — | 95 |
| Sorbitol | — | 90 | 50 | — | 75 | 5 |
| PEG-9 Stearate | 9 | 9 | 9 | 25 | 25 | 4 |
| Hydroxyethylcellulose | 1 | 1 | 1 | — | — | 1 |

The IPA-PFA gels prepared in Example 5, formulations A through F, were prepared via blending by mixing the ingredients listed above.

In independent experiments, each of the solid materials containing the MME-transport moiety solids and a scalpel and a self-contained biological indicator ampoule (MesaLabs, Bozeman, Mont.) containing a sterility assurance level (SAL) strip with a Geobacillus stearothermophilus population of 106 are placed inside of the process chamber of a vacuum sterilization chamber. The process chamber is then evacuated to 1×10−3 Torr while heat of 50° C. is evenly applied to the vacuum system. Once the base pressure is reached; the system is allowed to remain at a steady state pressure of about 1 Torr for 15 minutes. At the end of the 15 minutes, the chamber is vented with argon gas until it reaches atmospheric pressure. The scalpel and biological indicator ampoule are removed using well-established sterile techniques. The SAL strip is tested within the biological indicator by gently crushing the ampoule followed by incubating the ampoule for 24 hours at 60° C. After completion of the assay, the SAL is found to be acceptably sterile at a level of 10′.

### Example 55: Preparation of Silica Solid(s) and Gel(s) of MME-Transport Moiety (MeOH-11202)

Silica (Sigma Aldrich, St. Louis, Mo.), Polyethylene glycol Mn 400 (Sigma Aldrich, St. Louis, Mo.) and Polyethylene glycol Mn 1000 (Sigma Aldrich, St. Louis, Mo.) solid(s) and gel(s) containing 10% wt % MeOH—H2O2 are produced with the ceramic and polymer formulas set forth in Table 6 below. The MeOH—H2O2 gels prepared in Example 55, formulations A through F, were prepared via blending by mixing the ingredients listed above.

In independent experiments, each of the solid materials containing the MME-transport moiety gel or solid and scalpel and a self-contained biological indicator ampoule (MesaLabs, Bozeman, Mont.) containing a sterility assurance level (SAL) strip with a Geobacillus stearothermophilus population of 106 are placed inside of the process chamber of a vacuum sterilization chamber. The process chamber is then evacuated to 1×10-3 Torr while heat of 50 □C is evenly applied to the vacuum system. One the base pressure is reached; the system is allowed to remain at a steady state pressure of about 1 Torr for 15 minutes. At the end of the 15 minutes, the chamber is vented with argon gas until it reaches atmospheric pressure. The scalpel and biological indicator ampoule are removed using well-established sterile techniques. The SAL strip is tested within the biological indicator by gently crushing the ampoule followed by incubating the ampoule for 24 hours at 60° C. After the assay is completed the SAL is found to be acceptably sterile at a level of 10⁻⁶.

TABLE 6

| | % Concentration By Weight | | | | | |
|---|---|---|---|---|---|---|
| Ingredient | A | B | C | D | E | F |
| Silica | 100 | 75 | 75 | 50 | 25 | 25 |
| Polyethylene Glycol Mn 400 | — | 12 | 20 | 25 | 70 | — |
| Polyethylene Glycol Mn 1000 | — | — | 5 | 25 | 5 | 75 |

### Example 56: Drying of Endoscope Using MDE Liquid—Isopropanol

A 20 g MDE solution* containing isopropanol, 99.9% (Sigma Aldrich, St. Louis, Mo.) and an endosope burdened with 0 mL, 1 mL, 5 mL, 10 mL or 20 mL of deionized water are placed in the drying process chamber. The process chamber is then evacuated to a base pressure of 1 Torr while that of 40° C., 60° C. or 80° C. is evenly applied to the chamber. Once the base pressure is reached, the system is allowed of remain in a steady state of 1 Torr until at which point the pressure of the process chamber falls below 0.1 Torr. The time to dry is recorded as the time it takes to reach 0.1 Torr. At the end of each drying run, the process chamber is brought to atmosphere and the contents are removed. Time to dry versus liquid burden and temperature is shown in Table 7. *Not all MDE solution may be used during process run.

TABLE 7

| Total Liquid Burden (mL) | Temperature (° C.) | Time to Dry (Minutes) |
|---|---|---|
| 0 | 40 | 0 |
| 1 | 40 | 112 |
| 5 | 40 | 663 |
| 10 | 40 | 1398 |
| 20 | 40 | 3009 |
| 0 | 60 | 0 |
| 1 | 60 | 98 |
| 5 | 60 | 519 |
| 10 | 60 | 1293 |
| 20 | 60 | 2321 |
| 0 | 80 | 0 |
| 1 | 80 | 26 |
| 5 | 80 | 112 |
| 10 | 80 | 312 |
| 20 | 80 | 4317 |

### Example 57: Drying of Endoscope Using Mde Liquid—Methanol

A 20 g MDE solution* containing methanol, 99.9% (Sigma Aldrich, St. Louis, Mo.) and an endosope burdened with 0 mL, 1 mL, 5 mL, 10 mL or 20 mL of deionized water are placed in the drying process chamber. The process chamber is then evacuated to a base pressure of 1 Torr while that of 40° C., 60° C. or 80° C. is evenly applied to the chamber. Once the base pressure is reached, the system is allowed of remain in a steady state of 1 Torr until at which point the pressure of the process chamber falls below 0.1 Torr. The time to dry is recorded as the time it takes to reach 0.1 Torr. At the end of each drying run, the process chamber is brought to atmosphere and the contents are removed. Time to dry versus liquid burden and temperature is shown in Table 8. *Not all MDE solution may be used during process run.

TABLE 8

| Total Liquid Burden (mL) | Temperature (° C.) | Time to Dry (Minutes) |
|---|---|---|
| 0 | 40 | 0 |
| 1 | 40 | 133 |
| 5 | 40 | 645 |
| 10 | 40 | 1367 |
| 20 | 40 | 3245 |
| 0 | 60 | 0 |
| 1 | 60 | 98 |
| 5 | 60 | 443 |
| 10 | 60 | 1243 |
| 20 | 60 | 2214 |
| 0 | 80 | 0 |
| 1 | 80 | 32 |
| 5 | 80 | 198 |
| 10 | 80 | 533 |
| 20 | 80 | 1082 |

### Example 58: Drying of Endoscope Using Mde Liquid—Acetone

A 20 g MDE solution* containing acetone, 99.9% (Sigma Aldrich, St. Louis, Mo.) and an endosope burdened with 0 mL, 1 mL, 5 mL, 10 mL or 20 mL of deionized water are placed in the drying process chamber. The process chamber is then evacuated to a base pressure of 1 Torr while that of 40° C., 60° C. or 80° C. is evenly applied to the chamber. Once the base pressure is reached, the system is allowed of remain in a steady state of 1 Torr until at which point the pressure of the process chamber falls below 0.1 Torr. The time to dry is recorded as the time it takes to reach 0.1 Torr. At the end of each drying run, the process chamber is brought to atmosphere and the contents are removed. Time to dry versus liquid burden and temperature is shown in Table 9. *Not all MDE solultion may be used during process run.

TABLE 9

| Total Liquid Burden (mL) | Temperature (° C.) | Time to Dry (Minutes) |
|---|---|---|
| 0 | 40 | 0 |
| 1 | 40 | 134 |
| 5 | 40 | 703 |
| 10 | 40 | 1562 |
| 20 | 40 | 3323 |
| 0 | 60 | 0 |
| 1 | 60 | 154 |
| 5 | 60 | 709 |
| 10 | 60 | 1321 |
| 20 | 60 | 2769 |
| 0 | 80 | 0 |
| 1 | 80 | 157 |
| 5 | 80 | 309 |
| 10 | 80 | 601 |
| 20 | 80 | 982 |

### Example 59—Drying of Endoscope Using Mde Liquid—Ethyl Acetate

A 20 g MDE solution* containing ethyl acetate, 99.8% (Sigma Aldrich, St. Louis, Mo.) and an endosope burdened with 0 mL, 1 mL, 5 mL, 10 mL or 20 mL of deionized water are placed in the drying process chamber. The process chamber is then evacuated to a base pressure of 1 Torr while ehat of 40° C., 60° C. or 80° C. is evenly applied to the chamber. Once the base pressure is reached, the system is allowed of remain in a steady state of 1 Torr until at which point the pressure of the process chamber falls below 0.1 Torr. At the end of each drying run, the process chamber is brought to atmosphere and the contents are removed. Time to dry versus liquid burden and temperature is shown in Table 10. *Not all MDE solultion may be used during process run.

TABLE 10

| Total Liquid Burden (mL) | Temperature (° C.) | Time to Dry (Minutes) |
|---|---|---|
| 0 | 40 | 0 |
| 1 | 40 | 111 |
| 5 | 40 | 423 |
| 10 | 40 | 1009 |
| 20 | 40 | 2135 |
| 0 | 60 | 0 |
| 1 | 60 | 25 |
| 5 | 60 | 321 |
| 10 | 60 | 842 |
| 20 | 60 | 1029 |
| 0 | 80 | 0 |
| 1 | 80 | 15 |
| 5 | 80 | 113 |
| 10 | 80 | 431 |
| 20 | 80 | 892 |

### Example 60—Drying of Endoscope Using Mde Liquid—Acetonitrile

A 20 g MDE solution* containing acetonitrile, 99.9% (Sigma Aldrich, St. Louis, Mo.) and an endosope burdened with 0 mL, 1 mL, 5 mL, 10 mL or 20 mL of deionized water are placed in the drying process chamber. The process chamber is then evacuated to a base pressure of 1 Torr while ehat of 40° C., 60° C. or 80° C. is evenly applied to the chamber. Once the base pressure is reached, the system is allowed of remain in a steady state of 1 Torr until at which point the pressure of the process chamber falls below 0.1 Torr. The time to dry is recorded as the time it takes to reach 0.1 Torr. At the end of each drying run, the process chamber is brought to atmosphere and the contents are removed. Time to dry versus liquid burden and temperature is shown in Table 11. *Not all MDE solultion may be used during process run.

TABLE 11

| Total Liquid Burden (mL) | Temperature (° C.) | Time to Dry (Minutes) |
|---|---|---|
| 0 | 40 | 0 |
| 1 | 40 | 203 |
| 5 | 40 | 890 |
| 10 | 40 | 1892 |
| 20 | 40 | 4219 |
| 0 | 60 | 0 |
| 1 | 60 | 231 |
| 5 | 60 | 781 |
| 10 | 60 | 1578 |
| 20 | 60 | 2893 |
| 0 | 80 | 0 |
| 1 | 80 | 187 |
| 5 | 80 | 431 |
| 10 | 80 | 972 |
| 20 | 80 | 2003 |

### Example 61—Drying of Endoscope Using Mde Liquid—Acetic Acid

A 20 g MDE solution* containing acetic acid, 99% (Sigma Aldrich, St. Louis, Mo.) and an endosope burdened with 0 mL, 1 mL, 5 mL, 10 mL or 20 mL of deionized water are placed in the drying process chamber. The process chamber is then evacuated to a base pressure of 1 Torr while heat of 40° C., 60° C. or 80° C. is evenly applied to the chamber. Once the base pressure is reached, the system is allowed of remain in a steady state of 1 Torr until at which point the pressure of the process chamber falls below 0.1 Torr. The time to dry is recorded as the time it takes to reach 0.1 Torr. At the end of each drying run, the process chamber is brought to atmosphere and the contents are removed. Time to dry versus liquid burden and temperature is shown in Table 12. *Not all MDE solultion may be used during process run.

TABLE 12

| Total Liquid Burden (mL) | Temperature (° C.) | Time to Dry (Minutes) |
|---|---|---|
| 0 | 40 | 0 |
| 1 | 40 | 112 |
| 5 | 40 | 663 |
| 10 | 40 | 1398 |
| 20 | 40 | 3009 |
| 0 | 60 | 0 |
| 1 | 60 | 98 |
| 5 | 60 | 519 |
| 10 | 60 | 1293 |
| 20 | 60 | 2321 |
| 0 | 80 | 0 |
| 1 | 80 | 26 |
| 5 | 80 | 112 |
| 10 | 80 | 312 |
| 20 | 80 | 4317 |

### Example 62—Drying of Endoscope Using Mde Liquid—Ethylene Glycol Mono Methyl Ether

A 20 g MDE solution* containing ethylene glycol mono methyl ether, 99.9% (Sigma Aldrich, St. Louis, Mo.) and an endosope burdened with 0 mL, 1 mL, 5 mL, 10 mL or 20 mL of deionized water are placed in the drying process chamber. The process chamber is then evacuated to a base pressure of 1 Torr while ehat of 40° C., 60° C. or 80° C. is evenly applied to the chamber. Once the base pressure is reached, the system is allowed of remain in a steady state of 1 Torr until at which point the pressure of the process chamber falls below 0.1 Torr. The time to dry is recorded as the time it takes to reach 0.1 Torr. At the end of each drying run, the process chamber is brought to atmosphere and the contents are removed. Time to dry versus liquid burden and temperature is shown in Table 13. *Not all MDE solultion may be used during process run.

TABLE 13

| Total Liquid Burden (mL) | Temperature (° C.) | Time to Dry (Minutes) |
|---|---|---|
| 0 | 40 | 0 |
| 1 | 40 | 231 |
| 5 | 40 | 1093 |
| 10 | 40 | 2135 |
| 20 | 40 | 4589 |
| 0 | 60 | 0 |
| 1 | 60 | 221 |
| 5 | 60 | 981 |
| 10 | 60 | 2215 |
| 20 | 60 | 4333 |
| 0 | 80 | 0 |
| 1 | 80 | 201 |
| 5 | 80 | 1092 |
| 10 | 80 | 2333 |
| 20 | 80 | 4167 |

1. A method for providing a transport moiety to an enclosure, the method comprising:
exposing a mixture comprising a molecular mobility enhancer (MME) and the transport moiety to the enclosure at a sub-atmospheric pressure condition, thereby providing the transport moiety as a vapor in the enclosure.

2. The method of claim 1, wherein:
the exposing step provides the transport moiety and the MME as the vapor in the enclosure;
the exposing step results in transport of the transport moiety within the enclosure;
the exposing step results in contact of the transport moiety with surfaces of the enclosure and/or with surfaces of an article within the enclosure; or
the exposing step results in contact of the transport moiety with surfaces of the enclosure and/or with surfaces of an article within the enclosure.

3.-4. (canceled)

5. The method of claim 1 for sanitizing or sterilizing the enclosure and/or an article within the enclosure.

6.-7. (canceled)

8. The method of claim 1, wherein the transport moiety is a liquid or a solid at normal temperature and pressure (NPT, 20° C. and 760 torr).

9. (canceled)

10. The method of claim 1, wherein the MME is a liquid or a solid at normal temperature and pressure (NPT, 20° C. and 760 torr).

11. (canceled)

12. The method of claim 1, wherein the MME is one or more of an alcohol, alkane, carboxylic acid, ester, ether, or ketone.

13. The method of claim 12, wherein the MME is one or more of: a C₁-C₂₀ alcohol, C₅-C₂₀ alkane, C₁-C₂₀ carboxylic acid, C₃-C₂₀ ester, C₄-C₂₀ ether, or C₃-C₂₀ ketone.

14. The method of claim 1, wherein the MME has a vapor pressure equal to or greater than 10 torr at 20° C. and 760 torr or a vapor pressure equal to or greater than 100 torr at 20° C. and 760 torr.

15. (canceled)

16. The method of claim 1, wherein the transport moiety has a vapor pressure equal to or greater than 10 torr at 20° C. and 760 torr or a vapor pressure equal to or greater than 100 torr at 20° C. and 760 torr.

17. (canceled)

18. The method of claim 1, wherein the molecular mobility enhancer is a C1-C3 alcohol.

19. (canceled)

20. The method of claim 1, wherein the transport moiety is:
one or more of a peroxide, peroxyacid, alcohol, chlorine-containing compound, or a phenolic compound;
one or more of hydrogen peroxide, ethanol, isopropanol, hypochlorite, hypochlorous acid, chloride dioxide, ethylene oxide, propylene oxide, formaldehyde, glutaraldehyde, iodophor, ortho-phthaladehyde, ozone, peracetic acid, performic acid, phenol/phenate, or beta-propiolactone; or
one or more sanitizer and/or sterilizer.

21.-22. (canceled)

23. The method of claim 1, wherein the molecular mobility enhancer of the mixture is methanol and the transport moiety of the mixture is hydrogen peroxide.

24. The method of claim 1, further comprising:
heating the mixture during the exposing step;
providing a carrier gas flow in fluid communication with the mixture and flowing the carrier gas flow in fluid communication with the mixture into the enclosure; and/or
maintaining the enclosure at a pressure selected over the range of 0.1 torr to 200 torr for a time period selected from the range of 1 minute to 24 hours.

25.-26. (canceled)

27. The method of claim 1, wherein the exposing step comprises providing the mixture in fluid communication with the enclosure at sub-atmospheric condition, thereby providing for transport of the transport moiety into the enclosure.

28. The method of claim 1, wherein the exposing step comprises providing the mixture in the enclosure followed by decreasing the pressure of the enclosure to below 760 torr.

29. The method of claim 28, wherein the pressure of the enclosure is decreased to a pressure selected over the range of 0.1 torr to 200 torr.

30. The method of claim 1, wherein the enclosure is a vacuum chamber or a processing chamber.

31. (canceled)

32. The method of claim 1, wherein the enclosure is for disinfecting, sanitizing or sterilizing an article provided within the enclosure.

33. The method of claim 32, wherein the article provided within the enclosure is a medical device or component thereof.

34. (canceled)

35. The method of claim 33, wherein the medical device is an endoscope or component thereof.

36. A solid composition comprising an MME-transport moiety mixture and a carrier.

37. The composition of claim 36, wherein:
the carrier is a polymer, ceramic, glass, metallic, hybrid, or composite material or a mixture of such materials;
the transport moiety is a cleaning agent, a sanitizing agent, a sterilizing agent or a coating agent; and/or
the MME is an ether or an alcohol.

38.-40. (canceled)

41. The composition of claim 36, wherein:
the transport moiety is hydrogen peroxide, a peracid or a mixture thereof;
the MME is diethyl ether, methanol or isopropyl alcohol; and/or
the carrier is a polyamide, polysiloxane, a polyamide/polysiloxane blend, polyethylene glycol, a poly(DL-lactide-co-glycolide), a poly(DL-lactide-co-glycolide)-co-polyethylene glycol, a polyacrylamide gel, a polyacrylic acid gel, a poly(acrylamide-co-methacrylic acid) gel, maltodextrin, sorbitol, or a mixture of maltodextrin and sorbitol.

42.-47. (canceled)

48. The composition of claim 36 in the form of a powder, a wax, a gel, or a particle.

49. A method for delivery of a MME-transport moiety blend into an apparatus capable of achieving reduced pressure which comprises:
introducing a composition of claim 36 into the apparatus and reducing the pressure in at least a portion of the apparatus to generate a vapor comprising a transport moiety in at least a portion of the apparatus.

50. The method of claim 49, wherein the vapor comprises a transport moiety and a MME.

51. The method of claim 49, wherein an article to be treated is present in the apparatus and the vapor generated is in contact with the article.

52.-55. (canceled)

56. A method for removing undesired liquid from an article which comprises contacting the article with a vapor comprising a molecular drying enhancer (MDE), wherein the vapor is introducing into a sub-atmospheric environment containing the article; wherein the vapor is generated in a processing chamber containing the article and capable of achieving a reduced pressure environment from a liquid or solid in the processing chamber by reducing the pressure in at least a portion of the process chamber to a level that is sub-atmospheric.

57.-60. (canceled)

61. The method of claim 56, wherein the pressure in the processing chamber is reduced to 200 torr or less; or reduced to 10 torr or less; or reduced to 1 torr or less or reduced to 0.1 torr or less.

62.-64. (canceled)

65. The method of claim 56, wherein the MDE is selected from the group consisting of one or more optionally substituted hydrocarbons, ketones, nitriles, esters, ethers, glycolethers, aldehydes, carboxylic acids or siloxanes.

66.-67. (canceled)

68. The method of claim 56, wherein the MDE is a liquid at 20° C. and 1 atm (760 torr).

69. The method of claim 56, wherein the liquid to be removed is water.

70. The method of claim 56, wherein the MDE is selected from one or more of a C1-C3 alcohol, a C3-05 ketone, a C4-C6 ester, a C2-C6 nitrile, a C2-C6 carboxylic acid, or an ester of ethylene glycol.

71. (canceled)

72. The method of claim 56, wherein the MDE is selected from the group consisting of methanol, isopropanol, acetone, ethylacetate, acetonitrile, acetic acid and ethylene glycol monomethyl ester.

73. The method of claim 56, wherein the time to achieve drying ranges from 1 minute to 24 hours.

74.-75. (canceled)

76. The method of claim 56, wherein the process chamber is heated to a temperature ranging from above ambient temperature to 100° C.

77. (canceled)
